(19) United States
(12) Patent Application Publication
(10) Pub. No.: US 2025/0339399 A1
(43) Pub. Date: Nov. 6, 2025

(54) SUPRAMOLECULAR SELF-ASSEMBLY SYSTEM

(71) Applicants: Beijing Creatron Institute of Pharmaceutical Research Co., Ltd., Beijing (CN); Tianjin Creatron Biotechnology Co., Ltd., Tianjin (CN)

(72) Inventors: Huijuan JIA, Beijing (CN); Yanxin WANG, Beijing (CN); Yan LI, Beijing (CN)

(21) Appl. No.: 18/855,189

(22) PCT Filed: Apr. 8, 2022
(86) PCT No.: PCT/CN2022/085860

§ 371 Date: Dec. 27, 2024

(51) Int. Cl. A61K 31/353; A61K 31/56; A61K 31/7004; A61K 47/10; A61K 47/28; A61K 47/38

(52) U.S. Cl. CPC A61K 31/353; A61K 31/56; A61K 31/7004; A61K 47/10; A61K 47/28; A61K 47/38

(57) ABSTRACT

The present invention relates to a supramolecular self-assembly system, comprising the following ingredients:
(2) one or more carriers (or building blocks), which are water-soluble or at least soluble under pH≤8 conditions, where at least one carrier is amphiphilic with a hydrophobic group and a hydrophilic group; and
(3) one or more targets, preferably the targets are active ingredients such as drugs, diagnostic agents, biomarkers, vaccines, nutrients, or cosmetic active ingredients, and preferably in a free, salt, hydrate, or solvate form.

### TECHNICAL FIELD

The present invention belongs to the field of chemistry, and specifically relates to a delivery technology for active ingredients based on supramolecular self-assembly system.

### BACKGROUND

As a novel tool for building soft functional materials, supramolecular self-assembly has been widely used in the fields of materials science, biomedicine, fine chemicals, etc. Generally, molecular self-assembly, by definition, refers to the spontaneous formation of well-defined organized structures without the need for external assistance, where molecules act as the building blocks and weak forces, i.e. non-covalent interactions such as electrostatic interactions, dipole interaction, I-x stacking, hydrophobic and hydrophilic interactions, Van der Waals force and hydrogen bonds. Compared with covalent bond interactions, although these forces are weak, their collective interactions between different molecules with different functional groups of building blocks can produce structurally and chemically stable structures. Compared with self-assembly systems built by the same molecules, these dimensionally tunable soft structures exhibit many superior properties, such as memory, self-healing, and dynamic reversibility, and therefore, have been widely used in various fields including biomedicine (such as drug delivery, gene transfection, gene therapy, protein transport, tumor imaging, tissue engineering, and biomimetic simulation chemistry), nanotechnology (such as nanoreactors, catalytic carriers, and molecular imprinting), and functional materials. Supramolecular self-assembly also provides an attractive way to bridge the gap between natural and artificial materials and construct materials with novel functions, which is expected to break through the gaps that are difficult to overcome in many cutting-edge fields.

However, the current administration routes and drug delivery systems for many marketed drugs are not the best or most suitable.

### (1) Protein/Peptide Drugs are Administered Primarily by the Injection Route;

According to statistics, over 2,000 drugs approved by the FDA (Food and Drug Administration) are included in the PDR (Physician's Desk Reference) are delivered by injection, and more than 250 of them are injectable products, with injection is the only route of administration. In addition to non-therapeutic vaccines/antibody drugs, emergency drugs, and topical drugs, there are still over 100 peptide/protein drugs and some highly polar non-peptide drugs that can only be administered by intravenous, intramuscular, and subcutaneous injection. To achieve oral administration of peptide/protein drugs and highly polar non-peptide drugs, the following two technical problems must be solved:
1) Degradation of peptide/protein drugs by enzymes in the gastrointestinal tract;
2) Drugs with high molecular weight or polarity are difficult to penetrate the gastrointestinal mucosa and be absorbed into the blood.
   (2) The solubility and/or permeability of BCS (Biopharmaceutical Classification System) II/IV drugs become key limiting factors for their effective oral absorption. For BCS II and BCS IV drugs, the existing technologies mainly face the following challenges in developing their drug delivery systems:
1) Since BCS II and IV drugs are generally more lipophilic and their Clog P is generally greater than 2, the solubility in aqueous media is rarely low, especially when the clinical dose is high, the solubilization ability of the existing solubilization technologies such as salt formation, eutectic, nanocrystal, solid dispersion, cyclodextrin inclusion, lipid-based formulation, and self-microemulsion is very limited.
2) The existing technologies cannot overcome the pH change in digestive tract or the acid-base change of the microenvironment caused by the foods of the digestive tract or other drugs taken at the same time. As a result, the dissolved drug molecules interact with each other through hydrogen bonds, x-T stacking, and rapidly accumulated into solids, and the solids are excreted through feces, resulting in high PK (Pharmacokinetics) variation in vivo.

At present, the development of drug carrier materials and the research on chemical modification mostly focus on artificially synthesized or semi-synthesized polymers. Because synthetic polymer materials are not easy to degrade, low cell affinity and even certain toxicity, their application is limited to some extent. Meanwhile, in order to develop delivery systems suitable for different types of drugs, the diversity of optional types and structures of drug delivery carriers is also very limited.

### SUMMARY

The present invention provides a supramolecular self-assembly system. Specifically, the present invention involves the following:

1. A supramolecular self-assembly system, characterized by including the following ingredients:
(1) one or more carriers (or building blocks), which are water-soluble or at least soluble under pH≤8 conditions, where at least one carrier is amphiphilic with a hydrophobic group and a hydrophilic group; and
(1) one or more targets, preferably the targets are active ingredients such as drugs, diagnostic agents, biomarkers, vaccines, nutrients, or cosmetic active ingredients, and preferably in a free, salt, hydrate, or solvate form, where
preferably, the carrier is a compound with a flavonoid or terpenoid structure (preferably from natural sources).

2. The supramolecular self-assembly system according to item 1, characterized in that the supramolecular self-assembly system further includes hydroxypropyl methyl cellulose derivatives, preferably hydroxypropyl methyl cellulose acetate succinate (HPMCAS) or hydroxypropyl methyl cellulose (HPMC), and preferably, the supramolecular self-assembly system further includes one or more additional polymers A, which provide various non-covalent bond interactions for the targets, the carriers, and/or the hydroxypropyl methyl cellulose derivatives (such as HPMCAS), including but not limited to ion interaction, hydrogen bonding, hydrophobic interaction, dipole interaction, x-x stacking, Van der Waals force, and are dissoluble within a range of 1.0≤pH≤8.0.

3. The supramolecular self-assembly system according to any of items 1-2, characterized in that the carrier with the flavonoid or terpenoid structure from natural sources has at least 4, preferably at least 6 rotatable chemical bonds, at least 7 or more hydrogen donors, and at least 8 or more hydrogen acceptors, and more preferably, the carrier has at least 1 saccharide structure, such as monosaccharide, disaccharide, trisaccharide, tetrasaccharide, pentasaccharide, hexasaccharide, or a combination thereof.

4. The supramolecular self-assembly system according to any of items 1-3, characterized in that the compound with the flavonoid structure is selected from the group consisting of flavonoids, flavonols, flavanones (also known as dihydroflavones), flavanonols, isoflavones, anthocyanins, isoflavanones, chalcones, dihydrochalcones, aurones, flavans, and flavanols; the compound with the terpenoid structure refers to a compound derived from mevalonic acid and having a molecular skeleton based on an isoprene unit, such as a monoterpene, sesquiterpene, diterpene, triterpene, or tetraterpene compound.

5. The supramolecular self-assembly system according to any of items 1-4, characterized in that the polymer is selected from natural high molecular polymers and modified materials thereof, or artificially synthesized or semi-synthetic high molecular polymers, including but not limited to celluloses, homopolymers or copolymers, surfactants or emulsifiers.

6. The supramolecular self-assembly system according to any of items 1-5, characterized in that the target is selected from one or more of peptide drugs (such as cyclosporine, vitamin B12, voclosporin, 6-[(2S,3R,4R)-10-(acetylamino)-3-hydroxy-4-methyl-2-(methylamino) decanoic acid]-8-(N-methyl-D-alanine) cyclosporin A, reltecimod, balixafortide, relamorelin, 4F-benzoyl-TN14003, motixafortide, cyclo(L-arginyl-L-glutamyl-L-glutamylamido-L-serinyl-L-prolyl-L-α-glutamyl-L-histidine-L-glutamine), (5S,8S,10aR)-N-benzoyl-5-[(2S)-2-(methylamino) propionyl]amino) 3-(3-methylbutyryl)-6-oxo-1,2,4,5,8,9,10,10a-octahydropyrrole[1,2-a][1,5]diazocin-8-carboxamide, L-arginyl-L-isoleucine-L-histidine-L-methyl-L-alanyl-L-tyrosine-L-serine-L-lysyl-L-arginyl-O-phosphono-L-serineglycine-L-lysyl-L- prolyl-L-arginylglycine-L-tyrosine-L-alanyl-L-phenylalanine-L-isoleucine-L-α-glutamyl-L-tyrosine (Forigerimod), leuprorelin, batifiban, L-threonine-L-α-aspartic acid-L-leucine-L-glutamylamido-L-α-glutamyl-L-arginylglycine-L-α-aspartyl-L-asparaginyl-L-α-aspartyl-L-isoleucine-L-serinyl-L-prolyl-L-phenylalaninyl-L-serinylglycinyl-L-aspartyl-L-glutamylamido-L-prolyl-L-phenylalaninyl-L-lysyl-L-aspartic acid (Dentonin), (2S,5S,8S,11R,14S,20R)-N—((S)-1-amino-6-isopropylamino)-1-oxohexan-2-yl)-2-benzyl-11-(3-guanidinopropyl)-5-(4-hydroxybenzyl)-8-(4-(isopropylamino)butyl)-14-(naphth-2-ylmethyl)-3,6,9,12,15,18,23-heptyloxy-1,4,7,10,16,19-heptaazacyclotrichlorosilane-20-formamide (LY-2510924), (3S)-4-[[((2S)-5-amino-1-[[(2S,3R)-1-[[(2R)-1-[[(2R)-1-amino-1-oxoprop-2-yl]amino]-1-oxoprop-2-yl]amino]-3-hydroxy-1-oxobut-2-yl]amino]-1,5-dioxopent-2-yl]amino]-3-[[(2S)-2-[[(2S)-1-[(2S,3S)-2-[[(2S)-1-[(2S)-2-[(2R)-2-[[(2R)-2-aminopropionyl]amino]propionyl]amino]-4-methylpentanoyl]pyrrolidine-2-carbonyl]amino]-3-methylvaleryl]pyrrolidine-2-carbonyl]amino]-4-methylvaleryl]amino]-4-oxobutyric acid (SPX-101), disitertide, birinapant, glycyl-L-arginylglycyl-3-sulfo-L-alanyl-L-threonine-L-proline, cibinetide, veldoreotide, ozarelix, edratide, (2S)-2-[[[(2S)-4-carboxy-2-[[(2R)-2-[[2-[(2S)-3-carboxy-2-[[(2S)-2-formamido-4-methylthioalkylbutyryl]amino]propionyl]amino]acetyl]amino]-3-thioalkylpropionyl]amino]butyryl]amino]-4-methylvaleric acid, (2S)-2-[[((2S)-2-[(2S)-2-[[(2S,3R)-2-[[(2S)-2-[[(2S)-2-[[(2S)-2-[(2S)-2-[(2S)-2-[(2-acetamidoacetyl)amino]propionyl]amino]-5-amino-5-oxopentanoyl]amino]-3-phenylpropionyl]amino]-3-hydroxypropionyl]amino]-6-aminohexanoyl]amino]-3-hydroxybutyryl]amino]propionyl]amino]propionyl]amino]-6-aminohexanoic acid, (3S,6S,9S,12R,15S,18S,21S,24S,27R,30S,33S)-27-{[2-(dimethylamino)ethyl]thioalkyl}-30-ethyl-33-[(1R,2R,4E)-1-hydroxy-2-methylhexyl-4-alken-1-yl]-24-(2-hydroxy-2-methylpropyl)-1,4,7,10,12,15,19,25,28-nonylmethyl-6,9,18-tri(2-methylpropyl)-3,21-bis(prop-2-yl)-1,4,7,10,13,16,19,22,25,28,31-undecanoazatricyclododecane-2,5,8,11,14,17,20,23,26,29,32-undecene, (S)-1-((2S,5S,5S,8S,11S,14S)-18-amido-11-ethylpyrrolidine-2-carbonyl) pyrrolidine-2-carbonyl)-N-((2S,5S,5S,8S,11S,11S,14S)-18-amino-11-11-(S-sec-butyl)-14-carbamoyl-14-carbamoyl-8-8-(3-nitro-guanidyl)-1-(1-(1H-imidazol-5-yl-yl)-5-methyl-3-3,6,6,12-12-tetraoxoxy-4,4,7,7,10,13-tetraoctanooctadecane-13-octadecanooctan-2-2-yl-2-yl)amidomethyl-2-methyl-2-alk-alk-2-alk-yl)-3-(1H-imidazol-5-yl)-1-oxoprop-2-yl) pyrrolidine-2-carboxamide, cyclo[L-alanyl-L-serinyl-L-isoleucyl-L-prolyl-L-glutamylamido-L-lysyl-L-tyrosinyl-D-prolyl-L-prolyl-(2S)-2-aminodecanoyl-L-α-glutamyl-L-threonine], (4S)-4-{[((1S)-1-{[(1S)-1-{[(2S)-1-[(2S)-2-{[(1S)-1-{[(1S)-5-amino-1-{[((1S)-1-{[(1S)-1-{[(2S)-1-[(2S)-2-{[(1S)-4-carbamate-1-carboxy butyl]carbamoyl}pyrrolidin-1-yl]-4-methyl-1-oxopent-2-yl]carbamoyl}-2-carboxyethyl]carbamoyl}-2-methylpropyl]carbamoyl}pentyl]carbamoyl}-2-hydroxyethyl]carbamoyl}pyrrolidin-1-yl]-3-(1H-imidazol-5-yl)-1-oxoprop-2-yl]carbamoyl}-3-methylbutyl]carbamoyl}-2-phenylethyl]carbamoyl}-4-[(2S)-2,6-diaminohexamido]butyric acid, (2S)-1-[(2S)-2-cyclohexyl-2-[[((2S)-2-(methylamino) propionyl]amino]acetyl]-N-[2-(1,3-oxazol-2-yl)-4-phenyl-1,3-thiazol-5-yl]pyrrolidine-2-carboxamide, bortezomib, cyclo[L-alanyl-L-cysteinyl-L-serinyl-L-alanyl-D-prolyl-(2S)-2,4-diaminobutyryl-L-arginyl-L-tyrosinyl-L-cysteinyl-L-tyrosinyl-L-glutamylamido-L-lysinyl-D-prolinyl-L-prolinyl-L-tyrosinyl-L-histidine], (2→9)-disulfides, anidulafungin, atosiban, capreomycin, carbetocin, caspofungin, actinomycin, dalbavancin, romidepsin, octreotide, semaglutide, liraglutide, glucagon-like peptide 1, insulin calcitonin, central nervous system peptides, and protein drugs), BCS II class (low soluble and high osmotic) and BCSIV class (low soluble and low osmotic) drugs in biopharmaceutical classification systems (comprising but not limited to: aripiprazole, emtricitabine, bictegravir, lenalidomide, brexpiprazole, clotrimazole, clopidogrel, duloxetine, dapoxetine, dicyclomine, flecainide, indinavir, lamotrigine, lansoprazole, meclizine, nelfinavir, nevirapine, pioglitazone, chlorpromazine, quetiapine, raloxifen, rifabutin, ziprasidone, risperidone, rifampicin, selpercatinib, pemigatinib, ozanimod, osilodrostat, dasatinib, ruxolitinib, acalabrutinib, cediranib, dovitinib, sotorasib, adagrasib, motesanib, pazotinib, vardenafil, loperamide, lurasidone, alectinib, nintedanib, N-((7R,8R)-8-((2S,5S,8R,11S,14S,17S,20S,23R,26S,29S,32S)-5-ethyl-11,17,26,29-tetraisobutyl-14,32-diisopropyl-1,7,8,10,16,20,23,25,28,31-dodemethyl-3,6,9,12,15,18,21,24,27,30,33-undecyloxy-1,4,7,10,13,16,19,22,25,28,31-undecylazacyclotriazapolyglycos-2-yl)-8-hydroxy-7-methyloctyl) acetamide, ketoconazole, bosutinib, nilotinib, dabigatran etexilate, palbociclib, fingolimode, vincristine, vincamine, vinpocetine, edoxaban, pralsetinib, berotralstat, tirbanibulin, relugolix, pexidartinib, entrectinib, vandetanib, trilaciclib, tivozanib, rucaparib, ribociclib, tofacitinib, infigratinib, lorlatinib, niratinib, tepotinib, glasdegib, dacomitinib, enasidenib, cobimetinib, brigatinib, fedratinib, rimegepant, rosuvastatin, ethyl (3S)-8-{2-amino-6-[(1R)-1-(5-chloro[1,1′-biphenyl]-2-yl)-2,2,2-trifluoroethoxy]pyrimidin-4-yl}-2,8-diazaspiro[4.5]decane-3-carboxylate, tazemetostat, afatinib, tucatinib, abemaciclib, carvedilol, nebivolol, irbesartan, telmisartan, losartan, olanzapine, rupatadine, desloratadine, ritonavir, and verapamil; ripretinib, opicapone, vismodegib, vemurafenib, loratadine, riociguat, zanubrutinib, axitinib, orelabrutinib, mebendazole, norelgestromin, venetoclax, ticagrelor, ibrutinib, posaconazole, itraconazole, lenvatinib, macitentan, eltrombopag, donafenib, regorafenib, sorafenib, carfilzomib, rilpivirine, camptothecin, hydroxycamptothecin, methoxycamptothecin, nitrocamptothecin, aprepitant, selinexor, upadacitinib, umbralisib, sonidegib, sotorasib, talazoparib, lonafarnib, icotinib, dabrafenib, duvelisib, carfilzomib, capmatinib, bortezomib, binimetinib, avatrombopag, selumetinib, amprenavir, dexamethasone, methylprednisolone, prednisolone, cortisone, hydrocortisone, betamethasone, ivacaftor, teriflunomide, icaritin, olaparib, tolvaptan, pomalidomide, voriconazole, fluconazole, apixaban, vitamin K1, vitamin A, vitamin E, enzalutamide, chlorthalidone, etoposide, dutasteride, isradipine, butyphthalide, progesterone, rivaroxaban, tipranavir, spironolactone, warfarin, medroxyprogesterone, latanoprost, travoprost, bimatoprost, tafluprost, misoprostol, gemeprost, carboprost, latanoprost lactone diol, travoprost acid, travoprost, dinoprost, alprostadil, ezetimibe, felodipine, nifedipine, fenofibrate, celecoxib, tacrolimus, everolimus, rapamycin, carisoprodol, carbamazepine, paricalcitol, eldecalcitol, tacalcitol, doxercalciferol, calcipotriol, budesonide, vitamin D2, calcifediol, calciferol, calcitriol, alfacalcidol, seocalcitol, inecalcitol, falecalcitriol, maxacalcitol, griseofulvin, lopinavir, nabumetone, erdafitinib, allopregnenolone, afamelanotide, solriamfetol, pretomanid, oliceridine, foseltamivir, lurbinectedin, triheptanoin, tocotrienol, 4-[(1E,3S)-3-vinyl-3,7-dimethyl-1,6-octadien-1-yl]phenol, 7-hydroxy-3-[4-hydroxy-3-(3-methyl-2-buten-1-yl)phenyl]-4H-1-benzopyran-4-one, 3-[3-[(2E)-3,7-dimethyl-2,6-octadien-1-yl]-4-hydroxyphenyl]-7-hydroxy-4H-1-benzopyran-4-one, (2E)-1-[2,4-dihydroxy-3-(3-methyl-2-butenyl)phenyl]-3-(4-hydroxyphenyl)-2-propen-1-one, (6E,8E,10E,12E,14E,16E,18E,20E,22E,24E,26E)-2,6,10,14,19,23,27,31-octamethyl-2,6,8,10,12,14,16,18,20,22,24,26,30-diisoamyltriene, 2-[6-(2,4-dihydroxybenzoyl)-5-(2,4-dihydroxyphenyl)-3-methyl-2-cyclohexen-1-yl]-5a,10a-dihydro-1,3,5a,8-tetrahydroxy-10a-(3-methyl-2-buten-1-yl)-11H-benzofuran[3,2-b][1]benzopyran-11-one, (5aR,10aS)-2-[(1S,5S,6R)-6-(2,4-dihydroxybenzoyl)-5-(2,4-dihydroxyphenyl)-3-methyl-2-cyclohexen-1-yl]-5a,10a-dihydro-1,3,8,10a-tetrahydroxy-5a-(3-methyl-2-buten-1-yl)-11H-benzofuran[3,2-b][1]benzopyran-11-one, (2E)-3-(4-hydroxy-2-methoxyphenyl)-1-(4-methoxyphenyl)-2-propen-1-one, 2′,4,4′-trihydroxychalcone 4-(β-D-glucopyranoside), (E)-1-(2,4-dihydroxyphenyl)-3-(4-hydroxyphenyl) propyl-2-ene-1-one, (2E)-3-[5-(1,1-dimethyl-2-propen-1-yl)-4-hydroxy-2-methoxyphenyl]-1-(4-hydroxyphenyl)-2-propen-1-one, (2E)-3-[5-[(1S)-1,2-dimethyl-2-propen-1-yl]-4-hydroxy-2-methoxyphenyl]-1-(4-hydroxyphenyl)-2-propen-1-one, (2E)-3-(3,4-dihydroxy-2-methoxyphenyl)-1-[4-hydroxy-3-(3-methyl-2-buten-1-yl)phenyl]-2-propen-1-one, (2S)-2,3-dihydro-7-hydroxy-2-(4-hydroxyphenyl)-4H-1-benzopyran-4-one, 4′,7-dihydroxyflavanone 4′-β-D-glucopyranoside, 4-[5,7-dimethoxy-6-(3-methyl-2-buten-1-yl)-2H-1-benzopyran-3-yl]-1,3-benzenediyl, 4-[5,7-dimethoxy-6-(3-methyl-2-buten-1-yl)-2H-1-benzopyran-3-yl]-1,3-benzenediol, (2S)-2-[4-(β-D-glucopyranosyl)phenyl]-2,3-dihydro-7-hydroxy-4H-1-benzopyran-4-one, brassinin, carbamoylthioacid (1H-indol-3-ylmethyl)-methyl ester, 2-[3,4-dihydroxy-2,5-di(3-methyl-2-buten-1-yl)phenyl]-2,3-dihydro-5,7-dihydroxy-4H-1-benzopyran-4-one[UNK] (2R,3R)-2-(3,4-dihydroxyphenyl)-2,3-dihydro-3,5,7-trihydroxy-4H-1-benzopyran-4-one, (2R,3R)-2-(3,4-dihydroxyphenyl)-2,3-dihydro-3,5,7-trihydroxy-4H-1-benzopyran-4-one, (3S)-3-[2,4-dihydroxy-3-(3-methyl-2-buten-1-yl)phenyl]-2,3-dihydro-5,7-dihydroxy-4H-1-benzopyran-4-one, 4-[(3R)-3,4-dihydro-7-hydroxy-5-methoxy-6-(3-methyl-2-buten-1-yl)-2H-1-benzopyran-3-yl]-2-(3-methyl-2-buten-1-yl)-1,3-phenyldiol, 4-[(3R)-3,4-dihydro-8,8-dimethyl-2H,8H-benzo[1,2-b:3,4-b′]-bipyran-3-yl]-1,3-benzenediol, 4-[(3R)-3,4-dihydro-5,7-dimethoxy-6-(3-methyl-2-buten-1-yl)-2H-1-benzopyran-3-yl]-2-(3-methyl-2-buten-1-yl)-1,3-benzenediol, and 5,7-dihydroxy-3-(5-hydroxy-2,2-dimethyl-2H-1-benzopyran-6-yl)-4H-1-benzopyran-4-one; atorvastatin, simvastatin, lovastatin, pravastatin, fluvastatin, rosuvastatin, fosamprenavir, atovaquone, valsartan, candesartan cilexetil, fimasartan, eprosartan, olmesartan, diclofenac sodium, etodolac, furosemide, gemfibrozil, glimepiride, glipizide, glibenclamide, ibuprofen, indomethacin, meloxicam, naproxen, oxaprozin, doxorubicin, tafamidis, and eltrombopag), terpene lactones in natural products (such as artemisinin, parthenolide, thapsigargin, macrocarpal lactones A, B, C, D, and K, andrographolide, neoandrographolide, ginkgolides A, B, C, J, and K, bilobalide, jolkinolide B, nagilactone E, bruceantin, dichapetalin, limonin, triptolide, tripdiolide, celastrol, and celastrol), 7-ethyl-10-hydroxycamptothecin, irinotecan, paclitaxel, docetaxel, tanshinones (such as tanshinone IIA, dihydrotanshinone, cryptotanshinone, miltirone, and tanshinone I), curcumin, demethoxycurcumin, bis(demethoxycurcumin), flavonoids and biflavones (such as wogonin, baicalein, ginkgotin, ginkgetin, isoginkgetin, hinokiflavone, amentoflavone, xanthohumol, isoxanthohumol, demethylxanthohumol, naringenin, 8-isopentenyl naringenin, forskolin, 6-prenyl naringenin, 6,8-diprenyl naringenin, 6-geranyl naringenin, kurarinone, isokurarinone, and kurarinol), eurycomanone, 3,9-ethanol-1H,3H,7H-furan[3′,4′:2,3]cyclopentane[1,2-b]pyran-7-one, 4-(2,5-dihydro-3-methyl-5-oxo-2-furyl) hexahydro-3,8,9,11-tetrahydroxyl-4-methyl-10-methylene-, [3R-[3α,3αβ,4β(S*),5aα,8α,9α,9aR*,11R*]-, isobutyrylshikonin, acetylshikonin, deoxyshikonin, hesperidin, nobiletin, bavachinin, anwuligan, indirubin, psoralen, isopsoralen, psoralen dihydroflavone, psoralen isoflavone, vitamin A2, tretinoin, retinol derivatives, ponicidin, oridonin, scutellarin, tocopherol, artemisinin, gambogic acid, germacrone, curcumenone, curzerenone, neogambogic acid, isogambogic acid, betulinic acid, oleanolic acid, glycyrrhetinic acid, gymnemic acid IV, arjunolic acid, corosolic acid, ursolic acid, asiatic acid, 3-epicorosolic acid, pomolic acid, euscaphic acid, maslinic acid, ganoderic acid, tormentic acid, coenzyme Q10, cryptoxanthin, vitamin E, vitamin D, fullerene, icariin, icariin I, icariin II, icariin C, icariin B, and icariin A; cannabinols (such as cannabidiol, tetrahydrocannabinol, cannabinol, cannabichromene, (1′R,2′R)-4,5′-dimethyl-2′-(prop-1-en-2-yl)-1′,2′,3′,4′-tetrahydro-[1,1′-biphenyl]-2,6-diol, (1′R,2′R)-5′-methyl-2′-prop-1-en-2-yl)-4-propyl-1′,2′,3′,4′-tetrahydro-[1,1′-biphenyl]-2,6-diol, (1′R,2′R)-4-butyl-5′-methyl-2′-(prop-1-en-2-yl)-1′,2′,3′,4′-tetrahydro-[1,1′-biphenyl]-2,6-diol, (1′R,2′R)-2,6-dihydroxy-5′-methyl-4-pentyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-2′-(prop-1-en-2-yl)-4-propyl-1′,2′,3′,4′-tetrahydro-[1,1′-biphenyl]-3-carboxylic acid, (1′R,2′R)-6-methoxy-5′-methyl-4-pentyl-2′-(prop-1-en-2-yl)-1′,2′,3′,4′-tetrahydro-[1,1′-biphenyl]-2-ol, 5′-methyl-4-pentyl-2′-(prop-1-en-2-yl)-[1,1′-biphenyl]-2,6-diol, 5′-methyl-2′-(prop-1-en-2-yl)-4-propyl-[1,1′-biphenyl]-2,6-diol, (1R,6R)-2′,6′-dihydroxy-4′-pentyl-6-(prop-1-en-2-yl)-1,4,5,6-tetrahydro-[1,1′-biphenyl]-3-carboxylic acid, (1′R,2′R)-5′-(hydroxymethyl)-4-pentyl-2′-(prop-1-en-2-yl)-1′,2′,3′,4′-tetrahydro-[1,1′-biphenyl]-2,6-diol, (5aR,6S,9R,9aR)-6-methyl-3-pentyl-9-(prop-1-en-2-yl)-5a,6,7,8,9,9a-hexahydrodibenzo[b,d]furan-1,6-diol, (2S,3S,4S,5R)-3,4,5-trihydroxy-6-((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)tetrahydro-2H-pyran-2-carboxylic acid, 2-((1S,2S,5S)-5-methyl-2-(prop-1-en-2-yl)cyclohexyl)-5-((E)-styryl)phenyl-1,3-diol, 5-((E)-2-hydroxystyryl)-2-((1S,2S,5S)-5-methyl-2-(prop-1-en-2-yl)cyclohexyl)phenyl-1,3-diol, 5-(benzofuran-2-yl)-2-(1S,2S,5S)-5-methyl-2-(prop-1-en-2-yl)cyclohexyl)phenyl-1,3-diol, (1'S,2'S)-2′-(5-hydroxy-6-methylheptyl-1,6-dien-2-yl)-4,5′-dimethyl-1′,2′,3′,4′-tetrahydro-[1,1′-biphenyl]-2,6-diol, 3-phenyl-1-((1'S,2'S)-2,4,6-trihydroxy-5′-methyl-2′-(prop-1-en-2-yl)-1′,2′,3′,4′-tetrahydro-[1,1′-biphenyl]-3-yl) propan-1-one, (1'S,2'S)-5′-methyl-4-pentyl-2′-(propanediol-1-en-2-yl)-1′,2′,3′,4′-tetrahydro-[1,1′-biphenyl]-2,6-diol, (1'S,2'S)-2′-isopropyl-5′-methyl-4-pentyl-1′,2′,3′,4′-tetrahydro-[1,1′-biphenyl]-2,6-diol, 2-((1R,2S)-2-isopropyl-5-methylcyclohexyl)-5-pentylphenyl-1,3-diol, (1'S,2'S)-5′-(hydroxymethyl)-2′-isopropyl-4-pentyl-1′,2′,3′,4′-tetrahydro-[1,1′-biphenyl]-2,6-diol, (1′R,2'S)-5′-(hydroxymethyl)-2′-isopropyl-4-pentyl-1′,2′,3′,4′-tetrahydro-[1,1′-biphenyl]-2,6-diol, (1′R,2′R)-5′-methyl-4-(2-methyloctan-2-yl)-2′-(prop-1-en-2-yl)-1′,2′,3′,4′-tetrahydro-[1,1′-biphenyl]-2,6-diol, (1R,6R)-2′,6′-dihydroxy-4′-(2-methyloctan-2-yl)-6-(prop-1-en-2-yl)-1,4,5,6-tetrahydro-[1,1′-biphenyl]-3-carboxylic acid, (1′R,2′R)-5′-(hydroxymethyl)-4-(2-methyloctan-2-yl)-2′-(prop-1-en-2-yl)-1′,2′,3′,4′-tetrahydro-[1,1′-biphenyl]-2,6-diol, (1R,2R)-2′,6′-dimethoxy-5-methyl-4′-(2-methyloctan-2-yl)-2-(prop-1-en-2-yl)-1,2,3,4-tetrahydro-1,1′-biphenyl, (1'S,2'S)-2′-isopropyl-5′-methyl-4-(2-methyloctan-2-yl)-1′,2′,3′,4′-tetrahydro-[1,1′-biphenyl]-2,6-diol, 2-((1R,2S)-2-isopropyl-5-methylcyclohexyl)-5-(2-methyloctan-2-yl)phenyl-1,3-diol, ((1S,4S,5S)-4-(2,6-dimethoxy-4-(2-methyloctan-2-yl)phenyl)-6,6-dimethylbicyclo[3.1.1]hept-2-en-2-yl) methanol, ((1R,4R,5R)-4-(2,6-dimethoxy-4-(2-methyloctan-2-yl)phenyl)-6,6-dimethylbicyclo[3.1.1]hept-2-en-2-yl) methanol, 1-(3-((1′R,2′R)-2,6-dihydroxy-5′-methyl-2′-(prop-1-en-2-yl)-1′,2′,3′,4′-tetrahydro-[1,1′-biphenyl]-4-yl)methyl) azetidin-1-yl) ethanone, (1′R,2′R)-4-(2-(1H-1,2,3-triazol-1-yl)ethyl)-5′-methyl-2′-(prop-1-en-2-yl)-1′,2′,3′,4′-tetrahydro-[1,1′-biphenyl]-2,6-diol, 2-((1′R,2′R)-2,6-dihydroxy-5′-methyl-2′-(prop-1-en-2-yl)-1′,2′,3′,4′-tetrahydro-[1,1′-biphenyl]-4-yl)-1-morpholinoethanone, (1′R,2′R)-4-(4-hydroxybutyl)-5′-methyl-2′-(prop-1-en-2-yl)-1′,2′,3′,4′-tetrahydro-[1,1′-biphenyl]-2,6-diol, 4-((1′R,2′R)-2,6-dihydroxy-5′-methyl-2′-(prop-1-en-2-yl)-1′,2′,3′,4′-tetrahydro-[1,1′-biphenyl]-4-yl) butyric acid, (1′R,2′R)-4-(2-ethoxyethyl)-5′-methyl-2′-(prop-1-en-2-yl)-1′,2′,3′,4′-tetrahydro-[1,1′-biphenyl]-2,6-diol, (1′R,2′R)-3-chloro-5′-methyl-4-pentyl-2′-(prop-1-en-2-yl)-1′,2′,3′,4′-tetrahydro-[1,1′-biphenyl]-2,6-diol, (1′R,2′R)-3,5-dichloro-5′-methyl-4-pentyl-2′-(prop-1-en-2-yl)-1′,2′,3′,4′-tetrahydro-[1,1′-biphenyl]-2,6-diol, (1′R,2′R)-3-bromo-5′-methyl-4-pentyl-2′-(prop-1-en-2-yl)-1′,2′,3′,4′-tetrahydro-[1,1′-biphenyl]-2,6-diol, (1′R,2′R)-3,5-dibromo-5′-methyl-4-pentyl-2′-(prop-1-en-2-yl)-1′,2′,3′,4′-tetrahydro-[1,1′-biphenyl]-2,6-diol, (1′R,2′R)-3-iodo-5′-methyl-4-pentyl-2′-(prop-1-en-2-yl)-1′,2′,3′,4′-tetrahydro-[1,1′-biphenyl]-2,6-diol, (1′R,2′R)-3,5-diiodo-5′-methyl-4-pentyl-2′-(prop-1-en-2-yl)-1′,2′,3′,4′-tetrahydro-[1,1′-biphenyl]-2,6-diol, (1′R,2′R)-3-fluoro-5′-methyl-4-pentyl-2′-(prop-1-en-2-yl)-1′,2′,3′,4′-tetrahydro-[1,1′-biphenyl]-2,6-diol, 3-(acetoxy)-2-[(1R,6R)-6-(3-fluoroprop-1-en-2-yl)-3-methylcyclohex-2-en-1-yl]-5-pentylphenyl acetate, (1′R,2′R)-5′-(fluoromethyl)-4-pentyl-2′-(prop-1-en-2-yl)-1′,2′,3′,4′-tetrahydro-[1,1′-biphenyl]-2,6-diol, 1,3-dimethoxy-2-[(1R,6R)-3-methyl-6-prop-1-en-2-ylcyclohex-2-en-1-yl]-5-pentylbenzene, (1′R,2′R)-5′-methyl-4-pentyl-2′-(prop-1-en-2-yl)-1′,2′,3′,4′-tetrahydro-[1,1′-biphenyl]-2-ol, (1R,6R)-2′,6′-diacetoxy-4′-pentyl-6-(prop-1-en-2-yl)-1,4,5,6-tetrahydro-[1,1′-biphenyl]-3-carboxylic acid, 2-((1′R,2′R)-6-hydroxy-5′-methyl-4-pentyl-2′-(prop-1-en-2-yl)-1′,2′,3′,4′-tetrahydro-[1,1′-biphenyl]-2-yl)oxy) acetic acid, (1′R,2′R)-6-(3-aminopropoxy)-5′-methyl-4-pentyl-2′-(prop-1-en-2-yl)-1′,2′,3′,4′-tetrahydro-[1,1′-biphenyl]-2-ol, 2-[3-(cyanomethoxy)-2-[(1R,6R)-3-methyl-6-(prop-1-en-2-yl)cyclohex-2-en-1-yl]-5-pentylphenoxy]acetonitrile, 3-({[(diethylamino)methoxy]carbonyl}oxy)-2-[(1R,6R)-3-methyl-6-(prop-1-en-2-yl)cyclohex-2-en-1-yl]-5-pentylphenyl(diethylamino)methyl carbonate, 3-({2-[(tert-butyldimethylsilyl)oxy]acetoxy)-2-[(1R,6R)-3-methyl-6-(prop-1-en-2-yl)cyclohex-2-en-1-yl]-5-pentylphenyl 2-[(tert-butyldimethylsilyl)oxy]acetate, 3-(acetoxy)-2-[(1R,6R)-3-methyl-6-(3-oxoprop-1-en-2-yl)cyclohex-2-en-1-yl]-5-pentylphenyl acetate, 3-(acetoxy)-2-[(1R,6R)-3-methyl-4-oxo-6-(prop-1-en-2-yl)cyclohex-2-en-1-yl]-5-pentylphenyl acetate, 3-(acetoxy)-2-[(1R,6R)-4-(acetoxy)-3-methyl-6-(prop-1-en-2-yl)cyclohex-2-en-1-yl]-5-pentylphenyl acetate, 2-[(1R,2R)-2-[2,6-di(acetoxy)-4-pentenyl]-4-methylcyclohex-3-en-1-yl]prop-2-en-1-yl acetate, 3-hydroxy-2-[(1R,6R)-3-methyl-6-prop-1-en-2-ylcyclohex-2-en-1-yl]-5-pentylcyclohex-2,5-dien-1,4-dione, 2,5-cyclohexadien-1,4-dione, 2-hydroxy-3-((1R,6R)-3-methyl-6-(1-methylvinyl)-2-cyclohexen-1-yl)-6-pentyl-5-(butamino), 2,5-cyclohexadien-1,4-dione, 2-hydroxy-3-((1R,6R)-3-methyl-6-(1-methylvinyl)-2-cyclohexen-1-yl)-6-pentyl-5-((benzyl)amino), 5-methyl-4-[(1R,6R)-3-methyl-6-prop-1-en-2-ylcyclohex-2-en-1-yl]phenyl-1,3-diol, 4-[(1R,6R)-3-methyl-6-prop-1-en-2-ylcyclohex-2-en-1-yl]-5-pentylphenyl-1,3-diol, 2-[(2E)-3,7-dimethylocta-2,6-dienyl]-5-pentylphenyl-1,3-diol, 1-[(1R,2R,3R,4R)-3-(2,6-dihydroxy-4-pentylphenyl)-2-hydroxy-4-prop-1-en-2-ylcyclopentyl]ethanone.

7. The supramolecular self-assembly system according to any of items 1-6, characterized in that a mass ratio of the carrier (preferably carriers with the flavonoid or terpenoid structure) to the target is 0.003:1 to 250:1, preferably 0.01:1 to 200:1, and more preferably 0.015:1 to 150:1.

8. The supramolecular self-assembly system according to any of items 1-7, characterized in that a mass ratio of the carrier (preferably carriers with the flavonoid or terpenoid structure) to the polymer is 1:0 to 1:100, preferably 1:0 to 1:75, and more preferably 1:0 to 1:50.

9. The supramolecular self-assembly system according to any of items 1-8, characterized in that the carrier with the flavonoid structure is selected from hesperetin, naringenin, quercetin, kaempferol, isorhamnetin, myricetin, apigenin, luteolin, eriodictyol, diosmetin, genistein, baicalein, catechin, epicatechin, puerarin, isoprimin, tannic acid, chrysin, pelargonidin, cyanidin, delphinidin, peonidin, petunidin, malvidin, and saccharide derivatives thereof, such as flavonoid glycosides formed by connection with monosaccharides, disaccharides, trisaccharides, acylated saccharides, or tetrasaccharides, chalcones, dihydrochalcones, flavonols, isoprene compounds, and derivatives with saccharides.

10. The supramolecular self-assembly system according to any of items 1-8, characterized in that the carrier with the terpenoid structure is selected from compounds containing isoprene or isopentane, including but not limited to monoterpenes, cycloalkene ether terpenes, sesquiterpenes, diterpenes, triterpenes, and tetraterpenes.

11. The supramolecular self-assembly system according to item 2, characterized in that the polymer is selected from one or more of cellulose, starch, soluble starch, wheat starch, potato starch, cassava starch, gellan gum, maltodextrin, hyaluronic acid, zein, corn starch, tragacanth gum, arabic gum, alginic acid, sodium alginate, pectin, chitosan, arabinogalactan, polysaccharide or polysaccharide extract, xanthan gum, cyclodextrin, and derivatives thereof; the artificially synthesized or semi-synthesized polymer is selected from one or more of hydroxypropyl methyl cellulose, methyl cellulose, cellulose acetate, ethyl cellulose, hydroxypropyl cellulose, low-substituted hydroxypropyl cellulose, microcrystalline cellulose, carboxymethyl cellulose, carboxymethyl starch sodium, hydroxypropyl methyl cellulose acetate succinate, hydroxypropyl methyl cellulose phthalate, cross-linked carboxymethyl cellulose sodium or calcium, and silicified microcrystalline cellulose; and the polymer A is selected from one or more of polyethylene caprolactam-polyvinyl acetate-polyethylene glycol graft copolymer, copovidone, polyvinylpyrrolidone series, polyethylene glycol series, ethyl acrylate-methyl methacrylate-trimethylamine ethyl methacrylate chloride (1:2:0.2) copolymer, ethyl acrylate-methyl methacrylate-trimethylamine ethyl methacrylate chloride (1:2:0.1) copolymer, methacrylic acid-ethyl acrylate (1:1) copolymer, methacrylic acid-methyl methacrylate (1:1) copolymer, methacrylic acid-methyl methacrylate (1:2) copolymer, butyl methacrylate-dimethylaminoethyl methacrylate-methyl methacrylate (1:2:1) copolymer, ethyl acrylate-methyl methacrylate (2:1) copolymer, glycolide lactide copolymer series, carbomer, carbomer copolymer, polylactic acid-hydroxyglycolic acid copolymer, polylactic acid-glycollic acid copolymer, sorbitan trioleate, lauroyl polyoxyethylene glyceride, oleoyl polyoxyethylene glyceride, oleic acid polyoxyethylene ester, polysorbates (Tween20 and 80), poloxamer, vitamin E succinate polyethylene glycol ester (TPGS), stearic acid polyoxometalate, polyvinyl alcohol, polyammonium methacrylate, polyoxyethylene, polyoxyethylene castor oil, and polyoxyethylene hydrogenated castor oil.

12. The supramolecular self-assembly system according to any of items 1-11, characterized in that the target has a Log P or Log D7.4 of 0.8-17, 0-7 hydrogen donors, and 1-12 hydrogen acceptors, and is dissociated or non-dissociated; in the presence of a plurality of targets, there is an intermolecular interaction and/or an intramolecular interaction or no such interactions between the targets; preferably, the target is selected from the group consisting of nilotinib, nintedanib, lenvatinib, sorafenib, ticagrelor, apixaban, rivaroxaban, warfarin, lurasidone, curcumin, vitamin K1, macitentan, tacrolimus, cyclosporine, paclitaxel, docetaxel, ibrutinib, clopidogrel, fingolimode, enzalutamide, posaconazole, dabigatran etexilate, venetoclax, alectinib, palbociclib, naringenin, celecoxib, itraconazole, eltrombopag, griseofulvin, acalabrutinib, ezetimibe, felodipine, scutellarin, candesartan cilexetil, regorafenib, butyphthalide, coenzyme Q10, cannabidiol, tafluprost, lutein, vitamin E, vitamin A, and salts, hydrates, solvates, or eutectics thereof.

13. The supramolecular self-assembly system according to any of items 1-8, characterized in that the carrier with the flavonoid structure is selected from naringenin, hesperetin, catechin, epicatechin, quercetin, isoquercitrin, myricetin, eriodictin, and/or flavonoid glycosides, flavonol glycosides, and flavanols formed by connecting them to saccharides with a number of N (where N is greater than or equal to 1) and acylated saccharides, and/or chalcones (such as dihydrochalcones) and saccharide derivatives of chalcones (such as dihydrochalcones), such as derivatives formed by connecting them to saccharides with a number of N (where N is greater than or equal to 1).

14. The supramolecular self-assembly system according to any of items 1-8, characterized in that the carrier with the flavonoid structure is selected from naringin, hesperidin, epicatechin gallate, isoquercitrin, quercetin, myricetrin, epigallocatechin, tannic acid, neohesperidin dihydrochalcone, trilobatin, naringin dihydrochalcone, quercetin 3-rutinoside, and neohesperidin.

15. The supramolecular self-assembly system according to any of items 1-8, characterized in that the carrier with the terpenoid structure is selected from sweet tea, rubusoside, rebaudioside A, rebaudioside B, rebaudioside C, rebaudioside D, rebaudioside M, stevia, soyasaponin A1, soyasaponin Ba, soyasaponin I, soyasaponin II, soyasaponin III, glycyrrhizic acid and salts thereof, glycyrrhetinic acid, stevioside, stevioside ingredient extract (stevioside content ≥95%, where rebaudioside A ≥25), mogroside V, mogroside ingredient extract (containing mogroside V ≥30%, HPLC), asiaticoside, asiaticoside A, asiaticoside B, asiaticoside E, asiaticoside F, ginsenoside Rg1, ginsenoside Rb1, dioscin, mogroside IV, mogroside V, oat saponin A, oat saponin B, platycodin A, platycodin B, platycodin D, platycodin D2, platycodin D3, tenuigenin A, tenuigenin D, and tenuigenin D2.

16. The supramolecular self-assembly system according to item 2, characterized in that the polymer A is selected from one or more of polyethylene caprolactam-polyvinyl acetate-polyethylene glycol graft copolymer, hydroxypropyl methyl cellulose acetate succinate and polyethylene caprolactam-polyvinyl acetate-polyethylene glycol graft copolymer, hydroxypropyl methyl cellulose, sodium carboxymethyl cellulose, calcium carboxymethyl cellulose, polyvinyl pyrrolidone, copovidone, polyethylene glycol, cellulose acetate, hyaluronic acid, xanthan gum, methacrylic acid-methyl methacrylate copolymer (1:1), methacrylic acid-ethyl methacrylate copolymer (1:1), hydroxypropyl cellulose, polyoxyethylene-polyoxypropylene block copolymer, sodium dodecyl sulfate, TPGS, and polyacrylic acid.

17. A composition, including the supramolecular self-assembly system according to any of items 1-16, and preferably further including one or more of fillers, disintegrants, adhesives, lubricants, flow aids, emulsifiers, flavor enhancers or masking agents, surfactants, co-surfactants, and preservatives.

18. The composition according to item 17, being tablets, capsules, suspension, patch, cream, gel, emulsion, eye drops, injection, oral capsules, suppository, implants, powder; or being contained in parenteral nutrition liquid, enteral nutrition liquid, health products, functional beverages, and preservative and fresh-keeping products in the food and beverage industry; or being contained in perfume, gel, cream, emulsion, masks, and lipsticks in the cosmetics industry; or being contained in toothpaste, shampoo, conditioners, and hair cream in the field of fine chemicals; or being contained in diagnostic products, implant materials, and biosensors in the field of biomedicine.

19. A use of the supramolecular self-assembly system according to any of items 1-16 in drugs, cosmetics, food, diagnostic reagents, implants, or biosensors.

For example, the present invention aims to take advantage of the structural diversity, good biocompatibility, good safety and amphiphilicity of natural flavonoids and terpenoids, and the characteristics of providing more effective groups for molecular interactions at the same time, to construct a multivariate supramolecular self-assembly system with one or two pharmaceutical macromolecular excipients, target drugs or compounds. This supramolecular self-assembly system can be prepared using existing processes in the pharmaceutical industry according to a formula, and then mixed with other excipients acceptable in the pharmaceutical field to form a target dosage form, or combined with existing technologies such as solid dispersion, self-microemulsion, and lipid formulation technology to achieve efficient and safe delivery of target ingredients. This supramolecular self-assembly system built based on natural flavonoids or terpenoids and polymer excipients can avoid the degradation or efflux of target ingredients by enzymes before absorption in the digestive tract, and at the same time, through cooperative regulation, to control the possible molecular stacking of class BCS II and class IV insoluble drugs due to intermolecular hydrogen bonding interaction, electrostatic interaction, dipole interaction, T-T stacking, Van der Waals force, and hydrophobic effect caused by the molecular structures of the drugs. The newly built supramolecular self-assembly system has good water solubility. By adjusting the ratio of flavonoid or terpenoid carriers to drugs in the system, the stability and hydrophobicity of the finally built supramolecular self-assembly system are controlled, thereby improving drug delivery efficiency, reducing drug dosage, minimizing drug interactions, reducing adverse reactions to gastrointestinal mucosa, enhancing drug stability, and ultimately improving the safety, effectiveness, and compliance of long-term medication for patients.

In the present invention, the targets are sometimes referred to as “target guest molecules” or “guest molecules”. The polymers are also referred to as “high molecular polymers” or “high molecular building units”. The carriers are also referred to as “carrier building units”. The “supramolecular self-assembly system” is sometimes referred to as “supramolecular system”. The term “supramolecular self-assembly system” indicates that different molecules spontaneously assemble into ordered supramolecular aggregates of different sizes and shapes through a series of weak non-covalent interactions, such as hydrogen bonding, electrostatic interaction, dipole interaction, T-T stacking, Van der Waals force, and hydrophobic effect. A system built by the supramolecular aggregates is referred to as the supramolecular self-assembly system. (1) The targets applicable to the present invention include but are not limited to the following:

### 1) Peptide/Protein Drugs:

The peptide drugs include reltecimod, balixafortide, relamorelin, 4F-benzoyl-TN14003 motixafortide, cyclo(L-arginyl-L-glutamyl-L-glutamylamido-L-serinyl-L-prolyl-L-α-glutamyl-L-histidine-L-glutamine), (5S,8S,10aR)-N-benzoyl-5-[(2S)-2-(methylamino) propionyl]amino) 3-(3-methylbutyryl)-6-oxo-1,2,4,5,8,9,10,10a-octahydropyrrole[1,2-a][1,5]diazocin-8-carboxamide, L-arginyl-L-isoleucine-L-histidine-L-methyl-L-alanyl-L-tyrosine-L-serine-L-lysyl-L-arginyl-O-phosphono-L-serine glycine-L-lysyl-L-prolyl-L-arginyl glycine-L-tyrosine-L-alanyl-L-phenylalanine-L-isoleucine-L-α-glutamyl-L-tyrosine furimod, leuprorelin, batifiban, L-threonine-L-α-aspartic acid-L-leucine-L-glutamylamido-L-α-glutamyl-L-arginylglycine-L-α-aspartyl-L-asparaginyl-L-α-aspartyl-L-isoleucine-L-serinyl-L-prolyl-L-phenylalaninyl-L-serinylglycinyl-L-aspartyl-L-glutamylamido-L-prolyl-L-phenylalaninyl-L-lysyl-L-aspartic acid, (2S,5S,8S,11R,14S,20R)-N—((S)-1-amino-6-isopropylamino)-1-oxohexan-2-yl)-2-benzyl-11-(3-guanidinopropyl)-5-(4-hydroxybenzyl)-8-(4-(isopropylamino)butyl)-14-(naphth-2-ylmethyl)-3,6,9,12,15,18,23-heptyloxy-1,4,7,10,16,19-heptaazacyclotrichlorosilane-20-formamide, disitertide, (3S)-4-[[((2S)-5-amino-1-[[(2S,3R)-1-[[(2R)-1-[[(2R)-1-amino-1-oxoprop-2-yl]amino]-1-oxoprop-2-yl]amino]-3-hydroxy-1-oxobut-2-yl]amino]-1,5-dioxopent-2-yl]amino]-3-[(2S)-2-[[(2S)-1-[(2S,3S)-2-[[(2S)-1-[(2S)-2-[[(2R)-2-[(2R)-2-aminopropionyl]amino]propionyl]amino]-4-methylpentanoyl]pyrrolidine-2-carbonyl]amino]-3-methylvaleryl]pyrrolidine-2-carbonyl]amino]-4-methylvaleryl]amino]-4-oxobutyric acid, birinapant, glycyl-L-arginylglycyl-3-sulfo-L-alanyl-L-threonine-L-proline, cibinetide, veldoreotide, ozarelix, edratide, (2S)-2-[[[(2S)-4-carboxy-2-[[(2R)-2-[[2-[[(2S)-3-carboxy-2-[[(2S)-2-formamido-4-methylthioalkylbutyryl]amino]propionyl]amino]acetyl]amino]-3-thioalkylpropionyl]amino]butyryl]amino]-4-methylvaleric acid, (2S)-2-[[((2S)-2-[(2S)-2-[[(2S,3R)-2-[(2S)-2-[[(2S)-2-[(2S)-2-[[(2S)-2-[[(2S)-2-[(2-acetamidoacetyl)amino]propionyl]amino]-5-amino-5-oxopentanoyl]amino]-3-phenylpropionyl]amino]-3-hydroxypropionyl]amino]-6-aminohexanoyl]amino]-3-hydroxybutyryl]amino]propionyl]amino]propionyl]amino]-6-aminohexanoic acid, (3S,6S,9S,12R,15S,18S,21S,24S,27R,30S,33S)-27-{[2-(dimethylamino)ethyl]thioalkyl}-30-ethyl-33-[(1R,2R,4E)-1-hydroxy-2-methylhexyl-4-alken-1-yl]-24-(2-hydroxy-2-methylpropyl)-1,4,7,10,12,15,19,25,28-nonylmethyl-6,9,18-tri(2-methylpropyl)-3,21-bis(prop-2-yl)-1,4,7,10,13,16,19,22,25,28,31-undecanoazatricyclododecane-2,5,8,11,14,17,20,23,26,29,32-undecene, (S)-1-((2S,5S,5S,8S,11S,14S)-18-amido-11-ethylpyrrolidine-2-carbonyl) pyrrolidine-2-carbonyl)-N-((2S,5S,5S,8S,11S,11S,14S)-18-amino-11-11-(S-sec-butyl)-14-carbamoyl-14-carbamoyl-8-8-(3-nitro-guanidyl)-1-(1-(1H-imidazol-5-yl-yl)-5-methyl-3-3,6,6,12-12-tetraoxoxy-4,4,7,7,10,13-tetraoctanooctadecane-13-octadecanooctan-2-2-yl-2-yl)amidomethyl-2-methyl-2-alk-alk-2-alk-yl)-3-(1H-imidazol-5-yl)-1-oxoprop-2-yl) pyrrolidine-2-carboxamide, cyclo[L-alanyl-L-serinyl-L-isoleucyl-L-prolyl-L-glutamylamido-L-lysyl-L-tyrosinyl-D-prolyl-L-prolyl-(2S)-2-aminodecanoyl-L-α-glutamyl-L-threonine], (4S)-4-{[((1S)-1-{[(1S)-1-{[(2S)-1-[(2S)-2-{[(1S)-1-{[(1S)-5-amino-1-{[((1S)-1-{[(1S)-1-{[(2S)-1-[(2S)-2-{[(1S)-4-carbamate-1-carboxybutyl]carbamoyl}pyrrolidin-1-yl]-4-methyl-1-oxopent-2-yl]carbamoyl}-2-carboxyethyl]carbamoyl}-2-methylpropyl]carbamoyl}pentyl]carbamoyl}-2-hydroxyethyl]carbamoyl}pyrrolidin-1-yl]-3-(1H-imidazol-5-yl)-1-oxoprop-2-yl]carbamoyl}-3-methylbutyl]carbamoyl}-2-phenylethyl]carbamoyl}-4-[(2S)-2,6-diaminohexamido]butyric acid, (2S)-1-[(2S)-2-cyclohexyl-2-[[((2S)-2-(methylamino) propionyl]amino]acetyl]-N-[2-(1,3-oxazol-2-yl)-4-phenyl-1,3-thiazol-5-yl]pyrrolidine-2-carboxamide, bortezomib, cyclo[L-alanyl-L-cysteinyl-L-serinyl-L-alanyl-D-prolyl-(2S)-2,4-diaminobutyryl-L-arginyl-L-tyrosinyl-L-cysteinyl-L-tyrosinyl-L-glutamylamido-L-lysinyl-D-prolinyl-L-prolinyl-L-tyrosinyl-L-histidine], (2->9)-disulfides, anidulafungin, atosiban, capreomycin, carbetocin, caspofungin, actinomycin, dalbavancin, romidepsin, ciclosporin, vitamin B12, octreotide, semaglutide, liraglutide, glucagon-like peptide 1, insulin calcitonin, central nervous system peptides, and protein drugs.

### 2) the BCSII and BCSIV Drugs in the Biopharmaceutical Classification System Include but are not Limited to the Following:

Aripiprazole, emtricitabine, bictegravir, lenalidomide, brexpiprazole, clotrimazole, clopidogrel, duloxetine, dapoxetine, dicyclomine, flecainide, indinavir, lamotrigine, lansoprazole, meclizine, nelfinavir, nevirapine, pioglitazone, chlorpromazine, quetiapine, raloxifen, rifabutin, ziprasidone, risperidone, rifampicin, selpercatinib, pemigatinib, ozanimod, osilodrostat, dasatinib, ruxolitinib, acalabrutinib, cediranib, dovitinib, sotorasib, adagrasib, cannabidiol, tetrahydrocannabinol, motesanib, pazotinib, vardenafil, loperamide, lurasidone, alectinib, nintedanib, voclosporin, 6-[(2S,3R,4R)-10-(acetylamino)-3-hydroxy-4-methyl-2-(methylamino) decanoic acid]-8-(N-methyl-D-alanine)cyclosporin A, N-((7R,8R)-8-((2S,5S,8R,11S,14S,17S,20S,23R,26S,29S,32S)-5-ethyl-11,17,26,29-tetraisobutyl-14,32-diisopropyl-1,7,8,10,16,20,23,25,28,31-dodemethyl-3,6,9,12,15,18,21,24,27,30,33-undecyloxy-1,4,7,10,13,16,19,22,25,28,31-undecylazacyclotriazapolyglycos-2-yl)-8-hydroxy-7-methyloctyl) acetamide, ketoconazole, bosutinib, nilotinib, dabigatran etexilate, palbociclib, fingolimode, vincristine, vincamine, vinpocetine, edoxaban, pralsetinib, berotralstat, tirbanibulin, relugolix, pexidartinib, entrectinib, vandetanib, trilaciclib, tivozanib, rucaparib, ribociclib, tofacitinib, infigratinib, lorlatinib, niratinib, tepotinib, glasdegib, dacomitinib, enasidenib, cobimetinib, brigatinib, fedratinib, rimegepant, rosuvastatin, ethyl (3S)-8-{2-amino-6-[(1R)-1-(5-chloro[1,1′-biphenyl]-2-yl)-2,2,2-trifluoroethoxy]pyrimidin-4-yl}-2,8-diazaspiro[4.5]decane-3-carboxylate, tazemetostat, afatinib, tucatinib, abemaciclib, carvedilol, nebivolol, irbesartan, telmisartan, losartan, olanzapine, rupatadine, desloratadine, ritonavir, and verapamil; ripretinib, opicapone, vismodegib, vemurafenib, loratadine, riociguat, zanubrutinib, axitinib, orelabrutinib, mebendazole, norelgestromin, venetoclax, ticagrelor, ibrutinib, posaconazole, itraconazole, lenvatinib, macitentan, eltrombopag, donafenib, regorafenib, sorafenib, carfilzomib, rilpivirine, camptothecin, hydroxycamptothecin, methoxycamptothecin, nitrocamptothecin, aprepitant, selinexor, upadacitinib, umbralisib, sonidegib, sotorasib, talazoparib, lonafarnib, icotinib, dabrafenib, duvelisib, carfilzomib, capmatinib, bortezomib, binimetinib, avatrombopag, selumetinib, and amprenavir; terpene lactones in natural products, sesquiterpene lactone products, such as artemisinin, parthenolide, thapsigargin, macrocarpal lactones A, B, C, D, and K; diterpene lactones compounds, such as andrographolide, neoandrographolide, ginkgolides A, B, C, J, and K, bilobalide, jolkinolide B, nagilactone E, triptolide, 7-ethyl-10-hydroxycamptothecin, irinotecan, celastrol, paclitaxel, and paclitaxel derivatives, such as docetaxel), danshenketones, such as tanshinone IIA, dihydrotanshinone, cryptotanshinone, miltirone, and tanshinone I; triterpene lactone compounds, such as bruceantin, dichapetalin, and limonin. Curcumin, demethoxycurcumin, and bis(demethoxycurcumin); flavonoids and biflavones (such as wogonin, baicalein, ginkgotin, ginkgetin, isoginkgetin, hinokiflavone, amentoflavone, xanthohumol, isoxanthohumol, demethylxanthohumol, naringenin, 8-isopentenyl naringenin, forskolin, 6-prenyl naringenin, 6,8-diprenyl naringenin, 6-geranyl naringenin, kurarinone, isokurarinone, kurarinol, eurycomanone, 3,9-ethanol-1H,3H,7H-furan[3′,4′:2,3]cyclopentane[1,2-b]pyran-7-one, 4-(2,5-dihydro-3-methyl-5-oxo-2-furyl) hexahydro-3,8,9,11-tetrahydroxyl-4-methyl-10-methylene-, [3R-[3α,3αβ,4β(S*),5aα,8α,9α,9aR*,11R*]-, isobutyrylshikonin, acetylshikonin, deoxyshikonin, germacrone, curcumenone, curzerenone, hesperidin, nobiletin, bavachinin, psoralen, isopsoralen, psoralen dihydroflavone, psoralen isoflavone, dexamethasone, methylprednisolone, prednisolone, cortisone, hydrocortisone, betamethasone, ivacaftor, teriflunomide, icaritin, olaparib, tolvaptan, pomalidomide, voriconazole, fluconazole, apixaban, vitamin K1, vitamin A, vitamin E, vitamin A2, tretinoin, retinol derivatives, enzalutamide, ponicidin, oridonin, chlorthalidone, etoposide, dutasteride, isradipine, butyphthalide, progesterone, rivaroxaban, scutellarin, tipranavir, spironolactone, warfarin, medroxyprogesterone, latanoprost, travoprost, bimatoprost, tafluprost, ezetimibe, felodipine, nifedipine, fenofibrate, ciclosporin, celecoxib, tetrahydrocannabinol, cannabinol, cannabidiol, cannabichromene, tacrolimus, everolimus, rapamycin, carisoprodol, carbamazepine, paricalcitol, eldecalcitol, tacalcitol, doxercalciferol, calcipotriol, budesonide, vitamin D2, calcifediol, calciferol, calcitriol, alfacalcidol, seocalcitol, inecalcitol, falecalcitriol, maxacalcitol, griseofulvin, lopinavir, nabumetone, erdafitinib, allopregnenolone, afamelanotide, solriamfetol, pretomanid, taxol, totaxin, artemisinin, oliceridine, foseltamivir, lurbinectedin, triheptanoin, tocotrienol, 4-[(1E,3S)-3-vinyl-3,7-dimethyl-1,6-octadien-1-yl]phenol, 7-hydroxy-3-[4-hydroxy-3-(3-methyl-2-buten-1-yl)phenyl]-4H-1-benzopyran-4-one, 3-[3-[(2E)-3,7-dimethyl-2,6-octadien-1-yl]-4-hydroxyphenyl]-7-hydroxy-4H-1-benzopyran-4-one, (2E)-1-[2,4-dihydroxy-3-(3-methyl-2-butenyl)phenyl]-3-(4-hydroxyphenyl)-2-propen-1-one, (6E,8E,10E,12E,14E,16E,18E,20E,22E,24E,26E)-2,6,10,14,19,23,27,31-octamethyl-2,6,8,10,12,14,16,18,20,22,24,26,30-diisoamyltriene, 2-[6-(2,4-dihydroxybenzoyl)-5-(2,4-dihydroxyphenyl)-3-methyl-2-cyclohexen-1-yl]-5a,10a-dihydro-1,3,5a,8-tetrahydroxy-10a-(3-methyl-2-buten-1-yl)-11H-benzofuran[3,2-b][1]benzopyran-11-one, (5aR,10aS)-2-[(1S,5S,6R)-6-(2,4-dihydroxybenzoyl)-5-(2,4-dihydroxyphenyl)-3-methyl-2-cyclohexen-1-yl]-5a,10a-dihydro-1,3,8,10a-tetrahydroxy-5a-(3-methyl-2-buten-1-yl)-11H-benzofuran[3,2-b][1]benzopyran-11-one, (2E)-3-(4-hydroxy-2-methoxyphenyl)-1-(4-methoxyphenyl)-2-propen-1-one, 2′,4,4′-trihydroxychalcone 4-(β-D-glucopyranoside), (E)-1-(2,4-dihydroxyphenyl)-3-(4-hydroxyphenyl) propyl-2-ene-1-one, (2E)-3-[5-(1,1-dimethyl-2-propen-1-yl)-4-hydroxy-2-methoxyphenyl]-1-(4-hydroxyphenyl)-2-propen-1-one, (2E)-3-[5-[(1S)-1,2-dimethyl-2-propen-1-yl]-4-hydroxy-2-methoxyphenyl]-1-(4-hydroxyphenyl)-2-propen-1-one, (2E)-3-(3,4-dihydroxy-2-methoxyphenyl)-1-[4-hydroxy-3-(3-methyl-2-buten-1-yl)phenyl]-2-propen-1-one, (2S)-2,3-dihydro-7-hydroxy-2-(4-hydroxyphenyl)-4H-1-benzopyran-4-one, 4′,7-dihydroxyflavanone 4′-β-D-glucopyranoside, 4-[5,7-dimethoxy-6-(3-methyl-2-buten-1-yl)-2H-1-benzopyran-3-yl]-1,3-benzenediyl, 4-[5,7-dimethoxy-6-(3-methyl-2-buten-1-yl)-2H-1-benzopyran-3-yl]-1,3-benzenediol, (2S)-2-[4-(β-D-glucopyranosyl)phenyl]-2,3-dihydro-7-hydroxy-4H-1-benzopyran-4-one, brassinin, carbamoylthioacid (1H-indol-3-ylmethyl)-methyl ester, 2-[3,4-dihydroxy-2,5-di(3-methyl-2-buten-1-yl)phenyl]-2,3-dihydro-5,7-dihydroxy-4H-1-benzopyran-4-one [UNK] (2R,3R)-2-(3,4-dihydroxyphenyl)-2,3-dihydro-3,5,7-trihydroxy-4H-1-benzopyran-4-one, (2R,3R)-2-(3,4-dihydroxyphenyl)-2,3-dihydro-3,5,7-trihydroxy-4H-1-benzopyran-4-one, (3S)-3-[2,4-dihydroxy-3-(3-methyl-2-buten-1-yl)phenyl]-2,3-dihydro-5,7-dihydroxy-4H-1-benzopyran-4-one, 4-[(3R)-3,4-dihydro-7-hydroxy-5-methoxy-6-(3-methyl-2-buten-1-yl)-2H-1-benzopyran-3-yl]-2-(3-methyl-2-buten-1-yl)-1,3-phenyldiol, 4-[(3R)-3,4-dihydro-8,8-dimethyl-2H,8H-benzo[1,2-b: 3,4-b′]-bipyran-3-yl]-1,3-benzenediol, 4-[(3R)-3,4-dihydro-5,7-dimethoxy-6-(3-methyl-2-buten-1-yl)-2H-1-benzopyran-3-yl]-2-(3-methyl-2-buten-1-yl)-1,3-benzenediol, and 5,7-dihydroxy-3-(5-hydroxy-2,2-dimethyl-2H-1-benzopyran-6-yl)-4H-1-benzopyran-4-one; atorvastatin, simvastatin, lovastatin, pravastatin, fluvastatin, rosuvastatin, fosamprenavir, atovaquone, valsartan, candesartan cilexetil, fimasartan, eprosartan, olmesartan, diclofenac sodium, etodolac, furosemide, gemfibrozil, glimepiride, glipizide, glibenclamide, ibuprofen, indomethacin, meloxicam, naproxen, oxaprozin, doxorubicin, butyphthalide, tafamidis, eltrombopag, gambogic acid, neogambogic acid, isogambogic acid, betulinic acid, oleanolic acid, glycyrrhetinic acid, gymnemic acid IV, arjunolic acid, corosolic acid, ursolic acid, asiatic acid, 3-epicorosolic acid, pomolic acid, euscaphic acid, maslinic acid, ganoderic acid, tormentic acid, coenzyme Q10, cryptoxanthin, vitamin E, vitamin D, vitamin B12, fullerene, icariin, icariin I, icariin II, icariin C, icariin B, icariin A, (1′R,2′R)-4,5′-dimethyl-2′-(prop-1-en-2-yl)-1′,2′,3′,4′-tetrahydro-[1,1′-biphenyl]-2,6-diol, (1′R,2′R)-5′-methyl-2′-prop-1-en-2-yl)-4-propyl-1′,2′,3′,4′-tetrahydro-[1,1′-biphenyl]-2,6-diol, (1′R,2′R)-4-butyl-5′-methyl-2′-(prop-1-en-2-yl)-1′,2′,3′,4′-tetrahydro-[1,1′-biphenyl]-2,6-diol, (1′R,2′R)-2,6-dihydroxy-5′-methyl-4-pentyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-2′-(prop-1-en-2-yl)-4-propyl-1′,2′,3′,4′-tetrahydro-[1,1′-biphenyl]-3-carboxylic acid, (1′R,2′R)-6-methoxy-5′-methyl-4-pentyl-2′-(prop-1-en-2-yl)-1′,2′,3′,4′-tetrahydro-[1,1′-biphenyl]-2-ol, 5′-methyl-4-pentyl-2′-(prop-1-en-2-yl)-[1,1′-biphenyl]-2,6-diol, 5′-methyl-2′-(prop-1-en-2-yl)-4-propyl-[1,1′-biphenyl]-2,6-diol, (1R,6R)-2′,6′-dihydroxy-4′-pentyl-6-(prop-1-en-2-yl)-1,4,5,6-tetrahydro-[1,1′-biphenyl]-3-carboxylic acid, (1′R,2′R)-5′-(hydroxymethyl)-4-pentyl-2′-(prop-1-en-2-yl)-1′,2′,3′,4′-tetrahydro-[1,1′-biphenyl]-2,6-diol, (5aR,6S,9R,9aR)-6-methyl-3-pentyl-9-(prop-1-en-2-yl)-5a,6,7,8,9,9a-hexahydrodibenzo[b,d]furan-1,6-diol, (2S,3S,4S,5R)-3,4,5-trihydroxy-6-((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)tetrahydro-2H-pyran-2-carboxylic acid, 2-((1S,2S,5S)-5-methyl-2-(prop-1-en-2-yl)cyclohexyl)-5-((E)-styryl)phenyl-1,3-diol, 5-((E)-2-hydroxystyryl)-2-((1S,2S,5S)-5-methyl-2-(prop-1-en-2-yl)cyclohexyl)phenyl-1,3-diol, 5-(benzofuran-2-yl)-2-(1S,2S,5S)-5-methyl-2-(prop-1-en-2-yl)cyclohexyl)phenyl-1,3-diol, (1'S,2'S)-2′-(5-hydroxy-6-methylheptyl-1,6-dien-2-yl)-4,5′-dimethyl-1′,2′,3′,4′-tetrahydro-[1,1′-biphenyl]-2,6-diol, 3-phenyl-1-((1'S,2'S)-2,4,6-trihydroxy-5′-methyl-2′-(prop-1-en-2-yl)-1′,2′,3′,4′-tetrahydro-[1,1′-biphenyl]-3-yl) propan-1-one, (1'S,2'S)-5′-methyl-4-pentyl-2′-(propanediol-1-en-2-yl)-1′,2′,3′,4′-tetrahydro-[1,1′-biphenyl]-2,6-diol, (1'S,2'S)-2′-isopropyl-5′-methyl-4-pentyl-1′,2′,3′,4′-tetrahydro-[1,1′-biphenyl]-2,6-diol, 2-((1R,2S)-2-isopropyl-5-methylcyclohexyl)-5-pentylphenyl-1,3-diol, (1'S,2'S)-5′-(hydroxymethyl)-2′-isopropyl-4-pentyl-1′,2′,3′,4′-tetrahydro-[1,1′-biphenyl]-2,6-diol, (1′R,2'S)-5′-(hydroxymethyl)-2′-isopropyl-4-pentyl-1′,2′,3′,4′-tetrahydro-[1,1′-biphenyl]-2,6-diol, (1′R,2′R)-5′-methyl-4-(2-methyloctan-2-yl)-2′-(prop-1-en-2-yl)-1′,2′,3′,4′-tetrahydro-[1,1′-biphenyl]-2,6-diol, (1R,6R)-2′,6′-dihydroxy-4′-(2-methyloctan-2-yl)-6-(prop-1-en-2-yl)-1,4,5,6-tetrahydro-[1,1′-biphenyl]-3-carboxylic acid, (1′R,2′R)-5′-(hydroxymethyl)-4-(2-methyloctan-2-yl)-2′-(prop-1-en-2-yl)-1′,2′,3′,4′-tetrahydro-[1,1′-biphenyl]-2,6-diol, (1R,2R)-2′,6′-dimethoxy-5-methyl-4′-(2-methyloctan-2-yl)-2-(prop-1-en-2-yl)-1,2,3,4-tetrahydro-1,1′-biphenyl, (1'S,2'S)-2′-isopropyl-5′-methyl-4-(2-methyloctan-2-yl)-1′,2′,3′,4′-tetrahydro-[1,1′-biphenyl]-2,6-diol, 2-((1R,2S)-2-isopropyl-5-methylcyclohexyl)-5-(2-methyloctan-2-yl)phenyl-1,3-diol, ((1S,4S,5S)-4-(2,6-dimethoxy-4-(2-methyloctan-2-yl)phenyl)-6,6-dimethylbicyclo[3.1.1]hept-2-en-2-yl) methanol, ((1R,4R,5R)-4-(2,6-dimethoxy-4-(2-methyloctan-2-yl)phenyl)-6,6-dimethylbicyclo[3.1.1]hept-2-en-2-yl) methanol, 1-(3-((1′R,2′R)-2,6-dihydroxy-5′-methyl-2′-(prop-1-en-2-yl)-1′,2′,3′,4′-tetrahydro-[1,1′-biphenyl]-4-yl)methyl) azetidin-1-yl) ethanone, (1′R,2′R)-4-(2-(1H-1,2,3-triazol-1-yl)ethyl)-5′-methyl-2′-(prop-1-en-2-yl)-1′,2′,3′,4′-tetrahydro-[1,1′-biphenyl]-2,6-diol, 2-((1′R,2′R)-2,6-dihydroxy-5′-methyl-2′-(prop-1-en-2-yl)-1′,2′,3′,4′-tetrahydro-[1,1′-biphenyl]-4-yl)-1-morpholinoethanone, (1′R,2′R)-4-(4-hydroxybutyl)-5′-methyl-2′-(prop-1-en-2-yl)-1′,2′,3′,4′-tetrahydro-[1,1′-biphenyl]-2,6-diol, 4-((1′R,2′R)-2,6-dihydroxy-5′-methyl-2′-(prop-1-en-2-yl)-1′,2′,3′,4′-tetrahydro-[1,1′-biphenyl]-4-yl) butyric acid, (1′R,2′R)-4-(2-ethoxyethyl)-5′-methyl-2′-(prop-1-en-2-yl)-1′,2′,3′,4′-tetrahydro-[1,1′-biphenyl]-2,6-diol, (1′R,2′R)-3-chloro-5′-methyl-4-pentyl-2′-(prop-1-en-2-yl)-1′,2′,3′,4′-tetrahydro-[1,1′-biphenyl]-2,6-diol, (1′R,2′R)-3,5-dichloro-5′-methyl-4-pentyl-2′-(prop-1-en-2-yl)-1′,2′,3′,4′-tetrahydro-[1,1′-biphenyl]-2,6-diol, (1′R,2′R)-3-bromo-5′-methyl-4-pentyl-2′-(prop-1-en-2-yl)-1′,2′,3′,4′-tetrahydro-[1,1′-biphenyl]-2,6-diol, (1′R,2′R)-3,5-dibromo-5′-methyl-4-pentyl-2′-(prop-1-en-2-yl)-1′,2′,3′,4′-tetrahydro-[1,1′-biphenyl]-2,6-diol, (1′R,2′R)-3-iodo-5′-methyl-4-pentyl-2′-(prop-1-en-2-yl)-1′,2′,3′,4′-tetrahydro-[1,1′-biphenyl]-2,6-diol, (1′R,2′R)-3,5-diiodo-5′-methyl-4-pentyl-2′-(prop-1-en-2-yl)-1′,2′,3′,4′-tetrahydro-[1,1′-biphenyl]-2,6-diol, (1′R,2′R)-3-fluoro-5′-methyl-4-pentyl-2′-(prop-1-en-2-yl)-1′,2′,3′,4′-tetrahydro-[1,1′-biphenyl]-2,6-diol, 3-(acetoxy)-2-[(1R,6R)-6-(3-fluoroprop-1-en-2-yl)-3-methylcyclohex-2-en-1-yl]-5-pentylphenyl acetate, (1′R,2′R)-5′-(fluoromethyl)-4-pentyl-2′-(prop-1-en-2-yl)-1′,2′,3′,4′-tetrahydro-[1,1′-biphenyl]-2,6-diol, 1,3-dimethoxy-2-[(1R,6R)-3-methyl-6-prop-1-en-2-ylcyclohex-2-en-1-yl]-5-pentylbenzene, (1′R,2′R)-5′-methyl-4-pentyl-2′-(prop-1-en-2-yl)-1′,2′,3′,4′-tetrahydro-[1,1′-biphenyl]-2-ol, (1R,6R)-2′,6′-diacetoxy-4′-pentyl-6-(prop-1-en-2-yl)-1,4,5,6-tetrahydro-[1,1′-biphenyl]-3-carboxylic acid, 2-((1′R,2′R)-6-hydroxy-5′-methyl-4-pentyl-2′-(prop-1-en-2-yl)-1′,2′,3′,4′-tetrahydro-[1,1′-biphenyl]-2-yl)oxy) acetic acid, (1′R,2′R)-6-(3-aminopropoxy)-5′-methyl-4-pentyl-2′-(prop-1-en-2-yl)-1′,2′,3′,4′-tetrahydro-[1,1′-biphenyl]-2-ol, 2-[3-(cyanomethoxy)-2-[(1R,6R)-3-methyl-6-(prop-1-en-2-yl)cyclohex-2-en-1-yl]-5-pentylphenoxy]acetonitrile, 3-({[(diethylamino)methoxy]carbonyl}oxy)-2-[(1R,6R)-3-methyl-6-(prop-1-en-2-yl)cyclohex-2-en-1-yl]-5-pentylphenyl(diethylamino)methyl carbonate, 3-({2-[(tert-butyldimethylsilyl)oxy]acetoxy)-2-[(1R,6R)-3-methyl-6-(prop-1-en-2-yl)cyclohex-2-en-1-yl]-5-pentylphenyl 2-[(tert-butyldimethylsilyl)oxy]acetate, 3-(acetoxy)-2-[(1R,6R)-3-methyl-6-(3-oxoprop-1-en-2-yl)cyclohex-2-en-1-yl]-5-pentylphenyl acetate, 3-(acetoxy)-2-[(1R,6R)-3-methyl-4-oxo-6-(prop-1-en-2-yl)cyclohex-2-en-1-yl]-5-pentylphenyl acetate, 3-(acetoxy)-2-[(1R,6R)-4-(acetoxy)-3-methyl-6-(prop-1-en-2-yl)cyclohex-2-en-1-yl]-5-pentylphenyl acetate, 2-[(1R,2R)-2-[2,6-di(acetoxy)-4-pentenyl]-4-methylcyclohex-3-en-1-yl]prop-2-en-1-yl acetate, 3-hydroxy-2-[(1R,6R)-3-methyl-6-prop-1-en-2-ylcyclohex-2-en-1-yl]-5-pentylcyclohex-2,5-dien-1,4-dione, 2,5-cyclohexadien-1,4-dione, 2-hydroxy-3-((1R,6R)-3-methyl-6-(1-methylvinyl)-2-cyclohexen-1-yl)-6-pentyl-5-(butamino), 2,5-cyclohexadien-1,4-dione, 2-hydroxy-3-((1R,6R)-3-methyl-6-(1-methylvinyl)-2-cyclohexen-1-yl)-6-pentyl-5-((benzyl)amino), 5-methyl-4-[(1R,6R)-3-methyl-6-prop-1-en-2-ylcyclohex-2-en-1-yl]phenyl-1,3-diol, 4-[(1R,6R)-3-methyl-6-prop-1-en-2-ylcyclohex-2-en-1-yl]-5-pentylphenyl-1,3-diol, 2-[(2E)-3,7-dimethylocta-2,6-dienyl]-5-pentylphenyl-1,3-diol, 1-[(1R,2R,3R,4R)-3-(2,6-dihydroxy-4-pentylphenyl)-2-hydroxy-4-prop-1-en-2-ylcyclopentyl]ethanone.

The above targets may be free acids or free bases, or in the form of salts such as hydrochloride, sulfate, phosphate, fumarate, tartrate, hemifumarate, maleate, tartarate, bitartrate, methanesulfonate, citrate, tosylate, bromate, sulfite, carbonate, bicarbonate, malate, succinate, ethanesulfonate, acetate, and formate, or sodium salts, iron salts, potassium salts, etc.

The natural flavonoids selected for the present invention are a series of compounds formed by two benzene rings connected to each other through three carbon atoms, namely, a class of compounds with a C6-C3-C6 structure as the basic skeleton, and chalcone compounds formed by C3 ring opening on this basis, including but not limited to the following:

TABLE 1
Basic information of natural flavonoids

| Class | Isorhamnetin derivatives (0.45 mg/ml) | | | | |
|---|---|---|---|---|---|
| Struct- ural formul- la | 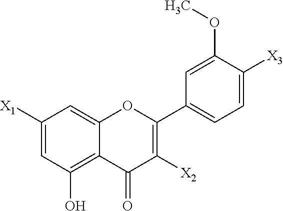 | | | | |
| | Unless otherwide specified, X₁, X₂, and X₃ are OH | | | | |
| Gener- | Isorhamnetin | Isorhamentin | Isorhamnetin 3- | Typhaneoside | Isorhamnetin |
| al | 3-glucoside | 3-O-β-D- | O-β- | | 3-O- |
| English | | rutinoside | gentiobioside | | galactoside |
| name | | | | | |
| CAS | 5041-82-7 | 604-80-8 | 17429-69-5 | 104472-68-6 | 6743-92-6 |
| X₂ | 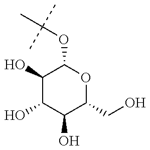 | 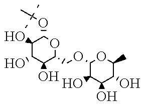 | 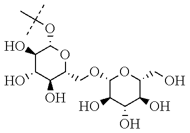 | 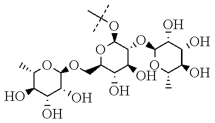 | 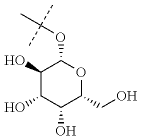 |
| English | 5,7-dihydroxy-2- | 5,7-dihydroxy-2- | 3-[(6-O-β-D- | 3- | (5,7- |
| chemi- | (4-hydroxy-3- | (4-hydroxy-3- | Glucopyranosyl-β- | [(3R,4S,5S,6R)- | dihydroxy- |
| cal | methoxyphenyl)- | methoxyphenyl)- | D- | 4,5-dihydroxy-3- | 2-(4- |
| name | 3-[(2R,5S,6R)- | 3- | glucopyranosyl)oxy]- | [(2R,3R,4R,5R,6S)- | hydroxy-3- |
| | 3,4,5-trihydroxy- | [(2S,3R,4S,5S,6R)- | 5,7-dihydroxy-2- | 3,4,5- | methoxyphe- |
| | 6- | 3,4,5- | (4-hydroxy-3- | trihydroxy-6- | nyl)-3- |
| | (hydroxymethyl)ox- | trihydroxy-6- | methoxyphenyl)- | methyloxan-2- | [3,4,5- |
| | an-2- | [[(2R,3R,4R,5R,6S)- | 4H-1-benzopyran- | yl]oxy-6- | trihydroxy- |
| | yl]oxychromen-4- | 3,4,5- | 4-one; | [[(2R,3R,4R,5R,6S)- | 6- |
| | one | trihydroxy-6- | | 3,4,5- | (hydroxymeth- |
| | | methyloxan-2- | | trihydroxy-6- | yl)oxo- |
| | | yl]oxymethyl]oxan- | | methyloxan-2- | 2- |
| | | 2- | | yl]oxymethyl]oxan- | yl]oxybenzo- |
| | | yl]oxychromen-4- | | 2-yl]oxy-5,7- | pyran-4- |
| | | one | | dihydroxy-2-(4- | one) |
| | | | | hydroxy-3- | |
| | | | | methoxyphenyl)chro- | |
| | | | | men-4-one | |
| M.W. | 478.4 | 624.5 | 640.5 | 770.7 | 478.4 |
| Form. | C22H22O12 | C28H32O16 | C28H32O17 | C34H42O20 | C22H22O12 |
| Gener- | Isorhamnetin 3- | Isorhamnetin 3- | Isorhamnetin 3-O- | Isorhamnetin | Isorhamnetin |
| al | O-neohesperidin | O-β-D- | α-rhamnoside | 3-O- | 3- |
| English | | glucuronide | | sophoroside | sophoroside |
| name | | | | | |
| CAS | 55033-90-4 | 36687-76-0 | 67068-82-0 | 53584-69-3 | 107740-46- |
| | | | | | 5 |
| X₂ | 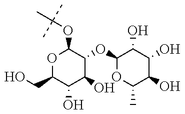 | 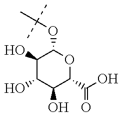 | 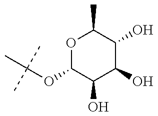 | 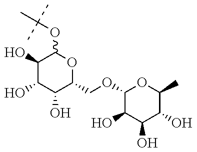 | 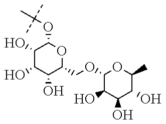 |
| English | 3- | (2S,3S,4S,5R, | 5,7-dihydroxy- | 5,7- | 5,7- [symbol] |
| chemi- | [(2S,3R,4S,5S,6R)- | 6S)-6-[5,7- | 2-(4-hydroxy-3- | dihydroxy-2- | 2-(4-5,7- |
| cal | 4,5- | dihydroxy-2- | methoxyphenyl)- | (4-hydroxy-3- | dihydroxy- |
| name | dihydroxy-6- | (4-hydroxy-3- | 3- | methoxyphenyl)- | 2-(4- |
| | (hydroxymeth- | methoxyphenyl)- | [(2S,3R,4R,5R, | yl)-3- | hydroxy-3- |
| | yl)-3- | 4- | 6S)-3,4,5- | [(3R,4S,5R,6R)- | methoxyphe- |
| | [(2S,3R,4R,5R, | oxobenzopyran- | trihydroxy-6- | 3,4,5- | nyl)-3- |
| | 6S)-3,4,5- | 3-yl]oxy- | methoxy-2- | trihydroxy-6- | [(2S,3R,4S, |
| | trihydroxy-6- | 3,4,5- | yl]oxybenzopyran- | [(2R,3R,4R,5R, | 5R,6R)- |
| | methoxy-2- | trihydroxyalko- | 4-one | 6S)-3,4,5- | 3,4,5- |
| | yl]oxy-2- | xy-2- | | trihydroxy-6- | trihydroxy- |
| | yl]oxy-5,7- | carboxylic | | methyloxan-2- | 6- |
| | dihydroxy-2- | acid | | yl]oxymethyl] | [(2R,3R,4R, |
| | (4-hydroxy-3- | | | oxy-2- | 5R,6S)- |
| | methoxyphenyl) | | | yl]oxybenzopy- | 3,4,5- |
| | yl)benzopyran- | | | ran-4-one | trihydroxy- |
| | 4-one | | | | 6- |
| | | | | | methyloxan- |
| | | | | | 2- |
| | | | | | yl]oxymeth- |
| | | | | | yl]oxyalk- |
| | | | | | 2- |
| | | | | | yl]oxybenzo- |
| | | | | | pyran-4- |
| | | | | | one |
| M.W. | 624.5 | 492.4 | 462.4 | 624.5 | 624.5 |
| Form. | C28H32O16 | C22H20O13 | | | C28H32O16 [symbol] |
| Gener- | Isorhamnetin | Isorhamnetin | Isorhamnetin | Isorhamnetin | Isorhamnetin |
| al | 7-O-glucoside | 7-O-α-L- | 3,4′-di-O-β-D- | 3,7-O- | 3-O- |
| English | | rhamnoside | glucoside | diglucoside | glucoside |
| name | | | | | 7-O- |
| | | | | | rhamnoside |
| CAS | 6743-96-0 | 17331-72-5 | 28288-98-4 | 6758-51-6 | 17331-71-4 |
| X₁ | 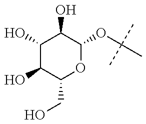 | 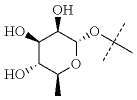 | OH | 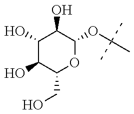 | 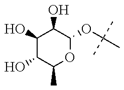 |
| X₂ | OH | OH | 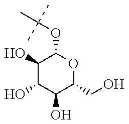 | 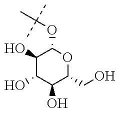 | 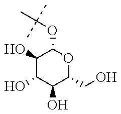 |
| X₃ | OH | OH | 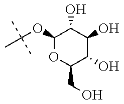 | OH | OH |
| English | 3,5- | 3,5- | 5,7-dihydroxy- | 5-hydroxy-2- | 5-hydroxy- |
| chemi- | dihydroxy-2- | dihydroxy-2- | 2-[3-methoxy- | (4-hydroxy-3- | 2-(4- |
| cal | (4-hydroxy-3- | (4-hydroxy-3- | 4- | methoxyphenyl)- | hydroxy-3- |
| name | methoxyphenyl)- | methoxyphenyl)- | [(2S,3R,4S,5S,6R)- | yl)-3,7- | methoxyphe- |
| | 7- | 7- | 3,4,5- | di[(2S,3R,4S, | nyl)-3- |
| | [(2S,3R,4S,5S, | [(2S,3R,4R,5R, | trihydroxy-6- | 5S,6R)-3,4,5- | [(2S,3R,4S, |
| | 6R)-3,4,5- | 6S)-3,4,5- | (hydroxymethyl) | trihydroxy-6- | 5S,6R)- |
| | trihydroxy-6- | trihydroxy-6- | oxy-2- | (hydroxymeth- | 3,4,5- |
| | (hydroxymeth- | methoxy-2- | yl]oxyphenyl]- | yl)oxo-2- | trihydroxy- |
| | yl)oxo-2- | yl]oxybenzopy- | 3- | yl]oxy]benzopy- | 6- |
| | yl]oxybenzopy- | ran-4-one | [(2S,3R,4S,5S,6R)- | ran-4-one | (hydroxymeth- |
| | ran-4-one | | 3,4,5- | | yl)oxy- |
| | | | trihydroxy-6- | | 2-yl]oxy-7- |
| | | | (hydroxymethyl) | | [(2S,3R,4R, |
| | | | oxy-2- | | 5R,6S)- |
| | | | yl]oxybenzopyran- | | 3,4,5- |
| | | | 4-one | | trihydroxy- |
| | | | | | 6- |
| | | | | | methyloxy- |
| | | | | | 2- |
| | | | | | yl]oxybenzo- |
| | | | | | pyran-4- |
| | | | | | one |
| M.W. | 478.4 | 462.4 | 640.5 | 640.5 | 624.5 |
| Form. | C22H22O12 | C22H22O11 | C28H32O17 | C28H32O17 | C28H32O16 |

| Class | Quercetin (CAS 117-39-5) derivatives EUF (Intrinsic solubility: 1.11) | | | | |
|---|---|---|---|---|---|
| Struct- ural formu- la | 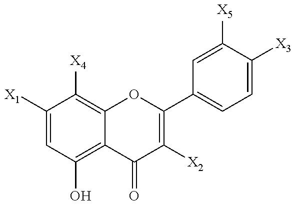 | | | | |
| | Unless otherwise specified, X₄ is H; X₁, X₃, and X₅ are OH | | | | |
| Gener- | Quercetin 3- | Quercetin 3- | Quercetin 3-O- | Quercetin 3- | Quercetin 3- |
| al | O-α-L- | O-β-D- | galactopyranoside | O-rhamnoside | O- |
| English | rhamnopyra- | glucopyranoside | (Hypericin | | glucuronide |
| name | nosyl-β-D- | (Isoquercitrin) | | | |
| | glucopyrano- | | | | |
| | side | | | | |
| CAS | 143016-74-4 | 482-35-9 | 482-36-0 | 522-12-3 | 22688-79-5 |
| X₂ | 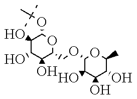 | 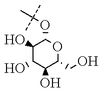 | 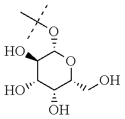 | 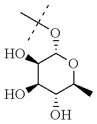 | 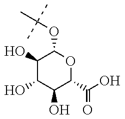 |
| English | 3- | 2-(3,4- | 2-(3,4- | 2-(3,4- | (2S,3S,4S,5R, |
| chemi- | [(2S,3R,4R, | dihydroxyphe- | dihydroxyphenyl)- | dihydroxyphe- | 6S)-6-[2- |
| cal | 5R,6S)-4,5- | nyl)-5,7- | 5,7- | nyl)-5,7- | (3,4- |
| name | dihydroxy- | dihydroxy-3- | dihydroxy-3- | dihydroxy-3- | dihydroxyphe- |
| | 6-methyl-3- | [(2S,3R,4S,5S, | [(2S,3R,4S,5R,6R)- | [(2S,3R,4R,5R, | nyl)-5,7- |
| | [(2S,3R,4S,5S, | 6R)-3,4,5- | 3,4,5- | 6S)-3,4,5- | dihydroxy-4- |
| | 6R)-3,4,5- | trihydroxy-6- | trihydroxy-6- | trihydroxy-6- | oxobenzopyran- |
| | trihydroxy- | (hydroxymeth- | (hydroxymethyl) | methoxy-2- | 3-yl]oxy- |
| | 6- | yl)oxo-2- | oxo-2- | yl]oxybenzopy- | 3,4,5- |
| | (hydroxymeth- | yl]oxybenzopy- | yl]oxybenzopyran- | ran-4-one | trihydroxyox- |
| | yl)oxo-2- | ran-4-one | 4-one | | ane-2- |
| | yl]oxo-2- | | | | carboxylic |
| | (3,4- | | | | acid |
| | dihydroxyphe- | | | | |
| | nyl)-5,7- | | | | |
| | dihydroxyben- | | | | |
| | zopyran-4- | | | | |
| | one | | | | |
| M.W. | 610.5 | 464.4 | 464.4 | 448.4 | 478.4 |
| Form. | C27H30O6 | C21H20O12 | C21H20O12 | C21H20O11 | C21H18O13 |
| Gener- | Quercetin 3- | Quercetin 3-α- | Quercetin-3-O- | α- | Quercetin 3- |
| al | O-β-D- | L- | α-L-arabinoside | glucopyranoru- | O-β-D- |
| English | glucofuranoside | arabinosurano- | | tin | xyloside |
| name | | side | | | |
| CAS | 21637-25-2 | 572-30-5 | 22255-13-6 | 130603-71-3 | 549-32-6 |
| X₂ | 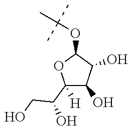 | 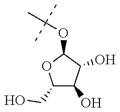 | 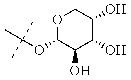 | 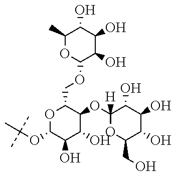 | 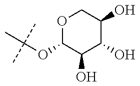 |
| English | 3- | 3- | 2-(3,4- | 2-(3,4- | 2-(3,4- |
| chemi- | [(2S,3R,4R, | [(2S,3R,4R,5S)- | dihydroxyphenyl)- | dihydroxyphe- | dihydroxyphe- |
| cal | 5R)-5-[(1R)- | 3,4- | 5,7- | nyl)-3- | enyl)-5,7- |
| name | 1,2- | dihydroxy-5- | dihydroxy-3- | [(2S,3R,4R,5S, | dihydroxy-3- |
| | dihydroxyeth- | (hydroxymeth- | [(2S,3R,4S,5)- | 6R)-3,4- | [(2S,3R,4S,5S)- |
| | yl]-3,4- | yl)oxo-2-(3,4- | 3,4,5- | dihydroxy-5- | 3,4,5- |
| | dihydroxyox- | dihydroxyphe- | trihydroxyoxy- | [(2R,3R,4S,5S, | trihydroxyoxy- |
| | azol-2- | nyl)-5,7- | 2- | 6R)-3,4,5- | 2- |
| | yl]oxy-2- | dihydroxyben- | yl]oxybenzopyran- | trihydroxy-6- | yl]oxybenzo- |
| | (3,4- | zopyran-4-one | 4-one | (hydroxymeth- | pyran-4-one |
| | dihydroxyphe- | | | yl)oxan-2- | |
| | nyl)-5,7- | | | yl]oxy-6- | |
| | dihydroxyben- | | | [(2R,3R,4R,5R, | |
| | zopyran-4- | | | 6S)-3,4,5- | |
| | one | | | trihydroxy-6- | |
| | | | | methyloxan-2- | |
| | | | | yl]oxy-5,7- | |
| | | | | dihydroxyben- | |
| | | | | zopyran-4-one | |
| M.W. | 464.4 | 434.3 | 434.4 | 772.7 | 434.3 |
| Form. | C21H20O12 | C20H18O11 | C20H18O11 | C33H40O21 | C20H18O11 |
| Gener- | Quercetin 7- | Quercetin 4′- | Quercetin 3,4′- | Quercetin 3- | Quercetin 3- |
| al | O-β- | O-β-D- | O-β-diglucoside | glucoside-7- | O-- |
| English | glucopyrano- | glucopyranoside | | glucoside | rutinoside |
| name | side | | | | |
| CAS | 491-50-9 | 20229-56-5 | 29125-80-2 | 6892-74-6 | 153-18-4 |
| X₁ | 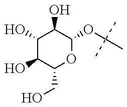 | OH | OH | 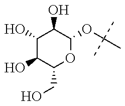 | OH |
| X₂ | OH | OH | 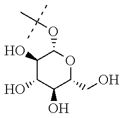 | 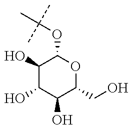 | 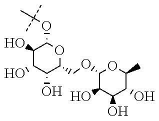 |
| X₃ | OH | 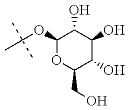 | 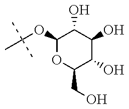 | OH | OH |
| English | 2-(3,4- | 3,5,7- | 5,7-dihydroxy- | 2-(3,4- | 2-(3,4- |
| chemi- | dihydroxyphe- | trihydroxy-2- | 2-[3-hydroxy-4- | dihydroxyphe- | dihydroxyphe- |
| cal | nyl)-5,7- | [3-hydroxy-4- | [(2S,3R,4S,5S,6R)- | nyl)-5- | enyl)-5,7- |
| name | dihydroxy- | [(2S,3R,4S,5S, | 3,4,5- | hydroxy-3,7- | dihydroxy-3- |
| | 3- | 3,4,5- | trihydroxy-6- | di[(2S,3R,4S,5S, | [(2S,3R,4S,5S, |
| | [(2S,3R,4S,5S, | trihydroxy-6- | (hydroxymethyl) | 6R)-3,4,5- | 6R)-3,4,5- |
| | 6R)-3,4,5- | (hydroxymeth- | oxan-2- | trihydroxy-6- | trihydroxy- |
| | trihydroxy- | yl)oxan-2- | yl]oxyphenyl]- | (hydroxymeth- | 6- |
| | 6- | yl]oxyphenyl] | 3- | yl)oxo-2- | [(2R,3R,4R, |
| | (hydroxymeth- | benzopyran-4- | [(2S,3R,4S,5S,6R)- | yl]oxy]benzopy- | 5R,6S)- |
| | yl)oxo-2- | one | 3,4,5- | ran-4-one | 3,4,5- |
| | yl]oxybenzo- | | trihydroxy-6- | | trihydroxy- |
| | pyran-4-one | | (hydroxymethyl) | | 6- |
| | | | oxan-2- | | methyloxan- |
| | | | yl]oxybenzopyran- | | 2- |
| | | | 4-one | | yl]oxymethyl] |
| | | | | | oxy-2- |
| | | | | | yl]oxybenzo- |
| | | | | | pyran-4-one |
| M.W. | 464.4 | 464.4 | 626.5 | 626.5 | 610.5 |
| Form. | C21H20O12 | C21H20O12 | C27H30O17 | C27H30O17 | C27H30O16 |
| Gener- | Quercetin 3- | Quercetin-3- | Gossypetn 8- | Quercetin 3- | Quercetin |
| al | O-glucoside- | O-sophoroside | glucoside | O-rutinoside- | 3,7-dis-O- |
| English | 7-O- | | | (1 → 2)-O- | rhamnoside |
| name | rhamnoside | | | rhamnoside | |
| CAS | 18016-58-5 | 18609-17-1 | 652-78-8 | 55696-57-6 | 28638-13-3 |
| X₁ | 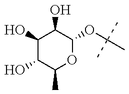 | OH | OH | OH | 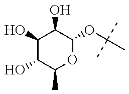 |
| X₂ | 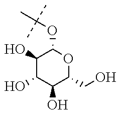 | 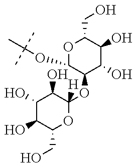 | OH | 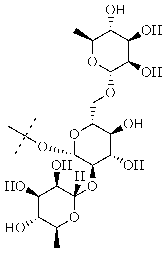 | 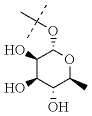 |
| X₄ | H | H | 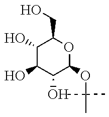 | H | H |
| English | 2-(3,4- | 3- | 2-(3,4- | 2-(3,4- | 2-(3,4- |
| chemi- | dihydroxyphe- | [(2S,3R,4S,5S, | dihydroxyphenyl)- | dihydroxyphe- | dihydroxyphe- |
| cal | nyl)-5- | 6R)-4,5- | 3,5,7- | nyl)-3- | nyl)-5- |
| name | hydroxy-3- | dihydroxy-6- | trihydroxy-8- | [(2S,3R,4S,5S, | hydroxy-3,7- |
| | [(2S,3R,4S,5S, | (hydroxymeth- | [(2S,3R,4S,5S,6R)- | 6R)-4,5- | di[(2S,3R,4R, |
| | 6R)-3,4,5- | yl)-3- | 3,4,5- | dihydroxy-3- | 5R,6S)- |
| | trihydroxy- | [(2S,3R,4S,5S, | trihydroxy-6- | [(2R,3R,4R,6S)- | 3,4,5- |
| | 6- | 6R)-3,4,5- | (hydroxymethyl) | 3,4,5- | trihydroxy- |
| | (hydroxymeth- | trihydroxy-6- | oxy-2- | trihydroxy-6- | 6-methoxy- |
| | yl)oxy-2- | (hydroxymeth- | yl]oxybenzopyran- | methoxy-2- | 2- |
| | yl]oxy-7- | yl)oxo-2- | 4-one | yl]oxy-6- | yl]oxy]benzo- |
| | [(2S,3R,4R, | yl]oxo-2-(3,4- | | [(2R,3R,4R,5R, | pyran-4- |
| | 5R,6S)- | dihydroxyphe- | | 6S)-3,4,5- | one |
| | 3,4,5- | nyl)-5,7- | | trihydroxy-6- | |
| | trihydroxy- | dihydroxyben- | | methoxy]oxy] | |
| | 6- | zopyran-4-one | | oxy]oxymethyl] | |
| | methyloxy- | | | oxy]oxy-5,7- | |
| | 2- | | | dihydroxyben- | |
| | yl]oxybenzo- | | | zopyran-4-one | |
| | pyran-4-one | | | | |
| M.W. | 610.5 | 626.5 | 480.4 | 772.7 | 594.5 |
| Form. | C27H30O16 | C27H30O17 | [symbol] | [symbol] | [symbol] |
| Gener- | Quercetin 3- | Qercetin 3′- | Quercetin 7-O- | Quercetin 3- | Quercetin 3- |
| al | neohesperidin | O-glucoside | rhamnoside | sambubioside | O[β-D- |
| English | | | | | glucosyl- |
| name | | | | | (1 → 6)-β-D- |
| | | | | | glucoside] |
| CAS | 32453-36-4 | 19254-30-9 | 22007-72-3 | 83048-35-5 | 7431-83-6 |
| X₁ | OH | OH | 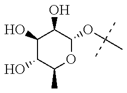 | OH | OH |
| X₂ | 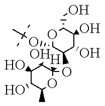 | OH | OH | 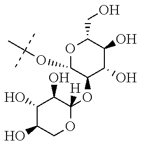 | 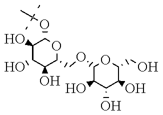 |
| X5 | OH | 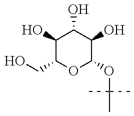 | OH | OH | OH |
| English | 3- | 3,5,7- | 2-(3,4- | 3- | 2-(3,4- |
| chemi- | [(2S,3R,4S,5S, | trihydroxy-2- | dihydroxyphenyl)- | [(2S,3R4S,5S, | dihydroxyphe- |
| cal | 6R)-4,5- | [4-hydroxy-3- | 5,7- | 6R)-4,5- | nyl)-5,7- |
| name | dihydroxy- | [(2S,3R,4S,5S, | dihydroxy-3- | dihydroxy-6- | dihydroxy-3- |
| | 6- | 6R)-3,4,5- | [(2S,3R,4R,5R, | (hydroxymeth- | [(2S,3R,4S,5S, |
| | (hydroxymeth- | trihydroxy-6- | 6S)-3,4,5- | yl)-3- | 6R)-3,4,5- |
| | yl)-3- | (hdyroxymeth- | trihydroxy-6- | [(2S,3R,4S,5R)- | trihydroxy- |
| | [(2S,3R,4R, | yl)oxy-2- | methoxy-2- | 3,4,5- | 6- |
| | 5R,6S)- | yl]oxyphenyl] | yl]oxybenzopyran- | trihydroxyoxo- | [(2R,3R,4S, |
| | 3,4,5- | benzopyran-4- | 4-one | 2-yl]oxo-2- | 5S,6R)- |
| | trihydroxy- | one | | yl]oxo-2-(3,4- | 3,4,5- |
| | 6-methoxy- | | | dihydroxyphe- | trihydroxy- |
| | 2-yl]ox-2- | | | nyl)-5,7- | 6- |
| | (3,4- | | | dihydroxyben- | (hydroxymeth- |
| | dihydroxyphe- | | | zopyran-4-one | yl)oxo-2- |
| | nyl)-5,7- | | | | yl]oxybenzo- |
| | dihydroxypyran-4- | | | | yl]oxo-2- |
| | zopyran-4- | | | yl]oxybenzo- | |
| | one | | | | pyran-4-one |
| M.W. | 610.5 | 464.4 | 448.4 | 596.5 | 626.5 |
| Form. | C27H30O16 | C21H19O12 | C21H20O11 | C26H28O16 | C27H30O17 |
| Gener- | Quercetin-3- | Quercetin 3- | Quercetin 7- | Quercetin 3- | Quercetin-3- |
| al | arabinoglucoside | O-(6″-O-α-L- | glucoronide | rhamnoside 7- | O-(6′-O- |
| English | | rhamnopyrano- | | glucoside | galloy) |
| name | | syl)-β-D- | | | acid)-β-D- |
| | | 7-O-β-D- | | | galactopyrano- |
| | | glucopyranoside | | | side |
| CAS | 23284-18-6 | 30311-61-6 | 38934-20-2 | 17306-45-5 | 53171-28-1 |
| X₁ | OH | 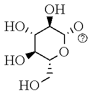 | 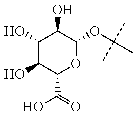 | 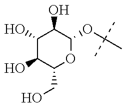 | OH |
| X₂ | 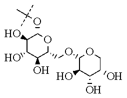 | 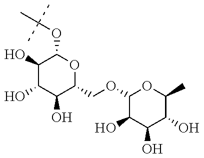 | OH | 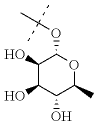 | 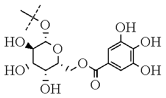 |
| English | 2-(3,4- | 2-(3,4- | (2S,3S,4S,5R,6S)- | 2-(3,4- | [(2R,3R,4S, |
| chemi- | dihydroxyphe- | dihydroxyphe- | 6-[2-(3,4- | dihydroxyphe- | 5R,6S)-6-[2- |
| cal | nyl)-5,7- | nyl)-5- | dihydroxyphenyl)- | nyl)-5- | (3,4- |
| name | dihydroxy- | hydroxy-7- | 5,7- | hydroxy-3- | dihydroxyphe- |
| | 3- | [(2S,3R,4S,5S, | dihydroxy-4- | [(2S,3R,4S,5S, | nyl)-5,7- |
| | [(2S,3R,4S,5S, | 6R)-3,4,5- | oxobenzopyran- | 6R)-3,4,5- | dihydroxy-4- |
| | 6R)-3,4,5- | trihydroxy-6- | 3-yl]oxy-3,4,5- | trihydroxy-6- | oxochromen- |
| | trihydroxy- | (hydroxymeth- | trihydroxyoxane | (hydroyxmeth- | 3-yl]oxy- |
| | 6- | yl)oxan-2- | 2-carboxylic | yl)oxy-2- | 3,4,5- |
| | [(2S,3R,4S,5S)- | yl]oxy-3- | acid | yl]oxy-7- | trihydroxyox- |
| | 3,4,5- | [(2S,3R,4S,5S, | | [(2S,3R,4R,5R, | an-2- |
| | trihydroxy- | 6R)-3,4,5- | | 6S)-3,4,5- | yl]methyl |
| | 2- | trihydroxy-6- | | trihydroxy-6- | 3,4,5- |
| | yl]oxymethyl] | methoxane] | | methyloxy-2- | trihydroxyben- |
| | oxy-2- | oxymethyl]ox- | | yl]oxybenzopy- | zoate |
| | yl]oxybenzo- | an-2- | | ran-4-one | |
| | pyran-4-one | yl]oxybenzopy- | | | |
| | | ran-4-one | | | |
| M.W. | 596.5 | 772.7 | 478.4 | 610.5 | 616.5 |
| Form. | C26H28O16 | C33H40O21 | C21H18O13 | C27H30O16 | C28H24O16 |
| Gener- | Quercetin 3- | Quercetin 3- | Quercetin 3-O- | Quercetin 3- | Quercetin 3- |
| al | O-glucosyl- | O-glucosyl- | glucosyl- | O-rhamnosyl- | O- |
| English | xyloside | rhamnosyl- | rhamnosyl- | galactoside | rhamnosyl- |
| name | | galactoside | glucoside | | (1 −> 2)- |
| | | | | | rhamosyl- |
| | | | | | (1 −> 6)- |
| | | | | | glucoside |
| CAS | N/A | 134953-93-8 | N/A | N/A | N/A |
| X₂ | 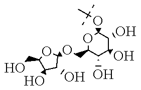 | 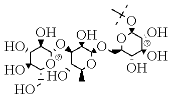 | 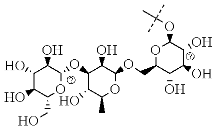 | 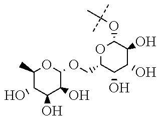 | 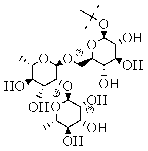 |
| English | 3- | 3- | 3- | 2-(3,4- | 3- |
| chemi- | [(2S,3R,4S,5S, | [(2S,3R,4S,5R, | [(2S,3R,4S,5R,6R)- | dihydroxyphe- | [(2S,3R,4S,5S, |
| cal | 6R)-6- | 6R)-6- | 6- | nyl)-5,7- | R)-6- |
| name | [(2R,3R,4R, | [(2R,3R,4R,5S, | [(2R,3R,4R,5S, | dihydroxy-3- | [(2R,3R,4R, |
| | 5R)-3,4- | 6S)-3,5- | 6S)-3,5- | [(2R,3S,4R,5S, | 5R,6S)-4,5- |
| | dihydroxy- | dihydroxy-6- | dihydroxy-6- | 6S)-3,4,5- | dihydroxy-6- |
| | 5- | methyl-4- | methyl-4- | trihydroxy-6- | methyl-3- |
| | (hydroxymeth- | [(2S,3R,4S,5S, | [(2S,3R,4S,5S,6R)- | [(2S,3S,4S,5S, | [(2S,3R,4R, |
| | yl)oxan-2- | 6R)-3,4,5- | 3,4,5- | 6R)-3,4,5- | 5R,6S)- |
| | yl]oxymethyl]- | trihydroxy-6- | trihydroxy-6- | trihydroxy-6- | 3,4,5- |
| | 3,4,5- | (hydroxymeth- | (hydroxymethyl) | methyloxan-2- | trihydroxy- |
| | trihydroxyox- | yl)oxo-2- | oxo-2-yl]oxo- | yl]oxymethyl] | 6- |
| | y-2-yl]oxy- | yl]oxo-2- | 2- | oxan-2- | methyloxy- |
| | 2-(3,4- | yl]oxophenyl)- | yl]oxophenyl)- | yl]oxybenzopy- | 2-yl]oxy-2- |
| | dihydroxyphe- | 5,7- | 5,7- | ran-4-one | (3,4- |
| | enyl)-5,7- | dihydroxyben- | dihydroxyben- | | dihydroxyphe- |
| | dihydroxyben- | zopyran-4-one | pyran-4-one | | nyl)-5,7- |
| | zopyran-4- | | | | dihydroxyben- |
| | one | | | | zopyran-4- |
| | | | | | one |
| M.W. | 596.5 | 772.7 | 772.7 | 610.5 | 756.7 |
| Form. | C26H28O16 | C33H40O21 | C33H40O21 | C27H30O16 | C33H40O20 |

| Class | Apigenin (520-36-5) derivatives −0.14 mg/ml | | | | |
|---|---|---|---|---|---|
| Struct- ural formu- la | 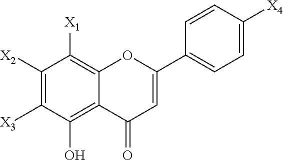 | | | | |
| | Unless otherwise specified, X₁ and X₃ are H; X₄ is OH | | | | |
| Gener- | Apigenin 7- | Apigenin 7-O- | Apigenin 7-O- | Apigenin-7- | Apigenin 7- |
| al | O-β-D- | β- | β-D- | O-rutinoside | O-β-D- |
| English | glucopyranoside | neohesperidin | glucuronide | | apiofuranosyl |
| name | | | | | (1 → 2)-β- |
| | | | | | D- |
| | | | | | glucopyranoside |
| CAS | 578-74-5 | 17306-46-6 | 29741-09-1 | 552-57-8 | 26544-34-3 |
| X₂ | 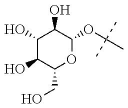 | 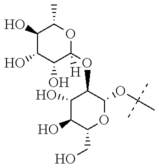 | 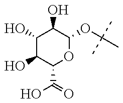 | 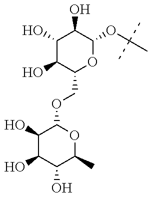 | 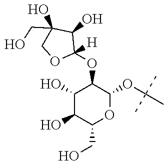 |
| English | 5-hydroxy-2- | 7- | (2S,3S,4S,5R, | 5-hydroxy-2- | 7- |
| chemi- | (4- | [(2S,3R,4S,5S, | 6)-3,4,5- | (4- | [(2S,3R,4S,5S, |
| cal | hydroxyphenyl)- | 6R)-4,5- | trihydroxy-6- | hydroxyphenyl)- | 6R)-3- |
| name | 7- | dihydroxy-6- | [5-hydroxy-2- | 7- | [(2S,3R,4R)- |
| | [(2S,3R,4S,5S, | (hydroxymeth- | (4- | [(2S,3R,4S,5S, | 3,4- |
| | 6R)-3,4,5- | yl)-3- | hydroxyphenyl)- | 6R)-3,4,5- | dihydroxy-4- |
| | trihydroxy-6- | [(3S,3R,4R,5R, | 4- | trihydroxy-6- | (hydroxymeth- |
| | yl)oxo-2- | 6S)-3,4,5- | oxobenzopyran- | [(2R,3R,4R,5R, | yl)oxazolidin- |
| | yl]oxybenzopy- | trihydroxy-6- | 7- | 6S)-3,4,5- | 2-yl]oxy- |
| | ran-4-one | methoxy-2- | yl]oxyoxan-2- | trihydroxy-6- | 4,5- |
| | | yl]oxy-2- | carboxylic | methoxy-2- | dihydroxy-6- |
| | | yl]oxy-5- | acid | yl]oxymethyl] | (hydroxymeth- |
| | | hydroxy-2-(4- | | oxy-2- | yl)oxy-2- |
| | | hydroxyphenyl) | | yl]oxybenzopy- | yl]oxy-5- |
| | | benzopyran- | | ran-4-one | hydroxy-2- |
| | | 4-one | | | (4- |
| | | | | | hydroxyphenyl) |
| | | | | | yl)benzopyran- |
| | | | | | 4-one |
| M.W. | 432.4 | 578.5 | 446.4 | 578.5 | 564.5 |
| Form. | C21H20O10 | C27H30O14 | C21H18O11 | C27H30O14 | C26H28O14 |
| Gener- | Apigenin 7- | Apigenin 7-O- | Ligustroflavone | Apigenin 8-C- | Apigenin |
| al | O-β-D | (6′′′-trans-p- | | β-D- | 6,8-di-C- |
| English | methylglucuro- | coumaroyl-β- | | glucopyranoside | glucopyranoside |
| name | nide | D- | | | |
| | | glucopyranoside | | | |
| CAS | 53538-13-9 | 105815-90-5 | 260413.62-5 | 3681-93-4 | 23666-13-9 |
| X₁ | H | H | H | 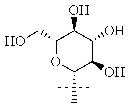 | 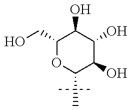 |
| X₂ | 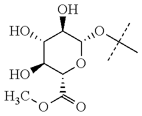 | 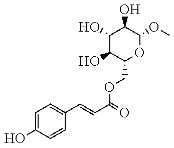 | 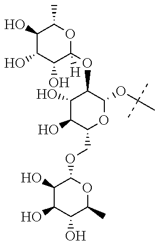 | OH | OH |
| X₃ | H | H | H | H | 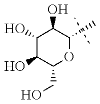 |
| English | Methyl | [(2R,3S,4S,5R, | 7- | 5,7- | 5,7- |
| chemi- | (2S,3S,4S,5R, | 6S)-3,4,5- | (((2S,3R,4S,5S, | dihydroxy-2- | dihydroxy-2- |
| cal | 3,4,5- | trihydroxy-6- | 6R)-4,5- | (4- | (4- |
| name | trihydroxy-6- | [5-hydroxy-2- | dihydroxy-3- | hydroxyphenyl)- | hydroxyphenyl)- |
| | [5-hydroxy-2- | (4- | (((2S,3R,4R,5R, | 8- | yl)-6,8- |
| | (4- | hydroxyphenyl)- | 6S)-3,4,5- | [(2S,3R,4R,5S, | di[(2S,3R,4R, |
| | hydroxphenyl)- | 4- | trihydroxy-6- | 6R)-3,4,5- | 5S,6R)- |
| | 4- | oxobenzopyran- | methyltetrahy- | trihydroxy-6- | 3,4,5- |
| | oxobenzopyran- | 7-yl]oxan-2- | dro-2H-pyran- | (hydroxymeth- | trihydroxy-6- |
| | 7- | yl]methyl(E)- | 2-yl)oxy)-6- | yl)oxy-2- | (hydroxymeth- |
| | yl]oxyoxan-2- | 3-(4- | (((((2R,3R,4R, | yl]benzopyran- | yl)oxo-2- |
| | carboxylate | 5R,6S)-3,4,5- | 5R,6S)-3,4,5- | yl]benzopyran- | 4-one |
| | | prop-2- | trihydroxy-6- | | |
| | | enoate | methyltetrahy- | | |
| | | | 2H-pyran- | | |
| | | | 2- | | |
| | | | yl)oxy)methyl) | | |
| | | | tetrahydro- | | |
| | | | 2H-pyran-2- | | |
| | | | yl)oxy)-5- | | |
| | | | hydroxy-2-(4- | | |
| | | | hydroxyphenyl)- | | |
| | | | 4H- | | |
| | | | benzopyran-4- | | |
| | | | one | | |
| M.W. | 460.4 | 578.5 | 724.7 | 432.4 | 594.5 |
| Form. | C22H20O11 | C30H26O12 | C33H40O18 | | [symbol] |

| | | | | |
|---|---|---|---|---|
| Gener- | Schaftoside, Api- | Apigenin 6-C-α-L- | Apigenin-6-C- | Apigenin 4′-O- |
| al | genin 8-C-α-L- | arabinoside 8-C-β- | glucoside-7-O- | glucoside |
| English | arabinoside 6-C- | D-glucoside | glucoside | glucoside |
| name | β-D-glusoide | | | |
| CAS | 51938-32-0 | 52012-29-0 | 20310-89-8 | 20486-34-4 |
| X₁ | 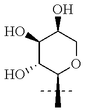 | 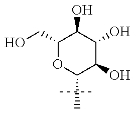 | H | H |
| X₂ | OH | OH | 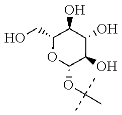 | OH |
| X₃ | 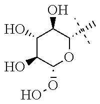 | 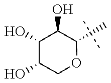 | 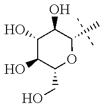 | H |
| X₄ | OH | OH | OH | 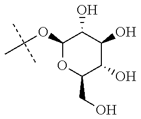 |
| English | 5,7-dihydroxy-2- | 5,7-dihydroxy-2- | 5-hydroxy-2-(4- | (5,7-dihydroxy-2- |
| chemi- | (4- | (4- | hydroxyphenyl)-6- | [4- |
| cal | hydroxyphenyl)- | hydroxyphenyl)-8- | [(2S,3R,4R,5S,6R)- | [(2S,3R,4S,5S,6R)- |
| name | 6- | [(2S,3R,4R,5S,6R)- | 3,4,5-trihydroxy- | 3,4,5- |
| | [(2S,3R,4R,5S,6R)- | 3,4,5-trihydroxy- | 6- | trihydroxy-6- |
| | 3,4,5- | 6- | (hydroxymethyl)ox- | (hydroxymethyl)o- |
| | trihydroxy-6- | (hydroxymethyl)o- | xan-2-yl]-7- | o-2- |
| | (hydroxymethyl) | xan-2-yl]-6- | [(2S,3R,4R,5S,6R)- | yl]oxyphenyl]ben- |
| | oxan-2-yl]-8- | [(2S,3R,4S,5S)- | 3,4,5-trihydroxy- | zopyran-4-one |
| | [(2S,3R,4S,5S)- | 3,4,5- | 6- | |
| | 3,4,5- | trihydroxyoxan-2- | (hydroxymethyl)o- | |
| | trihydroxyoxan- | yl]chromen-4-one | xan-2- | |
| | 2-yl]chromen-4- | | yl]oxybenzopyran- | |
| | one | | 4-one | |
| M.W. | 564.5 | 564.5 | 594.5 | 432.4 |
| Form. | C26H28O16 | C26H28O14 | C27H30O15 | C21H20O10 |

| Class | Myricetin (529-44-2) derivatives −3.14 mg/ml | | | | |
|---|---|---|---|---|---|
| Structu- ural formu- la | 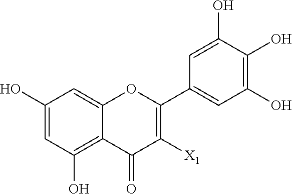 | | | | |
| Gener- | Myricitrin | Myricetin 3-O- | Myricetin 3- | Myricetin 3- | Myricetin 3- |
| al | | beta-D- | O-β-D- | O- | O-rutinoside) |
| English | | glucopyranoside | galactoside | glucuronide | |
| name | | | | | |
| CAS | 17912-87-7 | 19833-12-6 | 15648-86-9 | 77363-65-6 | 41093-68-9 |
| X₁ | 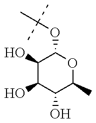 | 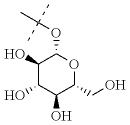 | 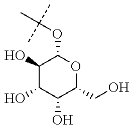 | 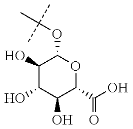 | 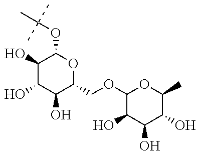 |
| English | 5,7- | 5,7-dihydroxy- | 5,7- | (2S,3S,4S,5R, | 5,7- |
| chemi- | dihydroxy-3- | 3- | dihydroxy-3- | 6S)-6-[5,7- | dihydroxy-2- |
| cal | [(2S,3R,4R,5R, | [(2S,3R,4S,5S,6R)- | [(2S,3R,4S,5R, | dihydroxy-4- | (3,4,5- |
| name | 6S)-3,4,5- | 3,4,5- | 6R)-3,4,5- | oxo-2-(3,4,5- | trihydroxyphe- |
| | trihydroxy-6- | trihydroxy-6- | trihydroxy-6- | trihydroxyphe- | nyl)-3- |
| | methoxy-2- | (hydroxymethyl) | (hydroxymeth- | nyl)benzopyran- | [(2S,5S)- |
| | yl]oxy-2- | oxy-2-yl]oxy- | yl)oxy-2- | 3-yl]oxy- | 3,4,5- |
| | (3,4,5- | 2-(3,4,5- | yl]oxy-2- | 3,4,5- | trihydroxy-6- |
| | trihydroxyphe- | trihydroxyphenyl) | (3,4,5- | trihydroxyoxo- | [(2R,4S,5R)- |
| | yl)benopyran- | benzopyran- | trihydroxyphe- | 2-carboxylic | 3,4,5- |
| | 4-one | 4-one | enyl)benzopy- | acid | trihydroxy-6- |
| | | | ran-4-one | | methyloxan- |
| | | | | | 2- |
| | | | | | yl]oxymethyl] |
| | | | | | oxy-2- |
| | | | | | yl]oxybenzo- |
| | | | | | pyran-4-one |
| M.W. | 464.4 | 480.4 | 480.4 | 494.4 | 626.5 |
| Form. | C21H20O12 | C21H20O13 | C21H20O13 | C21H18O14 | C27H30O17 |

| Class | Hesperetin (CAS 520-33-2) derivatives (EUF)-0.66 | | | |
|---|---|---|---|---|
| Struct- ural formu- la | 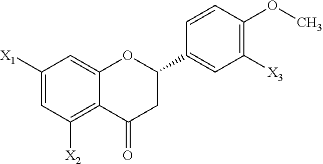 | | | |
| | Unless otherwise specified, X₂ and X₃ are OH | | | |
| Gener- | Hesperetin 7-O- | Hesperetin 7-O- | Hesperetin 7-O-β-D- | α-glucosyl |
| al | rutinoside) | neohesperidin | glucopyranoside | hesperidin |
| English | | | | |
| name | | | | |
| CAS | 520-26-3 | 13241-33-3 | 31712-49-9 | 161713-86-6 |
| X₁ | 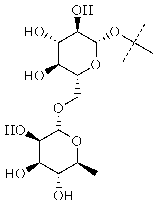 | 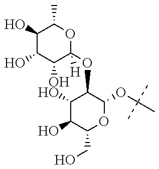 | 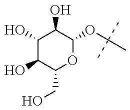 | 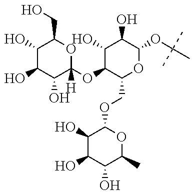 |
| English | (2S)-5-hydroxy- | (2S)-7- | (2S)-5-hydroxy-2- | (2)-7- |
| chemi- | 2-(3-hydroxy-4- | [(2S,3R,4S,5S,6R)- | (3-hydroxy-4- | [(2S,3R,4R,5S,6R)- |
| cal | methoxyphenyl)- | 4,5-dihydroxy-6- | methoxyphenyl)-7- | 3,4- |
| name | 7- | (hydroxymethyl)- | [(2S,3R,4S,5S,6R)- | dihydroxy-5- |
| | [(2S,3R,4S,5S,6R)- | 3- | 3,4,5-trihydroxy-6- | [(2R,3R,4S,5S,6R)- |
| | 3,4,5- | [(2S,3R,4R,5R,6S)- | (hydroxymethyl)oxo- | R)-3,4,5- |
| | trihydroxy-6- | 3,4,5-trihydroxy- | 2-yl]oxy-2,3- | trihydroxy-6- |
| | [(2R,3R,4R,5R,6S)- | 6-methoxy-2- | dihydrobenzopyran- | (hydroxymethyl) |
| | 3,4,5- | yl]oxy-2-yl]oxy-5- | 4-one | oxan-2-yl]oxy- |
| | trihydroxy-6- | hydroxy-2-(3- | | 6- |
| | methyloxan-2- | hydroxy-4- | | [(2R,3R,4R,5R, |
| | yl]oxymethyl]ox- | methoxyphenyl)- | | 6S)-3,4,5- |
| | y-2-yl]oxy-2,3- | 2,3- | | trihydroxy-6- |
| | dihydrobenzopyran- | dihydrobenzopyran- | | methyloxan-2- |
| | 4-one | 4-one | | yl]oxy]oxy-5- |
| | | | | hydroxy-2-(3- |
| | | | | hydroxy-4- |
| | | | | methoxyphenyl)- |
| | | | | 2,3- |
| | | | | dihydrobenzopy- |
| | | | | ran-4-one |
| M.W. | 610.6 | 610.6 | 464.4 | 772.7 |
| Form. | C28H34O15 | C28H34O15 | C22H24O11 | C34H44O20 |

| | | | |
|---|---|---|---|
| Gener- | Hesperetin 7-O-β-D- | Hesperetin 5-O- | Hesperetin 3′-O-β-D- |
| al | glucuronide | glucoside | glucuronide |
| English | | | |
| name | | | |
| CAS | 67322-08-1 | 69651-80-5 | 150985-66-3 |
| X₁ | 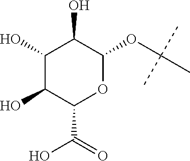 | OH | OH |
| X₂ | OH | 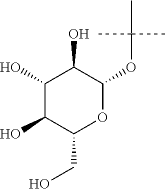 | OH |
| X₃ | OH | OH | 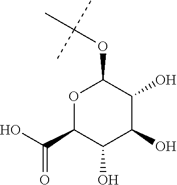 |
| English | (2S,3S,4S,5R,6S)- | (2S)-5-hydroxy-2-(3- | (2S,3S,4S,5R,6S)-6-(5- |
| chemi- | 3,4,5-trihydroxy-6- | hydroxy-4- | ((S)-5,7-dihydroxy-4- |
| cal | [(2S)-5-hydroxy-2-(3- | methoxyphenyl)-7- | oxobenzopyran-2-yl)-2- |
| name | hydroxy-4- | [(2S,3R,4S,5S,6R)-3,4,5- | methoxyphenoxy)-3,4,5- |
| | methoxyphenyl)-4-oxo- | trihydroxy-6- | trihydroxytetrahydro-2H- |
| | 2,3- | (hydroxymethyl)oxo-2- | pyran-2-carboxylic acid |
| | dihydrobenzopyran-7- | yl]oxy-2,3- | |
| | yl]oxy]oxo-2- | dihydrobenzopyran-4- | |
| | carboxylic acid | one | |
| M.W. | 478.4 | 464.4 | 478.40 |
| Form. | C22H22O12 | C22H24O11 | C22H22O12 |

| Class | Luteolin (CAS: 491-70-3) derivatives-0.40 | | | |
|---|---|---|---|---|
| Structu ural formu- la | 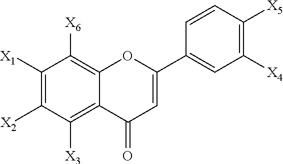 | | | |
| | Unless otherside specified, X₂ and X₆ are H; X₃, X₄, and X₅ are OH | | | |
| Gener- | Luteolin 7-O-β- | Luteolin-7-O-β- | Luteolin 7- | Luteolin 7- |
| al | D- | D-glucuronide | rutinoside | neohesperidin |
| English | glucopyranoside | | | |
| name | | | | |
| CAS | 5373-11-5 | 29741-10-4 | 20633-84-5 | 25694-72-8 |
| X₁ | 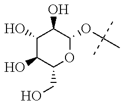 | 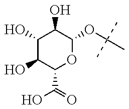 | 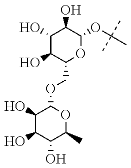 | 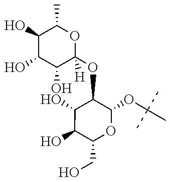 |
| English | 2-(3,4- | (2S,3S,4S,5R,6S)- | 2-(3,4- | 7- |
| chemi- | dihydroxyphenyl)- | 6-[2-(3,4- | dihydroxyphenyl)- | [(2S,3R,5S,6S,6R)- |
| cal | 5-hydroxy-7- | dihydroxyphenyl | 5-hydroxy-7- | 4,5-dihydroxy-6- |
| name | [(2S,3R,4S,5S,6R)- | 5-hydroxy-4- | [(2S,3R,4S,5S,6R)- | (hydroxymethyl)- |
| | 3,4,5- | oxochromen-7- | 3,4,5-trihydroxy- | 3- |
| | trihydroxy-6- | yl]oxy-3,4,5- | 6- | [(2S,3R,4R,5R,6S)- |
| | (hydroxymethyl) | trihydroxyoxane- | [[(2R,3R,4R,5R,6S)- | 3,4,5-trihydroxy- |
| | oxo-2- | 2-carboxylic acid | 3,4,5-trihydroxy- | 6-methoxy-2- |
| | yl]oxybenzopyran- | | 6-methyloxan-2- | yl]oxy-2-(3,4- |
| | 4-one | | yl]oxymethyl]oxan- | dihydroxyphenyl)- |
| | | | 2-yl]oxychromen- | 5-hydroxypyran-4- |
| | | | 4-one | one |
| M.W. | 448.4 | 462.4 | 594.5 | 594.5 |
| Form. | C21H20O11 | C21H18O12 | C27H30O15 | C27H30O15 |
| Gener- | luteolin-7-O- | Luteolin 6-C-β- | Luteolin 8-C-β-D- | Luteolin 6-C-β-D- |
| al | gentiobioside | D-glucoside | glucopyranoside | glucopyranoside-8- |
| English | | | | C-α-L- |
| name | | | | arabinopyranoside |
| CAS | 70855-41-3 | 4261-42-1 | 28608-75-5 | 59952-97-5 |
| X₁ | 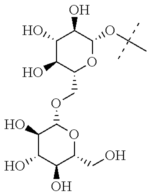 | OH | OH | OH |
| X₂ | H | 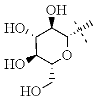 | H | 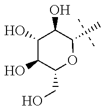 |
| X₆ | H | H | 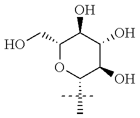 | 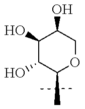 |
| English | 2-(3,4- | 2-(3,4- | 2-(3,4- | 2-(3,4- |
| chemi- | dihydroxyphenyl)- | dihydroxyphenyl)- | dihydroxyphenyl)- | dihydroxyphenyl)- |
| cal | 5-hydroxy-7- | 5,7-dihydroxy- | 5,7-dihydroxy-8- | 5,7-dihydroxy-6- |
| name | [(2S,3R,4S,5S,6R)- | 6- | [(2S,3R,4R,5S,6R)- | [(2S,3R,4R,5S,6R)- |
| | R)-3,4,5- | [(2S,3R,4R,5S,6R)- | 3,4,5-trihydroxy- | 3,4,5-trihydroxy- |
| | trihydroxy-6- | 3,4,5- | 6- | 6- |
| | [[(2R,3R,4S,5S,6R)- | trihydroxy-6- | (hydroxymethyl)ox- | (hydroxymethyl)oxy- |
| | R)-3,4,5- | (hdyroxymethyl) | y-2-yl]benzopyran- | 2-yl]-8- |
| | trihydroxy-6- | oxy-2- | 4-one | [(2S,3R,4S,5S)- |
| | (hydroxymethyl) | yl]benzopyran-4- | | 3,4,5- |
| | oxan-2- | one | | trihydroxyoxy-2- |
| | yl]oxymethyl]ox- | | | yl]benzopyran-4- |
| | an-2- | | | one |
| | yl]oxychromen- | | | |
| | 4-one | | | |
| M.W. | 610.5 | 448.4 | 448.4 | 580.5 |
| Form. | C27H30O16 | C21H20O11 | C21H20O11 | C26H28O15 |

| | | | | | |
|---|---|---|---|---|---|
| Gener- | Luteolin 4′-O- | Luteolin 5-O-β- | Luteolin 3′-β- | Luteolin 3′,7- | Luteolin 7- |
| al | β-D- | D- | D- | O-diglucoside | neohesperidin- |
| English | glucopyranoside | glucopyranoside | glucopyranoside | | 4′- |
| name | | | | | sophorodie |
| CAS | 6920-38-3 | 20344-46-1 | 5154-41-6 | 52187-80-1 | / |
| X₁ | OH | OH | OH | 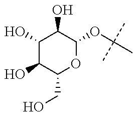 | 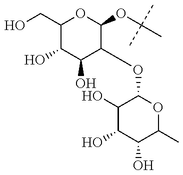 |
| X₃ | OH | 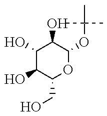 | OH | OH | OH |
| X₄ | OH | OH | 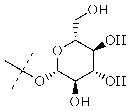 | 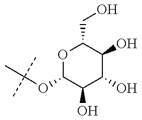 | OH |
| X₅ | 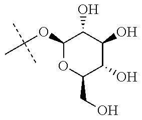 | OH | OH | OH | 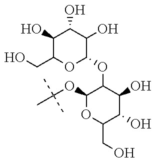 |
| English | 5,7- | 2-(3,4- | 5,7- | 5-hydroxy-2- | 2-[4- |
| chemi- | dihydroxy-2- | dihydroxyphenyl)- | dihydroxy-2- | [4-hydroxy-3- | [(2S,4S,5S)- |
| cal | [3-hydroxy-4- | yl)-5-hydroxy- | (4-hydroxy-3- | [(2S,3R,4S,5S, | 4,5- |
| name | [(2S,3R,4S,5S, | 7- | ((2S,3R,4S,5S | 6R)-3,4,5- | dihydroxy- |
| | 6R)-3,4,5- | [(2S,3R,4S,5S,6R)- | 6R)-3,4,5- | trihydroxy-6- | 6- |
| | trihydroxy-6- | 3,4,5- | trihydroxy-6- | (hydroxymeth- | (hydroxymeth- |
| | (hydroxymeth- | trihydroxy-6- | (hydroxymeth- | yl)oxan-2- | ethyl)-3- |
| | yl)oxy-2- | (hydroxymethyl) | yl)tetrahydro- | yl]oxyphenyl]- | [(2S,4S,5S)- |
| | yl]oxyphenyl] | oxo-2- | 2H-pyran-2- | 7- | 3,4,5- |
| | benzopyran-4- | yl]oxybenzopyran- | yl)oxy)phenyl)- | [(2S,3R,4S,5S, | trihydroxy- |
| | one | 4-one | 4H- | 6R)-3,4,5- | 6- |
| | | | benzopyran-4- | trihydroxy-6- | (hydroxymeth- |
| | | | one | (hydroxymeth- | ethyl)oxa- |
| | | | | yl)oxan-2- | 2-yl]oxa-2- |
| | | | | yl]oxybenzopy- | yl]oxa-2- |
| | | | | ran-4-one | yl]oxa-3- |
| | | | | | hydroxyphe- |
| | | | | | nyl]-7- |
| | | | | | [(2S,4S,5S)- |
| | | | | | 4,5- |
| | | | | | dihydroxy- |
| | | | | | 6- |
| | | | | | (hydroxymeth- |
| | | | | | yl)-3- |
| | | | | | [(2S,5R)- |
| | | | | | 3,4,5- |
| | | | | | trihydroxy- |
| | | | | | 6- |
| | | | | | methyloxa- |
| | | | | | 2-yl]oxa-2- |
| | | | | | yl]oxa-5- |
| | | | | | hydroxyben- |
| | | | | | zopyran-4- |
| | | | | | one |
| M.W. | 448.4 | 448.4 | 448.4 | 610.5 | 918.8 |
| Form. | C21H20O11 | C21H20O11 | C21H20O11 | C27H30O16 | |

| Class | Naringenin (CAS 480-41) derivatives, EUF-0.58 mg/ml | | | |
|---|---|---|---|---|
| Struct- ural formu- la | 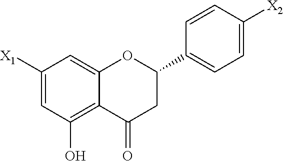 | | | |
| Gener- | Naringenin 7-O- | Naringenin 7-O- | Naringenin 7-β-D- | Naringenin 4′-O-β- |
| al | neohesperidin | ruinoside) | glucoside | D-glucopyranoside |
| English | (Naringin) | (Isonaringin) | | |
| name | | | | |
| CAS | 10236-47-2 | 14259-46-2 | 529-55-5 | 81202-36-0 |
| X₁ | 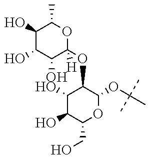 | 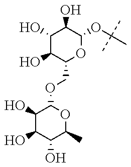 | 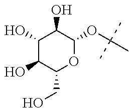 | OH |
| X₂ | OH | OH | OH | 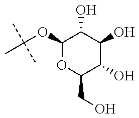 |
| English | (2S)-7- | (2S)-5-hydroxy- | (2S)-5-hydroxy-2- | (2S)-5,7- |
| chemi- | [(2S,3R,4S,5S,6R)- | 2-(4- | (4- | dihydroxy-2-[4- |
| cal | 4,5- | hydroxyphenyl)- | hydroxyphenyl)-7- | [(2S,3R,4S,5S,6R)- |
| name | dihydroxy-6- | 7- | [(2S,3R,4S,5S,6R) | 3,4,5-trihydroxy-6- |
| | (hydroxymethyl)- | [(2S,3R,4S,5S,6R)- | 3,4,5-trihydroxy- | (hydroxymethyl)ox- |
| | 3- | 3,4,5- | 6- | an-2- |
| | [(2S,3R,4R,5R,6S)- | trihydroxy-6- | (hydroxymethyl)o- | yl]oxyphenyl]-2,3- |
| | 3,4,5- | [(2R,3R,4R,5R,6S)- | xy-2-yl]oxy-2,3- | dihydrochrom-4- |
| | trihydroxy-6- | 3,4,5- | dihydrobenzopyran- | one |
| | methyloxy-2- | trihydroxy-6- | 4-one | |
| | yl]oxy-2-yl]oxy- | methyloxan-2- | | |
| | 5-hydroxy-2-(4- | yl]oxy]oxy-2- | | |
| | hydroxyphenyl)- | yl]oxy-2,3- | | |
| | 2,3- | dihydrobenzopyran- | | |
| | dihydrobenzopyran- | 4-one | | |
| | 4-one | | | |
| M.W. | 580.5 | 580.5 | 434.4 | 434.4 |
| Form. | C27H32O14 | C27H32O14 | C21H22O10 | C21H22O10 |

| | | |
|---|---|---|
| Class | Isosakuranetin (CAS 480-43-3) | |
| | derivatives-0.24 | Eriodictyol (CAS: 552-58-9) EUF-1.63 |
| Struct- ural formu- la | 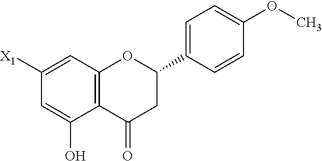 | 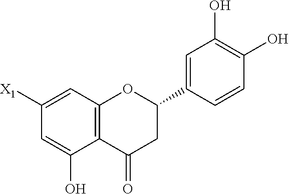 |
| | When X₁ is OH, Isosakuranetin | When X₁ is OH, Eriodictyol |

| | | | | |
|---|---|---|---|---|
| Gener- | (Poncirin) | Didymin | Neoeriocitrin | (Eriocitrin) |
| al | Isosakuranetin-7- | Isosakuranetin-7- | Eriodictyol 7-O- | Eriodictyol 7-O- |
| English | O-neohesperidin | O-rutinoside | neohesperidin | rutinoside |
| name | | | | |
| CAS | 14941-08-3 | 14259-47-3 | 13241-32-2 | 13463-28-0 |
| X₁ | 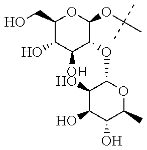 | 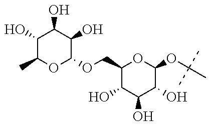 | 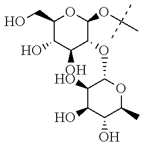 | 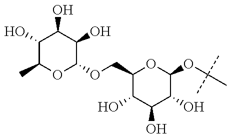 |
| English | (2S)-7- | (2S)-5-hydroxy- | (2S)-7- | (2S)-2-(3,4- |
| chemi- | [(2S,3R,4S,5S,6R)- | 2-(4- | [(2S,3R,4S,5S,6R)- | dihydroxyphenyl) |
| cal | 4,5- | methoxyphenyl)- | 4,5-dihydroxy-6- | 5-hydroxy-7- |
| name | dihydroxy-6- | 7- | (hydroxymethyl)-3- | [(2S,3R,4S,5S,6R)- |
| | (hydroxymethyl)- | [(2S,3R,4S,5S,6R)- | [(2S,3R,4R,5R,6S)- | 3,4,5- |
| | 3- | 3,4,5- | 3,4,5-trihydroxy-6- | trihydroxy-6- |
| | [(2S,3R,4R,5R,6S)- | trihydroxy-6- | methoxy-2-yl]oxy- | [(2R,3R,4R,5R,6S)- |
| | 3,4,5- | [(2R,3R,4R,5R,6S)- | 2-yl]oxy-2-(3,4- | 3,4,5- |
| | trihydroxy-6- | 3,4,5- | dihdyroxyphenyl)-5- | trihydroxy-6- |
| | methoxy-2- | trihydroxy-6- | hydroxy-2,3- | methyloxan-2- |
| | yl]oxy-2-yl]oxy- | methyloxy-2- | dihydrobenzopyran- | yl]oxymethyl]oxy- |
| | 5-hydroxy-2-(4- | yl]oxymethyl]ox- | 4-one | 2-yl]oxy-2,3- |
| | methoxyphenyl)- | y-2-yl]oxy-2,3- | | dihydrobenzopyran- |
| | 2,3- | dihydrobenzopyran- | | 4-one |
| | dihydrobenzopyran- | 4-one | | |
| | 4-one | | | |
| M.W. | 594.6 | 594.6 | 596.5 | 596.5 |
| Form. | C28H34O14 | C28H34O14 | C27H32O15 | C27H32O15 |

| Class | Diosmetin (520-34-3) derivatives | |
|---|---|---|
| Struct- ural formu- la | 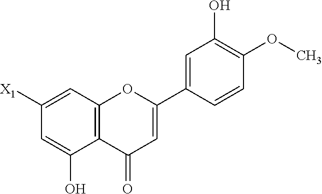 | |
| | When X₁ is OH, Diosmetin 520-34-3 | |
| Gener- | Diosmetin-7-O-neohesperidin | Diosmetin-7-O-rutinoside |
| al | | |
| English | | |
| name | | |
| CAS | 38665-01-9 | 520-27-4 |
| X₁ | 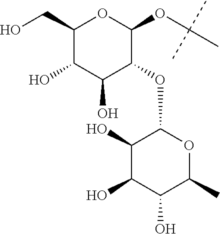 | 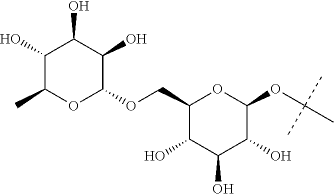 |
| English | 7-[(2S,3R,4S,5S,6R)-4,5-dihydroxy- | 5-hydroxy-2-(3-hydroxy-4- |
| chemi- | 6-(hydroxymethyl)-3- | methoxyphenyl)-7-[(2S,3R,4S,5S,6R)- |
| cal | [(2S,3R,4R,5R,6S)-3,4,5-trihydroxy- | 3,4,5-trihydroxy-6-[(2R,3R,4R,5R,6S)- |
| name | 6-methoxy-2-yl]oxy-2-yl]oxy-5- | 3,4,5-trihydroxy-6-methyloxan-2- |
| | hydroxy-2-(3-hydroxy-4- | yl]oxymethyl]oxy-2-yl]oxybenzopyran- |
| | methoxyphenyl)benzopyran-4-one | 4-one |
| M.W. | 608.5 | 608.5 |
| Form. | C28H32O15 | C28H32O15 |

| Class | Kaempferol (520-18-3) derivatives-0.40 | | | | |
|---|---|---|---|---|---|
| Structu- ural formul- la | 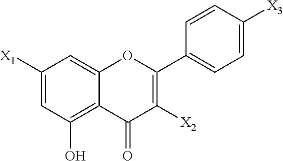 | | | | |
| | Unless otherwise specified, X₁ and X₃ are OH | | | | |
| Gener- | Kaempferol | Kaempferol-3- | Kaempferol 3- | Kaempferol 3- | Kaempferol |
| al | 3-O-glucoside | O-rutoside | O-α-L- | O-β-D- | 3-O-β-D- |
| English | | | rhamnoside | galactopyranoside | glucuronopy- |
| name | | | | | ranoside |
| CAS | 480-10-4 | 17650-84-9 | 482-39-3 | 23627-87-4 | 22688-78-4 |
| X₂ | 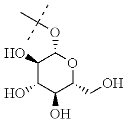 | 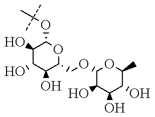 | 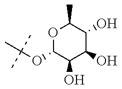 | 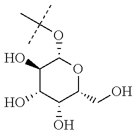 | 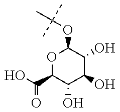 |
| English | 5,7- | 5,7- | 5,7- | 5,7-dihydroxy- | (2S,3S,4S,4R, |
| chemi- | dihydroxy-2- | dihydroxy-2- | dihydroxy-2- | 2-(4- | 6S)-6- |
| cal | (4- | (4- | (4- | hydroxyphenyl)- | [5,7- |
| name | hydroxyphenyl)- | hydroxyphenyl)- | hydroxyphenyl)- | 3- | dihydroxy- |
| | 3- | 3- | 3- | [(2S,3R,4S,5R, | 2-(4- |
| | [(2S,3R,4S,5S, | [(2S,3R,4S,5S, | [(2S,3R,4R,5R, | 6)-3,4,5- | hydroxyphe- |
| | 6R)-3,4,5- | 6R)-3,4,5- | 6S)-3,4,5- | trihydroxy-6- | nyl)-4- |
| | trihydroxy-6- | trihydroxy-6- | trihydroxy-6- | (hydroxymethyl) | oxobenzopy- |
| | (hydroxymeth- | [(2R,3R,4R,5R, | methoxy-2- | oxo-2- | ran-3- |
| | yl)oxo-2- | 6S)-3,4,5- | yl]oxybenzopy- | yl]oxybenzopyran- | yl]oxy- |
| | yl]oxybenzopy- | trihydroxy-6- | ran-4-one | 4-one | 3,4,5- |
| | ran-4-one) | methyloxy-2- | | | trihydroxyo- |
| | | yl]oxymethyl] | | | xan-2- |
| | | oxy]oxy-2- | | | carboxylic |
| | | yl]oxybenzopy- | | | acid |
| | | ran-4-one | | | |
| M.W. | 448.4 | 594.5 | 432.4 | 448.4 | 462.4 |
| Form. | C21H20O11 | C27H30O15 | C21H20O10- | C21H20O11 | C21H18O12 |
| Gener- | Kaempferol | Kaempferol 3- | Kaempferol 3- | Kaempferol 3- | Kaempferol |
| al | 3-O-β-D- | arabinofurano- | neohesperidin | O- | 3-O-(2,6-α- |
| English | sophoroside | side | | robinobioside | L- |
| name | | | | | rhamnosyl- |
| | | | | | β-D- |
| | | | | | glucopyrano- |
| | | | | | side |
| CAS | 19895-95-5 | 5041-67-8 | 32602-81-6 | 17297-56-2 | 55804-74-5 |
| X₂ | 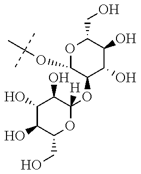 | 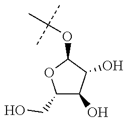 | 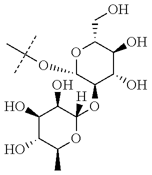 | 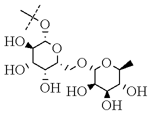 | 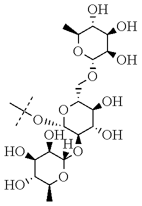 |
| English | 3- | 3- | 3- | 5,7-dihydroxy- | 3- |
| chemi- | [(2S,3R,4S,5S, | [(2S,3R,4R,5S)- | [(2S,3R,4S,5S, | 2-(4- | [(2S,3R,4S, |
| cal | 6R)-4,5- | 3,4- | 6R)-4,5- | hydroxyphenyl)- | 5S,6R)-4,5- |
| name | dihydroxy-6- | dihydroxy-5- | dihydroxy-6- | 3- | dihydroxy- |
| | yl)-3- | (hydroxymeth- | (hydroxymeth- | [(2S,3R,4S,5R, | 3- |
| | [(2S,3R,4S,5S, | yl)oxazolidin- | yl)-3- | 6R)-3,4,5- | [(2R,3R,4R, |
| | 3,4,5- | 2-yl]oxy-5,7- | [(2S,3R,4R,5R, | trihydroxy-6- | 5R,6S)- |
| | trihydroxy-6- | dihydroxy-2- | 6S)-3,4,5- | [(2R,3R,4R,5R, | 3,4,5- |
| | (hydroxymeth- | (4- | trihydroxy-6- | 6S)-3,4,5- | trihydroxy- |
| | yl)oxo-2- | hydroxyphenyl) | methoxy-2- | trihydroxy-6- | 6-methoxy- |
| | yl]oxo-2- | benzopyran- | yl]oxy-2- | methyloxy-2- | 2-yl]oxy-6- |
| | yl]oxo-5,7- | 4-one | yl]oxy-5,7- | yl]oxymethyl]o- | [(2R,3R,4R, |
| | dihydroxy-2- | | dihydroxy-2- | xy-2- | 5R,6S)- |
| | (4- | | (4- | yl]oxybenzopyran- | 3,4,5- |
| | hydroxyphenyl) | | hydroxyphenyl) | 4-one | trihydroxy- |
| | benzopyran- | | benzopyran- | | 6-methoxy- |
| | 4-one | | 4-one | | 2- |
| | | | | | yl]oxymeth- |
| | | | | | yl]oxy-2- |
| | | | | | yl]oxy-5,7- |
| | | | | | dihydroxy- |
| | | | | | 2-(4- |
| | | | | | hydroxyphe- |
| | | | | | nyl)benzopy- |
| | | | | | ran-4-one |
| M.W. | 610.5 | 418.3 | 594.5 | 594.5 | 740.7 |
| Form. | C27H30O16 | C20H18O10 | C27H30O15 | C27H30O15 | C33H40O19 |
| Gener- | Kaempferol | Kaempferol 3- | Kaempferol 3- | Kaempferol 3- | Kaempferol |
| al | 3-O- | O-(2,6-di-O- | O-β-D- | O- | 3,4′-O- |
| English | gentiobioside | α-L- | glucopyranoside- | sambubioside | diglucopyra- |
| name | | rhamnosyl)-β- | (1 → 2)-β- | | noside |
| | | D- | D- | | |
| | | galactopyrano- | galactopyrano- | | |
| | | side | side | | |
| CAS | 22149-35-5 | 109008-28-8 | 31512-06-8 | 27661-51-4 | 71939-16-7 |
| X₂ | 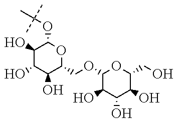 | 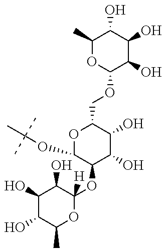 | 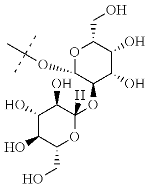 | 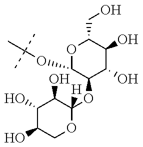 | 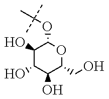 |
| X₃ | OH | OH | OH | OH | 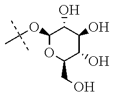 |
| English | 5,7- | 3-[4,5- | 3- | 3- | 5,7- |
| chemi- | dihydroxy-2- | dihydroxy-3- | [(2S,3R,4S,5R, | [(2S,3R,4S,5S, | dihydroxy- |
| cal | (4- | (3,4,5- | 6R)-4,5- | 6R)-4,5- | 3- |
| name | hydroxyphenyl)- | trihydroxy-6- | dihydroxy-6- | dihydroxy-6- | [(2S,3R,4S, |
| | 3- | methoxy-2- | (hydroxymeth- | (hydroxymethyl)- | 5S,6R)- |
| | [(2S,3R,4S,5S, | yl)oxy-6- | yl)-3- | 3- | 3,4,5- |
| | 6R)-3,4,5- | [(3,4,5- | [(2S,3R,4S,5S, | [(2S,3R,4S,5R)- | trihydroxy- |
| | trihydroxy-6- | trihydroxy-6- | 6R)-3,4,5- | 3,4,5- | 6- |
| | [(2R,3R,4S,5S, | methoxy-2- | trihydroxy-6- | trihydroxyoxo- | (hydroxymeth- |
| | 6R)-3,4,5- | yl)oxymethyl] | (hydroxymeth- | 2-yl]oxo-2- | ethyl)oxan- |
| | trihydroxy-6- | oxy-2-yl]oxy- | yl)oxo-2- | yl]oxo-5,7- | 2-yl]oxy-2- |
| | (hydroxymeth- | 5,7- | yl]oxo-5,7- | dihydroxy-2-(4- | [4- |
| | yl)oxo-2- | dihydroxy-2- | dihydroxy-2- | hydroxyphenyl) | [(2S,3R,4S, |
| | yl]oxymethyl] | (4- | (4- | benzopyran-4- | 5S,6R)- |
| | oxo-2- | hydroxyphenyl) | hydroxyphenyl) | one | 3,4,5- |
| | yl]oxybenzopy- | benzopyran- | benzopyran- | | trihydroxy- |
| | ran-4-one | 4-one | 4-one | | 6- |
| | | | | | (hydroxymeth- |
| | | | | | yl)oxan- |
| | | | | | 2- |
| | | | | | yl]oxyphenyl] |
| | | | | | benzopyran- |
| | | | | | 4-one |
| M.W. | 610.5 | 740.7 | 610.5 | 580.5 | 610.5 |
| Form. | C27H30O16 | C33H40O19 | C27H30O16 | C26H28O15 | C27H30O16 |
| Gener | Kaempferol | Kaempferol 7- | Kaempferol 3- | Kaempferol-7- | Kaempferol |
| al | 3,7-di-O-α-L- | O-β- | robinoside-7- | O-α-L- | 3,7-di-O-β- |
| English | rhamnoside | glucopyranoside | rhamnoside | rhamnosyl-3-O- | D- |
| name | | | | β-D- | glucopyrano- |
| | | | | glucopyranoside | side |
| CAS | 482-38-2 | 16290-07-6 | 301-19-9 | 2392-95-2 | 25615-14-9 |
| X₁ | 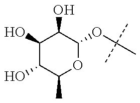 | 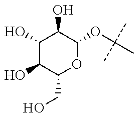 | 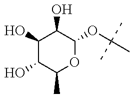 | 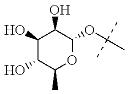 | 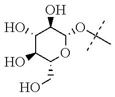 |
| X₂ | 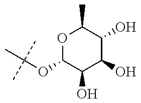 | OH | 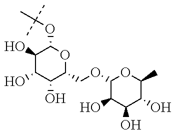 | 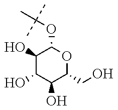 | 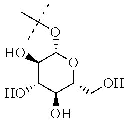 |
| English | 5-hydroxy-2- | 3,5- | 5-hydroxy-2- | (2S,3R,4R,5R,6S)- | 5-hydroxy- |
| chemi- | (4- | dihydroxy-2- | (4- | 2-[3,5- | 2-(4- |
| cal | hydroxyphenyl)- | (4- | hydroxyphenyl)- | dihydroxy-2-(4- | hydroxyphe- |
| name | 3,7- | hydroxyphenyl)- | 7- | hydroxyphenyl)- | nyl)-3,7- |
| | bis[(2S,3R,4R, | 7- | [(2S,3R,4R,5R, | 3- | di[(2S,3R,4S, |
| | 5R,6S)-3,4,5- | [(2S,3R,4S,5S, | 6S)-3,4,5- | [(2S,3R,4S,5S, | 5S,6R)- |
| | trihydroxy-6- | 6R)-3,4,5- | trihydroxy-6- | 6R)-3,4,5- | 3,4,5- |
| | methoxy-2- | trihydroxy-6- | methoxy-2- | trihydroxy-6- | trihydroxy- |
| | yl]oxy]benzo- | (hydroxymeth- | yl]oxy-3- | (hydroxymethyl) | 6- |
| | pyran-4-one | yl)oxo-2- | [(2S,3R,4S,5R, | oxan-2-yl]oxy- | (hydroxymeth- |
| | | yl]oxybenzopy- | 6R)-3,4,5- | 2,4- | ethyl)oxo- |
| | | ran-4-4-one | trihydroxy-6- | dihydrobenzopy- | 2- |
| | | | [[(2S,3R,4R,5R, | ran-7-yl]oxy]- | yl]oxy]benzo- |
| | | | 6S)-3,4,5- | 6-methyloxane- | pyran-4- |
| | | | trihydroxy-6- | 3,4,5-triol | one |
| | | | methoxy-2- | | |
| | | | yl]oxymethyl] | | |
| | | | oxy-2- | | |
| | | | yl]oxybenzopy- | | |
| | | | ran-4-one | | |
| M.W. | 578.5 | 448.4 | 740.7 | 598.5 | 610.5 |
| Form. | C27H30O14 | C21H20O11 | C33H40O19 | C27H34O15 | C27H30O16 |
| Gener- | Kaempferol | Kaempferol 3- | Kaempferol 3- | Kaempferol 3- | Kaempferol |
| al | 3-O-α-L- | O-rutoside-7- | O-β-turinoside | sophoroside-7- | 3-O- |
| English | rhamnoside | O-α-L- | 7-O-β- | rhamnoside | xylosyl- |
| name | | rhamnoside | glucoside | | rutinoside |
| CAS | 201-96-89-8 | 57526-56-4 | 34336-18-0 | 93098-79-4 | N/A |
| X₁ | 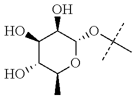 | 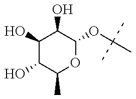 | 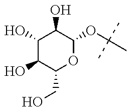 | 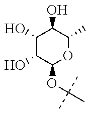 | OH |
| X₂ | OH | 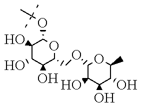 | 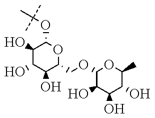 | 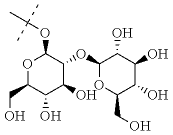 | 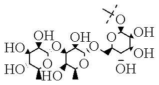 |
| English | 3,5- | 5-hydroxy-2- | 5-hydroxy-2- | 3- | 3- |
| chemi- | dihydroxy-2- | (4- | (4- | [(2S,3R,4S,5S, | [(2S,3R,4S, |
| cal | (4- | hydroxyphenyl)- | hydroxyphenyl)- | 6R)-4,5- | 5S,6R)-6- |
| name | hydroxyphenyl)- | 7- | 7- | dihydroxy-6- | [(2R,3R,4R, |
| | 7- | [(2S,3R,4R,5R, | [(3R,4S,5S,6R)- | (hydroxymethyl)- | 5S,6S)- |
| | [(2S,3R,4R,5R, | 6S)-3,4,5- | 3,4,5- | 3- | 3,5- |
| | 6S)-3,4,5- | trihydroxy-6- | trihydroxy-6- | [(2S,3R,4S,5S, | dihydroxy- |
| | trihydroxy-6- | methoxy-2- | (hydroxymeth- | 6R)-3,4,5- | 6-methyl-4- |
| | methoxy-2- | yl]oxy-3- | yl)oxo-2- | trihydroxy-6- | [(2S,3R,4R, |
| | yl]oxybenzopy- | [(2S,3R,4S,5S, | yl]oxo-3- | (hydroxymethyl) | 5R,6S)- |
| | ran-4-one | 6R)-3,4,5- | [(3R,4S,5S,6R)- | oxan-2- | 3,4,5- |
| | | trihydroxy-6- | 3,4,5- | yl]oxan-2- | trihydroxy- |
| | | methoxy-2- | trihydroxy-6- | yl]oxan-5- | 6-methoxy- |
| | | yl]oxy-2- | [(1R,2S,3R,4R)- | hydroxy-2-(4- | 2-yl]oxy-2- |
| | | yl]oxybenzopy- | 1,2,3- | hydroxyphenyl)- | yl]oxymeth- |
| | | ran-4-one | tetrahydroxypen- | 7- | yl]-3,4,5- |
| | | | tyl]oxo-2- | [(2S,3R,4R,5R, | trihydroxy- |
| | | | yl]oxybenzopy- | 6S)-3,4,5- | 2-yl]oxy- |
| | | | ran-4-one | hydroxy-6- | 5,7- |
| | | | | methyloxan-2- | dihydroxy- |
| | | | | yl]oxobenzopyran- | 2-(4- |
| | | | | 4-one | hydroxyphe- |
| | | | | | nyl)benzopy- |
| | | | | | ran-4-one |
| M.W. | 432.4 | 740.7 | 714.6 | 756.7 | 740.7 |
| Form. | C21H20O10 | C33H40O19 | C31H38O19 | C33H40O20 | C33H40O19 |
| Gener- | Kaempferol | Kaempferol 3- | Kaempferol 3- | Kaempferol 3- | Kaempferol |
| al | 3-O-glucosyl- | O-(6″- | O-(2′- | O-(2′- | 3-O- |
| English | rhamnosyl- | acetylgalactoside) | rhamnosylgala- | rhamnosyl-6′- | rhamnosyl- |
| name | glucoside | 7-O- | ctoside) 7-O- | acetylgalactoside) | rhamnosyl- |
| | | rhamnoside | rhamnoside | 7-O- | glucoside |
| | | | | rhamnoside | |
| CAS | N/A | N/A | N/A | N/A | N/A |
| X₁ | OH | 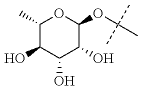 | 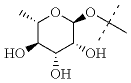 | 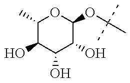 | OH |
| X₂ | 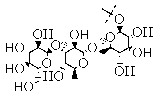 | 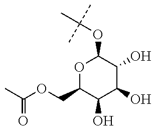 | 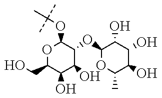 | 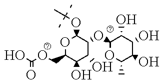 | 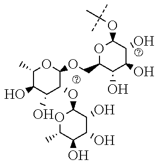 |
| English | 3- | [(2R,3R,4S,5R, | 3- | [(2R,3R,4S,5R, | 3- |
| chemi- | [(2S,3R,4S,5S | 6S)-3,4,5- | {(2S,3R,4S,5R, | 6S)-3,4- | {[(2S,3R,4S, |
| cal | 6R)-6- | trihydroxy-6- | 6R)-4,5- | dihydroxy-6- | 5S,6R)-6- |
| name | [(2R,3R,4R,5S, | [5-hydroxy-2- | dihydroxy-6- | {[5-hydroxy-2- | ({[(2R,3R,4R, |
| | 6S)-3,5- | (4- | (hydroxymeth- | (4- | 5R,6S)- |
| | dihydroxy-6- | hydroxyphenyl)- | yl)-3- | hydroxyphenyl)- | 4,5- |
| | methyl-4- | 4-oxo-7- | {(2S,3R,4R,5R, | 4-oxo-7- | dihydroxy- |
| | [(2S,3R,4S,5S, | [(2S,3R,4R,5R, | 6S)-3,4,5- | {[(2S,3R,4R,5R, | 6-methyl-3- |
| | 6R)-3,4,5- | 6S)-3,4,5- | trihydroxy-6- | 6S)-3,4,5- | {[(2S,3R,4R, |
| | trihydroxy-6- | trihydroxy-6- | methyloxy-2- | trihydroxy-6- | 5R,6S)- |
| | (hydroxymeth- | methoxy-2- | yl]oxy}-5- | methoxy-2- | 3,4,5- |
| | yl)oxan-2- | yl]oxybenzopy- | hydroxy-2-(4- | yl]oxy]-4H- | trihydroxy- |
| | yl]oxan-2- | ran-3-yl]oxo- | hydroxyphenyl)- | benzopyran-3- | 6- |
| | yl]oxymethyl]- | 2-yl]methyl- | 7- | yl]oxy}-5- | methyloxan- |
| | 3,4,5- | acetate | {(2S,3R,4R,5R, | {(2S,3R,4R,5R, | 2-yl]oxan- |
| | trihydroxyoxan- | | 6S)-3,5- | 3,4,5- | 2- |
| | 2-yl]oxy- | | trihydroxy-6- | trihydroxy-6- | yl]oxy}meth- |
| | 5,7- | | methyloxy]ox- | methoxy-2- | yl)-3,4,5- |
| | dihydroxy-2- | | yl}benzopyran- | yl]oxy}-2- | trihydroxyo- |
| | (4- | | 4-one | yl]methyl- | xy-2- |
| | hydroxyphenyl) | | | bicarbonate) | yl]oxy}- |
| | benzopyran- | | | | 5,7- |
| | 4-one | | | | dihydroxy- |
| | | | | | 2-(4- |
| | | | | | hydroxyphe- |
| | | | | | nyl)-4H- |
| | | | | | benzopyran- |
| | | | | | 4-one |
| M.W. | 756.7 | 636.6 | 740.7 | 784.7 | 740.7 |
| Form. | C33H40O20 | C29H36O12 | C33H40O19 | C34H40O21 | C33H40O19 |

| Class | Chrysin (480-40-0) derivatives | | | |
|---|---|---|---|---|
| Struct- ural formu- la | 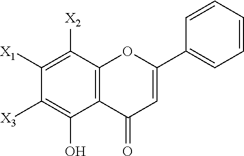 | | | |
| Gener- | Chrysin 7-O- | 5,7- | CHrysin | Chrysin |
| al | glucuronide | dihydroxyflavone | 6-C-arabinoside 8- | 6-C-glucoside 8-C- |
| English | | 7-O- | C-glucoside | arabinoside |
| name | | glucopyranoside | | |
| CAS | 35775-49-6 | 31025-53-3 | 185145-33-9 | 185145-34-0 |
| X₂ | 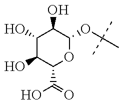 | 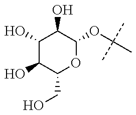 | OH | OH |
| X₂ | H | H | 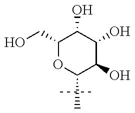 | 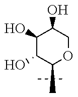 |
| X₃ | H | H | 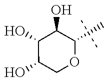 | 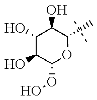 |
| English | (2S,3S,4S,5R, | 5-hydroxy-2- | 5,7-dihydroxy-2- | 5,7-dihydroxy-2- |
| chemi- | 6S)-3,4,5- | phenyl-7- | phenyl-8- | phenyl-6- |
| cal | trihydroxy-6- | [(2S,3R,4S,5S,6R)- | [(2S,3R,4R,5S,6R)- | [(2S,3R,4R,5S,6R)- |
| name | (5-hydroxy-4- | 3,4,5-trihydroxy- | 3,4,5-trihydroxy- | 3,4,5-trihydroxy-6- |
| | oxo-2- | 6- | 6-(hydroxymethyl) | (hydroxymethyl)/ |
| | phenylbenzopy- | (hydroxymethyl)ox- | oxa-2-yl]-6- | oxa-2-yl]-8- |
| | ran-7- | o-2- | [(2S,3R,4S,5S)- | [(2S,3R,4S,5S)- |
| | yl)oxo-2- | yl]oxybenzopyran- | 3,4,5- | 3,4,5-trihydroxyoxa- |
| | carboxylic | 4-one | trihydroxyoxa-2- | 2-yl]benzopyran-4- |
| | acid | | yl]benzopyran-4- | one |
| | | | one | |
| M.W. | 430.4 | 416.4 | 548.5 | 548.5 |
| Form. | C21H18O10 | C21H20O9 | C26H28O13 | C26H28O13 |

| Class | Genistein (446-74--0) derivatives | | | |
|---|---|---|---|---|
| Struct- ural formu- la | 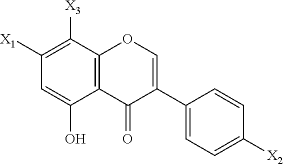 | | | |
| | Unless otherwise specified, X₂ is OH and X₃ is H | | | |
| Gener- | 5,4′- | 7-O-glucosyl-6′- | Genistein 6″-O- | Genistein-7β- |
| al | dihydroxyisoflavone- | malonylgenistein | acetate | glucuronide |
| English | 7-O-β-D- | | | |
| name | glucopyranoside | | | |
| CAS | 529-59-9 | 51011-05-3 | 73566-30-0 | 38482-81-4 |
| X₁ | 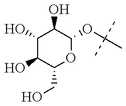 | 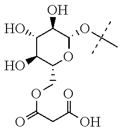 | 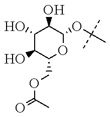 | 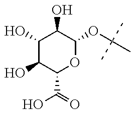 |
| English | 5-hydroxy-3-(4- | 3-oxo-3- | [(2R,3S,4S,5R,6S)- | (2S,3S,4S,5R,6S)- |
| chemi- | hydroxyphenyl)- | [(2R,3S,4S,5R,6S)- | 3,4,5-trihydroxy- | 3,4,5-trihydroxy-6- |
| cal | 7- | 3,4,5- | 6-[5-hydroxy-3-(4- | [5-hydroxy-3-(4- |
| name | [(2S,3R,4S,5S,6R)- | trihydroxy-6-[5- | hydroxyphenyl)-4- | hydroxyphenyl)-4- |
| | 3,4,5- | hydroxy-3-(4- | oxobenzopyran-7- | oxobenzopyran-7- |
| | trihydroxy-6- | hydroxyphenyl)- | yl]oxan-2- | yl]oxyosan-2- |
| | (hydroxymethyl) | 4- | yl]methy lacetate | carboxylic acid |
| | oxo-2- | oxobenzopyran- | | |
| | yl]oxybenzopyran- | 7-yl]oxo-2- | | |
| | 4-one | yl]methoxy] | | |
| | | propionic acid | | |
| M.W. | 432.4 | 518.4 | 474.4 | 446.4 |
| Form. | C21H20O10 | C24H22O13 | C23H22O11 | C21H18O11 |
| Gener- | 5′,7′-Dihydroxy- | Sophorabioside | Genistein 4′,7-di- | 8-C-β- |
| al | 4′- | | O-β-D-glucoside | Glucosylgenistein |
| English | glucosyloxyisofla- | | | |
| name | vone | | | |
| CAS | 152-95-4 | 2945-88-2 | 36190-98-4 | 66026-80-0 |
| X₁ | OH | OH | 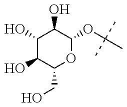 | OH |
| X₂ | 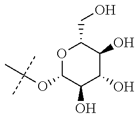 | 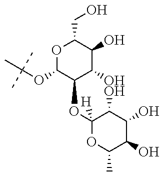 | 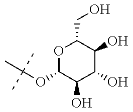 | OH |
| X₃ | H | H | H | 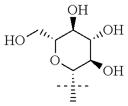 |
| English | 5,7-dihydroxy-3- | 3-[4- | 5-hydroxy-7- | 5,7-dihydroxy-3- |
| chemi- | [4- | [(2S,3R,4S,5S,6R)- | [(2S,3R,4S,5S,6R)- | (4-hydroxyphenyl)- |
| cal | [(2S,3R,4S,5S,6R)- | 4,5- | 3,4,5-trihydroxy- | 8- |
| name | 3,4,5- | dihydroxy-6- | 6- | [(2S,3R,4R,5S,6R)- |
| | trihydroxy-6- | (hydroxymethyl)- | (hydroxymethyl)o- | 3,4,5-trihydroxy- |
| | (hydroxymethyl) | 3- | xy-2-yl]oxy-3-[4- | 6- |
| | yl]oxyphenyl]ben- | [(2S,3R,4R,5R,6S)- | [(2S,3R,4S,5S,6R)- | (hydroxymethyl)ox- |
| | zopyran-4-one | 3,4,5- | 3,4,5-trihydroxy- | 2-yl]benzopyran- |
| | | trihydroxy-6- | 6- | 4-one |
| | | methoxy-2- | (hydroxymethyl)o- | |
| | | yl]oxy-2- | xy-2- | |
| | | 5,7- | yl]oxyphenyl]benzo- | |
| | | dihydroxybenzopy- | pyran-4-one | |
| | | ran-4-one | | |
| M.W. | 432.4 | 578.5 | 594.5 | 432.4 |
| Form. | C21H20O10 | C27H30O14 | C27H30O15 | C21H20O10 |

| Class | Baicalein (CAS 491-67-8) derivatives | |
|---|---|---|
| Gener- | Baicalein 7-O-β-D-glucorinide | Baicalein 6-O-glucuronide |
| al | | |
| English | | |
| name | | |
| CAS | 21967-41-9 | 35990-03-5 |
| Struct- ure | 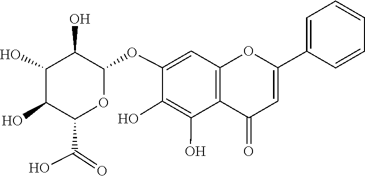 [SMILES: O=C(O)[C@H]1O[C@@H](Oc2cc3oc(-c4ccccc4)cc(=O)c3c(O)c2O)[C@H](O)[C@@H](O)[C@@H]1O] | 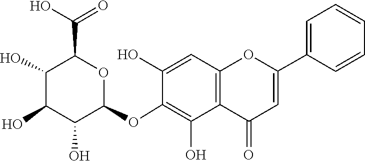 [SMILES: O=C(O)[C@H]1O[C@@H](Oc2c(O)cc3oc(-c4ccccc4)cc(=O)c3c2O)[C@H](O)[C@@H](O)[C@@H]1O] |
| English | (2S,3S,4S,5R,6S)-6-(5,6-dihydroxy- | (2S,3S,34S,5R,6S)-6-(5,7-dihydroxy-4- |
| chemi- | 4-oxo-2-phenylbenzopyran-7-yl)oxy- | oxo-2-phenylbenzopyran-6-yl)oxy-3,4,5- |
| cal | 3,4,5-trihydroxyoxo-2-carboxylic | trihydroxyoxo-2-carboxylic acid |
| name | acid | |
| M.W. | 446.4 | 446.4 |
| Form. | C21H18O11 | C21H18O11 |

| Class | (−)-Epicatechin(−)-derivatives | | |
|---|---|---|---|
| Gener- | Epicatechin gallate | Epigallocatechin ester | Epigallocatechin 3-gallate |
| al | | | |
| English | | | |
| name | | | |
| CAS | 1257-08-5 | 97-74-1 | 989-51-5 |
| Struct- ural formu- la | 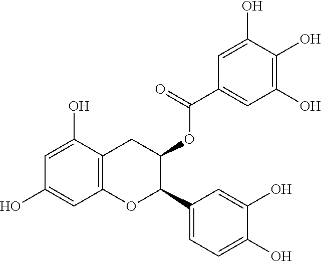 [SMILES: O=C(O[C@@H]1Cc2c(O)cc(O)cc2O[C@@H]1c1ccc(O)c(O)c1)c1cc(O)c(O)c(O)c1] | 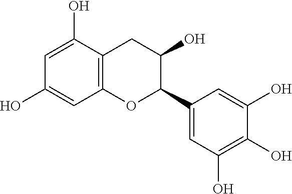 [SMILES: Oc1cc(O)c2c(c1)O[C@H](c1cc(O)c(O)c(O)c1)[C@H](O)C2] | 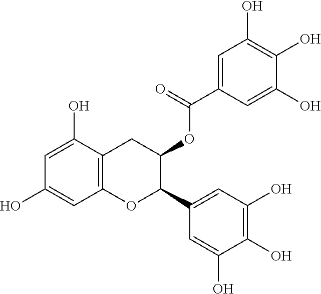 [SMILES: O=C(O[C@@H]1Cc2c(O)cc(O)cc2O[C@@H]1c1cc(O)c(O)c(O)c1)c1cc(O)c(O)c(O)c1] |
| English | [(2R,3R)-2-(3,4- | (2R,3R)-2-(3,4,5- | [(2R,3R)-5,7-dihydroxy-2- |
| chemi- | dihydroxyphenyl)-5,7- | trihydroxyphenyl)-3,4- | (3,4,5-trihydroxyphenyl)- |
| cal | dihydroxy-3,4-dihydro- | dihydro-2H_benzopyran- | 3,4-dihydro-2H- |
| name | 2H-benzopyran-3- | 3,5,7-triol | benzopyran-3-yl]3,4,5- |
| | yl]3,4,5- | | trihydroxybenzoate |
| | trihydroxybenzoate | | |
| M.W. | 442.4 | 306.3 | 458.4 |
| Form. | C22H18O10 | C15H14O7 | C22H18O11 |

| Class | (+)-Catechin(+)-derivatives | |
|---|---|---|
| Gener- | Catechin 3′-glucoside | |
| al | | |
| English | | |
| name | | |
| CAS | 105330-51-6 | |
| Struct- ural formu- la | 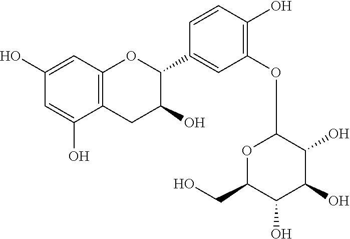 [SMILES: OC[C@H]1OC(Oc2cc([C@H]3Oc4cc(O)cc(O)c4C[C@@H]3O)ccc2O)[C@H](O)[C@@H](O)[C@@H]1O] | |
| English | (2R,3S)-2-(4-hydroxy-3-((2S,3R,4S,5S,6R)-3,4,5-trihdryoxy-6- | |
| chemi- | (hydroxymethyl)tetrahydro-2H-pyran-2-yl)oxy)phenyl)benzotetrahydropyran- | |
| cal | 3,5,7-triol | |
| name | | |
| M.W. | 452.4 | |
| Form. | C21H24O11 | |
| Class | Puerarin (CAS 3681-99-0) derivatives | |
| Gener- | Puerarin apioside | Puerarin 4′-O-β-D-glucopyranoside |
| al | | |
| English | | |
| name | | |
| CAS | 103654-60-8 | 117047-08-2 |
| Struct- ure | 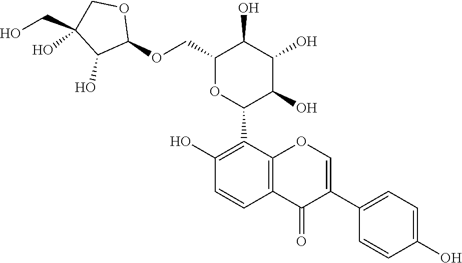 [SMILES: O=c1c(-c2ccc(O)cc2)coc2c([C@@H]3O[C@H](CO[C@@H]4OC[C@](O)(CO)[C@H]4O)[C@@H](O)[C@H](O)[C@H]3O)c(O)ccc12] | 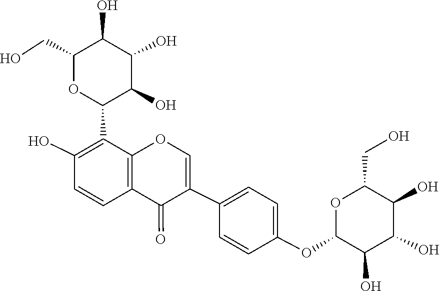 [SMILES: O=c1c(-c2ccc(O[C@@H]3O[C@H](CO)[C@@H](O)[C@H](O)[C@H]3O)cc2)coc2c([C@@H]3O[C@H](CO)[C@@H](O)[C@H](O)[C@H]3O)c(O)ccc12] |
| English | 8-[(2S,3R,4R,5S,6R)-6-[[(2R,3R,4R)- | 7-hydroxy-8-[(2S,3R,4R,5S,6R)- |
| chemi- | 3,4-dihydroxy-4- | 3,4,5-trihydroxy-6- |
| cal | (hydroxymethyl)oxolan-2- | (hydroxymethyl)oxan-2-yl]-3-[4- |
| name | yl]oxymethyl]-3,4,5-trihydroxyoxan-2- | [(2S,3R,4R,5S,6R)-3,4,5-trihydroxy- |
| | yl]-7-hydroxy-3-(4- | 6-(hydroxymethyl)oxan-2- |
| | hydroxyphenyl)chromen-4-one | yl]oxyphenyl]benzopyran-4-one |
| M.W. | 548.5 | 578.5 |
| Form. | C26H28O13 | C27H30O14 |

| Class | Chalcone, Dihydrochalcone | | | |
|---|---|---|---|---|
| Gener- | Phloretin | Phloridzin | Sieboldin | Aspalathin |
| al | | | | |
| English | | | | |
| name | | | | |
| CAS | 60-82-2 | 60-81-1 | 18777-73-6 | 6027-43-6 |
| Chemi- cal structu- ure | 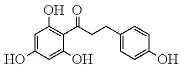 [SMILES: O=C(CCc1ccc(O)cc1)c1c(O)cc(O)cc1O] | 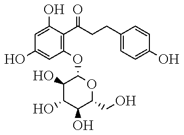 [SMILES: O=C(CCc1ccc(O)cc1)c1c(O)cc(O)cc1O[C@@H]1O[C@H](CO)[C@@H](O)[C@H](O)[C@H]1O] | 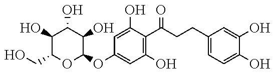 [SMILES: O=C(CCc1ccc(O)c(O)c1)c1c(O)cc(O[C@H]2O[C@H](CO)[C@@H](O)[C@H](O)[C@H]2O)cc1O] | 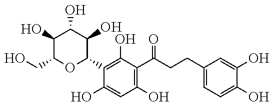 [SMILES: O=C(CCc1ccc(O)c(O)c1)c1c(O)cc(O)c([C@@H]2O[C@H](CO)[C@@H](O)[C@H](O)[C@H]2O)c1O] |
| English | 3-(4- | 1-[2,4-dihydroxy- | 1-(2,6- | 3-(3,4- |
| chemi- | hydroxyphenyl)-1- | 6- | dihydroxy-4- | dihydroxyphenyl)- |
| cal | (2,4,6- | [(2S,3R,4S,5S,6R)- | {[3,4,5- | 1-{2,4,6- |
| name | trihydroxyphenyl)pro- | 3,4,5-trihydroxy- | trihydroxy-6- | trihydroxy-3- |
| | pan-1-one | 6- | (hydroxymethyl) | [3,4,5- |
| | | (hydroxymethyl)o- | oxo-2- | trihydroxy-6- |
| | | xo-2- | yl]oxy}phenyl)- | (hydroxymethyl) |
| | | yl]oxophenyl]-3- | 3-(3,4- | oxo-2- |
| | | (4- | dihydroxyphenyl) | yl]phenyl]propan- |
| | | hydroxyphenyl)pro- | propan-1-one | 1-one |
| | | pan-1-one | | |
| M.W. | 274.3 | 436.4 | 452.4 | 452.4 |
| Form. | C15H14O5 | C12H24O10 | C21H24O11 | C21H24O11 |
| LogP | 3.8-3.9 | 0.98 | 1.32 | 0.83 |
| Sol | 3.07 mg/ml | 3.89 mg/ml | 11.65 mg/ml | 36.12 mg/ml |
| Gener- | Neosperidin | Trilobatin | Trilobatin 2″- | Naringin |
| al | dihydrochalcone) | | acetate | dihydrochalcone |
| English | | | | |
| name | | | | |

| | | | | |
|---|---|---|---|---|
| Chemi- cal struc- ture | 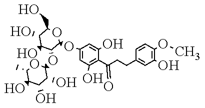 | 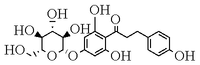 [SMILES: O=C(CCc1ccc(O)cc1)c1c(O)cc(O[C@@H]2O[C@H](CO)[C@@H](O)[C@H](O)[C@H]2O)cc1O] | 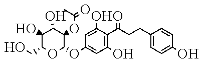 [SMILES: CC(=O)O[C@H]1[C@H](Oc2cc(O)c(C(=O)CCc3ccc(O)cc3)c(O)c2)O[C@H](CO)[C@@H](O)[C@@H]1O] | 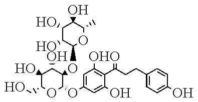 [SMILES: C[C@@H]1O[C@@H](O[C@H]2[C@H](Oc3cc(O)c(C(=O)CCc4ccc(O)cc4)c(O)c3)O[C@H](CO)[C@@H](O)[C@@H]2O)[C@H](O)[C@H](O)[C@H]1O] |

| | | | | |
|---|---|---|---|---|
| CAS | 20702-77-6 | 4192-90-9 | 647853-82-5 | 18916-17-1 |
| English | 1-[4- | 1-[2,6-dihydroxy- | [(2S,3R,4S,5S,6R)- | 1-[4- |
| chemi- | [(2S,3R,4S,5S,6R)- | 4- | (R)-2-[3,5- | [(2S,3R,4S,5S,6R)- |
| cal | 4,5-dihydroxy-6- | [(2S,3R,4S,5S,6R)- | dihydroxy-4-[3- | 4,5- |
| name | (hydroxymethyl)-3- | 3,4,5-trihydroxy- | (4- | dihydroxy-6- |
| | [(2S,3R,4R,5R,6S)- | 6- | hydroxyphenyl) | (hydroxymethyl)- |
| | 3,4,5-trihydroxy-6- | (hydroxymethyl)o- | propionyl]pheno- | 3- |
| | methoxy-2-yl]oxy- | xo-2- | xy]-4,5- | [(2S,3R,4R,5R,6S)- |
| | 2-yl]oxy-2,6- | yl]oxophenyl]-3- | dihydroxy-6- | 3,4,5- |
| | dihydroxyphenyl]-3- | (4- | (hydroxymethyl) | trihydroxy-6- |
| | (3-hydroxy-4- | hydroxyphenyl)pro- | oxy-3-yl]acetate | methoxy-2- |
| | methoxyphenyl)pro- | pan-1-one | | yl]oxy-2-yl]oxy- |
| | pan-1-one | | | 2,6- |
| | | | | dihydroxyphenyl]- |
| | | | | 3-(4- |
| | | | | hydroxyphenyl) |
| | | | | propan-1-one |
| M.W. | 612.6 | 436.4 | 478.4 | 582.5 |
| Form. | C28H36O15 | C21H24O10 | C23H26O11 | C27H34O14 |
| LogP | −1.05 | 1.63 | 2.07 | 0.90 |
| Sol | 104.87 mg/ml | 3.89 mg/ml | 2.08 mg/ml | 6.78 mg/ml |
| Gener- | Neoeriocitrin | 3- | 3- | 3- |
| al | dihydrochalcone | hydroxyphloretin | hydroxyphloretin | hydroxyphloretin |
| English | | | 2′-xyloside | 2′-glucoside |
| name | | | | |

| | | | | |
|---|---|---|---|---|
| Chemi- cal struc- ture | 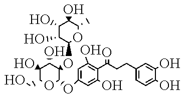 [SMILES: C[C@@H]1O[C@@H](O[C@H]2[C@H](Oc3cc(O)c(C(=O)CCc4ccc(O)c(O)c4)c(O)c3)O[C@H](CO)[C@@H](O)[C@@H]2O)[C@H](O)[C@H](O)[C@H]1O] | 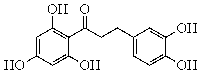 [SMILES: O=C(CCc1ccc(O)c(O)c1)c1c(O)cc(O)cc1O] | 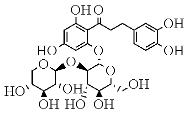 [SMILES: O=C(CCc1ccc(O)c(O)c1)c1c(O)cc(O)cc1O[C@@H]1O[C@H](CO)[C@@H](O)[C@H](O)[C@H]1O[C@@H]1OC[C@@H](O)[C@H](O)[C@H]1O] | 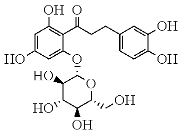 [SMILES: O=C(CCc1ccc(O)c(O)c1)c1c(O)cc(O)cc1O[C@@H]1O[C@H](CO)[C@@H](O)[C@H](O)[C@H]1O] |

| | | | | |
|---|---|---|---|---|
| CAS | 65520-51-6 | 57765-66-9 | N/A | N/A |
| English | 1-[4- | 3-(3,4- | 1-[2- | 1-(2,4- |
| chemi- | [(2S,3R,4S,5S,6R)- | dihydroxyphenyl)- | [(2S,3R,4S,5S,6R)- | dihydroxy-6- |
| cal | 4,5-dihydroxy-6- | 1-(2,4,6- | 4,5- | {(2S,3R,4S,5R,6R)- |
| name | (hydroxymethyl)-3- | trihydroxyphenyl) | dihydroxy-6- | 3,4,5- |
| | [(2S,3R,4R,5R,6S)- | propan-1-one | (hydroxymethyl)- | trihydroxy-6- |
| | 3,4,5-trihydroxy-6- | | (hydroxymethyl)- | oxo-2- |
| | methoxy-2-yl]oxy- | | [(2S,3R,4S,5R)- | yl]oxy}phenyl)- |
| | 2-yl]oxy-2,6- | | 3,4,5- | 3-(3,4- |
| | dihydroxyphenyl]-3- | | trihydroxyoxo-2- | dihydroxyphenyl) |
| | (3,4- | | yl]oxo-2-yl]oxo- | propan-1-one |
| | dihydroxyphenyl)pro- | | 4,6- | |
| | pan-1-one | | dihydroxyphenyl]- | |
| | | | 3-(3,4- | |
| | | | dihydroxyphenyl) | |
| | | | propan-1-one | |
| M.W. | 598.5 | 290.3 | 584.5 | 452.4 |
| Form. | C27H34O15 | C15H14O6 | C26H32O15 | C21H24O11 |
| LogP | 0.60 | 3.59 | −0.47 | 0.67 |
| Sol | 21.89 mg/ml | 8.59 mg/ml | 32.90 mg/ml | 11.65 mg/ml |

| | | |
|---|---|---|
| Gener- | Phloretin 2′-xyloside | Tannic acid |

| | | |
|---|---|---|
| al | | |
| English | | |
| name | | |
| CAS | N/A | 1401-55-4 |
| Chemi- cal struc- ture | | 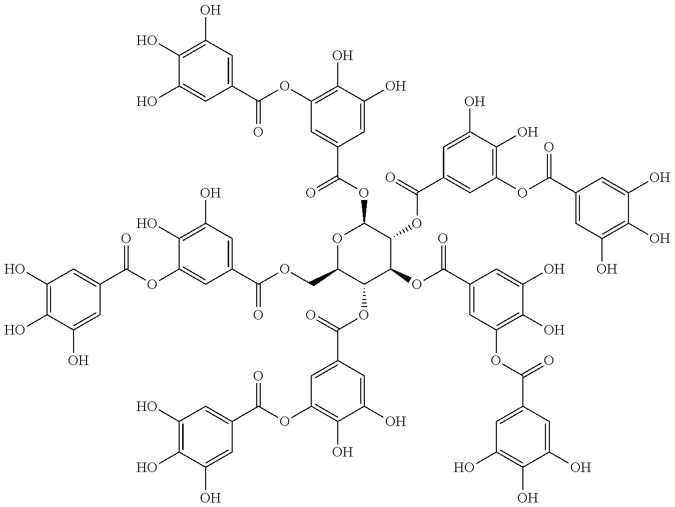 [SMILES: O=C(OC[C@H]1O[C@@H](OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1] |
| English | 1-[2- | 3,4,5-trihydroxybenzoic acid-2,3-dihydroxy-5- |
| chemi- | [(2S,3R,4S,5S,6R)- | [({[(2R,3R,4S,5R,6S)-3,4,5,6-tetra{[(4,5-dihydroxy-3- |
| cal | 4,5-dihydroxy-6- | {[(3,4,5- |
| name | (hydroxymethyl)-3- | trihydroxyphenyl)carbonyl]oxy}phenyl)carbonyl]oxy}- |
| | [(2S,3R,4S,5R)- | 3,4,5,6-tetrahydro-2H-pyran-2- |
| | 3,4,5- | yl]methyl}oxy)carbonyl]phenyl ester |
| | trihydroxyoxo-2- | |
| | yl]oxo-2-yl]oxo-4,6- | |
| | dihydroxyphenyl]-3- | |
| | (4- | |
| | hydroxyphenyl)propan- | |
| | 1-one | |
| M.W. | 568.5 | 1701.2 |
| Form. | C26H32O14 | C76H52O46 |
| LogP | −0.16 | |
| Sol | 10.26 mg/ml | |

[symbol]

indicates data missing or illegible when filed

The terpenoids selected for the present invention include but are not limited to the following:

TABLE 2
Basic information of natural terpenoids

| General | | |
|---|---|---|
| English | | |
| name | English chemical name | Chemical structure |
| Rubusoside 64849-39-4 | (1R,4S,5R,9S,10R,13S)- 13-{[(2S,3R,4S,5S,6R)- 3,4,5-trihydroxy-6- (hydroxymethyl)-3,4,5,6- tetrahydro-2H-pyran-2- yl]oxy}-5,9-dimethyl-14- methylidenetetracyclo[11.2. 1.01,10.04,9]hexadecane- 5-methanoic acid- (2S,3R,4S,5S,6R)-3,4,5- trihydroxy-6- | 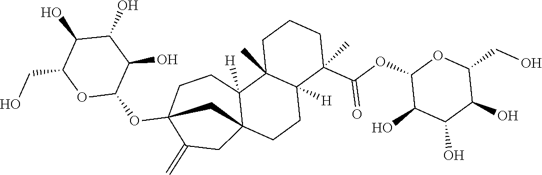 [SMILES: C=C1C[C@@]23CC[C@H]4[C@@](C)(CCC[C@@]4(C)C(=O)O[C@@H]4O[C@H](CO)[C@@H](O)[C@H](O)[C@H]4O)[C@@H]2CC[C@]1(O[C@@H]1O[C@H](CO)[C@@H](O)[C@H](O)[C@H]1O)C3] |
| | (hydroxymethyl)tetrahydro- | D,8,H-R,12 |
| | pyran-2-yl ester | Intrinsic solubility: 0.39 mg/ml |
| | | MW: 642.7 |
| Rebaudioside A EUF 58543-16-1 C44H70O23 | (1R,4S,5R,9S,10R,13S)- 13-{[(2S,3R,4S,5R,6R)-5- hydroxy-6- (hydroxymethyl)-3,4- bis{[(2S,3R,4S,5S,6R)- 3,4,5-trihydroxy-6- (hydroxymethyl)-3,4,5,6- tetrahydro-2H-pyran-2- yl]oxy}-3,4,5,6-tetrahydro- 2H-pyran-2-yl]oxy}- 5,9-dimethyl-14- methylidenetetracyclo [11.2.1.01,10.04,9] hexadecane- | 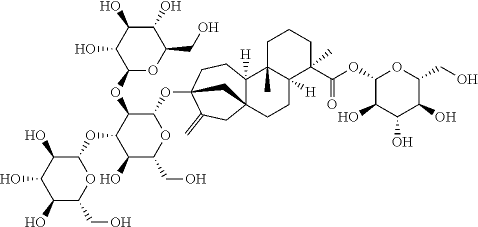 |
| | 5-methanoic acid- | Intrinsic solubility: 288.8 mg/ml |
| | (2S,3R,4S,5S,6R)-3,4,5- | H-D,14; H-R: 22 |
| | trihydroxy-6- | MW: 967.0 |
| | (hydroxymethyl)tetrahydro- | |
| | pyran-2-yl ester | |
| Rebaudioside B EUF 58543-17-2 | (1R,4S,5R,9S,10R,13S)- 13-{[(2S,3R,4S,5R,6R)-5- hydroxy-6- (hydroxymethyl)-3,4- bis{[(2S,3R,4S,5S,6R)- 3,4,5-trihydroxy-6- (hydroxymethyl)-3,4,5,6- tetrahydro-2H-pyran-2- yl]oxy}-3,4,5,6-tetrahydro- 2H-pyran-2-yl]oxy}-5,9- dimethyl-14- methylidenetetracyclo[11.2. 1.01,10.04,9]hexadecane- 5-methanoic acid | 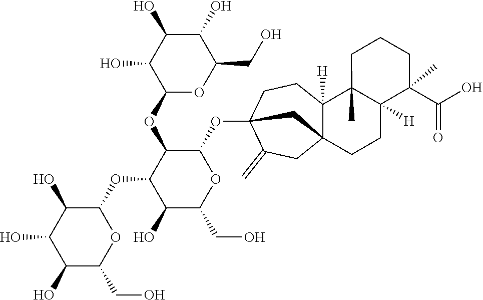 |
| | | Intrinsic solubility: 7.98 mg/ml |
| | | D,11; H-R: 18 |
| | | MW: 804.9 |
| Rebaudioside C EUF 63550-99-2 | (1R,4S,5R,9S,10R,13S)- 13-{[(2S,3R,4S,5R,6R)-5- hydroxy-6- (hydroxymethyl)-3- {[(2S,3R,4R,5R,6S)-3,4,5- trihydroxy-6-methyl- 3,4,5,6-tetrahydro-2H- pyran-2-yl]oxy}-4- {(2S,3R,4S,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}-5,9- | 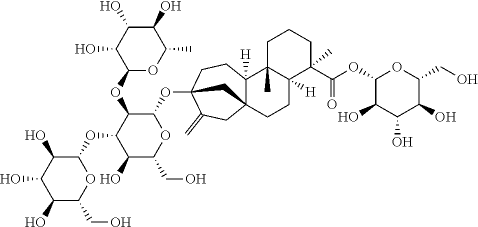 |
| | dimethyl-14- | Intrinsic solubility: 72.63 mg/ml |
| | methylidenetetracyclo[11.2. | D,13; H-R: 21 |
| | 1.01,10.04,9]hexadecane- | MW: 951.0 |
| | 5-methanoic acid- | |
| | (2S,3R,4S,5S,6R)-3,4,5- | |
| | trihydroxy-6- | |
| | (hydroxymethyl)tetrahydro- | |
| | pyran-2-yl ester | |
| Stevioside EUF 57817-89-7 | (1R,4S,5R,9S,10R,13S)- 13-{[(2S,3R,4S,5S,6R)- 4,5-dihydroxy-6- (hydroxymethyl)-3- {[(2S,3R,4S,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}-5,9- | 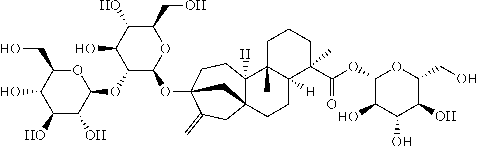 |
| | dimethyl-14- | D,11,H-R,17 |
| | methylidenetetracyclo[11.2. | Intrinsic solubility: 6.7 mg/ml |
| | 1.01,10.04,9]hexadecane- | MW: 804.9 |
| | 5-methanoic acid- | |
| | (2S,3R,4S,5S,6R)-3,4,5- | |
| | trihydroxy-6- | |
| | (hydroxymethyl)tetrahydro- | |
| | pyran-2-yl ester | |
| Soyasaponin A1 78693-94-4 | (2S,3S,4S,5R,6R)-3,4- dihydroxy-6- {[(3S,4aR,4S,6aR,6bS,8aR, 9S,10R,12aS,14aR,14bR)- 10-hydroxy-4- (hydroxymethyl)-9- {[(2S,3R,4S,5S)-3,5- dihydroxy-4- {[(2S,3R,4S,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}- 4,6a,6b,8a,11,11,14b- | 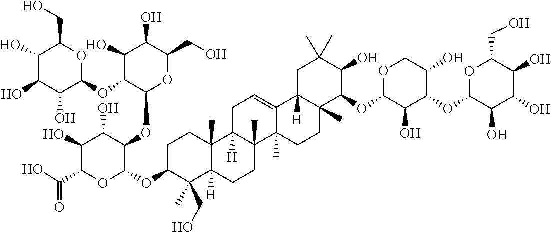 |
| | heptamethyl- | H-D,18,H-R,29 |
| | 1,2,3,4,4a,5,6,6a,6b,7,8,8a, | Intrinsic solubility: 151,826 mg/ml |
| | 9,10,11,12,12a,14,14a,14b- | MW: 1269.4 |
| | icosacyclohexano[1,2- | |
| | a]boran-3-yl]oxy}-5- | |
| | {[(2S,3R,4S,5R,6R)-4,5- | |
| | dihydroxy-6- | |
| | (hydroxymethyl)-3- | |
| | {[(2S,3R,4S,5S,6R)-3,4,5- | |
| | trihydroxy-6- | |
| | (hydroxymethyl)-3,4,5,6- | |
| | tetrahydro-2H-pyran-2- | |
| | yl]oxy}-3,4,5,6-tetrahydro- | |
| | 2H-pyran-2- | |
| | yl]oxy}tetrahydropyran-2- | |
| | methanoic acid | |
| Soyasaponin Ba 114590-20-4 | (2S,3S,4R,5R,6S)-3,4- dihydroxy-5- {[(2R,3R,4R,5S,6R)-4,5- dihydroxy-6- (hydroxymethyl)-3- {[(2R,3R,4R,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}-6- {[(3S,4aR,4R,6aR,6bS,8aR, 9S,12aR,14aR,14bS)-9- hydroxy-4- (hydroxymethyl)- 4,6a,6b,8a,11,11,14b- heptamethyl- 1,2,3,4,4a,5,6,6a,6b,7,8,8a, 9,10,11,12,12a,14,14a,14b- | 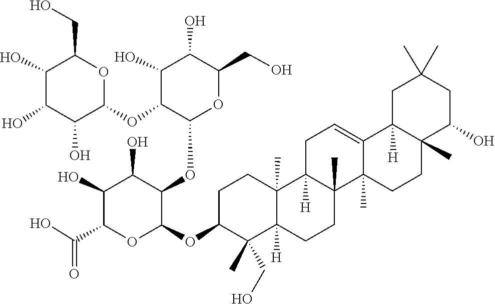 |
| | icosacyclohexano[1,2- | C48H78O19 |
| | a]boran-3- | 959.1 |
| | yl]oxy}tetrahydropyran-2- | H-D: 12 R-H: 19 |
| | methanoic acid | |
| Soyasaponin I 51330-27-9 | (2S,3S,4S,5R,6R)-3,4- dihydroxy-6- {[(3S,4aR,4S,6aR,6bS,8aR, 9R,12aS,14aR,14bR)-9- hydroxy-4- (hydroxymethyl)- 4,6a,6b,8a,11,11,14b- heptamethyl- 1,2,3,4,4a,5,6,6a,6b,7,8,8a, 9,10,11,12,12a,14,14a,14b- icosacyclohexano[1,2- a]boran-3-yl]oxy}-5- {[(2S,3R,4S,5R,6R)-4,5- dihydroxy-6- (hydroxymethyl)-3- {[(2S,3R,4R,5R,6S)-3,4,5- trihydroxy-6-methyl- 3,4,5,6-tetrahydro-2H- | 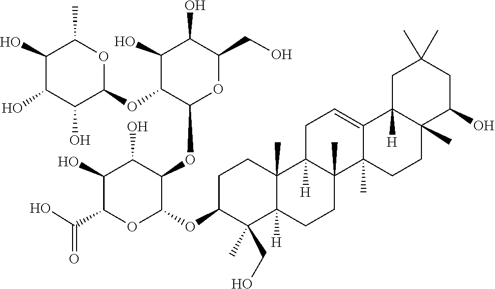 |
| | pyran-2-yl]oxy}-3,4,5,6- | C48H78O18 |
| | tetrahydro-2H-pyran-2- | 943.1 |
| | yl]oxy}tetrahydropyran-2- | H-D: 11 R-H: 18 |
| | methanoic acid | |
| Soyasaponin II 55319-36-3 | (2S,3S,4S,5R,6R)-3,4- dihydroxy-6- {[(3S,4aR,4S,6aR,6bS,8aR, 9R,12aS,14aR,14bR)-9- hydroxy-4- (hydroxymethyl)- 4,6a,6b,8a,11,11,14b- heptamethyl- 1,2,3,4,4a,5,6,6a,6b,7,8,8a, 9,10,11,12,12a,14,14a,14b- icosacyclohexano[1,2- a]boran-3-yl]oxy}-5- {[(2S,3R,4S,5S)-4,5- dihydroxy-3- {[(2S,3R,4R,5R,6S)-3,4,5- trihydroxy-6-methyl- 3,4,5,6-tetrahydro-2H- pyran-2-yl]oxy}-3,4,5,6- | 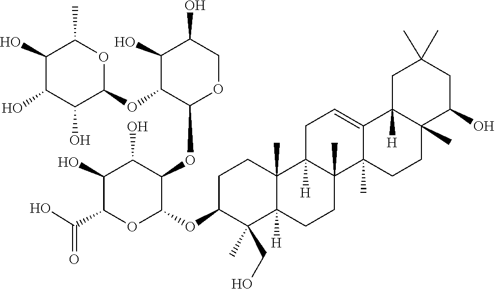 |
| | tetrahydro-2H-pyran-2- | C47H76O17 |
| | yl]oxy}tetrahydropyran-2- | 913.1 |
| | methanoic acid | H-D: 10 R-H: 17 |
| Soyasaponin III 55304-02-4 | (2S,3S,4S,5R,6R)-3,4- dihydroxy-6- {[(3S,4aR,4S,6aR,6bS,8aR, 9R,12aS,14aR,14bR)-9- hydroxy-4- (hydroxymethyl)- 4,6a,6b,8a,11,11,14b- heptamethyl- 1,2,3,4,4a,5,6,6a,6b,7,8,8a, 9,10,11,12,12a,14,14a,14b- icosacyclohexano[1,2- a]boran-3-yl]oxy}-5- {[(2S,3R,4S,5R,6R)-3,4,5- trihydroxy-6- (hydroxymethyl)-3,4,5,6- tetrahydro-2H-pyran-2- yl]oxy}tetrahydropyran-2- methanoic acid | 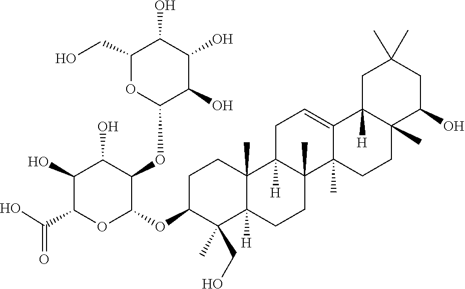 |
| | | C42H68O14 |
| | | 797.0 |
| | | H-D: 8 R-H: 14 |
| | | HLB: 29 |
| Glycyrrhizic acid 1405-86-3 EUF | (2S,3S,4S,5R,6S)-3,4- dihydroxy-5- {[(2R,3R,4S,5S,6S)-6- carboxy-3,4,5-trihydroxy- 3,4,5,6-tetrahydro-2H- pyran-2-yl]oxy}-6- {[(3S,4aR,6aR,6bS,8aS,11S, 12aR,14aR,14bS)-11- carboxy- 4,4,6a,6b,8a,11,14b- heptamethyl-14-oxo- 1,2,3,4,4a,5,6,6a,6b,7,8,8a, 9,10,11,12,12a,14,14a,14b- icosacyclohexano[1,2- a]boran-3- yl]oxy}tetrahydropyran-2- methanoic acid | 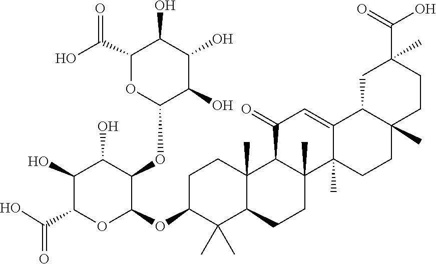 |
| | | H-D,8; H-R,16 |
| | | pH > 5.0: 159 mg/ml |
| | | MW: 822.9 |
| Oat saponin A | (2R,3S,4S,5R,6S)-2- (hydroxymethyl)-6- ({[(2′S,2S,4a′R,4b′S,6a′S,6b′R, 7′S,8′S,9a′S,10a′S,10b′S)- 2′-{[(2R,3R,4S,5S,6R)- 4-hydroxy-6- (hydroxymethyl)-3- {[(2S,3R,4R,5R,6S)-3,4,5- trihydroxy-6-methyl- 3,4,5,6-tetrahydro-2H- pyran-2-yl]oxy}-5- {[(2S,3R,4S,5S,6R)-3,4,5- trihydroxy-6- (hydroxymethyl)-3,4,5,6- tetrahydro-2H-pyran-2- | 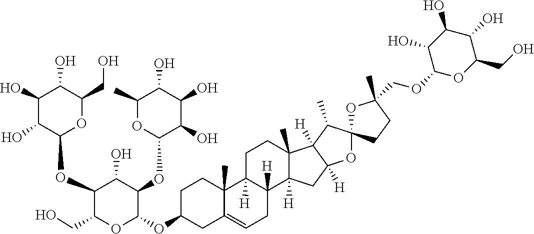 |
| | yl]oxy}-3,4,5,6-tetrahydro- | H-D,13,H-R,23 |
| | 2H-pyran-2-yl]oxy}- | Intrinsic solubility: 65.4 mg/ml |
| | 2,4a′,6a′,7′-tetramethyl- | MW: 1063.2 |
| | 2,2′,3,3′,4,4′,4a′,4b′,5′,6′,6a′, | |
| | 6b′,7′,9a′,10′,10a′,10b′,11′- | |
| | octadechydro-1′H- | |
| | spiro[furan-5,8′- | |
| | naphtho[2′,1′:4,5]indeno[2,1- | |
| | b]furan]-2- | |
| | yl]methyl}oxy)tetrahydro- | |
| | pyran-3,4,5-triol | |
| Mogroside V | (2R,3S,4S,5R,6R)-2- (hydroxymethyl)-6- ({[(2R,3S,4S,5R,6S)-3,4- dihydroxy-6-{[(3R, 6R)-2- hydroxy-6- [(1R,3aS,3bS,7S,9aR,9bR, 10R,11aR)-10-hydroxy-7- {[(2R,3R,4S,5S,6R)-3,4,5- trihydroxy-6- ({[(2R,3R,4S,5S,6R)-3,4,5- trihydroxy-6- (hydroxymethyl)-3,4,5,6- tetrahydro-2H-pyran-2- yl]oxy}methyl)-3,4,5,6- tetrahydro-2H-pyran-2- yl]oxy}-3a,6,6,9b,11a- pentamethyl- 2,3,3a,3b,4,6,7,8,9,9a,9b, 10,11,11a-tetrahydro-1H- cyclopentano[1,2- | 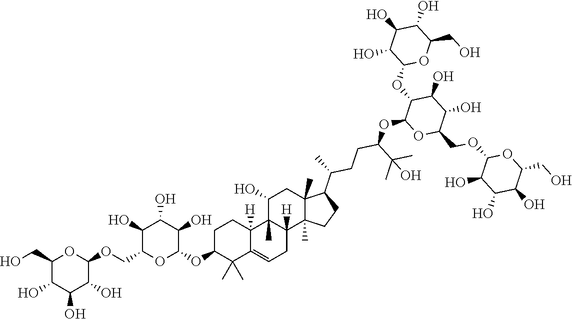 |
| | a]phenanthr-1-yl]-2- | D,19,H-R,29 |
| | methylhept-3-yl]oxy}-5- | Intrinsic solubility: 763808 mg/ml |
| | {(2R,3R,4S,5S,6R)-3,4,5- | MW 1287.4 |
| | trihydroxy-6- | |
| | (hydroxymethyl)-3,4,5,6- | |
| | tetrahydro-2H-pyran-2- | |
| | yl]oxy}-3,4,5,6-tetrahydro- | |
| | 2H-pyran-2- | |
| | yl]methyl}oxy)tetrahydro- | |
| | pyran-3,4,5-triol | |
| Mogroside IV | (2R,3S,4S,5R,6R)-2- (hydroxymethyl)-6- ({[(2R,3S,4S,5R,6R)-3,4,5- trihydroxy-6- {[(1R,3aS,3bS,7S,9aR,9bR, 10R,11aR)-10-hydroxy-1- [(2R,5R)-6-hydroxy-5- {[(2S,3R,4S,5S,6R)-4,5- dihydroxy-6- (hydroxymethyl)-3- {[(2S,3R,4S,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}-6- methylhept-2-yl]- 3a,6,6,9b,11a-pentamethyl- 2,3,3a,3b,4,6,7,8,9,9a,9b,10, 11,11a-tetradechydro-1H- cyclopentano[1,2- a]phenanthr-7-yl]oxy}- | 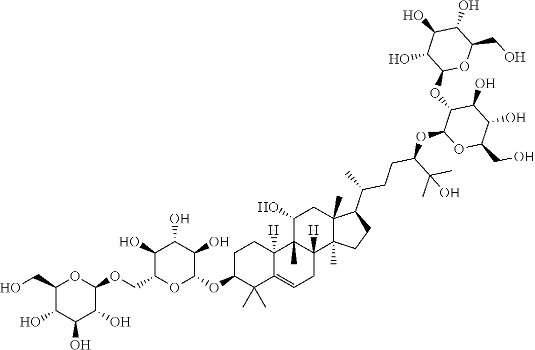 |
| | 3,4,5,6-tetrahydro-2H- | Intrinsic solubility: 3459 mg/ml |
| | pyran-2- | MW 1125.0 |
| | yl]methyl}oxy)tetrahydro- | D-H: 16 R-H: 24 |
| | pyran-3,4,5-triol | |
| Rebaudioside D EUF | (1R,4S,5R,9S,10R,13S)- 13-{[(2S,3R,4S,6S)-6- (hydroxymethyl)-4- {[(2R,3R,4S,5S,6R)-3,4,5- trihydroxy-6- (hydroxymethyl)-3,4,5,6- tetrahydro-2H-pyran-2- yl]oxy}-3- {[(2S,3R,4S,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}-5,9- dimethyl-14- methylidenetetracyclo [11.2.1.01,10.04,9] hexadecane- 5-methanoic acid- (2S,3R,4S,5S,6R)-4,5- dihydroxy-6- (hydroxymethyl)-3- | 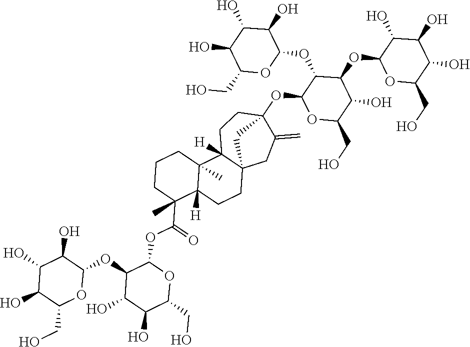 |
| | {[(2S,3R,4S,5S,6R)-3,4,5- | 63279-13-0, C₅₀H₈₀O₂₈ |
| | trihydroxy-6- | 1129.16 |
| | (hydroxymethyl)-3,4,5,6- | Intrinsic solubility: 31060 g/mL |
| | tetrahydro-2H-pyran-2- | D-H: 17 R-H: 27 |
| | yl]oxy}tetrahydropyran-2- | |
| | yl ester | |
| Rebaudioside M EUF | (1R,4S,5R,9S,10R,13S)- 13-{[(2S,3R,4S,5R,6R)-5- hydroxy-6- (hydroxymethyl)-3,4- bis{[(2S,3R,4S,5S,6R)- 3,4,5-trihydroxy-6- (hydroxymethyl)-3,4,5,6- tetrahydro-2H-pyran-2- yl]oxy}-3,4,5,6-tetrahydro- 2H-pyran-2-yl]oxy}-5,9- dimethyl-14- methylidenetetracyclo[11.2. 1.01,10.04,9]hexadecane- 5-methanoic acid- (2S,3R,4S,5R,6R)-5- hydroxy-6- (hydroxymethyl)-3,4- bis{[(2S,3R,4S,5S,6R)- 3,4,5-trihydroxy-6- (hydroxymethyl)-3,4,5,6- tetrahydro-2H-pyran-2- yl]oxy}tetrahydropyran-2- yl ester | 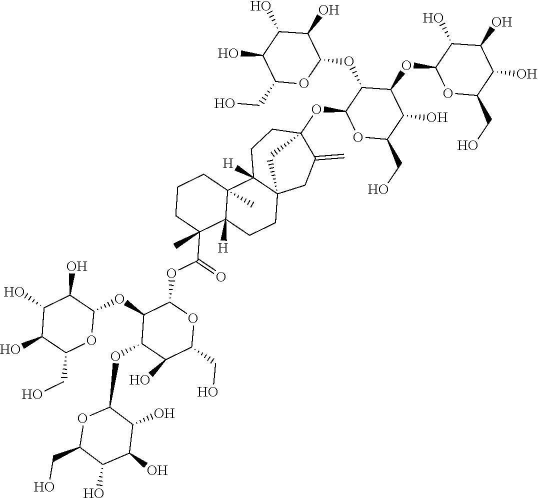 |
| | | 1220616-44-3, C₅₆H₉₀O₃₃ |
| | | 1291.30 |
| | | D-H: 20 R-H: 32 |
| | | Intrinsic solubility: 8453596 g/mL |
| Asiaticoside | (1S,2R,4aS,6aS,6bR,8aR, 9R,10R,11R,12aR,14bS)- 10,11-dihydroxy-9- (hydroxymethyl)- 1,2,6a,6b,9,12a- hexamethyl- 1,2,3,4,4a,5,6,6a,6b,7,8,8a, 9,10,11,12,12a,12b,13,14b- icosacyclohexano[1,2- a]boran-4a-methanoic acid- (2S,3R,4S,5S,6R)-3,4,5- trihydroxy-6- ({[(2R,3R,4R,5S,6R)-3,4- dihydroxy-6- (hydroxymethyl)-5- {[(2S,3R,4R,5R,6S)-3,4,5- trihydroxy-6-methyl- | 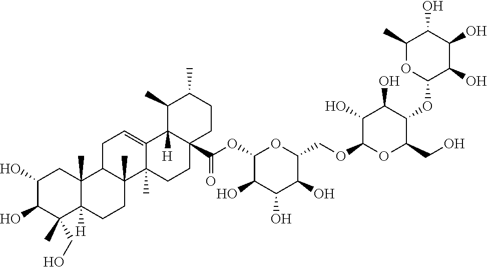 |
| | 3,4,5,6-tetrahydro-2H- | C₄₈H₇₈O₁₉ |
| | pyran-2-yl]oxy}-3,4,5,6- | 959.12 |
| | tetrahydro-2H-pyran-2- | D-H: 12 R-H: 18 |
| | yl]oxy}methyl)tetrahydro- | |
| | pyran-2-yl ester | |
| Asiaticoside A | (1S,2R,4aS,6aR,6bR,8aR,8R, 9R,10R,11R,12aR,14bS)- 8,10,11-trihydroxy-9- (hydroxymethyl)- 1,2,6a,6b,9,12a- hexamethyl- 1,2,3,4,4a,5,6,6a,6b,7,8,8a, 9,10,11,12,12a,12b,13,14b- icosacyclohexano[1,2- a]boran-4a-methanoic acid- (2S,3R,4S,5S,6R)-3,4,5- trihydroxy-6- ({[(2R,3R,4R,5S,6R)-3,4- dihydroxy-6- (hydroxymethyl)-5- {[(2S,3R,4R,5R,6S)-3,4,5- trihydroxy-6-methyl- | 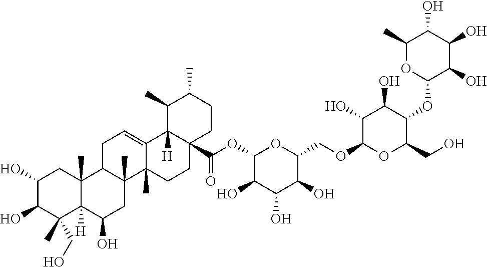 |
| | 3,4,5,6-tetrahydro-2H- | C₄₈H₇₈O₂₀ |
| | pyran-2-yl]oxy}-3,4,5,6- | 975.12 |
| | tetrahydro-2H-pyran-2- | D-H: 13 R-H: 19 |
| | yl]oxy}methyl)tetrahydro- | |
| | pyran-2-yl ester | |
| Asiaticoside B | (4aS,6aS,6bR,8aR,8R,9R, 10R,11R,12aR,14bS)- 8,10,11-trihydroxy-9- (hydroxymethyl)- 2,2,6a,6b,9,12a- hexamethyl- 1,2,3,4,4a,5,6,6a,6b,7,8,8a, 9,10,11,12,12a,12b,13,14b- icosacyclohexano[1,2- a]boran-4a-methanoic acid- (2S,3R,4S,5S,6R)-3,4,5- trihydroxy-6- ({[(2R,3R,4R,5S,6R)-3,4- dihydroxy-6- (hydroxymethyl)-5- {[(2S,3R,4R,5R,6S)-3,4,5- trihydroxy-6-methyl- | 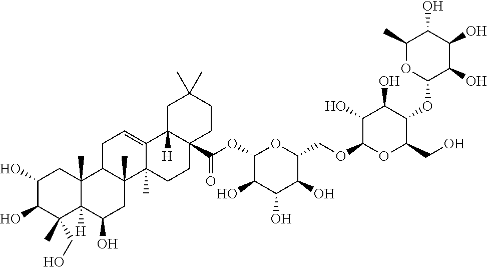 |
| | 3,4,5,6-tetrahydro-2H- | C₄₈H₇₈O₂₀ |
| | pyran-2-yl]oxy}-3,4,5,6- | 975.12 |
| | tetrahydro-2H-pyran-2- | D-H: 13 R-H: 19 |
| | yl]oxy}methyl)tetrahydro- | |
| | pyran-2-yl ester | |
| Asiaticoside F | (1S,2R,4aS,6aS,6bR,8aR, 9R,10S,12aR,14bS)-10- hydroxy-9- (hydroxymethyl)- 1,2,6a,6b,9,12a- hexamethyl- 1,2,3,4,4a,5,6,6a,6b,7,8,8a, 9,10,11,12,12a,12b,13,14b- icosacyclohexano[1,2- a]boran-4a-methanoic acid- (2S,3R,4S,5S,6R)-3,4,5- trihydroxy-6- ({[(2R,3R,4R,5S,6R)-3,4- dihydroxy-6- (hydroxymethyl)-5- {[(2S,3R,4R,5R,6S)-3,4,5- trihydroxy-6-methyl- | 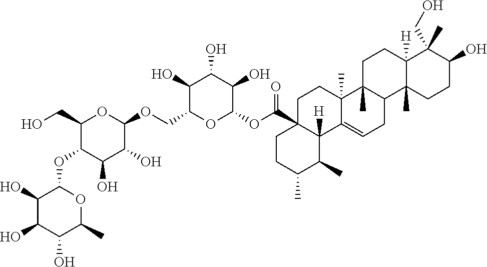 |
| | 3,4,5,6-tetrahydro-2H- | C₄₈H₇₈O₁₈ |
| | pyran-2-yl]oxy}-3,4,5,6- | 943.1 |
| | tetrahydro-2H-pyran-2- | D-H: 13 R-H: 19 |
| | yl]oxy}methyl)tetrahydro- | |
| | pyran-2-yl ester | |
| Asiaticoside E | (1S,2R,4aS,6aS,6bR,8aR, 9R,10R,11R,12aR,14bS)- 10,11-dihydroxy-9- (hydroxymethyl)- 1,2,6a,6b,9,12a- hexamethyl- 1,2,3,4,4a,5,6,6a,6b,7,8,8a, 9,10,11,12,12a,12b,13,14b- icosacyclohexano[1,2- a]boran-4a-methanoic acid-(2S,3R,4S,5S,6R)- 3,4,5-trihydroxy-6- ({[(2R,3R,4S,5S,6R)-3,4,5- trihydroxy-6- | 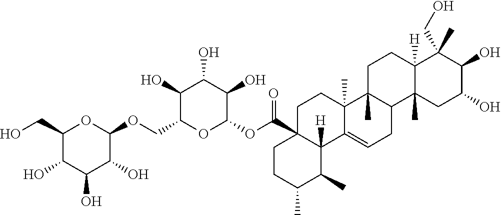 |
| | (hydroxymethyl)-3,4,5,6- | C₄₂H₆₈O₁₅ |
| | tetrahydro-2H-pyran-2- | 813.0 |
| | yl]oxy}methyl)tetrahydro- | D-H: 11 R-H: 17 |
| | pyran-2-yl ester | |
| Ginsenoside RG1 22427-39-0 | (2R,3S,4S,5R,6S)-2- (hydroxymethyl)-6-{[(2S)- 2- [(1S,3aR,3bR,5aR,5S,7S, 9aR,9bR,11aR,11R)-7,11- dihydroxy-5- {[(2R,3R,4S,5S,6R)-3,4,5- trihydroxy-6- (hydroxymethyl)-3,4,5,6- tetrahydro-2H-pyran-2- yl]oxy}-3a,3b,6,6,9a- pentamethylhexadechydro- 1H-cyclopentano[1,2- a]phenanthr-1-yl]-6- methylhept-5-en-2- yl]oxy}tetrahydropyran- 3,4,5-triol | 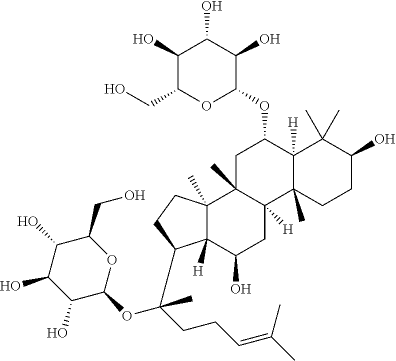 [SMILES: CC(C)=CCC[C@](C)(O[C@@H]1O[C@H](CO)[C@@H](O)[C@H](O)[C@H]1O)[C@H]1CC[C@]2(C)[C@@H]1[C@H](O)C[C@@H]1[C@@]3(C)CC[C@H](O)C(C)(C)[C@@H]3[C@@H](O[C@@H]3O[C@H](CO)[C@@H](O)[C@H](O)[C@H]3O)C[C@]12C] |
| | | C₄₂H₇₂O₁₄ |
| | | 801.0 |
| | | D-H: 10 R-H: 14 |
| Ginsenoside Rb1 41753-43-9 | (2S,3R,4S,5S,6R)-6- ({[(2R,3R,4S,5S,6R)-3,4,5- trihydroxy-6- (hydroxymethyl)-3,4,5,6- tetrahydro-2H-pyran-2- yl]oxy}methyl)-2-{(2S)-2- [(1S,3aR,3bR,5aR,7S,9aR, 9bR,11aR,11R)-11- hydroxy-7- {[(2R,3R,4S,5S,6R)-4,5- dihydroxy-6- (hydroxymethyl)-3- {[(2S,3R,4S,5S,6R)-3,4,5- trihydroxy-6- (hydroxymethyl)-3,4,5,6- tetrahydro-2H-pyran-2- yl]oxy}-3,4,5,6-tetrahydro- 2H-pyran-2-yl]oxy}- 3a,3b,6,6,9a- | 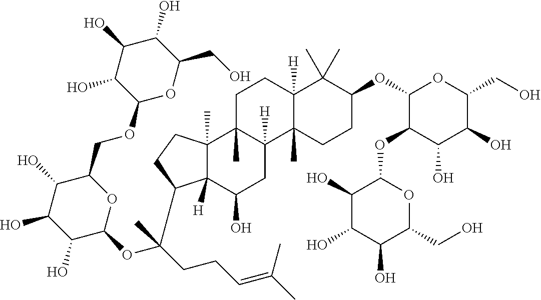 |
| | pentamethylhexadechydro- | C₅₄H₉₂O₂₃ |
| | 1H-cyclopentano[1,2- | 1109.3 |
| | a]phenanthr-1-yl]-6- | D-H: 15 R-H: 23 |
| | methylhept-5-en-2- | |
| | yl]oxy}tetrahydropyran- | |
| | 3,4,5-triol | |
| Glycyrrhetinic acid-3-O- glucuronide CAS 34096- 83-8 | (2S,3S,4S,5R,6R)-3,4,5- trihydroxy-6- {[(3S,4aR,6aR,6bS,8aS, 11S,12aR,14aR,14bS)-11- carboxy- 4,4,6a,6b,8a,11,14b- heptamethyl-14-oxo- 1,2,3,4,4a,5,6,6a,6b,7,8,8a, 9,10,11,12,12a,14,14a,14b- icosacyclohexano[1,2- a]boran-3- yl]oxy}tetrahydropyran-2- methanoic acid | 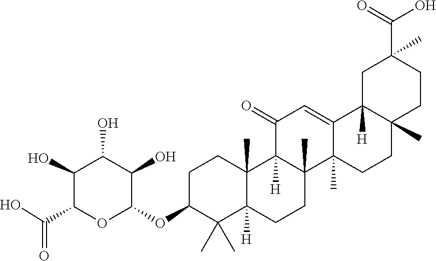 |
| | | C₃₆H₅₄O₁₀ |
| | | 646.8 |
| | | D-H: 5 R-H: 10 |
| Dioscin 19057-60-4 | (2S,3R,4R,5R,6S)-6- {[(2R,3R,4S,5S,6R)-4- hydroxy-6- (hydroxymethyl)-2- {[(2′R,2S,4aR,4bS,5′R,6aS, 6bR,7S,9aS,10aS,10bS)- 4a,5′,6a,7-tetramethyl- 1,2,3,3′,4,4′,4a,4b,5,5′,6,6′, 6a,6b,7,9a,10,10a,10b,11- icosahydrospiro[naphtho [2′,1′:4,5]indeno[2,1-b]furan- 8,2′-pyran]-2-yl]oxy}-5- {[(2S,3R,4R,5R,6S)-3,4,5- trihydroxy-6-methyl- 3,4,5,6-tetrahydro-2H- | 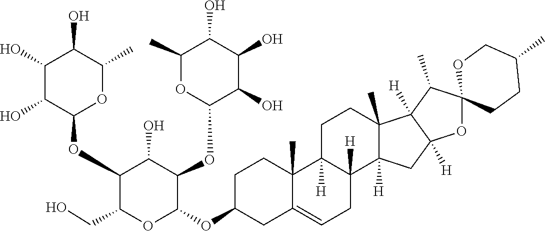 |
| | pyran-2-yl]oxy}-3,4,5,6- | C₄₅H₇₂O₁₆ |
| | tetrahydro-2H-pyran-3- | 869.0 |
| | yl]oxy}-2- | D-H: 8 R-H: 16 |
| | methyltetrahydropyran- | |
| | 3,4,5-triol | |
| Platycodin A | [(2S,3R,4S,5S)-3- [(2S,3R,4R,5R,6S)-3- acetoxy-5-[(2S,3R,4S,5R)- 4-[(2S,3R,4R)-3,4- dihydroxy-4- (hydroxymethyl)oxan-2- yl]oxy-3,5- dihydroxyloxan-2-yl]oxy- 4-hydroxy-6-methyloxan- 2-yl]oxy-4,5- dihydroxyloxan-2- yl](4aR,5R,6aR,6aS,6bR, | 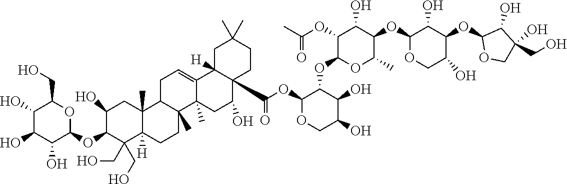 [SMILES: CC(=O)O[C@H]1[C@H](O[C@H]2[C@H](OC(=O)[C@]34CCC(C)(C)C[C@H]3C3=CCC5[C@@]6(C)C[C@H](O)[C@H](O[C@@H]7O[C@H](CO)[C@@H](O)[C@H](O)[C@H]7O)C(CO)(CO)[C@@H]6CC[C@@]5(C)[C@]3(C)C[C@H]4O)OC[C@H](O)[C@@H]2O)O[C@@H](C)[C@H](O[C@@H]2OC[C@@H](O)[C@H](O[C@@H]3OC[C@](O)(CO)[C@H]3O)[C@H]2O)[C@H]1O] |
| | 8aR,10R,11S,12aR,14bS)- | C₅₉H₉₄O₂₉ |
| | 5,11-dihydroxy-9,9- | 1267.36 |
| | bis(hydroxymethyl)- | D-H: 16 R-H: 27 |
| | 2,2,6a,6b,12a-pentamethyl- | |
| | 10-[(2R,3R,4S,6R)-3,4,5- | |
| | trihydroxy-6- | |
| | (hydroxymethyl)oxy-2- | |
| | yl]oxy- | |
| | 3,4,5,6,6a,7,8,8a,10,11,12, | |
| | 13,14b-tetradecene-4a- | |
| | carboxylate | |
| Platycodin B | [(2S,3R,4S,5S)-3- [(2S,3R,4R,5R,6S)-3- acetoxy-4-hydroxy-6- methyl-5-[(2S,3R,4S,5R)- 3,4,5-trihydroxyoxy-2- yl]oxy-2-yl]oxy-4,5- dihydroxyalkoxy-2- yl](4aR,5R,6aR,6aS,6bR, 8aR,10R,11S,12aR,14bS)- 5,11-dihydroxy-9,9-bis (hydroxymethyl)- 2,2,6a,6b,12a-pentamethyl- 10-[(2R,3R,4S,5S,6R)- | 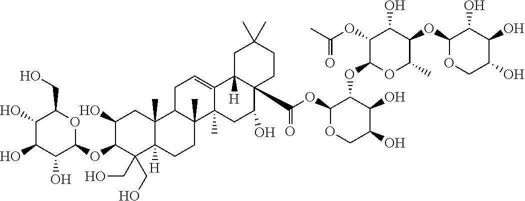 |
| | 3,4,5-trihydroxy-6- | C₅₄H₈₆O₂₅ |
| | (hydroxymethyl)oxy-2- | 1135.25 |
| | yl]oxy- | D-H: 14 R-H: 23 |
| | 1,3,4,5,6,6a,7,8,8a,10,11, | |
| | 12,13,14b-tetradecene-4a- | |
| | carboxylate | |
| Platycodin D | [(2S,3R,4S,5S)-3- [(2S,3R,4S,5R,6S)-5- [(2S,3R,4S,5R)-4- [(2S,3R,4R)-3,4- dihydroxy-4- (hydroxymethyl)oxan-2- yl]oxy-3,5-dihydroxyoxan- 2-yl]oxy-3,4-dihydroxy-6- methyloxan-2-yl]oxy]-4,5- dihydroxyoxan-2-yl] (4aR,5R,6aR,6aS,6bR,8aR, 10R,11S,12aR,14bS)-5,11- | 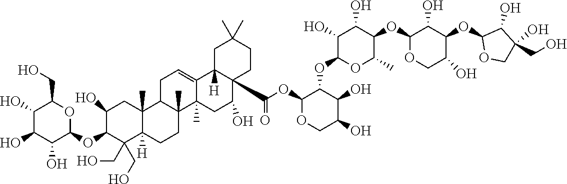 |
| | dihydroxy-9,9- | C₅₇H₉₂O₂₈ |
| | bis(hydroxymethyl)- | 1225.32 |
| | 2,2,6a,6b,12a-pentamethyl- | D-H: 17 R-H: 27 |
| | 10-[(2R,3R,4S,5S,6R)- | |
| | 3,4,5-trihydroxy-6- | |
| | (hydroxymethyl)oxan-2- | |
| | yl]oxy- | |
| | 1,3,4,6,6a,7,8a,10,11,12,13, | |
| | 14b-tetradecene-4a- | |
| | carboxylate | |
| Platycodin D2 | [(2S,3R,4S,5S)-3- [(2S,3R,4S,5R, 6S)-5- [(2S,3R,4S,5R)-4- [(2S,3R,4R)-3,4- dihydroxy-4- (hydroxymethyl)oxan-2- yl]oxy-3,5-dihydroxyoxan- 2-yl]oxy-3,4-dihydroxy-6- methyloxan-2-yl]oxy]- (4aR,5R,6aR,6aS,6bR,8aR, 10R,11S,12aR,14bS)-10- | 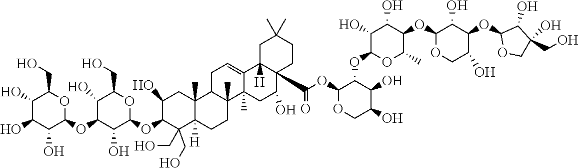 |
| | [(2R,3R,4S,5R,6R)-3,5- | C₆₃H₁₀₂O₃₃ |
| | dihydroxy-6- | 1387.46 |
| | (hydroxymethyl)-4- | D-H: 20 R-H: 32 |
| | [(2S,3R,4S,5S,6R)-3,4,5- | |
| | trihydroxy-6- | |
| | (hydroxymethyl)oxo-2- | |
| | yl]oxan-2-yl]oxo-5,11- | |
| | dihydroxy-9,9- | |
| | bis(hydroxymethyl)- | |
| | 2,2,6a,6b,12a-pentamethyl- | |
| | 1,3,4,5,6,6a,7,8,8a,10,11,12, | |
| | 13,14b-tetradecene-4a- | |
| | carboxylate | |
| Platycodin D3 | [(2S,3R,4S,5S)-3- [(2S,3R,4S,5R,6S)-5- [(2S,3R,4S,5R)-4- [(2S,3R,4R)-3,4- dihydroxy-4- (hydroxymethyl)oxan-2- yl]oxy-3,5-dihydroxyoxan- 2-yl]oxy-3,4-dihydroxy-6- methyloxan-2-yl]oxy-4,5- dihydroxyoxan-2- yl](4aR,5R,6aR,6BS,8aR, 10R,11S, hydroxy-149- dihydroxy-BS)12a- | 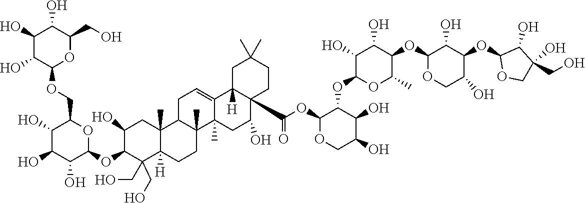 |
| | pentamethyl-10- | C₆₃H₁₀₂O₃₃ |
| | [(2R,3R,4S,5S,6R)-3,4,5- | 1387.46 |
| | trihydroxy-6- | D-H: 20 R-H: 32 |
| | [(2R,3R,4S,5S,6R)-3,4,5- | |
| | trihydroxy-6- | |
| | (hydroxymethyl)oxy-2- | |
| | yl]oxy]oxy- | |
| | 1,3,4,5,6,6a,7,8,8a,10,11, | |
| | 12,14b-tetradecene-4a- | |
| | carboxylate | |
| Tenuigenin A | (2S,3R,4S,4aR,6aR,6bR,8aS, 12aS,14mR,14bR)-8a- [(2S,3R,4S,5S,6R)-3- [(2S,3R,4S,5S,6S)-4- [(2S,3R,4R)-3,4- dihydroxy-4- (hydroxymethyl) oxazolidin-2-acyl]oxy-5- [(2S,3R,4R,5R)-3,4- dihydroxy-5- [(2S,3R,4S,5R,6R)-3,4,5- trihydroxy-6- (hydroxymethyl)oxo-2- yl]oxo-2-yl]oxo-3- hydroxy-6-methyloxo-2- yl]oxo-5-[(E)-3-(4- methoxyphenyl)prop-2- enyl]oxo-6-methyl-4- [(2S,3R,4R,5R,6S)-3,4,5- trihydroxy-6-methoxy-2- | 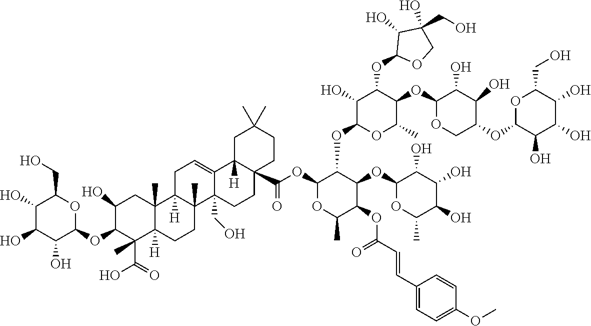 |
| | yl]oxy-2-yl]oxycarbonyl-2- | C₈₀H₁₂₀O₃₉ |
| | hydroxy-6b- | 1705.79 |
| | (hydroxymethyl)- | D-H: 20 R-H: 37 |
| | 4,6a,11,11,14b- | |
| | pentamethyl-3- | |
| | [(2R,3R,4S,5S,6R)-3,4,5- | |
| | trihydroxy-6- | |
| | (hydroxymethyl)oxy-2- | |
| | yl]oxy- | |
| | 1,2,3,4a,5,6,7,8,9,10,12, | |
| | 12a,14,14a-tetradecene-4- | |
| | carboxylic acid | |
| Tenuigenin D | [(2S,3R,4S,5R)-3- [(2S,3R,4S,5R,6S)-5- [(2S,3R,4S,5R)-4- [(2S,3R,4R)-3,4- dihydroxy-4- (hydroxymethyl)oxan-2- yl]oxy-3,5-dihydroxyoxan- 2-yl]oxy-3,4-dihydroxy-6- methyloxan-2-yl]oxy-4,5- dihydroxyoxan-2-yl]- (4aR,5R,6aR,6aS,6bR,8aR, 9R,10R,11S,12aR,14bS)- | 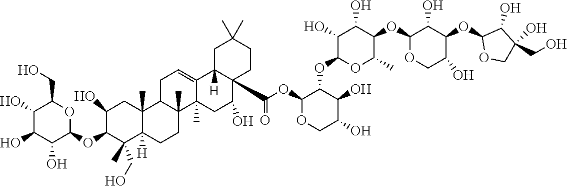 |
| | 5,11-dihydroxy-9- | C₅₇H₉₂O₂₇ |
| | (hydroxymethyl)- | 1209.32 |
| | 2,2,6a,6b,9,12a- | D-H: 16 R-H: 26 |
| | hexamethyl-10- | |
| | [(2R,3R,4S,5S,6R)-3,4,5- | |
| | trihydroxy-6- | |
| | (hydroxymethyl)oxy- | |
| | 1,3,4,5,6,6a,7,8,8a,10,11, | |
| | 12,14b-tetradecene-4a- | |
| | carboxylate | |
| Tenuigenin D2 | [(2S,3R,4S,5S)-3- [(2S,3R,4S,5R,6S)-5- [(2S,3R,4S,5R)-4- [(2S,3R,4R)-3,4- dihydroxy-4- (hydroxymethyl)oxan-2- yl]oxy-3,5-dihydroxyoxan- 2-yl]oxy-3,4-dihydroxy-6- methyloxan-2-yl]oxy-4,5- dihydroxyoxan-2-yl] (4aR,5R,6aR,6aS,6bR,8aR, | 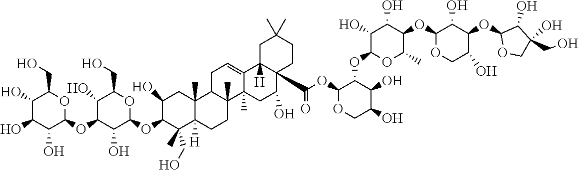 |
| | 9R,10R,11S,12aR,14bS)- | C₆₃H₁₀₂O₃₂ |
| | 10-[(2R,3R,4S,5R,6R)-3,5- | 1371.46 |
| | dihydroxy-6- | D-H: 19 R-H: 31 |
| | (hydroxymethyl)-4- | |
| | [(2S,3R,4S,5S,6R)-3,4,5- | |
| | trihydroxy-6- | |
| | (hydroxymethyl)oxo-2- | |
| | yl]oxo-5,11-dihydroxy-9- | |
| | (hydroxymethyl)- | |
| | 2,2,6a,6b,9,12a- | |
| | hexamethyl- | |
| | 1,3,4,5,6,6a,7,8,8a,10,11, | |
| | 12,13,14b-tetradecene-4a- | |
| | carboxylate | |

The high molecular polymers selected in the present invention include but are not limited to the following:

Natural high molecular polymers or modified materials, such as cellulose, starch, soluble starch, wheat starch, potato starch, cassava starch, Gellan gum, maltodextrin, hyaluronic acid, corn gluten, corn starch, tragacanth gum, arabic gum, alginic acid, sodium alginate, pectin, chitosan, arabinogalactan, polysaccharides or polysaccharide extracts, xanthan gum, cyclodextrin and derivatives thereof;

### the Semi-Synthetic High Molecular Polymers Include:

2) Celluloses: hydroxypropyl methylcellulose, methyl cellulose, acetate cellulose, ethyl cellulose, hydroxypropyl cellulose, low-substituted hydroxypropyl cellulose, microcrystalline cellulose, carboxymethyl cellulose, carboxymethyl starch sodium, hydroxypropyl methylcellulose acetate succinate, hydroxypropyl methylcellulose phthalate, cross-linked carboxymethyl cellulose sodium or calcium, and silicified microcrystalline cellulose;
3) Artificially synthesized high molecular polymers: preferred polyethylene caprolactam-polyvinyl acetate-polyethylene glycol graft copolymer, copovidone, polyvinyl pyrrolidone series, polyethylene glycol series, ethyl acrylate-methyl methacrylate-trimethylamine ethyl methacrylate chloride (1:2:0.2) copolymer, ethyl acrylate-methyl methacrylate-trimethylamine ethyl methacrylate chloride (1:2:0.1) copolymer, methacrylic acid-ethyl acrylate (1:1) copolymer, methacrylic acid-methyl methacrylate (1:1) copolymer, methacrylic acid-methyl methacrylate (1:2) copolymer, butyl methacrylate-dimethylaminoethyl methacrylate-methyl methacrylate (1:2:1) copolymer, ethyl acrylate-methyl methacrylate (2:1) copolymer, glycolide lactide copolymer series, carbomer, carbomer copolymer, polylactic acid-hydroxyglycolic acid copolymer, polylactic acid, and polylactic acid-glycollic acid copolymer.
4) Surfactants or emulsifiers: sorbitan trioleate, lauroyl polyoxyethylene glyceride, oleoyl polyoxyethylene glyceride, oleic acid polyoxyethylene ester, sodium dodecyl sulfate, polysorbate (Tween20, 80), poloxamer, vitamin E succinate polyethylene glycol ester (TPGS), stearic acid polyoxometalate, polyvinyl alcohol, polyammonium methacrylate, polyoxyethylene, polyoxyethylene castor oil, polyoxyethylene hydrogenated castor oil, etc. Sorbitan trioleate, lauroyl polyoxyethylene glyceride, polysorbate, sodium dodecyl sulfate, and poloxamer are preferred in the present invention.

The supramolecular self-assembly system based on natural building blocks, provided by the present invention, can be formulated into drug formulations or dietary supplements suitable for mammalian medical or health purposes. The compositions can be routinely present in unit dosage forms and prepared by any method in the pharmaceutical field. The supramolecular self-assembly system built by the present invention includes one or more active ingredients of a therapeutic effective dose, one or more high molecular polymers and one or more natural building blocks in the system, as well as one or more inert excipients or additives acceptable in the fields of pharmacy, food, etc., any other therapeutic ingredients, stabilizers, etc. The compositions prepared from other acceptable excipients or additives in the pharmaceutical or food industry and the supramolecular self-assembly system including target guest ingredients, polymer building blocks, and natural building blocks, include formulations suitable for oral delivery, rectal delivery, local delivery, nasal delivery, ocular delivery, or parenteral delivery (including intraperitoneal, intravenous, subcutaneous, or intramuscular injection). On this basis, the present invention provides a delivery system comprising the supramolecular self-assembly system described above, as well as acceptable carriers, excipients, diluents, adjuvants, mediators, or combinations thereof in the pharmaceutical or food science fields. The dosage form of the delivery system may be selected from suitable dosage forms familiar to those skilled in the art, such as injection, lyophilized powder for injection, oral solid preparation, oral liquid preparation, oral suspension, external patch, gel, cream, dry suspension, eye drops, eye paste, and parenteral nutrition.

Preferably, the oral solid preparation of the present invention is selected from ordinary tablets or capsules, sustained-release tablets or capsules, controlled-release tablets or capsules, granules/dry suspensions, films, rapidly disintegrating oral tablets, sublingual tablets, oral cavity Capsular patches, etc.

Specifically, for oral therapeutic or healthcare applications, the oral solid preparation may be combined with one or more other excipients and used in the form of swallowable tablets, buccal tablets, sugar-coated tablets, capsules, elixirs, suspensions, syrups, powders, etc. The excipients can be (but are not limited to): adhesives, such as hydroxypropyl cellulose, povidone, or hydroxypropyl methyl cellulose; fillers, such as microcrystalline cellulose, pre-gelatinized starch, starch, mannitol, or lactose; disintegrating agents, such as cross-linked carboxymethyl cellulose sodium, cross-linked povidone, or sodium starch glycolate; lubricants, such as magnesium stearate, stearic acid, or other metal stearates; sweeteners, such as sucrose, fructose, lactose, or aspartame; and/or seasonings, such as peppermint, wintergreen oil, or cherry flavorings. When the dosage form is capsule, in addition to the above types of materials, the delivery system may further include liquid carriers, such as vegetable oil or polyethylene glycol. Various other materials may exist in a coating form or in other forms that alter the physical form of the solid dosage form. For example, tablets, pills, or capsules may be coated with gelatin, polymers, wax, lac, sugar, etc. Of course, any material used for preparing any dosage form will typically be pharmaceutically acceptable and substantially non-toxic in the amounts used.

The solution or emulsion used for parenteral, intradermal or subcutaneous administration may include the following ingredients: sterile diluent, such as water for injection, saline solution, oil, polyethylene glycol, glycerin, propylene glycol, or other synthetic solvents; antibacterial agents, such as benzyl alcohol or methylparaben; antioxidants, such as ascorbic acid or sodium bisulfite; chelating agents, such as ethylenediaminetetraacetic acid; buffers, such as acetate, citrate, or phosphate, and agents used for regulating tension, such as sodium chloride or glucose. The pH regulators, such as hydrochloric acid or sodium hydroxide. The gastrointestinal preparations may be enclosed in glass or plastic ampoules, disposable syringes, or multi-dose vials, and prepared as injections, lyophilized powder for injection or infusions.

The composition used for rectal administration can manifest as a suppository with a suitable matrix containing, for example, cocoa butter or salicylate esters.

For nasal or inhalation administration, the compounds used according to the present invention are suitable for delivery in the form of a spray formed by compressing the package or using an aerosolizer, and suitable propellants such as dichlorodifluoromethane, trichlorofluoromethane, dichlorotetrafluoroethane, carbon dioxide or other suitable gases are used. When pressurized spray is used, the dosage unit can be determined by providing a valve for delivering the metered quantity. Gelatin capsules and blister packs used for inhalers or nebulizers, for example, can be formulated to contain a powder mixture of the compound and suitable powder base materials (such as lactose or starch).

For ocular delivery, the present invention is based on the target substance (active ingredient) in the supramolecular self-assembly delivery system for any eye disease or disorder treatment or providing health benefits to the eye, to maintain the active ingredient or healthy ingredient in contact with the eye surface for a sufficient period of time to allow the active substance or healthy ingredient to penetrate the cornea and internal regions of the eye, including, for example, the anterior chamber, posterior chamber, vitreous body, aqueous humor, lens fluid, iris/ciliary body, lens, choroid/retina, and sclera. Pharmaceutically acceptable excipients for eye use may, for example be, ointments, vegetable oils, or enclosed materials. The supramolecular self-assembly system of the present invention can also be directly injected into vitreous fluid and aqueous humor or subtenon injection after production and quality well controlled.

For transdermal or skin delivery, the supramolecular self-assembled delivery system of the present invention can be prepared into gel, patch, tincture, ointment, cream, spray, etc.

For enteral nutrition liquid, the target (active ingredient) in the supramolecular self-assembly-based delivery system of the present invention can be fat-soluble vitamins, including but not limited to vitamin A, vitamin K1, vitamin D, vitamin E, or carotenoids. The fat-soluble vitamins can be first prepared into the supramolecular self-assembly system according to the technical solution in the present invention, lyophilized, and then combined with minerals, trace elements, water-soluble vitamins, proteins, various amino acids, fats, carbohydrates, triglycerides, and water according to any enteral nutrition solution preparation process.

Another application of the supramolecular self-assembly-based delivery system built in the invention can be used in the beverage industry, such as sports drinks and sugar-free drinks suitable for diabetics and sugar controlled people. The present invention provides a stable enough self-assembly system for fat-soluble vitamins and other nutrients, improves the stability of effective ingredients, and provides low-calorie healthy drinks that do not raise blood sugar levels.

The supramolecular self-assembly delivery system built in the present invention can also be used in any situation that requires delivery, such as pesticides, insecticides, disinfectants, shampoos, laundry detergents, cleaning products, cosmetics, paints, printing and dyeing, with good biocompatibility, and reduced environmental pollution.

The dosages of the targets or active ingredients, high molecular building block, and natural building block in the supramolecular self-assembly-based delivery system can be specifically selected according to the unit dose of the target in the composition, the chemical structure, the number of hydrogen donors and hydrogen acceptors in the chemical structure, the oil-water partition coefficient, the physiological partition coefficient, etc.

### Unexpected Effects Achieved by the Present Invention are as Follows:

(1) Strong universality. By finely regulating the types and amount of high molecular polymers and carriers (such as flavonoids or terpenoids) in the building blocks of the supramolecular self-assembly system, the system is suitable for all targets with different physical and chemical properties, such as hydrophilic, hydrophobic, large or small molecular, dissociated or non-dissociated targets;
(2) Compared with a self-assembly system based on a single polymer, the supramolecular self-assembly system in the present invention can provide a delivery system with structural diversity, good biocompatibility, good safety, and capabilities of providing more effective functional groups; (3) Compared with a self-assembly system built by a single polymer, the supramolecular self-assembly system in the present invention is easier to form a stable supramolecular self-assembly system. The multi-chiral feature of flavonoid or terpenoid molecules, with multiple freely rotatable chemical bonds, can coordinate with the effective groups on polymers and performing molecular recognition, which can provide a relatively stable space for target molecules with different chemical and spatial structures, prevent molecular stacking caused by interactions between target molecules or structure limitations of single polymer involves, and improving the solubility, stability, transmembrane transport ability, and targeting of the active ingredients;
(3) Compared with a self-assembly system built by a single polymer, higher encapsulation efficiency, and better stability are achieved, meanwhile, the intake of high molecular polymers in long-term administration is greatly reduced; (5) Unexpected effects can be achieved with lower mass concentration, and the lower production cost of drugs;
(6) The supramolecular self-assembly system of the present invention has amphiphilic properties of natural cell membranes, for example for oral delivery, which can reduce the degradant of peptide/protein drugs by pepsin and trypsin, and or the metabolism induced by P450 enzymes and or ester hydrolase, and or effluxinduced by P-gp, thereby improve delivery efficiency; (7) Irritation or adverse reaction toxicity caused by direct contact between chemotherapy drug molecules and delivery sites is reduced, direct contact between in vivo diagnostic reagents and blood is also reduced, and the present invention is suitable for the development of pharmaceutical products or in vivo diagnostic reagents for various administration routes; (8) Medicines for children and elderly people with swallowing difficulties, such as oral solutions, dry suspensions, quickly disintegrating oral tablets, dispersible tablets, sublingual tablets, and capsule administration, can be developed, which not only provide the above unexpected effects, but also have the effect of masking and/or correcting flavors without additional masking and/or correcting agents; (9) Structures of building blocks such as flavonoids and terpenoids are widely found in vegetables, fruits, grains, or edible herbal plants, the safety has been verified, and the long-term consume has no safety hazards and have good biocompatibility;
(10) In addition to unexpected effects through synergistic effects with high molecular polymers at lower doses, the daily intake of polymers can be reduced;
(11) The carriers selected for the present invention, such as flavonoids or terpenoids building blocks, have mature commercial sources, and the unit costs are even lower than those of artificially synthesized polymer excipients, so the present invention has good industrialization prospects.

### BRIEF DESCRIPTION OF THE DRAWINGS

FIG. 1 shows synergistic regulation curves of different supramolecular self-assembly systems on Nilotinib molecule stacking.

FIG. 2 shows synergistic regulation curves of different supramolecular self-assembly systems on Nintedanib molecule stacking.

FIG. 3 shows synergistic regulation curves of different supramolecular self-assembly systems on Sorafenib molecule stacking.

FIG. 4 shows synergistic regulation curves of different supramolecular self-assembly systems on Ticagrelor molecule stacking.

FIG. 5 shows synergistic regulation curves of different supramolecular self-assembly systems on Apixaban molecule stacking.

FIG. 6 shows synergistic regulation curves of different supramolecular self-assembly systems on Rivaroxaban molecule stacking.

FIG. 7 shows synergistic regulation curves of different supramolecular self-assembly systems on Curcumin molecule stacking.

FIG. 8 shows synergistic regulation curves of different supramolecular self-assembly systems on Ibrutinib molecule stacking.

FIG. 9 shows synergistic regulation curves of different supramolecular self-assembly systems on Palbociclib molecule stacking.

FIG. 10 shows synergistic regulation curves of different supramolecular self-assembly systems on Ezetimibe molecule stacking.

FIG. 11 shows synergistic regulation curves of different supramolecular self-assembly systems on Ticagrelor molecule stacking.

FIG. 12 shows synergistic regulation curves of different supramolecular self-assembly systems on Rivaroxaban molecule stacking.

FIG. 13 shows synergistic regulation curves of different supramolecular self-assembly systems on Apixaban molecule stacking.

FIG. 14 shows synergistic regulation curves of different supramolecular self-assembly systems on Ibrutinib molecule stacking.

FIG. 15 shows synergistic regulation curves of different supramolecular self-assembly systems on Dabigatran Etexilate molecule stacking.

FIG. 16 shows synergistic regulation curves of different supramolecular self-assembly systems on Lenvatinib molecule stacking.

FIG. 17 shows synergistic regulation curves of different supramolecular self-assembly systems on Curcumin molecule stacking.

FIG. 18 shows synergistic regulation curves of different supramolecular self-assembly systems on Sorafenib molecule stacking.

FIG. 19 shows synergistic regulation curves of different supramolecular self-assembly systems on Nintedanib molecule stacking.

FIG. 20 shows synergistic regulation curves of different supramolecular self-assembly systems on Docetaxel molecule stacking.

FIG. 21 shows synergistic regulation curves of different supramolecular self-assembly systems on Lurasidone hydrochloride molecule stacking.

FIG. 22 shows synergistic regulation curves of different supramolecular self-assembly systems on Dabigatran Etexilate molecule stacking.

FIG. 23 shows synergistic regulation curves of different supramolecular self-assembly systems on Ticagrelor molecule stacking.

FIG. 24 shows synergistic regulation curves of different supramolecular self-assembly systems on Cyclosporine molecule stacking.

FIG. 25 shows synergistic regulation curves of different supramolecular self-assembly systems on Fingolimode molecule stacking.

FIG. 26 shows synergistic regulation curves of different supramolecular self-assembly systems on Macitentan molecule stacking.

FIG. 27 shows synergistic regulation curves of different supramolecular self-assembly systems on Tacrolimus molecule stacking.

FIG. 28 shows synergistic regulation curves of different supramolecular building units on Palbociclib molecule stacking.

FIG. 29 shows synergistic regulation curves of different supramolecular self-assembly systems on Enzalutamide molecule stacking.

FIG. 30 shows synergistic regulation curves of building units with different mass concentrations on Docetaxel molecule stacking.

FIG. 31 shows synergistic regulation curves of building units with different mass concentrations on Paclitaxel molecule stacking.

FIG. 32 shows synergistic regulation curves of building units with different mass concentrations on Curcumin molecule stacking.

FIG. 33 shows synergistic regulation curves of mass concentration changes of building units on Nintedanib molecule stacking.

FIG. 34 shows synergistic regulation curves of mass concentration changes of building units on Palbociclib molecule stacking.

FIG. 35 shows synergistic regulation curves of different supramolecular self-assembly systems on Felodipine molecule stacking.

FIG. 36 shows synergistic regulatory effects of different polymer models on Nilotinib molecular stacking.

FIG. 37 shows synergistic regulation curves of quaternary supramolecular self-assembly systems on Apixaban molecule stacking.

FIG. 38 shows synergistic regulation curves of quaternary supramolecular self-assembly systems on Clopidogrel molecule stacking.

FIG. 39 shows synergistic regulation curves of different supramolecular self-assembly systems on Naringenin molecule stacking.

FIG. 40 shows synergistic regulation curves of quaternary supramolecular self-assembly systems on Posaconazole molecule stacking.

FIG. 41 shows synergistic regulation curves of different supramolecular self-assembly systems on Warfarin molecule stacking.

FIG. 42 shows synergistic regulation curves of different supramolecular self-assembly systems on Vitamin K1 molecule stacking.

FIG. 43 shows synergistic regulation curves of different supramolecular self-assembly systems on Eltrombopag molecule stacking.

FIG. 44 shows regulation effect curves of natural building units with high mass concentrations in comparative examples on guest molecule stacking.

FIG. 45 shows synergistic regulation effect curves of the same building block on the stacking of different guest molecules.

FIG. 46 shows molecular stacking curves of four different guest molecules in their initial medium.

FIG. 47 shows synergistic regulation effect curves of mass concentration changes of building units on the stacking of different guest molecules.

FIG. 48 shows synergistic regulation curves of the same supramolecular self-assembly system on the stacking of different guest molecules.

FIG. 49 shows synergistic regulation curves of different supramolecular self-assembly systems on Macitentan molecule stacking.

FIG. 50 shows synergistic regulation curves of different supramolecular self-assembly systems on Butyphthalide molecule stacking.

FIG. 51 shows synergistic regulation curves of different supramolecular self-assembly systems on Coenzyme Q10 molecule stacking.

FIG. 52 shows synergistic regulation curves of quaternary supramolecular self-assembly systems on cannabidiol molecule stacking.

FIG. 53 shows synergistic regulation curves of ternary supramolecular self-assembly systems on Cannabidiol molecule stacking.

FIG. 54 shows synergistic regulation curves of ternary supramolecular self-assembly systems built by polymer 103 on Cannabidiol molecule stacking.

FIG. 55 shows synergistic regulation curves of ternary supramolecular self-assembly systems built by different polymers and the same carrier on Cannabidiol molecule stacking.

FIG. 56 shows synergistic regulation curves of ternary supramolecular self-assembly systems built by different polymers and carriers on Nintedanib molecule stacking.

FIG. 57 shows synergistic regulation curves of different supramolecular self-assembly systems on Lurasidone hydrochloride molecule stacking.

FIG. 58 shows synergistic regulation curves of different supramolecular self-assembly systems on Posaconazole molecule stacking.

FIG. 59 shows synergistic regulation curves of different supramolecular self-assembly systems on Tafluprost molecule stacking.

FIG. 60 shows effects of different supramolecular self-assembly systems on system concentrations of Vitamin A, E, and Lutein after incubation for 6 hours.

### DETAILED DESCRIPTION

In order to make the objectives, technical solutions and advantages of the present invention clearer, the technical solutions in the present invention will be clearly and completely described below in conjunction with the accompanying drawings in the present invention. Apparently, the described embodiments are some but not all of the embodiments of the present invention. All other embodiments obtained by those of ordinary skill in the art based on the embodiments in the present invention without any creative effort fall within the scope of protection of the present invention. Specific technologies or conditions unmarked in the examples follow the technologies or conditions described in the documents of this field or the product specifications. Instruments unmarked with manufacturers are conventional products that can be purchased by proper channels. Described methods are conventional methods unless otherwise specified, and raw materials can be obtained from publicly available commercial channels unless otherwise specified. For the convenience of subsequent expression and drawing, the foregoing polymers, combination polymers, and natural building blocks are numbered. Numbering is shown in Table 3:

TABLE 3
Numbering of building units and ligands in self-assembly systems

| | Polymer-single | | Carrier-natural | |
|---|---|---|---|---|
| Number | polymer | Number | building unit | Ligand CAS |
| 101 | Polyvinyl caprolactam- | 301 | Naringin | 10236-47-2 |
| | polyvinyl acetate- | | | |
| | polyethylene glycol | | | |
| | graft co-polymer | | | |
| 102 | Hydroxypropyl | 302 | Hesperidin | 520-26-3 |
| | methylcellulose acetate | | | |
| | succinate (H, L, M) | | | |
| 103 | Hydroxypropyl | 303 | (—)-Epigallocatechin | 989-51-5 |
| | methylcellulose | | 3-gallate(EGCG) | |
| 104 | Sodium carboxymethyl | 304 | Isoquercitrin | 482-35-9 |
| | cellulose | | | |
| 105 | Polyethylene glycol | 305 | Quercetin | 117-39-5 |
| 106 | Polyvinyl pyrrolidone | 306 | Myricitrin | 17912-87-7 |
| 107 | Copovidone | 307 | Rebaudioside B | 58543-17-2 |
| 108 | Cellulose acetate | 308 | Rebaudioside C | 63550-99-2 |
| 109 | Methacrylic acid-methyl | 309 | (—)-Epigallocatechin | 970-74-1 |
| | methacrylate copolymer | | (EGC) | |
| 110 | Methacrylic acid-ethyl | 310 | Neohesperidin | 20702-77-6 |
| | methacrylate copolymer | | dihydrochalcone | |
| 111 | Hydroxypropyl cellulose | 311 | Trilobatin | 4192-90-9 |
| 112 | Cyclodextrin | 312 | Naringin | 18916-17-1 |
| | | | dihydrochalcone | |
| 113 | Tween (polyoxyethylene | 313 | Rebaudioside A | 58543-16-1 |
| | and polyoxypropylene | | | |
| | block copolymer) | | | |
| 114 | Sodium dodecyl sulfate | 314 | Glycyrrhizic acid | 1405-86-3 |
| 115 | Vitamin E polyethylene | 315 | Stevioside | 57817-89-7 |
| | glycol succinate, TPGS | | | |
| combination polymers | | 315-1 | Steviol | \ |
| | | | glycosides 95% | |
| 201 | 102 (H, M, L) + 101 | 316 | Tannic Acid | 1401-55-4 |
| 202 | 102 (H, M, L) + 107 | 317 | Quercetin | 153-18-4 |
| | | | 3-rutinoside | |
| 203 | 102 (H, M, L) + | 318 | Neohesperidin | 13241-33-3 |
| | 106 (K25-K90) | | | |
| 204 | 102 (H, M, L) + | 319 | Mogroside V | 88901-36-4 |
| | 103 (E3, E5) | | | |
| 205 | 102 (H, M, L) + 104 | 319-1 | Mogroside V | \ |
| | | | 30% extract | |
| 206 | 102 + 109 (S, L, E) | 320 | Rebaudioside D | 63279-13-0 |
| 207 | 101 + 103 (E3, E5) | | | |
| | | 321 | Rebaudioside M | 1220616-44-3 |

TABLE 4
Basic information and ADI for human use of natural building blocks
selected in the present invention

| | | | | Acceptable |
|---|---|---|---|---|
| | | | | daily |
| | General | | | intake |
| Number | name | D-H or R-H | Chemical structure/Formula/MW | (ADI) |
| 301 | Naringin | D-H: 8, R- H: 14, RBC (rotatable chemical bonds): 6 | 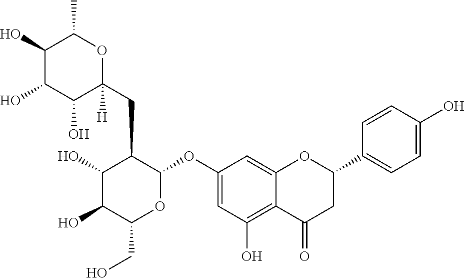 [SMILES: C[C@@H]1O[C@@H](C[C@H]2[C@H](Oc3cc(O)c4c(c3)O[C@H](c3ccc(O)cc3)CC4=O)O[C@H](CO)[C@@H](O)[C@@H]2O)[C@H](O)[C@H](O)[C@H]1O] | 19 mg/kg bw/day bw: body weight |
| | | | C27H32O14 | |
| | | | 580.5 | |
| 302 | Hesperidin | D-H: 8, R- H: 15, RBC is 7 | 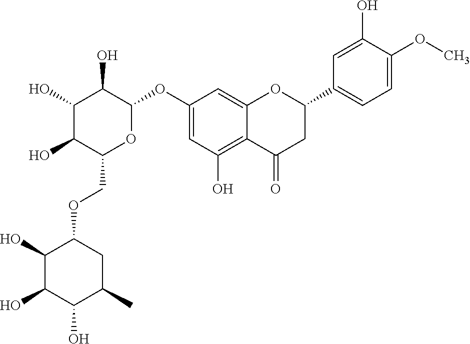 [SMILES: COc1ccc([C@@H]2CC(=O)c3c(O)cc(O[C@@H]4O[C@H](CO[C@@H]5C[C@@H](C)[C@H](O)[C@@H](O)[C@H]5O)[C@@H](O)[C@H](O)[C@H]4O)cc3O2)cc1O] | 16 mg/kg bw/day |
| | | | C28H34O15 | |
| | | | 610.6 | |
| 303 | (−)− Epigall ocatech in 3- gallate (EGCG) | D-H: 8, R- H: 11, RBC is 4 | 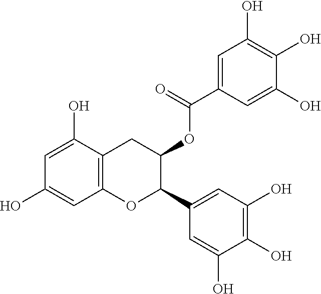 [SMILES: O=C(O[C@@H]1Cc2c(O)cc(O)cc2O[C@@H]1c1cc(O)c(O)c(O)c1)c1cc(O)c(O)c(O)c1] | 135-270 mg/person/ day |
| | | | C22H18O11 | |
| | | | 458.4 | |
| 304 | Isoquercitrin | D-H: 8, R- H: 12, RBC is 4 | 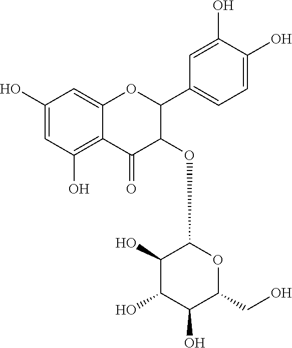 [SMILES: O=C1c2c(O)cc(O)cc2OC(c2ccc(O)c(O)c2)C1O[C@@H]1O[C@H](CO)[C@@H](O)[C@H](O)[C@H]1O] | 3.3 mg/kg bw/day |
| | | | C21H20O12 | |
| | | | 464.4 | |
| 305 | Quercetin | D-H: 5, R- H: 7, RBC is 1 | 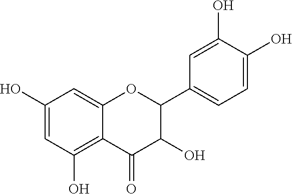 [SMILES: O=C1c2c(O)cc(O)cc2OC(c2ccc(O)c(O)c2)C1O] | 26 mg/kg bw/day |
| | | | C15H10O7 | |
| | | | 302.2 | |
| 306 | Myricitrin | D-H: 8, R- H: 12, RBC is 3 | 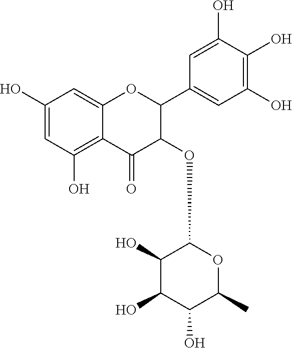 [SMILES: C[C@@H]1O[C@@H](OC2C(=O)c3c(O)cc(O)cc3OC2c2cc(O)c(O)c(O)c2)[C@H](O)[C@H](O)[C@H]1O] | 14 mg/kg bw/day |
| | | | C21H20O12 | |
| | | | 464.4 | |
| 309 | Epigall ocatech in (EGC) | H: 6, R-H: 7, RBC is 1 | 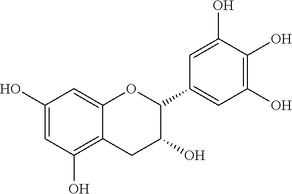 | 1.83 mg/kg bw/day |
| | | | C15H14O7 | |
| | | | 306.3 | |
| 310 | Neohes peridin dihydro chalcone | H: 9, R-H: 15, RBC is 10 | 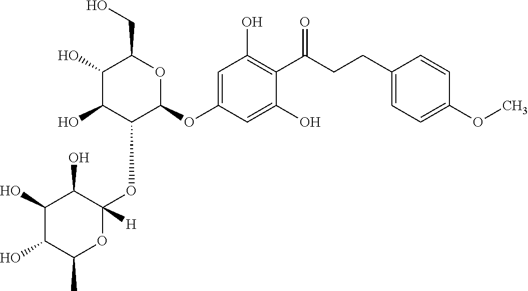 [SMILES: COc1ccc(CCC(=O)c2c(O)cc(O[C@@H]3O[C@H](CO)[C@@H](O)[C@H](O)[C@H]3O[C@@H]3O[C@@H](C)[C@H](O)[C@@H](O)[C@H]3O)cc2O)cc1] | 3-5 mg/kg bw/day |
| | | | C28H36O15 | |
| | | | 612.6 | |
| 311 | Trilobatin | D-H: 7, R- H: 10, RBC is 7 | 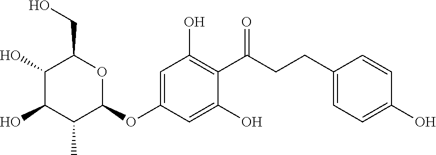 [SMILES: C[C@H]1[C@H](Oc2cc(O)c(C(=O)CCc3ccc(O)cc3)c(O)c2)O[C@H](CO)[C@@H](O)[C@@H]1O] | 15 mg/person/ day |
| | | | C21H24O10 | |
| | | | 436.4 | |
| 312 | Naringin dihydro chalcone | D-H: 9, R- H: 14, RBC is 9 | 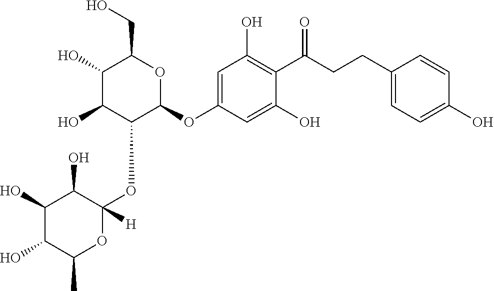 [SMILES: C[C@@H]1O[C@@H](O[C@H]2[C@H](Oc3cc(O)c(C(=O)CCc4ccc(O)cc4)c(O)c3)O[C@H](CO)[C@@H](O)[C@@H]2O)[C@H](O)[C@H](O)[C@H]1O] | 41 mg/person/ day |
| | | | C27H34O14 | |
| | | | 582.5 | |
| 317 | Quercetin 3- rutinoside | D-H: 10, R- H: 16, RBC is 6 | 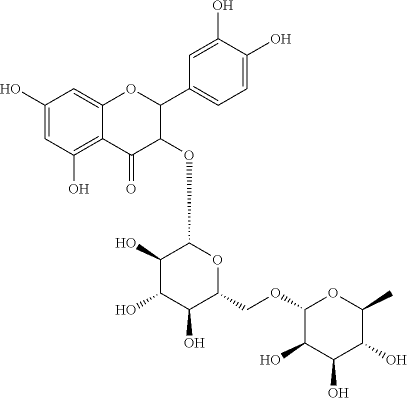 [SMILES: C[C@@H]1O[C@@H](OC[C@H]2O[C@@H](OC3C(=O)c4c(O)cc(O)cc4OC3c3ccc(O)c(O)c3)[C@H](O)[C@@H](O)[C@@H]2O)[C@H](O)[C@H](O)[C@H]1O] | 16 mg/kg bw/day |
| | | | C27H30O16 | |
| | | | 610.5 | |
| 318 | Neohes peridin | D-H: 8, R- H: 15, RBC is 7 | 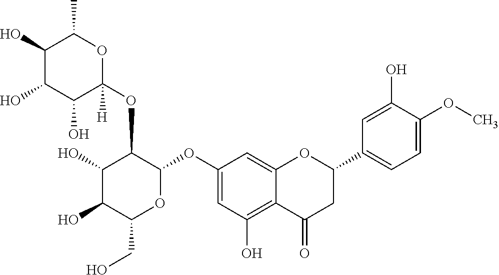 [SMILES: COc1ccc([C@@H]2CC(=O)c3c(O)cc(O[C@@H]4O[C@H](CO)[C@@H](O)[C@H](O)[C@H]4O[C@@H]4O[C@@H](C)[C@H](O)[C@@H](O)[C@H]4O)cc3O2)cc1O] | 5 mg/kg bw/day |
| | | | C28H34O15 | |
| | | | 610.6 | |
| 316 | Tannic acid | D-H: 25, R- H: 46, RBC is 31 | 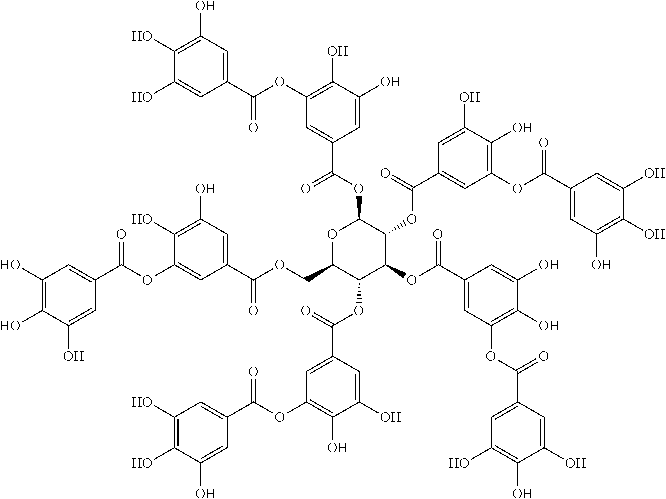 [SMILES: O=C(OC[C@H]1O[C@@H](OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1] | 15 mg/kg bw/day |
| | | | C76H52O46 | |
| | | | 1701.2 | |
| 314 | Glycyrrhizic acid and its ammonium salt | D-H: 8, R- H: 16, RBC is 7 | 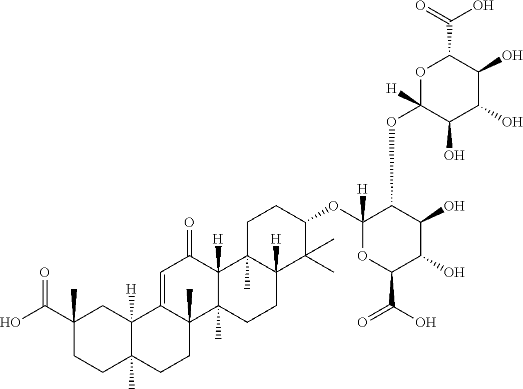 | 2 mg/kg bw/day |
| | | | C42H62O16 | |
| | | | 822.9 | |
| 315 | Stevioside | D-H: 11, R- H: 18, RBC is 10 | 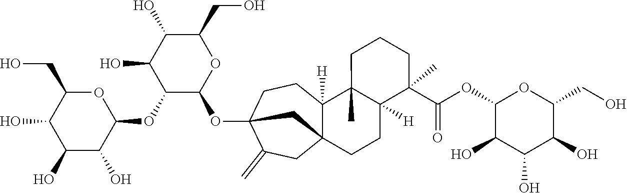 | 4 mg/kg bw/day |
| | | | C38H60O18 | |
| | | | 804.9 | |
| 315- | Steviol | Steviol glycosides 90% extract: total steviol glycoside | | 0-2 mg/kg |
| 1 | glycosides | content ≥ 95%, stevioside ≥ 55%, rebaudioside A ≥ 25 | | bw/day |
| | Extract | | | |
| | 95% | | | |
| 313 | Rebaud ioside A | D-H: 14, R- H: 22, RBC is 13 | 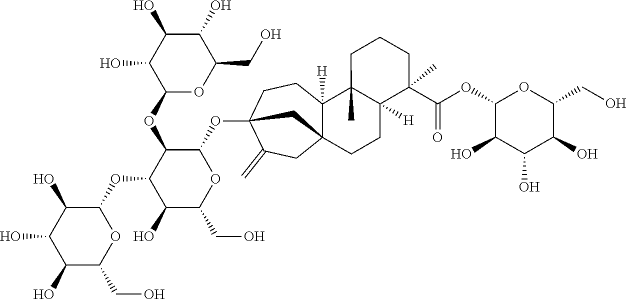 | 0-6 mg/kg bw/day |
| | | | C44H70O23 | |
| | | | 967.0 | |
| 307 | Rebaud ioside B | D-H: 11, R- H: 18, RBC is 10 | 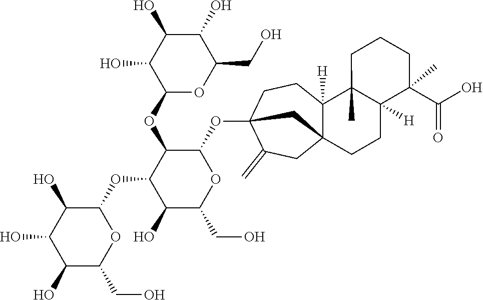 | 6 mg/kg bw/day |
| | | | C38H60O18 | |
| | | | 804.9 | |
| 308 | Rebaud ioside C | D-H: 13, R- H: 22, RBC is 12 | 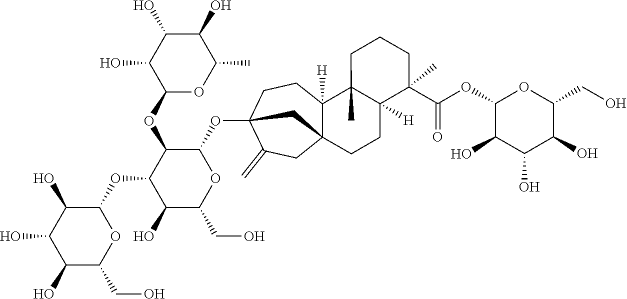 | 6 mg/kg bw/day |
| | | | C44H70O22 | |
| | | | 951.0 | |
| 320 | Rebaud ioside D | D-H: 17, R- H: 28, RBC is 16 | 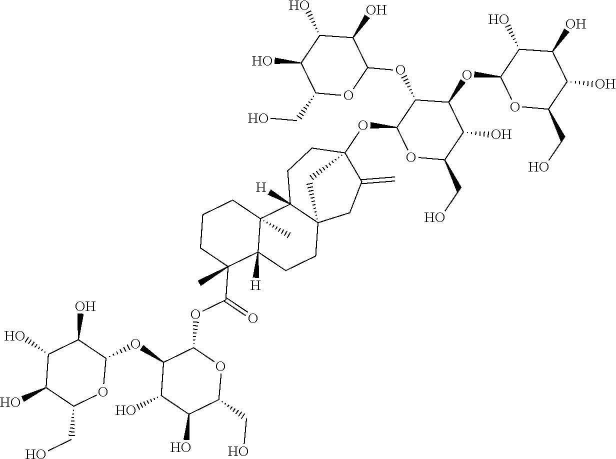 | 3-5 mg/kg bw/day |
| | | | C50H80O28 | |
| | | | 1129.2 | |
| 321 | Rebaud ioside M | D-H: 20, R- H: 33, RBC is 19 | 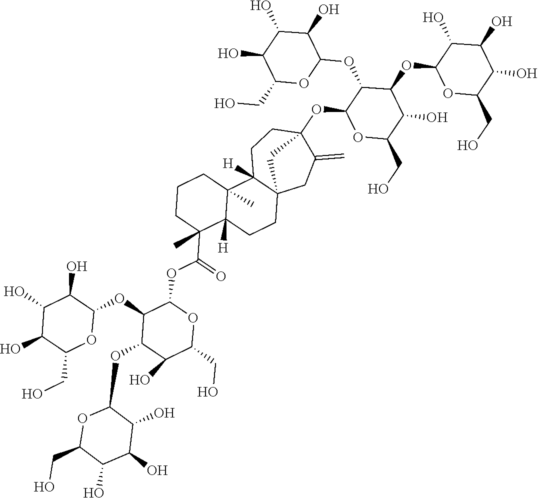 | 4 mg/kg bw/day |
| | | | C56H90O33 | |
| | | | 1291.3 | |
| 319 | Mogroside V | D-H: 19, R- H: 29, RBC is 10 | 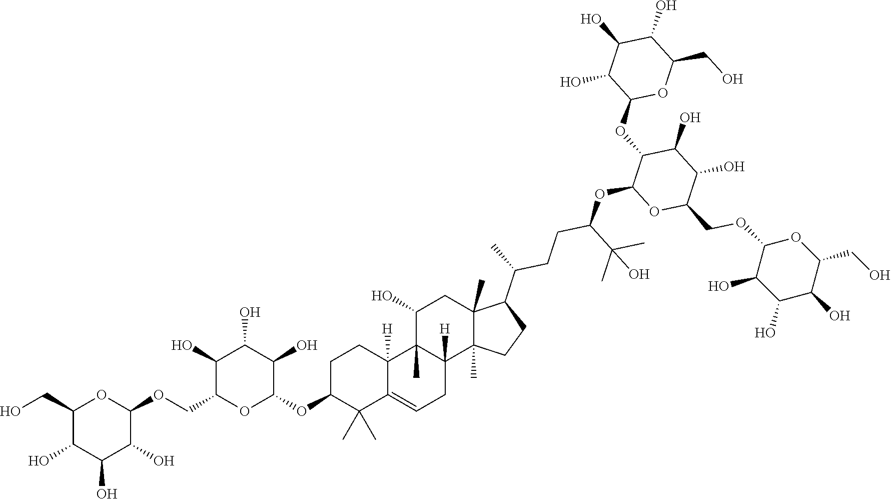 | 0-2.5 mg/kg bw/day |
| | | | C60H102O29 | |
| | | | 1287.4 | |
| 319- | Mogroside V | Mogroside V 30% extract: measured mogroside V 35.78%, | | 0-8 mg/kg |
| 1 | 30% | 11-oxomogroside: 5.31%: 5.31%, siamenoside I: 3.27% | | bw/day |
| | extract | | | |

The above natural building blocks have been listed as GRAS materials by FDA or EU as food additives or flavoring agents (sweeteners) or masking agents, and are widely used as flavoring agents in the fields of beverages, food processing, dairy processing, meat processing, health products, dietary supplements, cosmetics, etc., with acceptable daily intake(ADI) limits specified. Although there have been no reports on their use as regulation or synergistic regulation targets and/or polymers of self-assembly systems, they are safe for mammals or humans as long as they are used within the ADI range.

Suppliers of targets or guest molecules and special materials used in the present invention are shown separately in Table 5 and Table 6.

TABLE 5
Suppliers information of targets

| Number | Target | Chemical structure | Supplier |
|---|---|---|---|
| 1 | Free Nilotinib base and hydrochloride | 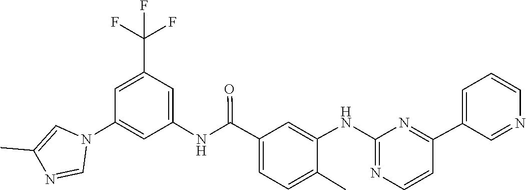 [SMILES: Cc1cn(-c2cc(NC(=O)c3ccc(C)c(Nc4nccc(-c5cccnc5)n4)c3)cc(C(F)(F)F)c2)cn1] | Shandong Lixin Pharmaceutical Co., Ltd. |
| 2 | Nintedanib | 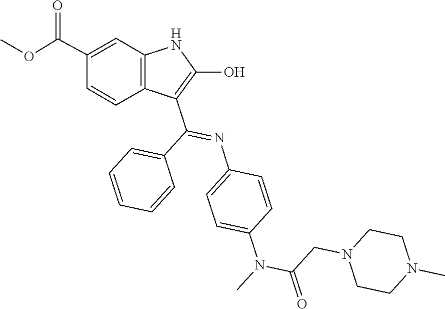 | Pinghu Aibai Chemical Co., Ltd. |
| 3 | Lenvatinib | 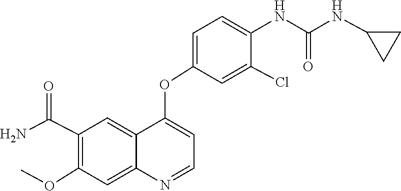 [SMILES: COc1cc2nccc(Oc3ccc(NC(=O)NC4CC4)c(Cl)c3)c2cc1C(N)=O] | Beijing Creatron Pharmaceutical Research Institute Co., Ltd. |
| 4 | Sorafenib | 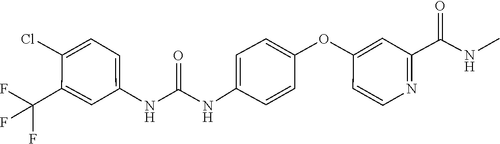 [SMILES: CNC(=O)c1cc(Oc2ccc(NC(=O)Nc3ccc(Cl)c(C(F)(F)F)c3)cc2)ccn1] | Pinghu Aibai Chemical Co., Ltd. |
| 5 | Ticagrelor | 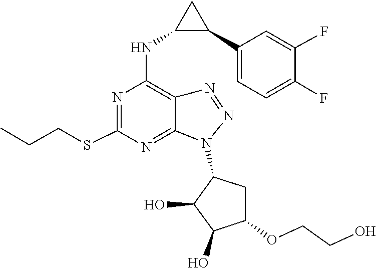 | Hubei Jusheng Technology Co., Ltd. |
| 6 | Apixaban | 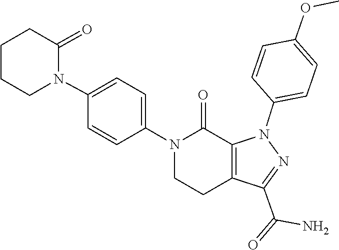 [SMILES: COc1ccc(-n2nc(C(N)=O)c3c2C(=O)N(c2ccc(N4CCCCC4=O)cc2)CC3)cc1] | Suzhou Carbonwell Pharmaceutical Technology Co., Ltd. |
| 7 | Rivaroxaban | 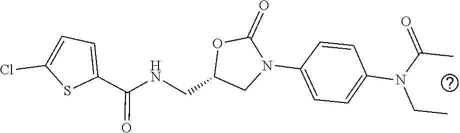 [SMILES: CCN(C(C)=O)c1ccc(N2C[C@H](CNC(=O)c3ccc(Cl)s3)OC2=O)cc1] | Anhui Lianchuang Pharmaceutical Co., Ltd. |
| 8 | Warfarin | 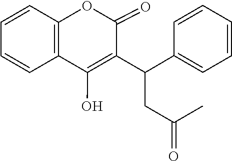 [SMILES: CC(=O)CC(c1ccccc1)c1c(O)c2ccccc2oc1=O] | Pinghu Aibai Chemical Co., Ltd. |
| 9 | Lurasidone, Lurasidone Hydrochloride | 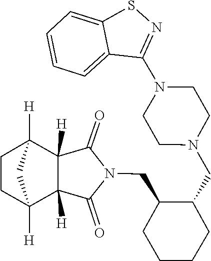 [SMILES: O=C1[C@@H]2[C@H]3CC[C@H](C3)[C@@H]2C(=O)N1C[C@@H]1CCCC[C@H]1CN1CCN(c2nsc3ccccc23)CC1] | Jinan Jianfeng Chemical Co., Ltd. |
| 10 | Curcumin | 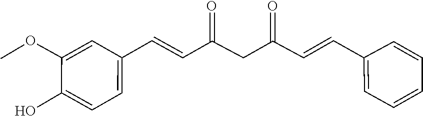 [SMILES: COc1cc(/C=C/C(=O)CC(=O)/C=C/c2ccccc2)ccc1O] | Xi′an Dongfeng Biotechnology Co., Ltd. |
| 11 | Vitamin K1 | 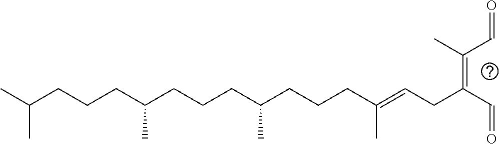 [SMILES: C/C(C=O)=C(/C=O)C/C=C(\C)CCC[C@H](C)CCC[C@H](C)CCCC(C)C] | Pinghu Aibai Chemical Co., Ltd. |
| 12 | Macitentan | 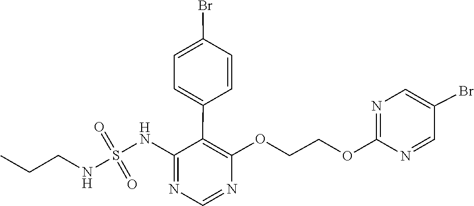 [SMILES: CCCNS(=O)(=O)Nc1ncnc(OCCOc2ncc(Br)cn2)c1-c1ccc(Br)cc1] | Shanghai Macklin Biochemical Technology Co., Ltd. |
| 13 | Tacrolimus | 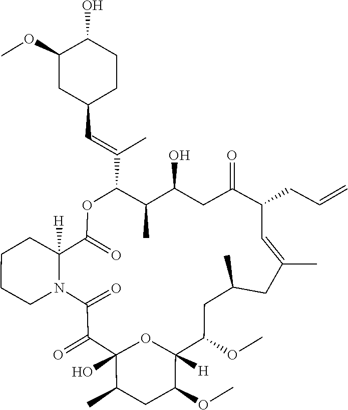 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC] | Hubei Jusheng Technology Co., Ltd. |
| 14 | Cyclosporine | 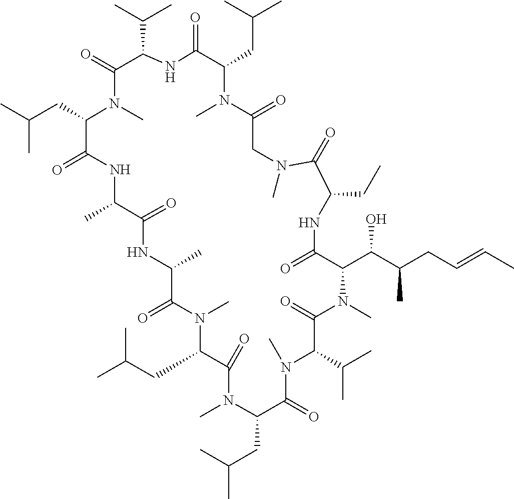 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | Beijing Innochem Technology Co., Ltd. |
| 15 | Paclitaxel | 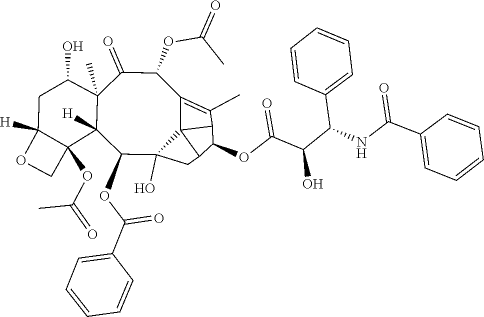 | Fujian South Pharmaceutical Co., Ltd. |
| 16 | Docetaxel | 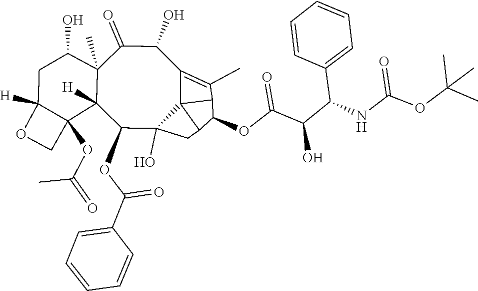 | Aladdin Reagent (Shanghai) Co., Ltd. |
| 17 | Ibrutinib | 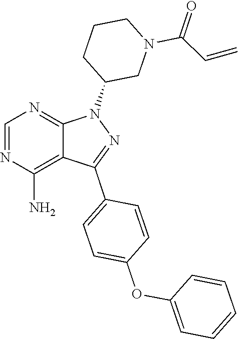 [SMILES: C=CC(=O)N1CCC[C@@H](n2nc(-c3ccc(Oc4ccccc4)cc3)c3c(N)ncnc32)C1] | SyncoZymes (Shanghai) Co., Ltd. |
| 18 | Clopidogrel | 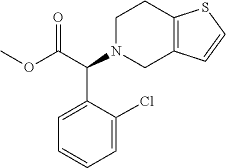 [SMILES: COC(=O)[C@H](c1ccccc1Cl)N1CCc2sccc2C1] | Jiangsu Aikon Biopharmaceutical R&D Co., Ltd. |
| 19 | Fingolimode | 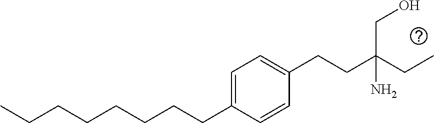 [SMILES: CCCCCCCCc1ccc(CCC(N)(CC)CO)cc1] | Jiangsu Aikon Biopharmaceutical R&D Co., Ltd. |
| 20 | Enzalutamide | 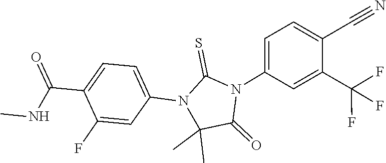 [SMILES: CNC(=O)c1ccc(N2C(=S)N(c3ccc(C#N)c(C(F)(F)F)c3)C(=O)C2(C)C)cc1F] | Pinghu Aibai Chemical Co., Ltd. |
| 21 | Posaconazole | 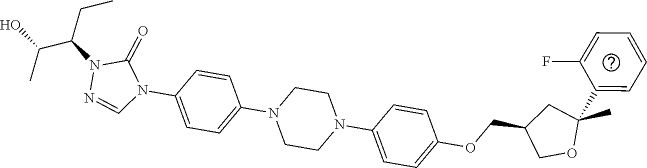 [SMILES: CC[C@H]([C@H](C)O)n1ncn(-c2ccc(N3CCN(c4ccc(OC[C@@H]5CO[C@@](C)(c6ccccc6F)C5)cc4)CC3)cc2)c1=O] | Pingdingshan Kaimeicheng Biotechnology Co., Ltd. |
| 22 | Dabigatranetexilate | 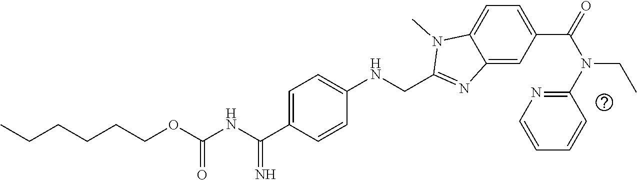 [SMILES: CCCCCCOC(=O)NC(=N)c1ccc(NCc2nc3cc(C(=O)N(CC)c4ccccn4)ccc3n2C)cc1] | Pinghu Aibai Chemical Co., Ltd. |
| 23 | Venetoclax | 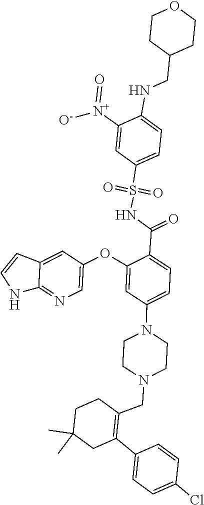 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1] | Jiangsu Aikon Biopharmaceutical R&D Co., Ltd. |
| 24 | Alectinib | 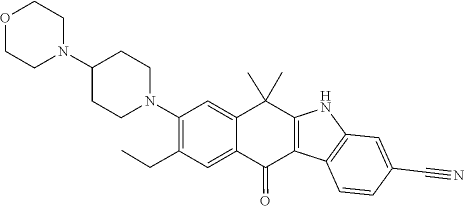 [SMILES: CCc1cc2c(cc1N1CCC(N3CCOCC3)CC1)C(C)(C)c1[nH]c3cc(C#N)ccc3c1C2=O] | Jiangsu Aikon Biopharmaceutical R&D Co., Ltd. |
| 25 | Palbociclib | 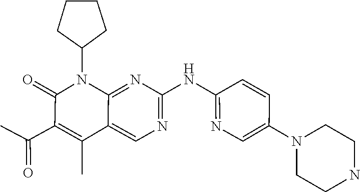 [SMILES: CC(=O)c1c(C)c2cnc(Nc3ccc(N4CCNCC4)cn3)nc2n(C2CCCC2)c1=O] | Pinghu Aibai Chemical Co., Ltd. |
| 26 | Naringenin * | 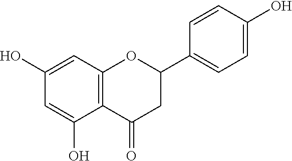 [SMILES: O=C1CC(c2ccc(O)cc2)Oc2cc(O)cc(O)c21] | Beijing J&K Scientific Co., Ltd. |
| 27 | Celecoxib | 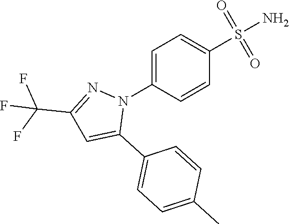 [SMILES: Cc1ccc(-c2cc(C(F)(F)F)nn2-c2ccc(S(N)(=O)=O)cc2)cc1] | Aladdin Reagent (Shanghai) Co., Ltd. |
| 28 | Itraconazole | 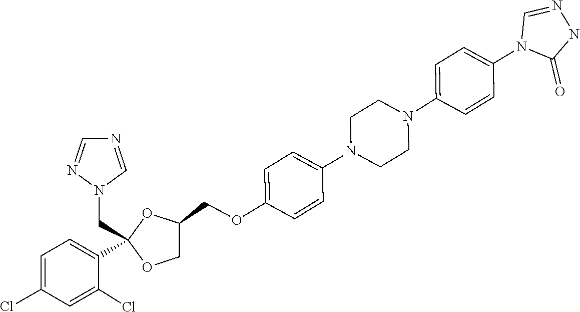 | Beijing Innochem Technology Co., Ltd. |
| 29 | Eltrombopag | 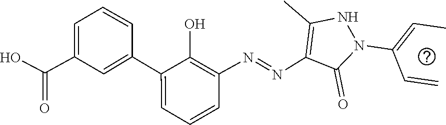 [SMILES: C=C/C(=C\C)n1[nH]c(C)c(/N=N/c2cccc(-c3cccc(C(=O)O)c3)c2O)c1=O] | Jiangsu Aikon Biopharmaceutical R&D Co., Ltd. |
| 30 | Ezetimibe | 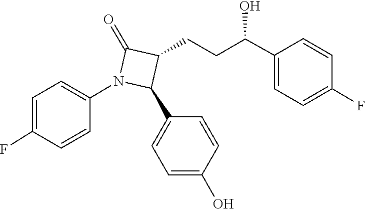 [SMILES: O=C1[C@H](CC[C@H](O)c2ccc(F)cc2)[C@@H](c2ccc(O)cc2)N1c1ccc(F)cc1] | Aladdin Reagent (Shanghai) Co., Ltd. |
| 31 | Griseofulvin | 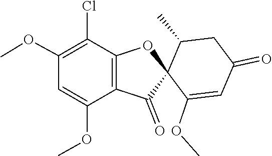 [SMILES: COC1=CC(=O)C[C@@H](C)[C@]12Oc1c(Cl)c(OC)cc(OC)c1C2=O] | Aladdin Reagent (Shanghai) Co., Ltd. |
| 32 | Candesartan Cilexetil | 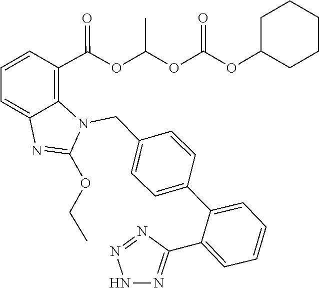 [SMILES: CCOc1nc2cccc(C(=O)OC(C)OC(=O)OC3CCCCC3)c2n1Cc1ccc(-c2ccccc2-c2nn[nH]n2)cc1] | Aladdin Reagent (Shanghai) Co., Ltd. |
| 33 | Felodipine | 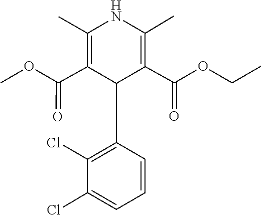 [SMILES: CCOC(=O)C1=C(C)NC(C)=C(C(=O)OC)C1c1cccc(Cl)c1Cl] | Aladdin Reagent (Shanghai) Co., Ltd. |
| 34 | Scutellarin | 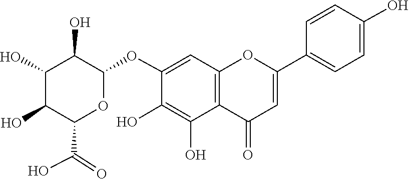 [SMILES: O=C(O)[C@H]1O[C@@H](Oc2cc3oc(-c4ccc(O)cc4)cc(=O)c3c(O)c2O)[C@H](O)[C@@H](O)[C@@H]1O] | Chuxiong Yunzhi Pharmaceutical Co., Ltd. |
| 36 | Acalabrutinib | 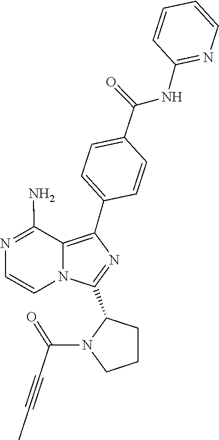 [SMILES: CC#CC(=O)N1CCC[C@H]1c1nc(-c2ccc(C(=O)Nc3ccccn3)cc2)c2c(N)nccn12] | Shanghai CoolPharm Biotechology Co., Ltd. |
| 37 | Regorafenib | 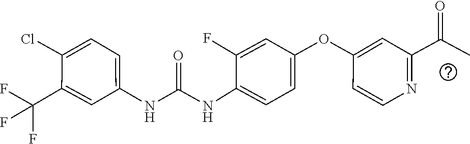 [SMILES: CC(=O)c1cc(Oc2ccc(NC(=O)Nc3ccc(Cl)c(C(F)(F)F)c3)c(F)c2)ccn1] | Pinghu Aibai Chemical Co., Ltd. |
| 38 | Butylphthalide | 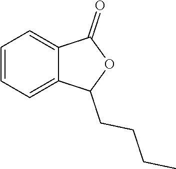 | Pinghu Aibai Chemical Co., Ltd. |
| 40 | Coenzyme Q10 | 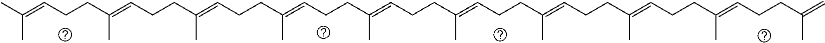 [SMILES: C=C(C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C] | Energy Chemical Co., Ltd. |
| 41 | Cannabidiol | 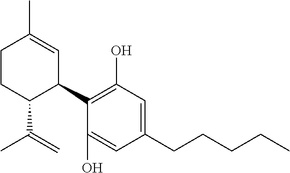 [SMILES: C=C(C)[C@@H]1CCC(C)=C[C@H]1c1c(O)cc(CCCCC)cc1O] | Yunnan Ghemp Biotechnology Co., Ltd. |
| 42 | Tafluprost | 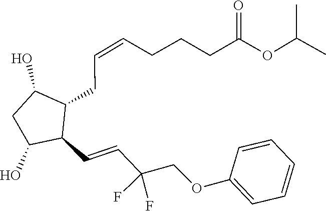 [SMILES: CC(C)OC(=O)CCC/C=C\C[C@@H]1[C@@H](/C=C/C(F)(F)COc2ccccc2)[C@H](O)C[C@@H]1O] | Wuhan Biocar Pharmaceutical Co., Ltd. |
| 43 | Lutein | 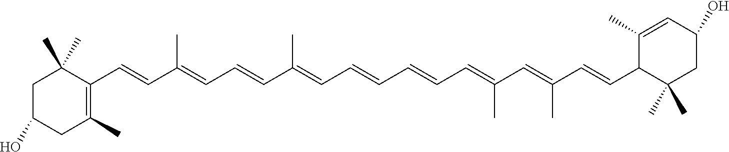 | Anhui Zesheng Technology Co., Ltd. |
| 44 | Vitamin A | 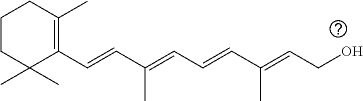 [SMILES: CC1=C(/C=C/C(C)=C/C=C/C(C)=C/CO)C(C)(C)CCC1] | Guangzhou Kangdier |
| 45 | Vitamin E | 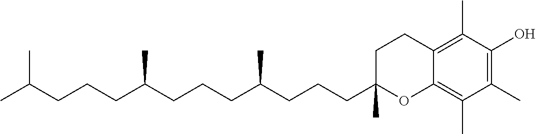 [SMILES: Cc1c(C)c2c(c(C)c1O)CC[C@@](C)(CCC[C@H](C)CCC[C@H](C)CCCC(C)C)O2] | Biotechnology Co., Ltd. |

Sources of the polymers and building blocks used in the present invention are as follows:

TABLE 6
Suppliers of polymers and building blocks

| Number | Polymers/building blocks | Suppliers |
|---|---|---|
| 1 | Hydroxypropyl methylcellulose E series | Dow Chemical |
| 2 | Hydroxypropyl methylcellulose K100LV | METHOCEL |
| 3 | Polyethylene caprolactam-polyvinyl | BASF SE |
| | acetate-polyethylene glycol | |
| | graft copolymer | |
| 4 | Hydroxypropyl methylcellulose | Shin-Etsu Chemical |
| | acetate succinate, H\M\L | Co., Ltd. |
| 5 | Hydroxypropyl methylcellulose | Shin-Etsu Chemical |
| | phthalate HP55 | Co., Ltd. |
| 6 | Hydroxypropyl cellulose | Nippon Soda CO |
| 7 | Ethyl cellulose | Tianjin Xinyue Kangda |
| | | Pharmaceutical Co., Ltd. |
| 8 | Poloxamer | BASF SE |
| 9 | Methacrylic acid-methyl methacrylate | Shanghai Changwei |
| | (1:1) copolymer | |
| 10 | Methacrylic acid-methyl methacrylate | Shanghai Changwei |
| | (1:2) copolymer | |
| 11 | Methacrylic acid-ethyl methacrylate | Evonik Specialty Chemicals |
| | (1:1) copolymer | (Shanghai) Co., Ltd. |
| 12 | Methacrylic acid-ethyl methacrylate | Evonik Specialty Chemicals |
| | (1:2) copolymer | (Shanghai) Co., Ltd. |
| 13 | Polyvinyl pyrrolidone series | Ashland |
| 14 | Polyethylene glycol series | Dow Chemical |
| 15 | Sodium dodecyl sulfate | Hunan Jiudian Hongyang |
| | | Pharmaceutical Co., Ltd. |
| 16 | Polysorbate (Tween20, 80) | Hunan Er-Kang Pharmaceutical |
| | | Co., Ltd. |
| 17 | Copovidone | Ashland |
| 18 | Carboxymethyl cellulose calcium | NICHRIN CHEMICAL |
| | | INDUSTRIES, IND. |
| 19 | Sodium carboxymethyl cellulose | Hunan Er-Kang Pharmaceutical |
| | | Co., Ltd. |
| 20 | Xanthan gum | ACROS |
| 21 | Naringin | Anhui Zesheng Technology |
| | | Co., Ltd. |
| 22 | Hesperidin | Anhui Zesheng Technology |
| | | Co., Ltd. |
| 23 | Myricetin | Anhui Zesheng Technology |
| | | Co., Ltd. |
| 24 | Isoquercitrin | Hebei Zhentian Food |
| | | Additives Co., Ltd. |
| 25 | Quercetin | Jiangsu Aikon |
| | | Biopharmaceutical R&D |
| | | Co., Ltd. |
| 26 | Myricitrin | Jiangsu Aikon |
| | | Biopharmaceutical R&D |
| | | Co., Ltd. |
| 27 | Rebaudioside B | Shanghai Yuanye |
| | | Biotechnology Co., Ltd. |
| 28 | Rebaudioside C | Shanghai Yuanye |
| | | Biotechnology Co., Ltd. |
| 29 | Epigallocatechin (EGC) | Anhui Zesheng Technology |
| | | Co., Ltd. |
| 30 | Neohesperidin dihydrochalcone | Shanghai Macklin Biochemical |
| | | Technology Co., Ltd. |
| 31 | Trilobatin | Nanjing Dolon Biotechnology |
| | | Co., Ltd. |
| 32 | Naringin dihydrochalcone | Jiangsu Aikon |
| | | Biopharmaceutical R&D |
| | | Co., Ltd. |
| 33 | Rebaudioside A | Aladdin Reagent (Shanghai) |
| | | Co., Ltd. |
| 34 | Glycyrrhizic acid | Anhui Zesheng Technology |
| | | Co., Ltd. |
| 35 | Stevioside | Qufu Shengren |
| | | Pharmaceutical Co., Ltd. |
| 36 | Steviol glycosides 95% | Qufu Shengren |
| | | Pharmaceutical Co., Ltd. |
| 37 | Tannic Acid | Beijing J&K Scientific |
| | | Co., Ltd. |
| 38 | Quercetin 3-rutinoside | Shanghai Macklin Biochemical |
| | | Technology Co., Ltd. |
| 39 | Neohesperidin | Anhui Zesheng Technology |
| | | Co., Ltd. |
| 40 | Mogroside V | Wuhan Guanying |
| | | Biotechnology Co., Ltd. |
| 41 | Mogroside V 30% extract | Wuhan Guanying |
| | | Biotechnology Co., Ltd. |
| 42 | Rebaudioside D | Shanghai Yuanye |
| | | Biotechnology Co., Ltd. |
| 43 | Rebaudioside M | Shanghai Yuanye |
| | | Biotechnology Co., Ltd. |
| 44 | D-α-tocopheryl polyethylene | BASF SE |
| | glycol succinate (TPGS) | |

### Comparative Example 1

pH 2.0 hydrochloric acid, pH 4.5 acetate, and pH 6.5, pH 6.8, or pH 7.4 phosphate buffer solutions were prepared. The selection of each initial medium followed the principle that the solubility in the initial medium of the selected targets (or active ingredients) should low enough, but should meet the detection sensitivity and accuracy requirements of the corresponding analytical methods. Each buffer as initial media was placed in a 50 ml test tube with a stopper, shaken in a constant temperature air shaker at 37° C.±0.5° C., with an amplitude of 200 rpm, and incubated for 1 hour. Take 0.5 ml of concentrated solution of each target (ensuring that the final built system was supersaturated), add it dropwise into the 50 ml of incubated initial medium of each target (control the final organic solvent concentration in 50 ml buffer does not exceed 1%), ultrasonic at 37° C. to uniform dispersion, and continue to shake in 37° C. constant temperature air shaker, the amplitude is 200 rpm. Samples were taken at 0.5 h, 3 h and 6 h after incubation, centrifuged at 37° C., 13000 rpm for 5 minutes, and the supernatant was diluted at least 10 times with the mobile phase under the target analyte analytical method, filtered, the initial filtrate was discarded, and the continued filtrate was used as the test solution for injection analysis. The corresponding HPLC method under each target object item in Table 8 was used to determine the content of each target substance. When the solubility of the target in the initial medium is below the quantitative limit of the analytical method, a small amount of surfactant, such as sodium dodecyl sulfate (SDS), Tween20, or Tween80, can be added appropriately.

In order to elaborate on the technical scheme of the present invention and highlight the advantages of technology, some representative target molecules are selected in Table 7 for the description of the technical scheme. The number of hydrogen acceptors and the hydrogen donors of each target molecule were obtained by drug chemical structure analysis, the Log D7.4 or Log P of dissociated drugs was obtained by literature research, and the molecular weight was calculated based on the drug formula. The selected target molecules were representative. Some targets have intramolecular interactions, some guests have intermolecular interactions, and some guests have both intramolecular and intermolecular interactions. The target guest molecules Log P or Log D covered 0.8 to 9.2 and structurally covered dissociated drugs (weak acids, weak bases) and non-dissociated drugs (neutral drugs), with 0-7 hydrogen donors and 2-12 hydrogen acceptors. The target molecules covered small molecular compounds, peptides, etc.

TABLE 7
Basic information of target molecules

| | | | | Intramolecular or |
|---|---|---|---|---|
| General English | H-D and | LogD_{7.4} or | Molecular | intermolecular |
| name | H-R | Log P | weight | hydrogen bonding |
| Nilotinib | H-D, 2, H-R, 6 | 5.35 | 529.5 | Intramolecular or |
| | | | | intermolecular |
| Nintedanib | H-D, 2, H-R, 6 | 2.75 | 539.6 | Intramolecular or |
| | | | | intermolecular |
| Lenvatinib | H-D, 3, H-R, 4 | 2.52 | 426.9 | Intermolecular |
| Sorafenib | H-D, 3, H-R, 3 | 4.34 | 464.8 | Intramolecular or |
| | | | | intermolecular |
| Ticagrelor | H-D, 4, H-R, 9 | 2.88 | 321.3 | Intramolecular or |
| | | | | intermolecular |
| Apixaban | H-D, 1, H-R, 5 | LogP 1.83 | 459.5 | Intermolecular |
| Rivaroxaban | H-D, 1, H-R, 5 | LogP 1.90 | 435.9 | Intermolecular |
| Warfarin | H-D, 1, H-R, 3 | LogP 5.56 | 330.3 | Intermolecular |
| Lurasidone | H-D, 0, H-R, 5 | 3.43 | 492.7 | None |
| Curcumin | H-D, 2, H-R, 6 | LogP 4.12 | 368.4 | Intramolecular or |
| | | | | intermolecular |
| Vitamin K1 | H-D, 0, H-R, 2 | LogP 9.16 | 450.7 | None |
| Macitentan | H-D, 2, H-R, 9 | LogP 3.55 | 574.3 | Intermolecular |
| Tacrolimus | H-D, 3, H-R, 11 | LogP 5.59 | 804.0 | Intramolecular or |
| | | | | intermolecular |
| Cyclosporine | H-D, 5, H-R, 12 | LogP 3.64 | 1202.6 | Intramolecular |
| Paclitaxel | H-D, 4, H-R, 10 | LogP 3.54 | 853.9 | Intramolecular |
| Docetaxel | H-D, 5, H-R, 10 | LogP 2.92 | 807.9 | Intramolecular |
| Ibrutinib | H-D, 1, H-R, 5 | 3.63 | 440.5 | Intermolecular |
| Clopidogrel | H-D, 0, H-R, 2 | 4.11 | 419.9 | None |
| Fingolimode | H-D, 3, H-R, 4 | LogP 2.12 | 307.5 | Intramolecular or |
| | | | | intermolecular |
| Enzalutamide | H-D, 1, H-R, 3 | 4.16 | 464.4 | Intermolecular |
| Posaconazole | H-D, 1, H-R, 9 | 5.41 | 700.8 | Intermolecular |
| Dabigatran | H-D, 2, H-R, 8 | 4.59 | 627.8 | Intramolecular or |
| Etexilate | | | | intermolecular |
| Venetoclax | H-D, 3, H-R, 10 | 6.88 | 868.4 | Intermolecular |
| Alectinib | H-D, 1, H-R, 5 | 4.75 | 482.6 | Intermolecular |
| Palbociclib | H-D, 2, H-R, 8 | 1.31 | 447.5 | Intermolecular |
| Naringenin | H-D, 3, H-R, 5 | LogP 2.71 | 272.3 | Intramolecular or |
| | | | | intermolecular |
| Celecoxib | H-D, 1, H-R, 3 | 4.01 | 381.4 | Intermolecular |
| Itraconazole | H-D, 0, H-R, 9 | 7.31 | 705.6 | None |
| Eltrombopag | H-D, 3, H-R, 7 | 1.28 | 442.5 | Intramolecular or |
| | | | | intermolecular |
| Griseofulvin | H-D, 0, H-R, 6 | 2.17 | 352.8 | No hydrogen |
| | | | | bonding |
| Acalabrutinib | H-D, 2, H-R, 6 | 2.56 | 399.5 | Intermolecular |
| Ezetimibe | H-D, 2, H-R, 3 | 4.56 | 409.4 | Intermolecular |
| Felodipine | H-D, 1, H-R, 5 | 3.90 | 382.4 | Intermolecular |
| Scutellarin | H-D, 7, H-R, 12 | 0.80 | 462.4 | Intramolecular or |
| | | | | intermolecular |
| Candesartan | H-D, 2, H-R, 7 | 5.31 | 610.7 | Intermolecular |
| Cilexetil | | | | |
| Regorafenib | H-D, 3, H-R, 5 | 4.49 | 482.8 | Intermolecular |
| Butylphthalide | H-D, 0, H-R, 1 | 3.36 | 190.2 | None |
| Coenzyme Q10 | H-D, 0, H-R, 4 | 17.20 | 863.3 | None |
| Cannabidiol | H-D, 2, H-R, 2 | 6.32 | 314.5 | None |
| Tafluprost | H-D, 2, H-R, 4 | 4.25 | 452.5 | Intermolecular |
| Lutein | H-D, 2, H-R, 2 | 8.55 | 568.9 | None |
| Vitamin A | H-D, 1, H-R, 1 | 4.69 | 286.5 | None |
| Vitamin E | H-D, 1, H-R, 2 | 10.50 | 430.7 | None |

The above drugs may be in a free base or acid state or in a form of salts thereof, and most of the targets used in the present invention were tested in a free form.

The above targets were incubated at 37° C.±0.5° C. for different time in an initial medium without any polymer. Each solid target formed by molecular stacking was removed by centrifugation, and the supernatant was analyzed using the HPLC method under each target item (see Table 8). The concentrations of each target at different time in the initial medium are shown in Table 9. The theoretical addition concentration of each target when its supramolecular self-assembly system was built was determined by its maximum dose in clinical use. After addition, a large number of target molecules quickly stacked and precipitated in a solid form. The target molecules that can remain in the solution or system for a long time can be effectively utilized.

The targets were quantitatively determined by high-performance liquid chromatography, which used a Agilent 1260 high-performance liquid chromatograph (HPLC), equipped with a G4212B DAD detector, a G1311B quaternary low-pressure pump, a G1316A column temperature box, and G1330B and G1329B automatic temperature controlled samplers.

Chromatographic conditions used for quantitative analysis on each target are shown in the following table:

TABLE 9
Concentration of the guest molecule in the solution measured
after different incubated time (37° C.) in the initial medium

| | | | Concentration of guest | | |
|---|---|---|---|---|---|
| | | Theoretical | molecule measured after | | |
| | | concentration | different incubated | | |
| English | Initial | after | time μg/mL | | |
| name | medium | addition | 0.5 h | 3 h | 6 h |
| Nilotinib | 0.05% SDS in pH | 450 μg/ml | 35.2 | 24.2 | 17.9 |
| | 6.8 phosphate | | | | |
| | buffer | | | | |
| Nintedanib | pH 6.8 phosphate | 500 μg/ml | 7.2 | 5.7 | 7.3 |
| | buffer | | | | |
| Lenvatinib | pH 6.8 phosphate | 200 μg/ml | 7.6 | 1.7 | 0.9 |
| | buffer | | | | |
| Sorafenib | 0.1% SDS in pH | 800 μg/ml | 98.5 | 4.1 | 4.4 |
| | 6.8 phosphate | | | | |
| | buffer | | | | |
| Ticagrelor | 0.05% SDS in pH | 800 μg/ml | 11.7 | 11.9 | 7.8 |
| | 6.8 phosphate | | | | |
| | buffer | | | | |
| Apixaban | pH 6.8 phosphate | 800 μg/ml | 49.8 | 48.7 | 48.0 |
| | buffer | | | | |
| Rivaroxaban | pH 6.8 phosphate | 1000 μg/ml | 39.6 | 61.4 | 70.7 |
| | buffer | | | | |
| Warfarin | pH 5.0 phosphate | 600 μg/ml | 32.1 | 27.9 | 28.0 |
| | buffer | | | | |
| Lurasidone | pH 6.8 phosphate | 250 μg/ml | 7.3 | 10.7 | 3.2 |
| hydrochlorate | buffer | | | | |
| Curcumin | pH 6.8 phosphate | 1200 μg/ml | 1.7 | 9.4 | 8.6 |
| | buffer | | | | |
| Vitamin K1 | pH 6.8 phosphate | 1000 μg/ml | 71.6 | 45.6 | 8.0 |
| | buffer | | | | |
| Macitentan | pH 6.8 phosphate | 650 μg/ml | 35.0 | 33.8 | 28.0 |
| | buffer | | | | |
| Tacrolimus | pH 4.5 acetate | 400 μg/ml | 77.4 | 8.2 | 17.3 |
| | buffer | | | | |
| Cyclosporine | pH 6.8 phosphate | 400 μg/ml | 27.1 | 75.5 | 41.1 |
| | buffer | | | | |
| Paclitaxel | 0.1% Tween80 in | 600 μg/ml | 3.0 | 3.0 | 3.9 |
| | pH 6.8 phosphate | | | | |
| | buffer | | | | |
| Docetaxel | 0.1% Tween80 in | 600 μg/ml | 15.4 | 14.9 | 14.2 |
| | pH 6.8 phosphate | | | | |
| | buffer | | | | |
| Ibrutinib | 0.05% Tween 20 in | 850 μg/ml | 16.5 | 17.9 | 18.4 |
| | pH 6.8 phosphate | | | | |
| | buffer | | | | |
| Clopidogrel | pH 4.5 acetate | 1000 μg/ml | 48.9 | 39.6 | 52.0 |
| bisulfate | buffer | | | | |
| Fingolimode | pH 7.4 phosphate | 700 μg/ml | 5.5 | 6.8 | 2.1 |
| | buffer | | | | |
| Enzalutamide | 0.2% SDS in pH | 650 μg/ml | 62.3 | 57.1 | 59.0 |
| | 6.8 phosphate | | | | |
| | buffer | | | | |
| Posaconazole | pH 6.5 phosphate | 400 μg/ml | 1.2 | 2.0 | 9.0 |
| | buffer | | | | |
| Dabigatran | pH 6.8 phosphate | 500 μg/ml | 1.5 | 9.5 | 8.9 |
| Etexilate | buffer | | | | |
| Venetoclax | pH 6.8 phosphate | 300 μg/ml | 19.8 | 12.4 | 9.6 |
| | buffer | | | | |
| Alectinib | pH 6.8 phosphate | 280 μg/ml | 2.5 | 10.2 | 2.2 |
| | buffer | | | | |
| Palbociclib | pH 6.8 phosphate | 450 μg/ml | 140.4 | 34.8 | 31.6 |
| | buffer | | | | |
| Naringenin) | pH 6.8 phosphate | 1600 μg/ml | 169.9 | 126.7 | 129.1 |
| | buffer | | | | |
| Celecoxib | pH 2.0 | 1000 μg/ml | 20.5 | 5.4 | 4.2 |
| | hydrochloric acid | | | | |
| Itraconazole | 0.05% SDS in pH | 400 μg/ml | 10.7 | 2.9 | 2.7 |
| | 6.8 phosphate | | | | |
| | buffer | | | | |
| Eltrombopag | pH 6.8 phosphate | 750 μg/ml | 19.4 | 5.1 | 4.5 |
| | buffer | | | | |
| Griseofulvin | 0.1% SDS in pH | 1000 μg/ml | 35.6 | 27.4 | 24.1 |
| | 6.8 phosphate | | | | |
| | buffer | | | | |
| Ezetimibe | 0.05% SDS in pH | 200 μg/ml | 6.6 | 6.2 | 6.4 |
| | 6.8 phosphate | | | | |
| | buffer | | | | |
| Candesartan | pH 4.5 acetate | 1000 μg/ml | 3.8 | 2.1 | 2.0 |
| Cilexetil | buffer | | | | |
| Felodipine | 0.05% Tween 20 in | 700 μg/ml | 18.1 | 17.8 | 20.4 |
| | pH 6.8 phosphate | | | | |
| | buffer | | | | |
| Scutellarin | pH 6.8 phosphate | 600 μg/ml | 45.3 | 41.7 | 43.1 |
| | buffer | | | | |
| Cryptotanshinone | pH 6.8 phosphate | 300 μg/ml | 1.5 | 2.1 | 2.5 |
| | buffer | | | | |
| Acalabrutinib | pH 7.4 phosphate | 667 μg/ml | 106.0 | 87.6 | 87.6 |
| | buffer | | | | |
| Regorafenib | 0.1% SDS in pH | 500 μg/ml | 2.3 | 2.7 | 2.4 |
| | 6.8 phosphate | | | | |
| | buffer | | | | |
| Butylphthalide | pH 6.5 phosphate | 1500 μg/ml | 252.2 | 246.4 | 246.0 |
| | buffer | | | | |
| Coenzyme Q10 | pH 6.8 phosphate | 200 μg/ml | 1.3 | 2.3 | 0.4 |
| | buffer | | | | |
| Cannabidiol | 0.05% SDS in pH | 1200 μg/ml | 11.4 | 11.8 | 8.5 |
| | 6.8 phosphate | | | | |
| Tafluprost | pH 7.4 phosphate | 800 μg/ml | 18.6 | 9.3 | 6.8 |
| | buffer | | | | |
| Lutein | pH 6.8 phosphate | 300 μg/ml | 0.0 | 0.0 | 0.0 |
| | buffer | | | | |
| Vitamin A | pH 6.8 phosphate | 200 μg/ml | 57.2 | 51.1 | 53.0 |
| | buffer | | | | |
| Vitamin E | pH 6.8 phosphate | 400 μg/ml | 20.9 | 17.9 | 11.9 |
| | buffer | | | | |

The above results showed that most of the target molecules rapidly stacked to form solids and precipitated within 0.5 h due to intermolecular interactions over incubation time, and little target molecules re-dissolved in the solution over incubation time. Compared with the theoretical addition concentration, each target molecule stacked severely after being incubated in the initial medium for 6 hours.

### Comparative Example 2

Appropriate amounts of polymers 101-115 were weighed respectively, and each polymer was prepared into a solution having a polymer mass concentration of approximately 0.5% (0.5 g polymer per 100 mL) using the initial medium corresponding to each target. Experiments were conducted according to the steps in Comparative Example 1, and quantitative analysis was carried out using the analytical method for each target to investigate the regulation ability of each binary supramolecular self-assembly system built from 0.5% polymer and target on guest molecules stacking. The stronger the molecular recognition and synergistic regulation ability in the binary supramolecular self-assembly system, the more stable the supramolecular self-assembly system formed. As time went on, the guest molecule stacking decreased, and the concentration of the guest molecule in the solution tended to be constant.

An experimental scheme was shown in Table 10. The theoretical concentration of each target in the binary supramolecular self-assembly system remained consistent with Table 9.

TABLE 10
Experimental scheme for building binary self-assembly systems from
polymers and targets

| Comparative Example | | Mass concentration of polymer 0.5% (W/V) | | | | | | | | | | |
|---|---|---|---|---|---|---|---|---|---|---|---|---|
| No. | Targets | 101 | 102 | 103 | 104 | 111 | 106 | 107 | 109 | 110 | 105 | 115 |
| 2001 | Nilotinib | √¹ | √ | √ | √ | — | √ | √ | — | —² | — | — |
| 2002 | Nintedanib | √ | √ | √ | — | — | √ | √ | √ | — | — | — |
| 2003 | Lenvatinib | √ | √ | √ | √ | — | √ | √ | — | — | √ | — |
| 2004 | Sorafenib | √ | √ | √ | √ | — | √ | √ | — | — | √ | — |
| 2005 | Ticagrelor | √ | √ | √ | — | √ | √ | √ | √ | √ | — | √ |
| 2006 | Apixaban | √ | √ | √ | √ | √ | √ | √ | — | — | — | — |
| 2007 | Rivaroxaban | √ | √ | √ | √ | — | √ | √ | √ | — | — | — |
| 2008 | Warfarin | √ | √ | √ | — | √ | √ | √ | √ | √ | √ | √ |
| 2009 | Lurasidone | √ | √ | √ | — | — | √ | √ | — | — | — | — |
| 2010 | Curcumin | √ | √ | √ | — | — | √ | √ | √ | √ | √ | — |
| 2011 | Vitamin K1 | √ | √ | √ | — | √ | √ | √ | √ | √ | √ | √ |
| 2012 | Macitentan | √ | √ | √ | — | √ | √ | √ | √ | √ | √ | √ |
| 2013 | Tacrolimus | √ | √ | √ | — | — | — | √ | √ | — | √ | — |
| 2014 | Cyclosporine | √ | √ | √ | — | √ | √ | √ | √ | √ | √ | √ |
| 2015 | Paclitaxel | √ | √ | √ | — | √ | √ | √ | √ | √ | — | √ |
| 2016 | Docetaxel | √ | √ | √ | — | √ | √ | √ | √ | √ | — | √ |
| 2017 | Ibrutinib | √ | √ | √ | √ | — | √ | √ | — | — | — | — |
| 2018 | Clopidogrel | √ | √ | √ | — | √ | √ | √ | √ | √ | — | — |
| 2019 | Fingolimode | √ | √ | √ | — | — | √ | √ | √ | √ | √ | — |
| 2020 | Enzalutamide | √ | √ | √ | √ | — | √ | √ | √ | — | — | — |
| 2021 | Posaconazole | √ | √ | √ | — | √ | √ | √ | √ | √ | — | √ |
| 2022 | Dabigatran | √ | √ | √ | — | — | √ | √ | √ | √ | — | — |
| | Etexilate | | | | | | | | | | | |
| 2023 | Venetoclax | √ | √ | √ | — | — | √ | √ | √ | — | — | — |
| 2024 | Alectinib | √ | √ | √ | — | √ | √ | √ | √ | √ | — | √ |
| 2025 | Palbociclib | √ | √ | √ | √ | — | √ | √ | √ | — | √ | — |
| 2026 | Naringenin* | √ | √ | √ | — | √ | √ | √ | √ | √ | — | √ |
| 2027 | Celecoxib | √ | √ | √ | — | √ | √ | √ | √ | √ | √ | √ |
| 2028 | Itraconazole | √ | √ | √ | √ | — | √ | √ | — | — | — | — |
| 2029 | Eltrombopag | √ | √ | √ | — | — | √ | √ | — | — | — | — |
| 2030 | Ezetimibe | √ | √ | √ | √ | — | √ | — | — | — | — | — |
| 2031 | Griseofulvin | √ | √ | √ | — | √ | √ | √ | √ | √ | √ | √ |
| 2032 | Candesartan | √ | √ | √ | — | √ | √ | √ | √ | √ | √ | √ |
| | Cilexetil | | | | | | | | | | | |
| 2033 | Felodipine | √ | √ | √ | — | — | √ | √ | √ | — | — | — |
| 2034 | Scutellarin | √ | √ | √ | — | √ | √ | √ | √ | √ | — | √ |
| 2036 | Acalabrutinib | √ | √ | √ | — | √ | √ | √ | √ | √ | √ | √ |
| 2037 | Regorafenib | √ | √ | √ | — | √ | √ | √ | √ | √ | — | √ |
| 2038 | Butylphthalide | √ | √ | √ | — | — | √ | √ | √ | — | — | √ |
| 2039 | Cannabidiol | √ | √ | √ | — | — | √ | √ | √ | √ | — | — |
| 2040 | Tafluprost | √ | √ | √ | √ | — | — | √ | — | — | — | — |
| 2041 | Lutein | — | √ | — | — | — | — | — | — | — | — | — |
| 2042 | Vitamin A | — | √ | — | — | — | — | — | — | — | — | — |
| 2044 | Vitamin E | — | √ | — | — | — | — | — | — | — | — | — |

Note:
¹“V” conducted the experiment;
²“--” did not conduct the experiment.

Comparative Examples 2001 to 2044 investigated the effects of polymer built binary supramolecular self-assembly systems incubated for different time on the stacking of targets. The higher concentration and long-time immobility of the target measured in the solution indicated better synergistic regulation effect between the target and the polymer. The results are shown in the table below. If the same polymer involved multiple types, only the ones with the best effect are listed in the table.

TABLE 11
Comparison of synergistic regulation abilities of polymers on the stacking
of targets

| | | Concentration of the target in the solution measured | | | | | | |
|---|---|---|---|---|---|---|---|---|
| Comparative Example | | after different incubation time, μg/ml | | | | | | |
| No. | Targets | 101 | 102 | 103 | 104 | 106 | 107 | 110 |
| 2001 | Nilotinib | √¹ | √ | √ | √ | √ | √ | —² |
| | 0.5 h | 117.0 | 96.7 | 125.2 | 77.5 | 121.2 | 107.2 | — |
| | 3 h | 145.8 | 188.9 | 159.3 | 84.9 | 81.8 | 62.3 | — |
| | 6 h | 149.9 | 189.9 | 181.5 | 61.4 | 50.0 | 52.3 | — |
| 2002 | Nintedanib | √ | √ | √ | — | √ | √ | — |
| | 0.5 h | 80.8 | 75.8 | 65.0 | 25.3 | 46.8 | 58.2 | — |
| | 3 h | 75.2 | 68.2 | 51.5 | 30.2 | 44.1 | 56.2 | — |
| | 6 h | 65.8 | 70.1 | 48.2 | 38.9 | 42.5 | 54.2 | — |
| 2003 | Lenvatinib | √ | √ | √ | √ | √ | √ | — |
| | 0.5 h | 45.5 | 76.8 | 12.9 | 37.8 | 7.3 | 68.2 | — |
| | 3 h | 29.3 | 26.2 | 11.3 | 42.7 | 7.9 | 9.7 | — |
| | 6 h | 28.6 | 27.8 | 7.6 | 67.5 | 6.5 | 6.1 | — |
| 2004 | Sorafenib | √ | √ | √ | √ | √ | √ | — |
| | 0.5 h | 741.5 | 770.6 | 602.5 | 108.9 | 765.3 | 784.3 | — |
| | 3 h | 751.6 | 151.8 | 61.5 | 4.3 | 571.3 | 432.2 | — |
| | 6 h | 759.3 | 102.9 | 38.5 | 3.7 | 310.3 | 244.4 | — |
| 2005 | Ticagrelor | √ | √ | √ | — | √ | √ | √ |
| | 0.5 h | 132.5 | 169.8 | 19.2 | 11.9 | 16.8 | 48.3 | 17.0 |
| | 3 h | 47.2 | 166.0 | 20.4 | 9.4 | 17.4 | 52.1 | 19.2 |
| | 6 h | 46.6 | 161.4 | 17.5 | 9.1 | 17.3 | 45.5 | 19.1 |
| 2006 | Apixaban | √ | √ | √ | √ | √ | √ | — |
| | 0.5 h | 160.8 | 222.4 | 352.3 | 78.6 | 98.7 | 226.9 | — |
| | 3 h | 149.0 | 221.1 | 139.7 | 55.3 | 76.4 | 232.6 | — |
| | 6 h | 169.9 | 222.0 | 114.6 | 43.7 | 78.3 | 227.2 | — |
| 2007 | Rivaroxaban | √ | √ | √ | √ | √ | √ | — |
| | 0.5 h | 119.9 | 87.4 | 122.8 | 40.0 | 45.9 | 41.5 | — |
| | 3 h | 114.0 | 75.0 | 97.4 | 35.7 | 41.9 | 37.3 | — |
| | 6 h | 111.5 | 70.3 | 72.5 | 36.9 | 39.9 | 37.0 | — |
| 2008 | Warfarin | √ | √ | √ | — | √ | √ | √ |
| | 0.5 h | 315.9 | 84.5 | 189.2 | — | 220.3 | 185.1 | 13.1 |
| | 3 h | 212.3 | 76.5 | 189.1 | — | 112.8 | 188.1 | 21.0 |
| | 6 h | 124.4 | 68.7 | 181.4 | — | 23.2 | 56.0 | 20.5 |
| 2009 | Lurasidone | √ | √ | √ | — | √ | √ | — |
| | 0.5 h | 13.2 | 15.3 | 7.0 | — | 4.1 | 3.2 | — |
| | 3 h | 13.5 | 12.7 | 5.2 | — | 3.1 | 2.4 | — |
| | 6 h | 9.4 | 13.4 | 4.0 | — | 1.8 | 1.7 | — |
| 2010 | Curcumin | √ | √ | √ | — | √ | √ | √ |
| | 0.5 h | 219.7 | 747.8 | 76.1 | — | 102.6 | 107.4 | 6.0 |
| | 3 h | 120.1 | 643.4 | 52.9 | — | 118.1 | 8.3 | 8.3 |
| | 6 h | 50.3 | 576.7 | 32.7 | — | 90.3 | 7.7 | 10.0 |
| 2011 | Vitamin K1 | √ | √ | √ | — | √ | √ | √ |
| | 0.5 h | 347.6 | 372.5 | 347.2 | — | 62.0 | 83.0 | 466.4 |
| | 3 h | 369.7 | 238.6 | 347.3 | — | 68.4 | 163.7 | 365.6 |
| | 6 h | 339.5 | 269.2 | 326.4 | — | 16.7 | 38.5 | 382.5 |
| 2012 | Macitentan | √ | √ | √ | — | √ | √ | √ |
| | 0.5 h | 56.0 | 56.5 | 34.3 | — | 41.5 | 46.5 | 28.7 |
| | 3 h | 75.2 | 71.5 | 28.5 | — | 35.6 | 43.5 | 34.7 |
| | 6 h | 46.2 | 89.5 | 27.7 | — | 32.5 | 39.3 | 33.5 |
| 2013 | Tacrolimus | √ | √ | √ | — | √ | √ | — |
| | 0.5 h | 224.0 | 63.4 | 46.8 | — | 78.6 | 68.7 | — |
| | 3 h | 88.8 | 60.3 | 58.7 | — | 65.2 | 62.8 | — |
| | 6 h | 161.3 | 55.7 | 6.0 | — | 5.2 | 13.2 | — |
| 2014 | Cyclosporine | √ | √ | √ | — | √ | √ | √ |
| | 0.5 h | 88.1 | 30.9 | 16.0 | — | 18.9 | 18.3 | 13.5 |
| | 3 h | 110.0 | 33.6 | 13.9 | — | 24.6 | 22.4 | 15.8 |
| | 6 h | 146.3 | 36.1 | 12.9 | — | 26.9 | 23.0 | 23.9 |
| 2015 | Paclitaxel | √ | √ | √ | — | √ | √ | √ |
| | 0.5 h | 217.6 | 147.5 | 8.7 | — | 2.9 | 9.5 | 4.3 |
| | 3 h | 246.9 | 11.3 | 10.4 | — | 2.6 | 10.6 | 2.3 |
| | 6 h | 235.8 | 14.5 | 21.2 | — | 4.2 | 15.9 | 2.2 |
| 2016 | Docetaxel | √ | √ | √ | — | √ | √ | √ |
| | 0.5 h | 318.8 | 317.2 | 127.8 | — | 27.7 | 58.2 | 14.3 |
| | 3 h | 124.7 | 241.1 | 112.8 | — | 20.3 | 41.0 | 11.7 |
| | 6 h | 69.1 | 233.4 | 113.6 | — | 18.1 | 33.8 | 11.9 |
| 2017 | Ibrutinib | √ | √ | √ | √ | √ | √ | — |
| | 0.5 h | 48.5 | 168.1 | 44.1 | 13.4 | 33.8 | 47.9 | — |
| | 3 h | 93.5 | 176.4 | 17.8 | 13.7 | 65.9 | 53.1 | — |
| | 6 h | 95.3 | 154.5 | 17.1 | 14.3 | 68.4 | 56.0 | — |
| 2018 | Clopidogrel | √ | √ | √ | — | √ | √ | √ |
| | 0.5 h | 415.1 | 29.1 | 69.1 | — | 37.0 | 57.0 | 64.7 |
| | 3 h | 421.5 | 28.5 | 69.6 | — | 39.3 | 37.8 | 69.3 |
| | 6 h | 361.7 | 26.1 | 64.5 | — | 39.5 | 36.5 | 64.4 |
| 2019 | Fingolimode | √ | √ | √ | — | √ | √ | √ |
| | 0.5 h | 1.0 | 8.6 | 22.6 | — | 1.2 | 1.4 | 140.7 |
| | 3 h | 2.8 | 21.5 | 37.9 | — | 2.5 | 2.1 | 189.0 |
| | 6 h | 2.2 | 24.9 | 44.5 | — | 2.1 | 5.2 | 211.9 |
| 2020 | Enzalutamide | √ | √ | √ | — | √ | √ | — |
| | 0.5 h | 272.9 | 90.1 | 102.4 | — | 150.2 | 236.9 | 53.8 |
| | 3 h | 221.8 | 75.5 | 88.4 | — | 131.1 | 151.4 | 45.8 |
| | 6 h | 193.8 | 72.2 | 84.4 | — | 128.2 | 137.8 | 43.0 |
| 2021 | Posaconazole | √ | √ | √ | — | √ | √ | √ |
| | 0.5 h | 182.0 | 96.9 | 11.4 | — | 6.8 | 3.5 | 4.6 |
| | 3 h | 181.7 | 95.1 | 6.2 | — | 9.1 | 7.3 | 6.0 |
| | 6 h | 83.4 | 89.6 | 7.1 | — | 5.3 | 2.8 | 12.8 |
| 2022 | Dabigatran | √ | √ | √ | — | √ | √ | √ |
| | Etexilate | | | | | | | |
| | 0.5 h | 102.6 | 47.6 | 9.3 | 6.8 | 2.5 | 12.0 | 5.2 |
| | 3 h | 45.2 | 79.5 | 2.8 | 3.6 | 2.9 | 0.8 | 0.1 |
| | 6 h | 61.3 | 32.4 | 2.1 | 2.7 | 0.6 | 1.1 | 0.6 |
| 2023 | Venetoclax | √ | √ | √ | — | √ | √ | — |
| | 0.5 h | 233.6 | 151.6 | 78.6 | — | 232.2 | 152.2 | 385.5 |
| | 3 h | 249.5 | 254.9 | 72.9 | — | 250.5 | 153.1 | 534.6 |
| | 6 h | 252.6 | 272.1 | 59.5 | — | 156.8 | 156.8 | 566.2 |
| 2024 | Alectinib | √ | √ | √ | — | √ | √ | √ |
| | 0.5 h | 45.8 | 51.5 | 1.0 | — | 1.4 | 1.5 | 1.9 |
| | 3 h | 31.0 | 47.5 | 0.9 | — | 2.1 | 0.8 | 1.6 |
| | 6 h | 9.4 | 45.9 | 1.3 | — | 1.9 | 2.9 | 1.3 |
| 2025 | Palbociclib | √ | √ | √ | √ | √ | √ | — |
| | 0.5 h | 163.1 | 205.7 | 259.9 | 105.4 | 131.3 | 103.5 | — |
| | 3 h | 89.0 | 167.0 | 126.5 | 156.4 | 60.3 | 32.0 | — |
| | 6 h | 85.5 | 154.0 | 109.6 | 144.8 | 58.2 | 27.0 | — |
| 2026 | Naringenin* | √ | √ | √ | — | √ | √ | √ |
| | 0.5 h | 412.1 | 456.4 | 230.7 | — | 469.5 | 127.5 | 112.7 |
| | 3 h | 214.9 | 389.1 | 231.0 | — | 462.5 | 130.9 | 79.0 |
| | 6 h | 126.4 | 383.3 | 233.5 | — | 478.8 | 139.1 | 77.6 |
| 2027 | Celecoxib | √ | √ | √ | — | √ | √ | √ |
| | 0.5 h | 224.3 | 19.6 | 31.1 | — | 49.0 | 36.4 | 6.4 |
| | 3 h | 62.2 | 11.1 | 32.2 | — | 30.1 | 35.4 | 11.1 |
| | 6 h | 54.4 | 10.6 | 31.9 | — | 26.3 | 35.3 | 4.8 |
| 2028 | Itraconazole | √ | √ | √ | √ | √ | √ | — |
| | 0.5 h | 47.9 | 69.7 | 14.1 | 31.5 | 43.3 | 13.5 | — |
| | 3 h | 4.8 | 34.5 | 8.6 | 7.1 | 8.3 | 1.7 | — |
| | 6 h | 3.0 | 24.7 | 1.9 | 3.1 | 6.4 | 1.2 | — |
| 2029 | Eltrombopag | √ | √ | √ | — | √ | √ | √ |
| | 0.5 h | 65.4 | 181.5 | 48.6 | — | 250.3 | 254.6 | 5.2 |
| | 3 h | 70.0 | 151.6 | 67.1 | — | 246.9 | 256.3 | 4.9 |
| | 6 h | 67.3 | 135.9 | 72.0 | — | 242.7 | 255.3 | 4.1 |
| 2030 | Ezetimibe | — | √ | √ | √ | — | — | — |
| | 0.5 h | — | 109.4 | 8.4 | 6.8 | — | — | — |
| | 3 h | — | 117.2 | 6.9 | 3.6 | — | — | — |
| | 6 h | — | 138.9 | 0.9 | 2.7 | — | — | — |
| 2031 | Griseoful vin | √ | √ | √ | — | √ | √ | √ |
| | 0.5 h | 221.0 | 24.1 | 51.8 | — | 67.9 | 109.2 | 27.1 |
| | 3 h | 187.9 | 23.2 | 39.8 | — | 62.8 | 93.9 | 23.6 |
| | 6 h | 171.7 | 28.6 | 39.1 | — | 60.7 | 88.6 | 26.3 |
| 2032 | Candesartan | √ | √ | √ | — | √ | √ | √ |
| | Cilexetil | | | | | | | |
| | 0.5 h | 135.3 | 47.0 | 91.6 | — | 103.1 | 189.1 | 4.7 |
| | 3 h | 34.5 | 43.8 | 110.5 | — | 16.2 | 33.7 | 18.3 |
| | 6 h | 18.4 | 4.9 | 120.7 | — | 18.8 | 28.8 | 3.3 |
| 2033 | Felodipine | √ | √ | √ | — | √ | √ | — |
| | 0.5 h | 144.8 | 459.9 | 18.9 | — | 33.5 | 51.2 | — |
| | 3 h | 152.8 | 472.0 | 18.9 | — | 30.2 | 26.7 | — |
| | 6 h | 147.5 | 459.4 | 22.2 | — | 23.6 | 11.8 | — |
| 2034 | Scutellarin | √ | √ | √ | — | √ | √ | √ |
| | 0.5 h | 210.4 | 122.5 | 40.4 | — | 57.4 | 61.7 | 95.9 |
| | 3 h | 125.2 | 102.0 | 42.8 | — | 60.8 | 41.7 | 87.3 |
| | 6 h | 110.2 | 102.2 | 42.8 | — | 54.3 | 43.1 | 78.3 |
| 2036 | Acalabrutinib | √ | √ | √ | — | √ | √ | √ |
| | 0.5 h | 209.1 | 566.6 | 292.1 | — | 184.1 | 497.8 | 427.6 |
| | 3 h | 193.9 | 580.9 | 321.4 | — | 124.3 | 171.6 | 429.7 |
| | 6 h | 197.8 | 608.2 | 246.4 | — | 159.4 | 167.1 | 460.8 |
| 2037 | Regorafenib | √ | √ | √ | — | √ | √ | √ |
| | 0.5 h | 319.9 | 243.9 | 250.2 | — | 207.1 | 203.3 | 3.2 |
| | 3 h | 328.1 | 192.1 | 224.5 | — | 173.6 | 133.9 | 2.9 |
| | 6 h | 331.8 | 181.0 | 62.2 | — | 175.5 | 225.5 | 1.9 |
| 2038 | Butylphthalide | √ | √ | √ | — | √ | √ | V¹⁰⁹ |
| | 0.5 h | 555.9 | 258.9 | 234.7 | — | 240.7 | 242.4 | 240.5 |
| | 3 h | 229.5 | 245.3 | 230.8 | — | 244.6 | 240.0 | 253.9 |
| | 6 h | 230.7 | 244.5 | 233.8 | — | 241.9 | 243.7 | 252.1 |
| 2039 | Cannabidiol | √ | √ | √ | — | √ | √ | √ |
| | 0.5 h | 282.6 | 307.3 | 54.8 | — | 148.1 | 109.6 | 168.9 |
| | 3 h | 293.7 | 323.6 | 55.1 | — | 149.4 | 97.2 | 8.6 |
| | 6 h | 342.5 | 333.1 | 55.7 | — | 155.7 | 95.7 | 7.3 |
| 2040 | Tafluprost | √ | √ | √ | √ | — | √ | — |
| | 0.5 h | 57.6 | 52.9 | 14.6 | 26.7 | — | 34.5 | — |
| | 3 h | 44.8 | 66.1 | 23.1 | 24.2 | — | 36.8 | — |
| | 6 h | 23.1 | 67.3 | 27.4 | 17.3 | — | 35.2 | — |
| 2041 | Lutein | — | √ | — | — | — | — | — |
| | 0.5 h | — | 16.3 | — | — | — | — | — |
| | 3 h | — | 16.9 | — | — | — | — | — |
| | 6 h | — | 16.0 | — | — | — | — | — |
| 2042 | Vitamin A | — | √ | — | — | — | — | — |
| | 0.5 h | — | 80.5 | — | — | — | — | — |
| | 3 h | — | 80.2 | — | — | — | — | — |
| | 6 h | — | 75.7 | — | — | — | — | — |
| 2043 | Vitamin E | — | √ | — | — | — | — | — |
| | 0.5 h | — | 28.6 | — | — | — | — | — |
| | 3 h | — | 57.9 | — | — | — | — | — |
| | 6 h | — | 36.4 | — | — | — | — | — |

In Comparative Examples 2001 to 2043, different polymer molecules built binary supramolecular self-assembly systems together with targets respectively. Compared with the concentration of targets in initial media, different polymer molecules had different synergistic regulation effects on the stacking of different targets, which was not only related to a chemical structure of a target, hydrogen donor and acceptor groups in the chemical structure, dissociability under the initial medium conditions, and hydrophobicity, etc., but also related to the strength of non-covalent bond interaction forces formed by effective functional groups in the corresponding polymer building block. Only when the non-covalent bond force between the target and the polymer was stronger than the force between the target molecules, the order of supramolecular self-assembly of the target can be disrupted, and a supramolecular self-assembly system based on the polymer and the target can be re-built. The mass concentration of the polymer added in the current supramolecular self-assembly system was 0.5%, and the theoretical concentration of the target was added based on the maximum dose for clinical application, calculated by the volume 100 ml of gastrointestinal fluid in mammals such as humans according to oral gastrointestinal administration. To achieve supersaturation of the target concentration without molecular stacking, at least 500 mg of polymer needed to be added to the product formula. However, the maximum daily doses of the targets were different, and their frequencies of administration were also different. For safety, process, cost, and user compliance considerations, it was not realistic to contain such a large amount of polymer per unit dose in clinical applications, unless the unit dose of active ingredients was very low. The binary supramolecular self-assembly systems built from various polymers, except for acalabrutinib and venetoclax, in the above comparative examples were relatively stable and had strong synergistic regulation effects on the targets. The binary supramolecular self-assembly systems built from other targets and single polymers had certain regulation effects on the stacking of target molecules, but significantly lower than expected.

### Examples 1-10

Ternary supramolecular self-assembly systems based on different polymers, carrier building blocks, and targets were built according to the experimental scheme in Table 12, to investigate the influence of synergistic regulation of the carrier building blocks and the polymers on the stacking of target molecules, as well as the stability of the supramolecular self-assembly systems jointly built by them over incubation time. The initial medium, quantitative analysis method, pre-treatment method, and initial theoretical concentration of the target, corresponding to each target molecule in the experiment, were consistent with those in Comparative Examples 1 and 2. After a target was added to the system, the target was uniformly dispersed by ultrasonication at 37° C. for half an hour and then shaken in a 37° C. constant temperature air shaker, and samples were collected after different incubation time, i.e. 0.5 h, 1 h, 2 h, 3 h, 4 h, and 6 h.

TABLE 12
Experimental design scheme for Examples 1-10

| | | Mass concentration of polymer and carrier | | | | | | | | |
|---|---|---|---|---|---|---|---|---|---|---|
| | | building block added (%) | | | | | | | | |
| Example No. | Target molecules | 101/107 | 102 | 103 | 104 | 301 | | 302 | | 316 |
| 1 | Nilotinib | — | 0.25 | — | — | 0.25 | — | — | — | — |
| | | — | 0.25 | — | — | — | — | 0.25 | — | — |
| | | — | — | 0.25 | — | — | — | 0.25 | — | — |
| | | — | — | — | 0.5 | — | — | — | — | — |
| | | — | — | — | 0.25 | — | — | 0.25 | — | — |
| | | — | — | — | — | 0.25 | — | — | — | — |
| | | — | — | — | — | — | — | 0.25 | — | — |
| 2 | Nintedanib | — | 0.25 | — | — | 0.25 | 0.3 | — | — | — |
| | | — | 0.25 | — | — | — | — | 0.25 | — | — |
| | | — | — | 0.25 | — | 0.25 | — | — | — | — |
| | | — | — | 0.25 | — | — | — | 0.25 | — | — |
| | | — | — | — | 0.25 | 0.25 | — | — | — | — |
| | | — | — | — | 0.25 | — | — | 0.25 | — | — |
| 3 | Sorafenib | 0.25 | — | — | — | 0.5 | — | — | — | — |
| | | 0.25 | — | — | — | — | — | 0.5 | — | — |
| | | 0.25(107) | 0.25 | — | — | — | 0.5 | — | — | — |
| | | 0.25(107) | 0.25 | — | — | — | — | — | 0.5 | — |
| | | — | 0.25 | 0.25 | — | — | 0.5 | — | — | — |
| | | — | 0.25 | 0.25 | — | — | — | — | 0.5 | — |
| 4 | Ticagrelor | — | 0.25 | — | — | 0.25 | 0.5 | — | — | — |
| | | — | 0.25 | — | — | — | — | 0.25 | — | — |
| | | — | — | — | 0.25 | 0.25 | — | — | — | — |
| | | — | — | — | 0.25 | — | — | 0.25 | — | — |
| 5 | Apixaban | — | — | — | — | 0.5 | — | — | — | — |
| | | — | 0.25 | — | — | 0.25 | — | — | — | — |
| | | — | 0.25 | — | — | — | — | 0.25 | — | — |
| | | — | — | 0.25 | — | 0.25 | — | — | — | — |
| | | — | — | — | 0.25 | — | — | 0.25 | — | — |
| | | — | — | — | — | — | — | — | 0.5 | — |
| 6 | Rivaroxaban | — | — | — | — | — | — | — | — | 2.5 |
| | | — | — | — | — | — | — | — | 2.5 | — |
| | | — | — | — | — | — | 2.5 | — | — | — |
| | | — | 0.25 | — | — | 0.2 | 0.5 | — | — | — |
| 7 | Curcumin | — | 0.25 | — | — | — | — | 0.25 | 0.5 | — |
| | | — | 0.25 | 0.25 | — | — | — | 0.25 | — | — |
| | | — | 0.25 | — | 0.25 | — | — | 0.25 | — | — |
| 8 | Ibrutinib | — | 0.25 | — | — | — | — | — | — | — |
| | | — | 0.25 | — | — | 0.25 | — | — | — | — |
| | | — | 0.25 | — | — | — | — | 0.25 | — | — |
| | | — | — | 0.25 | — | — | — | — | — | — |
| | | — | 0.25 | 0.25 | — | — | 0.5 | — | — | — |
| | | — | — | 0.25 | — | — | — | 0.25 | — | — |
| | | — | — | — | 0.25 | — | — | — | — | — |
| | | — | 0.25 | — | 0.25 | 0.25 | — | — | — | — |
| | | — | — | — | 0.25 | — | — | 0.25 | — | — |
| | | — | — | — | — | — | 0.5 | — | — | — |
| 9 | Palbociclib | — | 0.25 | — | — | — | — | — | — | — |
| | | — | 0.25 | — | — | 0.25 | — | — | — | — |
| | | — | 0.25 | — | — | — | — | 0.25 | — | — |
| | | — | — | 0.25 | — | — | — | — | — | — |
| | | — | 0.25 | 0.25 | — | 0.25 | — | — | — | — |
| | | — | — | 0.25 | — | — | — | 0.25 | — | — |
| | | — | — | — | 0.25 | — | — | — | — | — |
| | | — | — | — | 0.25 | — | — | 0.25 | — | — |
| | | — | — | — | — | 0.25 | — | — | — | — |
| | | — | — | — | — | — | — | 0.25 | — | — |
| 10 | Ezetimibe | — | 0.25 | — | — | 0.25 | — | — | — | — |
| | | — | 0.25 | — | — | — | — | 0.25 | — | — |
| | | — | — | 0.25 | — | 0.25 | — | — | — | — |
| | | — | — | 0.25 | — | — | — | 0.25 | — | — |
| | | — | — | — | 0.25 | 0.25 | — | — | — | — |
| | | — | — | — | 0.25 | — | — | 0.25 | — | — |
| | | — | — | — | — | — | 0.5 | — | — | — |
| | | — | — | — | — | — | — | — | 0.5 | — |

The results of Examples 1 to 10 are shown in FIGS. 1-10, respectively. The results of binary self-assembly systems built by target guest molecules and single polymers, as well as the data in the initial media, came from Comparative Examples 1 and 2.

Explanation: 102 polymer includes H (G/F), L (G/F), and M (G/F) types, where G represents a granule grade, F represents a fine powder grade, and G/F has no effect on the present invention. The following 102H represents that the corresponding example adopts 102H type, and so on; 103 polymer includes E3LV series and E5LV series, and the following 103E3 represents that the corresponding example adopts 103 E3LV series; 104 carboxymethyl cellulose includes sodium and calcium salts, but refers to sodium salts unless otherwise specified.

Example 1 and FIG. 1: According to Comparative Examples 1 and 2, among binary supramolecular self-assembly systems built by Nilotinib with 0.5% polymers 102H, 103E3 and 104 respectively, compared with the initial medium, the binary supramolecular self-assembly systems built by polymers 102H and 104 respectively had stronger synergistic regulation effects on target guest molecule stacking, and the concentrations of the guest molecule in their solutions measured at 6 hours were about 8.5 times and 2.7 times that of the initial medium. Among ternary supramolecular self-assembly systems built by polymers 102H, 103E3 and 104, the target molecule, and carrier building block 302, the ternary supramolecular self-assembly system built by polymers 102H and 103E3 had a significant synergistic regulation effect on guest molecule stacking, and was significantly better than the ternary supramolecular self-assembly systems built by respective polymers in the synergistic regulation effect, where the ternary supramolecular self-assembly system built by polymers 102H and 103E3 had a solution concentration of 391.1 μg/mL measured at 6 hours and an encapsulation rate of 97.8% for the guest molecule and can exist stably. A ternary supramolecular self-assembly system built by the carrier building block 301 and polymer 102H showed a slow growth trend from 0.5 hour to 6 hours, the 6-hour system still did not reach equilibrium, and the concentration of the guest molecule in its solution was 227.2 μg/mL, which was 10 times that of the initial medium.

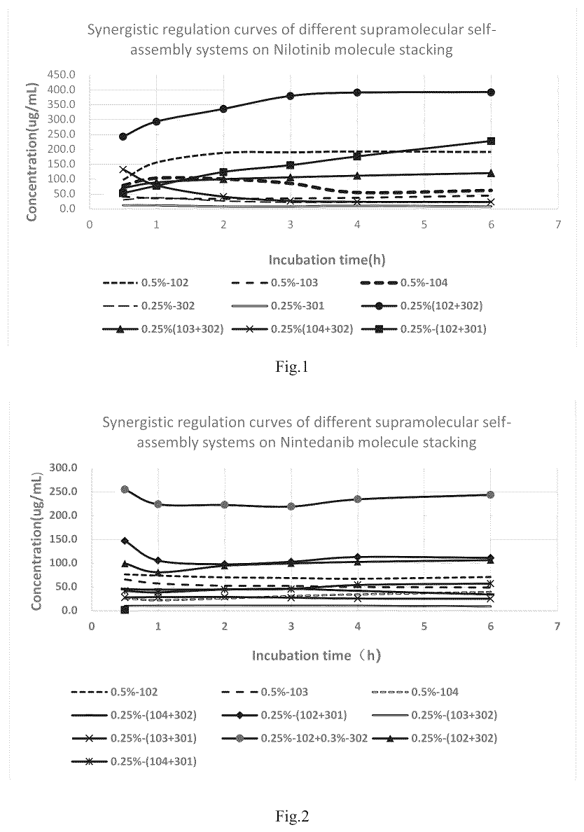

Example 2 and FIG. 2: According to Comparative Examples 1 and 2, binary supramolecular self-assembly systems built by Nintedanib with 0.5% polymer 102H or 103E5 or 104 respectively had certain synergistic regulation effect on guest molecule stacking after incubation for 6 hours compared with that of initial medium, where the concentration of the guest molecule in the solution of the binary supramolecular self-assembly system built by 0.5% 102H, measured at 6 hours, was 70.1 μg/mL, which was 9.6 times that of the initial medium. Among ternary supramolecular self-assembly systems built by a natural building unit 301, the guest molecule, and 0.25% 102H, 103 E5 and 104 respectively, the ternary supramolecular self-assembly system jointly built by 102H and 301 exhibited significant synergistic regulation on guest molecule stacking, and the concentration of the guest molecule in the solution measured at 6 hours was 110.5 μg/mL, which was 15.1 times that of the initial medium. Among ternary supramolecular self-assembly systems built by a natural building block 302, a target and polymers respectively, the ternary supramolecular self-assembly system jointly built by 102H and 302 exhibited significantly synergistic regulation on guest molecule stacking, and the concentration of the guest molecule at 6 hours was 105.6 μg/mL, which was 14.5 times that of the initial medium. By adjusting the mass percentage of 302 in the ternary supramolecular self-assembly system to 0.3%, the concentration of the guest molecule measured at 6 hours was 242.9 μg/mL, which was 33.3 times that of the initial medium.

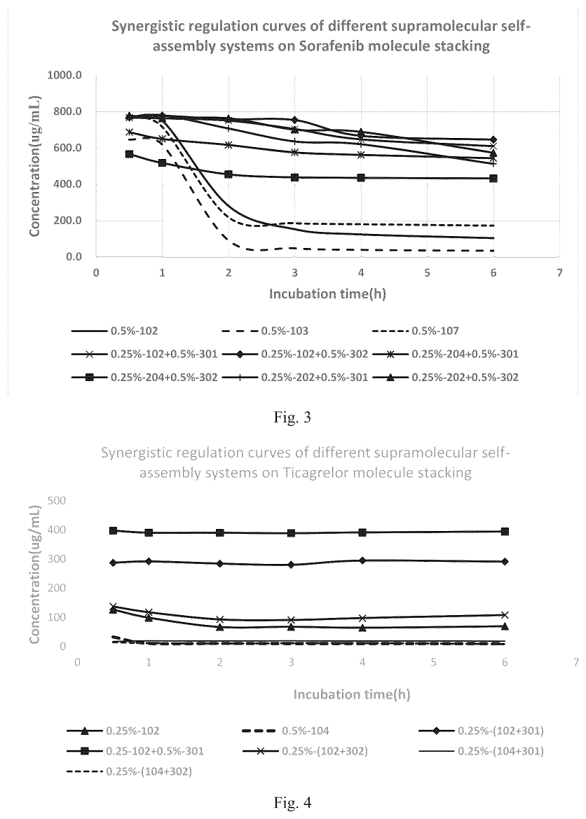

Example 3 and FIG. 3: According to Comparative Examples 1 and 2, binary supramolecular self-assembly systems built by a guest Sorafenib with 0.5% polymer 102H, 103E5 or 107 respectively had a weak synergistic regulation effect on Sorafenib molecule stacking, the Sorafenib molecule rapidly stacked after 2 hours, and the concentrations of the guest molecule measured at 6 hours were 102.9, 33.4 and 171.6 μg/mL, which were 18.4 times, 6.0 times, and 30.6 times that of the initial medium, respectively. Among ternary supramolecular self-assembly systems built by 0.5% carrier building units 301 and 302 with 0.25% polymer 102H respectively, the concentrations of the Sorafenib molecule measured at 6 hours were 609.8 μg/mL and 644.6 μg/mL, which were 108.7 times and 115 times that of the initial medium, respectively; among quaternary supramolecular self-assembly systems built by composite polymer 204 (102H+103 E5) with 0.5% natural building block 301 or 302, and the guest molecule, the concentrations of Sorafenib measured at 6 hours were 543.3 μg/mL and 431.5 μg/mL, which were 96.8 times and 76.9 times that of the initial medium, respectively; among quaternary supramolecular self-assembly systems built by composite polymer 202 (102H+107) with 0.5% natural building unit 301 or 302 and the guest molecule, the concentrations of Sorafenib in their solutions measured at 6 hours were 512.2 μg/mL and 572.8 μg/mL, which were 91.3 times and 102.1 times that of the initial medium, respectively; and these concentrations were significantly higher than the solution concentration achieved by the binary system built by 0.5% polymer 103E5 or 107 and Sorafenib at 6 hours, respectively.

Example 4 and FIG. 4: According to Comparative Examples 1 and 2, a guest molecule Ticagrelor and 0.5% 102M or 104 built a binary supramolecular self-assembly system respectively. Compared with initial medium, the binary supramolecular self-assembly system built by 0.5% 102M and the guest had certain regulation effect on guest molecule stacking, but the regulation effect was weak. Compared with the initial medium, a ternary supramolecular self-assembly system built by 301 or 302, the guest molecule, and 0.25% 104 had no regulation effect on guest molecule stacking. Ternary supramolecular self-assembly systems built by 301 or 302, the guest molecule, and 0.25% 102M had a significant synergistic regulation effect on guest molecule stacking, where the synergistic regulation effect of the ternary system built by 0.25% 301+0.25% 102M and the guest molecule was stronger than that of 0.25% 302 and 0.25% 102M, and the concentrations of the guest molecule measured at 6 hours were be 290.8 and 107.2 μg/mL, which were 26 times and 9.7 times that of the initial medium, respectively. By continuing to increase the mass concentration of 301, the supramolecular self-assembly system built by 0.5% 301, 0.25% 102M and the guest molecule achieved the strongest synergistic regulation on guest molecule stacking and became stable, and the concentration of the guest molecule at 6 hours was 394 μg/mL, which was 35 times that of the initial medium.

Example 5 and FIG. 5: According to Comparative Examples 1 and 2, among binary supramolecular self-assembly systems built by 0.5% 102H, 0.5% 103E5, and 0.5% 104 with a guest molecule respectively, the binary supramolecular self-assembly system built by 102H and 103E5 had a significant synergistic regulation effect on guest molecule stacking; in the binary supramolecular self-assembly system built by 0.5% 102H and the guest molecule, the concentration of Apixaban measured at 6 hours was 327.6 μg/mL, which was 6.7 times that of the initial medium. Without the addition of polymers, among binary supramolecular self-assembly systems built by 0.5% 301 and 302 with the guest molecule respectively, the binary supramolecular self-assembly system built by 301 had a significant synergistic regulation effect on guest molecule stacking, and the concentration of the guest molecule measured at 6 hours was 366.6 μg/mL, which was 7.5 times that of the initial medium. 302 almost had no regulation effect on guest molecule stacking. Ternary supramolecular self-assembly systems built by 0.25% 102H with 301 or 302 and the guest molecule respectively had a significant synergistic regulation effect on guest molecule stacking compared with the binary system built by initial medium or polymer, where the concentrations of the guest molecule in measured at 6 hours were 334.8 μg/mL and 312.6 μg/mL, which were equivalent to those of 0.5% polymer 102H alone. Ternary supramolecular self-assembly systems built by 0.25% 104 and 302, or 0.25% 103E5 and 302, and the guest molecule respectively did not show any advantages over 0.5% polymer 103E5 or 104.

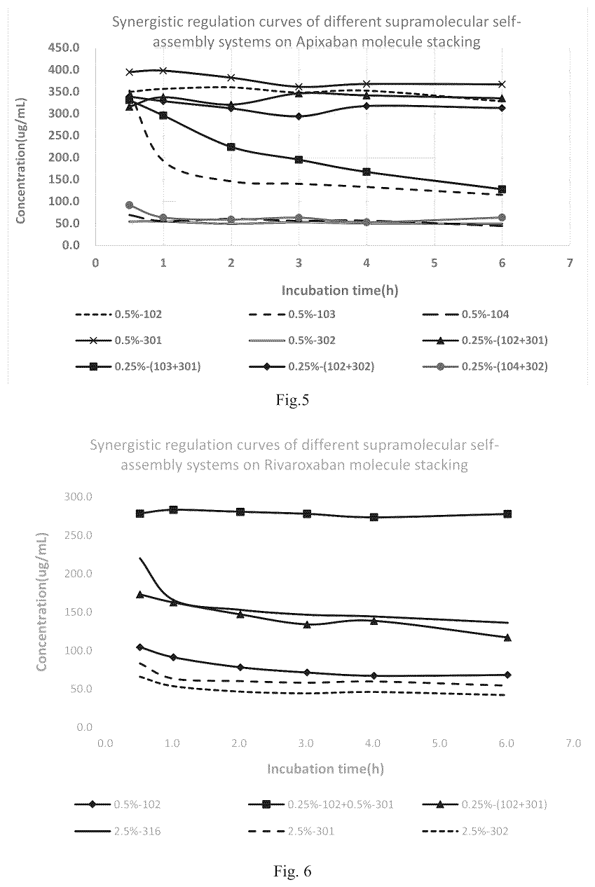

Example 6 and FIG. 6: According to Comparative Examples 1 and 2, among binary supramolecular self-assembly systems built by Rivaroxaban with 2.5% 301 or 302 and 0.5% 102H respectively, compared with initial medium, the binary supramolecular self-assembly system built by those three had a weak regulation effect on guest molecule stacking. A ternary supramolecular self-assembly system built by 0.5% 301, 0.25% 102H and the guest molecule had a significant synergistic regulation effect on guest molecule stacking and can be stable, and the concentration of the guest molecule measured at 6 hours was 277.6 μg/mL, which was 9.4 times that of the initial medium. By reducing the mass concentrations of the natural building blocks, the synergistic regulation effect of the ternary supramolecular self-assembly system built by 0.25% 301, 0.25% 102H, and guest molecules on guest molecule stacking was also weakened, but was the same as the binary system built by 2.5% 316, where the concentrations of guest molecules in their solutions measured at 6 hours were 117-136 μg/mL, which was about 4-5 times that of the initial medium. In binary supramolecular self-assembly systems built by 2.5% natural building blocks 301 and 302 and guest molecules respectively, the concentrations of guest molecules in their solutions measured at 6 hours were about 2 times that of the initial medium.

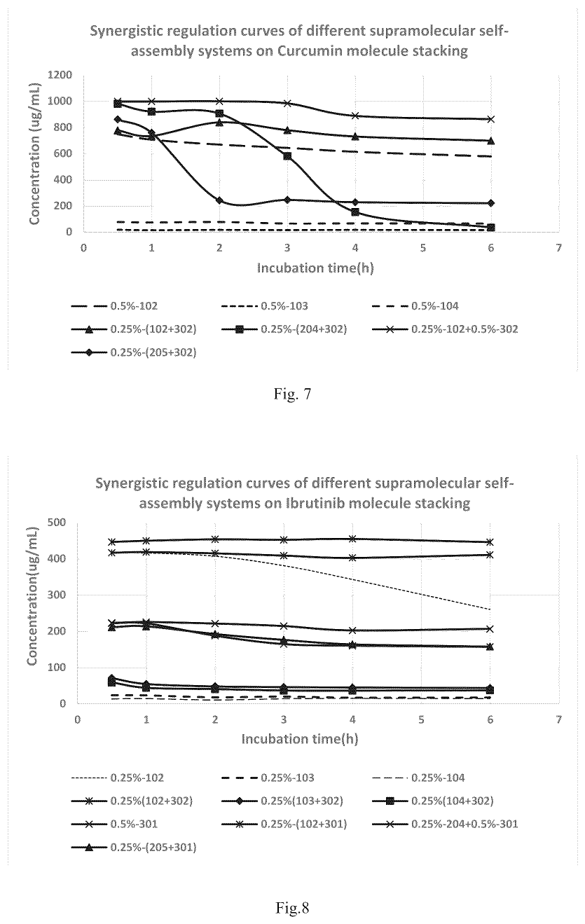

Example 7 and FIG. 7: According to Comparative Examples 1 and 2, 0.5% 102M, 103E5, and 104, with a guest molecule Curcumin, built binary supramolecular self-assembly systems respectively, where the supramolecular self-assembly system built by 0.5% 102M and the guest molecule had a relatively strong synergistic regulation effect on guest molecule stacking, but was unstable, and exhibited slow stacking over incubation time, and the concentration of the guest molecule in the 6-hour solution was 576.6 μg/mL, which was 67 times that of the initial medium; the binary supramolecular self-assembly systems built by 0.5% 103E5 and 104 almost had no synergistic regulation effect on guest molecule stacking. Among ternary or quaternary supramolecular self-assembly systems built by natural building block 302 with 0.25% 102M, 204 (102M+103E5) or 205 (102M+104) and a guest molecule, the ternary supramolecular self-assembly system built by 0.25% 102M and 302 exhibited strongest synergistic regulation on guest molecule stacking and remained stable for 6 hours; by continuing to increase the mass percentage of 302 in the ternary supramolecular self-assembly system to 0.5%, its synergistic regulation ability was significantly enhanced, and the concentration of the guest molecule measured at 6 hours was 862.1 μg/mL, which was 100.6 times that of the initial medium; the quaternary supramolecular self-assembly system built by 0.25% 204 or 205 with 302 and the guest molecule showed that the guest molecule packed over incubation time, and in particular, the quaternary supramolecular self-assembly system built by composite polymer 204 showed that the concentration of the guest molecule was 579.0 g/mL in the 3-hour system solution and rapidly decreased to 34.2 μg/mL after 6 hours, indicating that there was insufficient polymer providing a hydrophobic effect in the system.

Example 8 and FIG. 8: According to Comparative Examples 1 and 2, among binary supramolecular self-assembly systems built by a guest molecule Ibrutinib with 0.25% 102H, 103E5, or 104 respectively, compared with initial medium an initial medium, the binary supramolecular self-assembly systems built by 103E5 and 104, except for 102H, had no synergistic regulation effect on guest molecule stacking; and the binary supramolecular self-assembly system built by 0.25% 102H had a significant synergistic regulation effect on guest molecule stacking, but the guest molecule slowly packed over time, and the concentration of the guest molecule in the solution measured at 6 hours was 260.2 μg/mL, which was 14 times that of the initial medium. A binary supramolecular self-assembly system built by the guest molecule and 0.5% 301 tended to be stable after incubation for 6 hours, and the concentration of the guest molecule in the 6-hour system was 156.9 μg/mL, which was 8.5 times that of the initial medium. Among ternary or quaternary supramolecular self-assembly systems built by the guest, 302, and 102H or 204 (102H+103E5) or 205 (102H+104) respectively, the ternary supramolecular self-assembly system built by 302 and 102H showed the strongest synergistic regulation effect and became stable by molecular recognition and synergistic regulation, and the concentration of the guest molecule in the system measured at 6 hours was 410.7 μg/mL, which was 22 times that of the initial medium. A ternary supramolecular self-assembly system built by 302, 103E5 or 104, and the guest molecule showed no significant synergistic regulation effect; ternary or quaternary supramolecular self-assembly systems built by 301 and 102H, 204 or 205 as well as the guest molecule had a very significant synergistic regulation effect on guest molecule stacking, where the ternary supramolecular self-assembly system built by 102H, 301 and the guest molecule quickly became stable by molecular recognition and synergistic regulation, and the concentration of the guest molecule in the system measured at 6 hours was 446.0 μg/mL, which was about 25 times that of the initial medium. A ternary supramolecular self-assembly system built by 0.5% 301 with 0.25% 204 and the guest molecule, or by 0.25% 301 with 0.25% 205 and the guest molecule, affected by molecular recognition and synergistic regulation, had a lower encapsulation rate for the guest molecule than that of the ternary system built by 301 and 102H, but the two self-assembly systems can be quickly formed, and the concentrations of the guest molecule in the systems measured at 6 hours were 206.4 μg/mL and 157.4 μg/mL, which were 11 times and 8.6 times that of the initial medium, respectively.

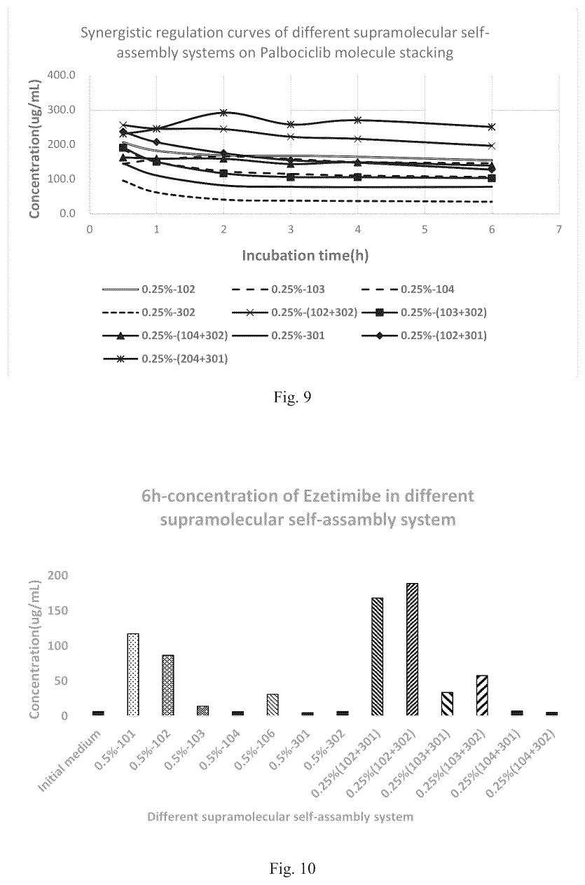

Example 9 and FIG. 9: According to Comparative Examples 1 and 2, binary supramolecular self-assembly systems built by a guest Palbociclib with 102H or 103E5 or 104 respectively can be quickly stable by molecular recognition, and the concentrations of the guest molecule in the systems measured at 6 hours were 154.0 μg/mL, 105.4 μg/mL, and 144.8 μg/mL, which were 4.9 times, 3.3 times, and 4.6 times that of initial medium, respectively. In binary supramolecular self-assembly systems built by 0.25% 301 or 302 with the guest molecule respectively, the concentrations of the guest molecule measured at 6 hours were 77.0 μg/mL and 34.2 μg/mL respectively, indicating a weak synergistic regulation effect. In ternary supramolecular self-assembly systems built by 0.25% 302 with 0.25% 102H or 103E5 or 104 and the guest molecule, the concentrations of the guest molecule measured at 6 hours were 195.5 μg/mL, 102.3 μg/mL, and 138.3 μg/mL, respectively, and except for the ternary supramolecular self-assembly system built by 102H, the ternary supramolecular self-assembly systems built by 103E5 and 104 did not show a significant synergistic regulation effect. In ternary or quaternary supramolecular self-assembly systems built by 301 with 102H or composite polymer 204 (103E5+102H) and the guest molecule, the concentrations of the guest molecule measured at 6 hours were 126.9 μg/mL and 250.4 μg/mL, which were 4.0 times and 7.9 times that of the initial medium, respectively.

Example 10 and FIG. 10: According to Comparative Examples 1 and 2, binary supramolecular self-assembly systems built by 0.5% 101 or 0.5% 102H with a guest molecule Ezetimibe showed a significant synergistic regulation effect on the guest molecule, and the concentrations of the guest molecule in the systems measured at 6 hours were 117.2 g/mL and 86.6 μg/mL, which were 18 times and 13.5 times that of initial medium, respectively. Ternary supramolecular self-assembly systems built by 0.25% polymer 102H with 301 or 302 and the guest molecule can quickly become stable by molecular recognition and synergistic regulation, and the concentrations of the guest molecule in the systems measured at 6 hours were 168.1 μg/mL and 188.6 μg/mL, which were 26 times and 29.5 times that of the initial medium, respectively. Ternary supramolecular self-assembly systems built by 103E5, 104 with 301 or 302 and the guest molecule had a relatively weak synergistic regulation effect on guest molecule stacking.

### Examples 11-20

Natural building units were selected from 310, 312, 313, 317, 314, 311, and 318, and experiments were conducted at the mass concentrations (W/V %) designed according to the following table. Others were the same as those in Examples 1-10 and Comparative Examples 1 and 2. The quantitative analysis method for the concentration of each target guest molecule was the same as before.

TABLE 13
Scheme design of Examples 11-22

| | Target | Mass concentration | | | | Mass concentration of natural | | | | | | |
|---|---|---|---|---|---|---|---|---|---|---|---|---|
| Example | guest | of polymer (%) | | | | building unit added (%) | | | | | | |
| No. | molecule | 101/107 | 102 | 103 | 104 | 310 | 312 | 313 | 318 | 311/319 | 314 | 317 |
| 11 | Ticagrelor | — | 0.25 | — | — | 0.25 | — | — | — | — | — | — |
| | | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | — | — | — | 0.5 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 12 | Rivaroxaban | — | — | 0.25 | — | 0.3 | — | — | — | — | — | — |
| | | — | — | 0.25 | — | — | 0.3 | — | — | — | — | — |
| | | — | — | 0.25 | — | — | — | 0.5 | — | — | — | — |
| | | — | — | 0.25 | — | — | — | — | 0.3 | — | — | — |
| | | — | — | — | — | 0.3 | — | — | — | — | — | — |
| | | — | — | — | — | — | 0.3 | — | — | — | — | — |
| | | — | — | 0.25 | — | — | — | — | — | 0.1³ | — | — |
| | | — | — | 0.25 | — | — | — | — | — | 0.3³ | — | — |
| | | — | — | — | — | — | — | 0.5 | — | — | — | — |
| | | — | — | — | — | — | — | — | 0.3 | — | — | — |
| 13 | Apixaban | — | 0.25 | — | — | 0.25 | — | — | — | — | — | — |
| | | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | 0.25 | 0.25 | — | 0.25 | — | — | — | — | — | — |
| | | — | 0.25 | 0.25 | — | — | 0.25 | — | — | — | — | — |
| | | — | 0.25 | 0.25 | — | — | — | 0.25 | — | — | — | — |
| | | — | 0.25 | 0.25 | — | — | — | — | 0.25 | — | — | — |
| | | — | — | — | — | — | — | — | — | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 14 | Ibrutinib | — | 0.25 | — | — | 0.25 | — | — | — | — | — | — |
| | | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | 0.25 | — | — | — | — | 0.5 | — | — | — | — |
| | | — | 0.25 | — | — | — | — | — | 0.5 | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 15 | Dabigatran | — | 0.25 | — | — | 0.25 | — | — | — | — | — | — |
| | Etexilate | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 16 | Lenvatinib | — | 0.25 | — | — | 0.25 | — | — | — | — | — | — |
| | | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | — | — | 0.25 | 0.25 | — | — | — | — | — | — |
| | | — | — | — | 0.25 | — | 0.25 | — | — | — | — | — |
| | | — | — | — | 0.25 | — | — | 0.25 | — | — | — | — |
| | | — | — | — | 0.25 | — | — | — | 0.25 | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 17 | Curcumin | — | 0.25 | — | — | — | — | 0.25 | — | — | — | — |
| | | — | 0.25 | — | — | — | — | — | 0.25 | — | — | — |
| | | — | 0.25 | — | — | — | — | — | — | — | 0.25 | — |
| | | — | 0.25 | — | — | — | — | — | — | — | — | 0.25 |
| | | — | — | — | — | — | — | 0.25 | — | — | — | — |
| | | — | — | — | — | — | — | — | 0.25 | — | — | — |
| | | — | — | — | — | — | — | — | — | — | 0.25 | — |
| | | — | — | — | — | — | — | — | — | — | — | 0.25 |
| 18 | Sorafenib | — | 0.25 | — | — | 0.25 | — | — | — | — | — | — |
| | | — | 0.25 | — | — | — | — | — | — | 0.25 | — | — |
| | | — | 0.25 | — | — | — | — | 0.25 | — | — | — | — |
| | | — | 0.25 | — | — | — | — | — | 0.25 | — | — | — |
| | | — | — | — | — | 0.25 | — | — | — | — | — | — |
| | | — | — | — | — | — | — | — | — | 0.25 | — | — |
| | | — | — | — | — | — | — | 0.25 | — | — | — | — |
| | | — | — | — | — | — | — | — | 0.25 | — | — | — |
| 19 | Nintedanib | — | 0.25 | — | — | 0.25 | — | — | — | — | — | — |
| | | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 20 | Docetaxel | — | 0.25 | — | — | 0.25 | — | — | — | — | — | — |
| | | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | — | 0.25 | — | 0.25 | — | — | — | — | — | — |
| | | — | — | 0.25 | — | — | 0.25 | — | — | — | — | — |
| | | — | — | 0.25 | — | — | — | 0.25 | — | — | — | — |
| | | — | — | 0.25 | — | — | — | — | 0.25 | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| 21 | Lurasidone | — | 0.25 | — | — | 0.25 | — | — | — | — | — | — |
| | hydrochloride | — | 0.25 | — | — | — | 0.25 | — | — | — | — | — |
| | | — | 0.25 | — | — | — | — | 0.25 | — | — | — | — |
| | | — | 0.25 | — | — | — | — | — | 0.25 | — | — | — |
| | | — | — | — | — | 0.25 | — | — | — | — | — | — |
| | | — | — | — | — | — | 0.25 | — | — | — | — | — |
| | | — | — | — | — | — | — | 0.25 | — | — | — | — |
| | | — | — | — | — | — | — | — | 0.25 | — | — | — |
| | | 0.25 | — | — | — | — | — | — | — | — | 0.25¹ | — |
| | | 0.25 | — | — | — | — | — | — | — | — | — | 0.2 |
| | | — | — | — | — | — | — | — | — | — | — | 5² |
| 22 | Dabigatran | 0.25 | 0.25 | — | — | 0.25 | — | — | — | — | — | — |
| | Etexilate | — | — | — | — | — | — | — | — | — | — | — |
| | | 0.25 | 0.25 | — | — | — | 0.25 | — | — | — | — | — |
| | | 0.25 | 0.25 | — | — | — | — | 0.25 | — | — | — | — |
| | | 0.25 | 0.25 | — | — | — | — | — | 0.25 | — | — | — |

Note:
¹represents carrier 301, ²represents carrier 302, and ³represents carrier 319.

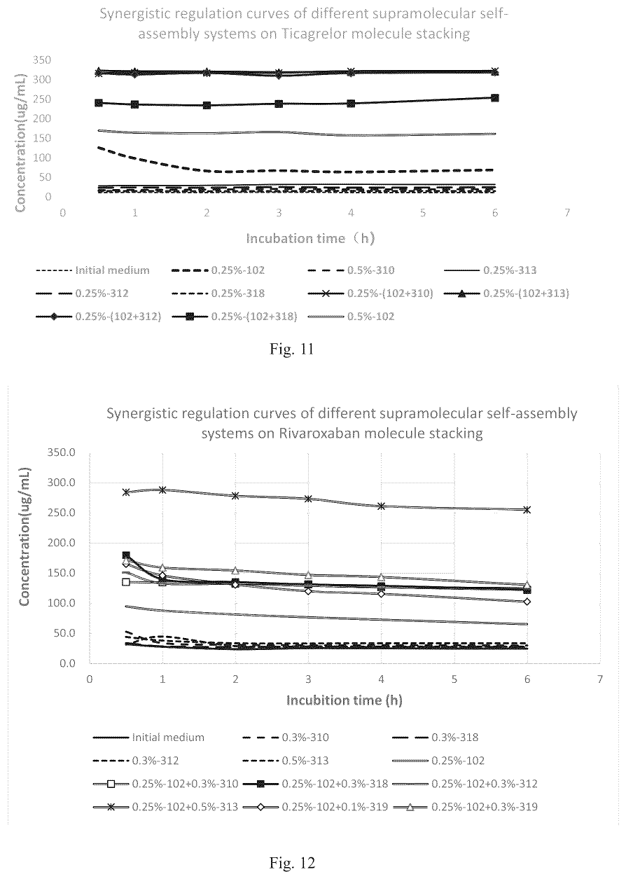

The experimental results are shown in FIG. 11 to FIG. 22, respectively.

Example 11 and FIG. 11: According to Comparative Examples 1 and 2, Ticagrelor was used as a target molecule, natural building blocks were selected from 310, 312, 313 and 318, and a polymer was 102M. Among binary supramolecular self-assembly systems built by a guest molecule Ticagrelor with 0.25% 102M, or Ticagrelor with 0.25% 310, 312, 313 and 318 respectively, after incubation for 6 hours, the solution concentration of the binary supramolecular self-assembly system built by Ticagrelor and 102M was about 6 times that of initial medium; binary systems built by natural building blocks had a slightly weaker regulation effect on guest molecule stacking than the binary system built by 102M, about 1.5 to 3 times that of the initial medium; ternary supramolecular self-assembly systems built by the natural building blocks, the guest molecule, and 102M had a significant synergistic regulation effect on guest molecule stacking, where the synergistic regulation effect and encapsulation rate of the ternary self-assembly supramolecular self-assembly systems built by 310, 312, and 313 were basically the same, and the concentrations of the guest molecule in their solution measured at 6 hours were 318.5 to 322.3 μg/mL, which were 29 times that of the initial medium; the ternary supramolecular self-assembly system built by 318 also exhibited relatively strong synergistic regulation on guest molecule stacking, but its encapsulation rate was slightly lower than those of the other three, the concentration of the guest molecule in its solution measured at 6 hours was 253.7 μg/mL, and its encapsulation rate was significantly higher than that of the binary supramolecular self-assembly systems built by 0.5% 102 and the guest molecule.

Example 12 and FIG. 12: According to Comparative Examples 1 and 2, Rivaroxaban was used as a target guest, a polymer building unit was selected from 102H type, and natural building blocks were selected from 310, 312, 313, 318 and 319. Binary supramolecular self-assembly systems built by Rivaroxaban with 0.3% 310, 312, and 318 or 0.5% 313 respectively had a relatively weak synergistic regulation effect on guest molecule stacking, the guest molecule in each system quickly packed at 0.5 hour, and the concentrations of the guest molecule in the binary supramolecular self-assembly systems measured at 6 hours were 24.5 to 33.4 μg/mL, which was equivalent to that in initial medium. Ternary supramolecular self-assembly systems built by the guest molecule, 102H and natural building blocks respectively exhibited a significant synergistic regulation effect, the solution concentrations of the ternary supramolecular self-assembly systems measured at 6 hours were between 102.5 to 225.1 μg/mL, the ternary supramolecular self-assembly systems remained stable within 6 hours, and the ternary supramolecular self-assembly system built by 0.25% 102H, 0.5% 313 and the guest had a stronger synergistic regulation ability. Among the ternary supramolecular self-assembly systems built by 319 of different concentrations and 0.25% 102H, the synergistic regulation effect of the ternary system built by 0.3% high-concentration 319 was slightly stronger than that built by 0.1% 319, but did not exhibit linear enhancement.

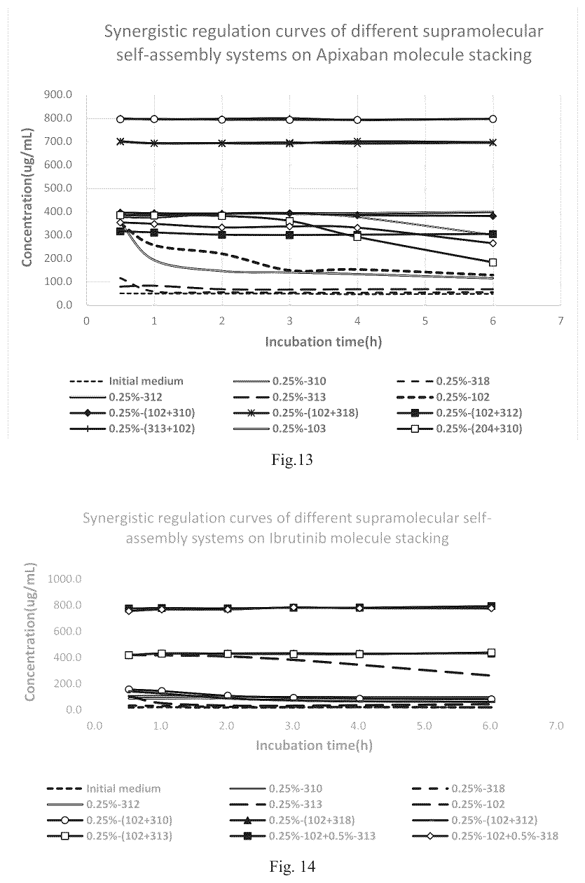

Example 13 and FIG. 13: According to Comparative Examples 1 and 2, Apixaban was used as a target molecule; polymer building blocks were selected from 102H, 103E5, and composite polymer 204 (103E5+102H); and natural building blocks were selected from 310, 312, 313 and 318. Binary supramolecular self-assembly systems built by the guest with 0.25% 102H or 103E5 exhibited certain synergistic regulation effects on guest molecule stacking, but the guest molecule exhibited a slow stacking trend over incubation time, and the concentrations of their solutions measured at 6 hours were 127.7 μg/mL and 114.6 μg/mL, which were 2.7 times and 2.4 times that of initial medium. Among binary supramolecular self-assembly systems built by the guest molecule with 310, 312, 313 and 318 respectively, the binary supramolecular self-assembly system built by 310 and 312 had stronger synergistic regulation effects on guest molecule stacking, and the concentrations of the guest molecule in their solutions measured at 6 hours were 300.0 μg/mL and 397.9 μg/mL, which were 6.3 times and 8.2 times that of the initial medium. Ternary supramolecular self-assembly systems built by 310, 312, 313 and 318, 102H and the guest molecule respectively had significant synergistic regulation effects on guest molecule stacking, can become stable, and were significantly better than any binary supramolecular self-assembly system. The encapsulation rates of the guest molecule in the supramolecular self-assembly systems built by ligands 310 and 312 were significantly lower than those in the ternary supramolecular self-assembly systems built by ligands 313 and 318, and the encapsulation rates of the latter were close to 100%. The synergistic regulation effects of quaternary supramolecular self-assembly systems built by the guest molecule, 0.25% composite polymer 204, and 310, 312, 313 or 318 were similar to those of the ternary systems built by 102H and natural building blocks, but both were significantly higher than that of the binary system built by 0.5% 103E5 and the guest molecule.

Example 14 and FIG. 14: According to Comparative Examples 1 and 2, Ibrutinib was used as a target molecule, 102H was used as a polymer building unit, and natural building blocks were selected from 310, 312, 313 and 318. A binary supramolecular self-assembly system built by the guest with 0.25% 102H had a significant synergistic regulation effect on guest molecule stacking, and the concentration of the guest molecule in its solution measured at 6 hours was 260.2 μg/mL, which was 14.5 times that of initial medium. Binary supramolecular self-assembly systems built solely by the natural building blocks, except for 318, had certain synergistic regulation effects on guest molecule stacking and can quickly reach stability within 6 hours. Among ternary supramolecular self-assembly systems built by 310, 312, 313 and 318 with the guest molecule and 102H respectively, the ternary supramolecular self-assembly systems built by 310 and 312 with 102H did not show synergistic regulation effects; the synergistic regulation effects of the ternary supramolecular self-assembly systems built by 313 and 318 on guest molecule stacking were significantly enhanced, and the concentrations of the guest molecule in their solutions measured at 6 hours were 436.6 μg/mL and 429.9 μg/mL, which were better than the binary systems built by each. By further increasing the mass percentages of 313 and 318 in the ternary systems, their synergistic regulation effects achieved the best, the built ternary supramolecular self-assembly systems were also the most stable, and the concentrations of the guest molecule in their solutions measured at 6 hours were 792.1 μg/mL and 774.7 μg/mL, which were 43 times and 42 times that of the initial medium, where the ternary supramolecular self-assembly system built by 313 basically achieved a 100% encapsulation rate of the guest molecule.

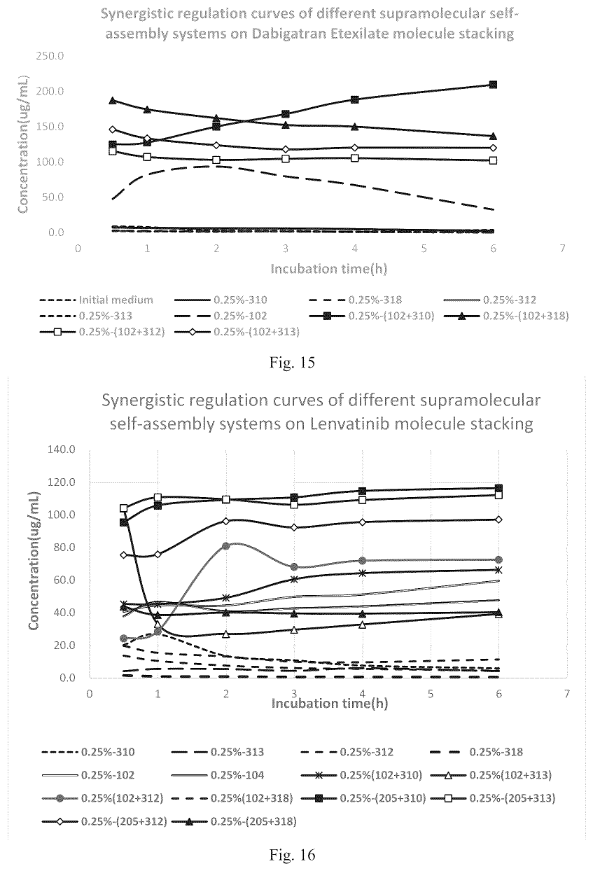

Example 15 and FIG. 15: According to Comparative Examples 1 and 2, Dabigatran Etexilate was used as a target molecule, a polymer building unit was selected from 102H, and natural building blocks were selected from 310, 312, 313 and 318. Binary supramolecular systems built by Dabigatran Etexilate with 310, 312, 313 and 318 did not exhibit synergistic regulation effects on guest molecule stacking; a supramolecular system built by Dabigatran Etexilate with 102H had a significant synergistic regulation effect on Dabigatran Etexilate molecule stacking, and the concentration of the guest molecule in the solution measured at 6 hours was 32.4 μg/mL, which was 162 times that of initial medium; ternary supramolecular self-assembly systems built by Dabigatran Etexilate, 0.25% 102H and natural building blocks respectively showed significant synergistic regulation effects on Dabigatran Etexilate molecule stacking compared to the binary supramolecular self-assembly systems and the initial medium, and the concentrations of the guest molecule in their solutions measured at 6 hours were 102.0 to 209.2 μg/mL, which were 510 to 1046 times that of the initial medium.

Example 16 and FIG. 16: Free Lenvatinib base was used as a target molecule; polymer building blocks were selected from 102H, 104 and composite polymer 205 (102H+104); and natural building blocks were selected from 310, 312, 313 and 318. Compared with initial medium, binary supramolecular self-assembly systems built by Lenvatinib with 102H or 104 had significant synergistic regulation effects on Lenvatinib molecule stacking and ultimately became stable, and the concentrations of the guest molecule in their solutions measured at 6 hours were 59.5 μg/mL and 47.6 μg/mL. The synergistic regulation effects of the binary supramolecular self-assembly systems built by 312 and 310 with the guest molecule were slightly stronger than those built by 313 and 318, but lower than those of the binary supramolecular self-assembly systems built by 102H and 104. The synergistic regulation effects of ternary supramolecular self-assembly systems built by each natural building unit with 102H and the guest molecule, except for 318, were better than those of the binary systems, and the concentrations of the guest molecule in their solutions measured at 6 hours were about 39.2 to 72.5 μg/mL. The synergistic regulation effects of quaternary supramolecular self-assembly systems built by each natural building unit with 0.25% 205 and the guest molecule on guest molecule stacking were significantly enhanced, and particularly for ligands 310 and 313, the concentrations of the guest molecule in their solutions measured at 6 hours were 116.4 μg/mL and 112.1 μg/mL respectively, which were significantly higher than those of the binary supramolecular self-assembly systems.

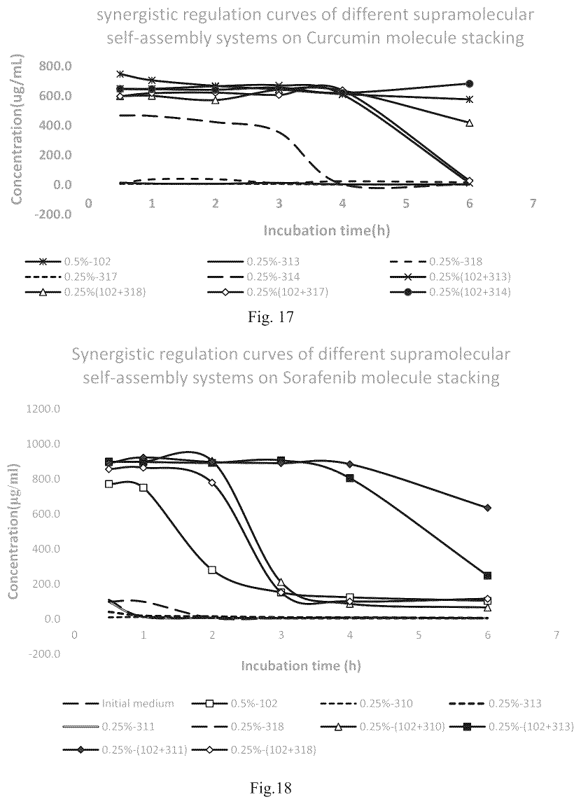

Example 17 and FIG. 17: Curcumin was used as a target molecule, a polymer building unit was selected from 102H, and natural building blocks were selected from 313, 318, 317 and 314. Among binary supramolecular self-assembly systems built by the guest molecule with 313, 314, 317 and 318, each binary supramolecular self-assembly system had almost no regulation effect on guest molecule stacking except for the system built by 314. The synergistic regulation effects of ternary supramolecular self-assembly systems built by the natural building blocks, the polymer and the guest molecule on guest molecule stacking were significantly better than those of the binary systems, where the concentrations of the guest molecule in the 4-hour ternary supramolecular self-assembly systems built by ligands 313 and 317 were about 609.8 μg/mL and 638.6 μg/mL which were significantly higher than those in the binary supramolecular self-assembly systems. However, the guest molecule rapidly packed within 4-6 hours, indicating that the ternary supramolecular self-assembly systems built by 313 and 317 were unstable. The ternary supramolecular self-assembly systems built by 318 and 314 had relatively strong synergistic regulation effects on guest molecule stacking, and were stable within 6 hours.

Example 18 and FIG. 18: Free Sorafenib base was used as a target molecule; natural building blocks were 310, 311, 313 and 318; and polymer was 102H. Compared with initial medium, a binary supramolecular self-assembly system built by 0.5% 102H and the guest molecule Sorafenib exhibited severe molecule stacking within 4 to 6 hours, and the average concentration of the guest molecule in the solution measured at this time was 110 μg/mL. Binary supramolecular self-assembly systems built by the natural building blocks 310, 311, 312 and 318 almost had no synergistic regulation effects on guest molecule stacking. Among ternary supramolecular self-assembly systems built by the natural building blocks, 102H, and the guest molecule respectively, the ternary systems built by 310 and 318 exhibited significant synergistic regulation effects within 2 hours, and the concentrations of the guest molecule in their solutions at this time were 903.1 μg/mL and 778.1 μg/mL, which were significantly better than those of the binary systems; but then the guest molecule rapidly packed, and the concentrations of the guest molecule in their solutions measured at 6 hours were 65.6 μg/mL and 115.4 μg/mL. The concentrations of 4-hour solutions of ternary supramolecular self-assembly systems built by 311 and 313 were 804.8 μg/mL and 884.2 μg/mL, the molecule slowly packed within 4 to 6 hours, and the concentrations of the solutions measured at 6 hours were 246.9 μg/mL and 634.8 μg/mL respectively. Ternary self-assembly systems built by 311 and 313 with polymer 102H respectively exhibited stronger synergistic regulation effects on guest molecule stacking.

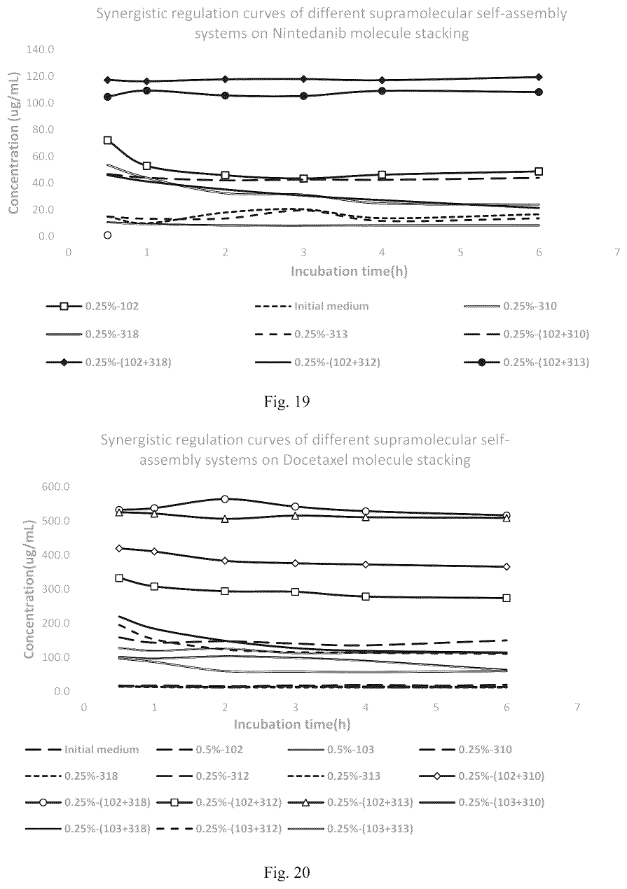

Example 19 and FIG. 19: Free Nintedanib base was used as a target molecule; natural building blocks were selected from 310, 312, 313 and 318; and polymer was 0.25% 102H. Among binary supramolecular self-assembly systems built by the guest molecule with the polymer or the guest molecule with the natural building blocks, the binary systems built by the polymer with the guest molecule or the guest molecule with 312 had certain synergistic regulation effects on guest molecule stacking, and their solution concentrations measured at 6 hours were about twice that of initial medium (44.7 to 48.7 μg/mL). Among ternary supramolecular self-assembly systems built by the 0.25% polymer 102H, the guest molecule and the natural building blocks, except for 312, all had significant synergistic regulation effects, and the concentrations of the guest molecule in their solutions measured at 6 hours were 108.1 to 119.4 μg/mL, which were significantly better than the regulation effect of any binary supramolecular self-assembly system.

Example 20 and FIG. 20: Docetaxel was used as a target molecule, polymers were selected from 0.25% 102H and 0.25% 103E5, and natural building blocks were selected from 0.25% 310, 0.25% 318, 0.25% 312, and 0.25% 313. Binary self-assembly systems built by 0.25% 310, 312, 313 and 318 did not exhibit significant synergistic regulation effects on guest molecule stacking, which was consistent with that of initial medium; Ternary self-assembly systems built by the guest and 0.25% 102H with the natural building blocks respectively exhibited significant synergistic regulation effects, and the solution concentrations of the guest molecule in the ternary supramolecular self-assembly systems measured at 6 hours were 273.9 to 516.3 μg/mL, which were significantly higher than that of the binary self-assembly system built by 0.5% 102H or 0.5% 103E5; and ternary supramolecular self-assembly systems built by 0.25% 103E5 with 310, 312, 313 or 318 and the guest respectively did not exhibit synergistic regulation effects on guest molecule stacking compared to the individual action of 0.5% 103E5.

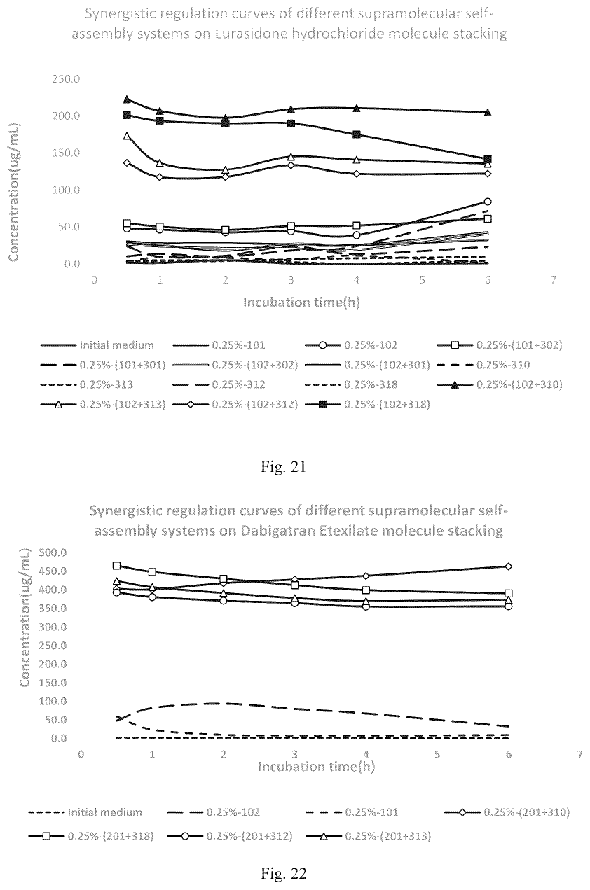

Example 21 and FIG. 21: Lurasidone hydrochloride was used as a target molecule, polymers were selected from 101 and 102H, and natural building blocks were selected from 301, 302, 310, 312, 313 and 318. The guest molecule and 0.25% polymer 101 or 0.25% polymer 102H built a binary self-assembly system respectively, the concentrations of the guest molecule in their solutions measured at 6 hours were 32.4 μg/mL and 84.4 μg/mL respectively, and compared with initial medium, the two polymers exhibited significant synergistic regulation effects on guest molecule stacking. Among binary self-assembly systems built by 0.25% 310, 312, 313, and 318 with the guest respectively, only 312 had a relatively strong synergistic regulation effect, and the concentration of the guest in the system measured at 6 hours was 71.5 μg/mL. There was no significant difference in synergistic regulation between ternary self-assembly systems built by 0.25% 101 and 301 or 302 and the binary system built by 0.25% 101 and the guest. Ternary self-assembly systems built by 301 or 302, 102H, and the guest molecule did not exhibit synergistic regulation effects on guest molecule stacking. Ternary self-assembly systems built by the natural building blocks 318, 310, 312 and 313, 0.25% 102H, and the guest molecule all exhibited significant synergistic regulation effects, and the concentrations of the guest molecule in their solutions measured at 6 hours were 141.8 μg/mL, 204.9 μg/mL, 122.1 μg/mL, and 135.7 μg/mL respectively, where the supramolecular self-assembly system built by 310 and 102H exhibited the strongest synergistic regulation effect.

Example 22 and FIG. 22: Dabigatran Etexilate was used as a target molecule, polymer building unit was 0.25% 201 (102H+101), and natural building blocks were selected from 0.25% 310, 312, 313 and 318. Comparing the results of Example 15 with quaternary self-assembly systems built by 0.25% composite polymer 201 with 310, 312, 313 and 318 and the guest molecule respectively, the self-assembly system built by each natural building unit and the composite polymer had a significant synergistic regulation effect on guest molecule stacking, the concentrations of the guest molecule in their solutions measured at 6 hours were 356 μg/mL to 463 μg/mL, which were significantly higher than that of initial medium and the results of Example 15, and the formed supramolecular self-assembly systems were stable and did not slow molecular stacking at 6 hours.

### Example 23

Ticagrelor was used as a target molecule; natural building blocks were selected from: Steviol glycosides extract containing 25% of Rebaudioside A from STEVIOL GLYCOSIDES, Ste. and Mogroside, Mog. [No. 315-1, where Steviol glycosides 90%: total Steviol glycosides content ≥95%, Stevioside ≥55%, Rebaudioside A ≥25], 0.25% 315, Mogroside extract Mog.30 [containing mogroside V about 30%, No. 319-1, actually measured: Mogroside V: 35.78%, 11-Oxo-mogroside: 5.31%, Siamenoside I: 3.27%], and 0.25% 319; and a polymer was 0.25% 102H. Experiments were conducted according to Comparative Examples 1 and 2, and the quantitative analysis method for Ticagrelor was the same as before. Experiments were conducted according to the following scheme:

TABLE 14
Design scheme for Example 23

| | | Mass con- | | | | |
|---|---|---|---|---|---|---|
| | | centration | | | | |
| Exam- | Target | of | Stevioside Extract | | Mogroside √ | |
| ple | guest | polymer % | Reba.A 25³ | Stev.98 | Mog.30⁴ | Mog.95 |
| No. | molecule | 102H | 315-1 | 315 | 319-1 | 319 |
| 23 | Ticagrelor | 0.25 | 1.0 | — | — | — |
| | | 0.25 | — | 0.25 | — | — |
| | | 0.25 | — | — | 1.0 | — |
| | | 0.25 | — | — | — | 0.25 |
| | | — | 1.0 | — | — | — |
| | | — | — | 0.25 | — | — |
| | | — | — | — | 1.0 | — |
| | | — | — | — | — | 0.25 |

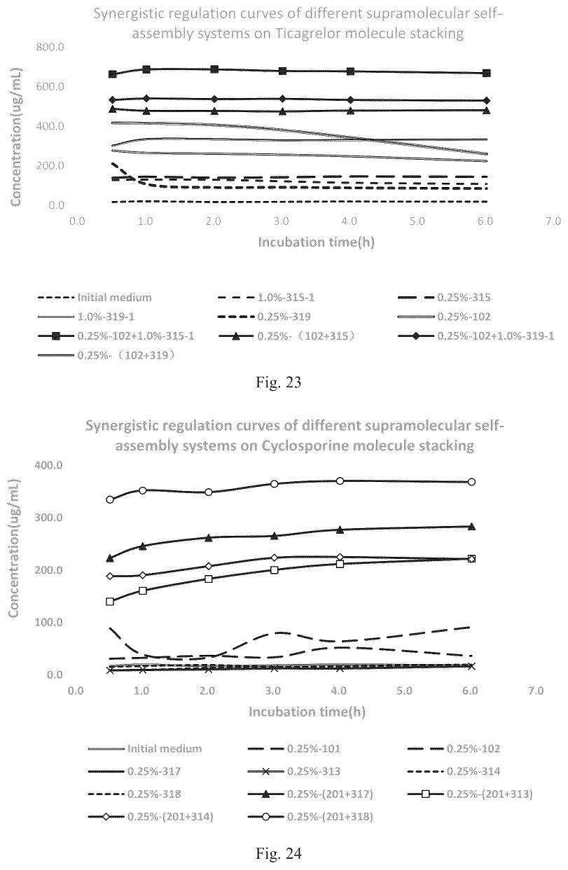

The experimental results are shown in FIG. 23. Ticagrelor as a target molecule built self-assembly systems with 1.0% 315-1, 1.0% 319-1, 0.25% 315, and 0.25% 319 respectively, and compared with initial medium, the concentrations of the guest molecule in their solutions measured at 6 hours were 84.9 to 224.0 μg/mL, which were significantly higher than that of the initial medium (18.4 μg/mL); the guest molecule and 0.25% 102H built a self-assembly system, and the concentration of the guest molecule in its solution measured at 6 hours was 260.2 μg/mL, which was approximately 14 times that of the initial medium; the guest molecule, 0.25% 102H, and the natural building blocks built ternary self-assembly systems respectively with synergistic regulation effects, and the concentration of the guest molecule in each solution measured at 6 hours was 333.3 to 669.1 μg/mL, which was significantly higher than that of the binary self-assembly system. The synergistic regulation effects of ternary self-assembly systems built by 1.0% 319-1 or 1.0% 315-1 with 102H and the guest molecule respectively on guest molecule stacking were significantly better than those of ternary self-assembly systems built by 0.25% 319 or 0.25% 315 with the guest and 102H respectively.

### Example 24

Cyclosporine a target molecule built supramolecular self-assembly systems with 0.25% composite polymer (102H+101) and natural building blocks 313, 314, 317 or 318, respectively. The synergistic regulation effect of each system on cyclosporine molecule stacking was investigated. The experimental scheme was shown in the table below, the experimental steps were the same as those in Comparative Examples 1 and 2, and the quantitative determination method for Cyclosporine was the same as before.

TABLE 15
Design scheme for Example 24

| | | | | Mass percentage of natural | | | |
|---|---|---|---|---|---|---|---|
| Example | Target guest | Polymer-201 | | building unit added (%) | | | |
| No | molecule | 102H | 101 | 313 | 314 | 317 | 318 |
| 24 | Cyclosporine | 0.25 | 0.25 | 0.25 | — | — | — |
| | | 0.25 | 0.25 | — | 0.25 | — | — |
| | | 0.25 | 0.25 | — | — | 0.25 | — |
| | | 0.25 | 0.25 | — | — | — | 0.25 |
| | | — | — | 0.25 | — | — | — |
| | | — | — | — | 0.25 | — | — |
| | | — | — | — | — | 0.25 | — |
| | | — | — | — | — | — | 0.25 |

The experimental results are shown in FIG. 24. Compared with initial medium, binary self-assembly systems built by the guest molecule and the natural building blocks had weak synergistic regulation ability on guest molecule stacking; the synergistic regulation abilities of quaternary self-assembly systems built by the guest molecule with composite polymer 0.25% 201 and the natural building blocks on guest molecule stacking were significantly enhanced, and the concentrations of the guest molecule in their solutions measured at 6 hours were 221.2 to 368.4 μg/mL, which were 12 to 20 times that of the initial medium, where the quaternary supramolecular self-assembly system built by 0.25% 318, 0.25% 201, and the guest molecule had the best synergistic regulation ability on guest molecule stacking.

### Example 25

Fingolimode was used as a target molecule, a polymer was composite polymer 206 (102H+109 S), and natural building blocks were selected from 310, 311, 317 or 318. Experiments were conducted according to the following table, the experimental steps were the same as those in Comparative Examples 1 and 2, and the quantitative determination method for Fingolimode was the same as before.

TABLE 16
Design scheme for Example 25

| Exam- | Target | | | Mass percentage of natural | | | |
|---|---|---|---|---|---|---|---|
| ple | guest | Polymer | | building unit added (%) | | | |
| No | molecule | 102H | 109S | 310 | 311 | 317 | 318 |
| 25 | Fingolimode | 0.25 | 0.25 | 0.25 | — | — | — |
| | | 0.25 | 0.25 | — | 0.25 | — | — |
| | | 0.25 | 0.25 | — | — | 0.25 | — |
| | | 0.25 | 0.25 | — | — | — | 0.25 |
| | | | — | 0.25 | — | — | — |
| | | | — | — | 0.25 | — | — |
| | | | — | — | — | 0.25 | — |
| | | | — | — | — | — | 0.25 |

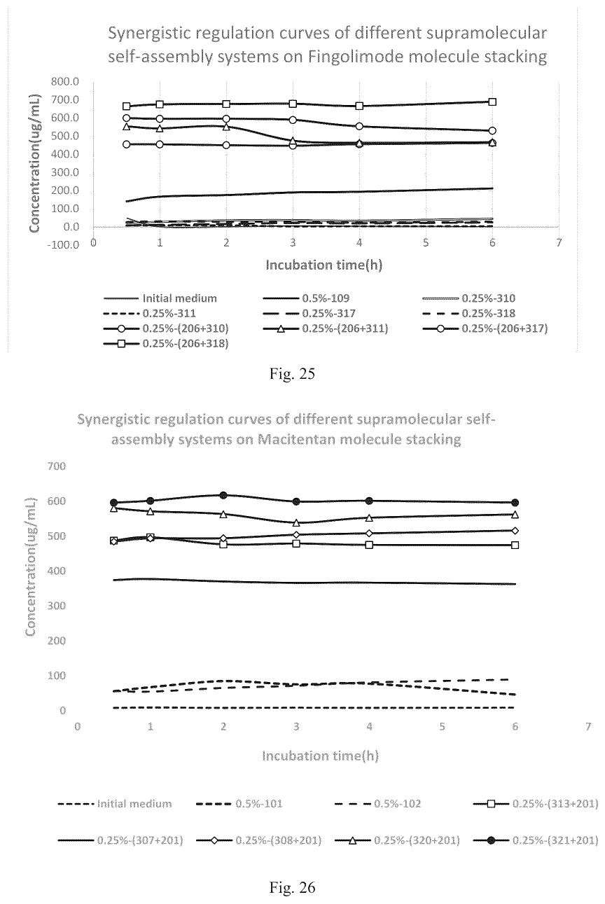

According to FIG. 25, compared with initial medium (37° C.-6 h, concentration of the guest molecule: 1.0 μg/mL), a supramolecular system built by the guest molecule and 0.5% 109 (S type) exhibited significant synergistic regulation on guest molecule stacking, and the concentration of the guest molecule in its solution measured at 6 hours was 212 μg/mL, which was 212 times that of the initial medium. When the guest molecule built binary supramolecular self-assembly systems with 0.25% 310, 311, 317, and 318, except for 0.25% 311, the other three natural building blocks had a significant impact on guest molecule stacking, and compared with the initial medium, the concentration of the guest molecule in each system measured at 6 hours was 24 to 44 times that of the initial medium. The synergistic regulation effects of quaternary supramolecular self-assembly systems built by the natural building blocks, 0.25% 206, the guest molecule, and 0.25% the above natural building unit were better than those of the binary supramolecular self-assembly systems built by 0.5% 109S and the guest or 0.25% natural building blocks and the guest; the concentrations of the guest molecule in the solutions of the quaternary supramolecular self-assembly systems measured at 6 hours were 462.3 to 688.7 μg/mL, and stable self-assembly systems can be formed.

### Example 26

Macitentan was used as a target molecule, 0.25% composite polymer 201 (102H+101) was used as a polymer building block, and natural building blocks were selected from 307, 308, 313, 320, and 321. The experimental scheme was shown in the table below, the experimental steps were the same as those in Comparative Examples 1 and 2, and the quantitative determination method for Macitentan was the same as before.

TABLE 17
Design scheme for Example 26

| | Target | Mass percentage of building | | | | | | |
|---|---|---|---|---|---|---|---|---|
| Example | guest | unit added (%) | | | | | | |
| No | molecule | 101 | 102H | 307 | 308 | 313 | 320 | 321 |
| 26 | Macitentan | 0.25 | 0.25 | 0.25 | — | — | — | |
| | | 0.25 | 0.25 | — | 0.25 | — | — | |
| | | 0.25 | 0.25 | — | — | 0.25 | — | |
| | | 0.25 | 0.25 | — | — | — | 0.25 | |
| | | 0.25 | 0.25 | | | | | 0.25 |

The experimental results are shown in FIG. 26. Binary supramolecular self-assembly systems built by the guest Macitentan with 0.5% 101 or 0.5% 102H were incubated at 37° C. for 6 hours, and the measured concentrations of the guest molecule in their solution were 46.2 μg/mL and 89.5 μg/mL, which were 4.3 times and 10.4 times that of initial medium, respectively. Quaternary supramolecular self-assembly systems built by 0.25% composite polymer 201 with 0.25% 307, 308, 313, 320 or 321, and the guest molecule all had significant synergistic regulation ability, and the concentrations of the guest molecule in the solutions of the systems measured at 6 hours were sequentially 362.5, 516.0, 474.6, 562.4 and 596.3 μg/mL, which were 43.2 to 71.0 times that of the initial medium, respectively. By finely adjusting the composition of the supramolecular self-assembly systems, the systems can maintain hydrogen bonding, electrostatic interaction, hydrophobic interaction, or other non-covalent interaction required for stability with the guest molecule to achieve equilibrium, which was more conducive to building stable supramolecular self-assembly systems.

### Example 27

Tacrolimus as a target molecule built ternary supramolecular self-assembly systems with polymer 102H and natural building blocks 307, 308, 313, 320, or 321, respectively. The experimental scheme was shown in the table below, the experimental steps were the same as those in Comparative Examples 1 and 2, and the quantitative determination method for Tacrolimus was the same as before.

TABLE 18
Design scheme for Example 27

| | Target | Mass percentage of each | | | | | |
|---|---|---|---|---|---|---|---|
| Example | guest | building unit added (%) | | | | | |
| No | molecule | 102H | 307 | 308 | 313 | 320 | 321 |
| 27 | Tacrolimus | 0.25 | 0.25 | — | — | — | — |
| | | 0.25 | — | 0.25 | — | — | — |
| | | 0.25 | — | — | — | 0.25 | — |
| | | 0.25 | — | — | — | — | 0.25 |
| | | — | 0.5 | — | — | — | — |
| | | — | — | 0.5 | — | — | — |
| | | — | — | — | — | 0.5 | — |
| | | — | — | — | — | — | 0.5 |

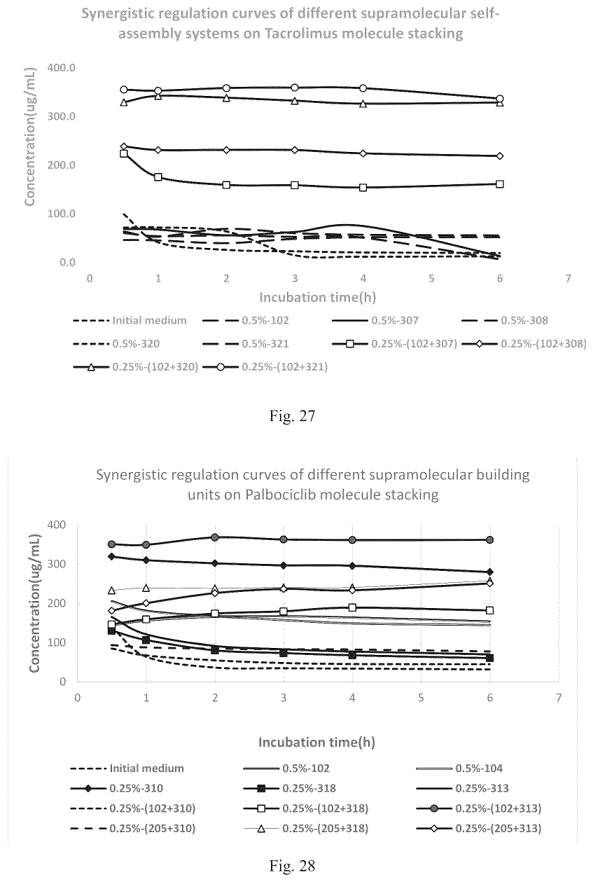

The experimental results are shown in FIG. 27.

Compared with initial medium, binary supramolecular systems built by 0.5% 102H or 0.5% 307, 308, 320, and 321 respectively with Tacrolimus showed that both the polymer and the natural building blocks had no significant synergistic regulation effects on guest molecule stacking. The guest molecule and 0.25% 102H built ternary supramolecular self-assembly systems with ligands 307, 308, 320 and 321, respectively, and the concentrations of the guest molecule in their solutions measured at 6 hours were 161.3, 219.1, 328.9, and 337.2 μg/mL, respectively, where the ternary supramolecular self-assembly systems built by 320 and 321 exhibited relatively strong synergistic regulation effects and can be stable for a long time.

### Example 28

Palbociclib as a target molecule, and 0.25% composite polymer 205 (102H+104), or 102H built quaternary supramolecular self-assembly systems with natural building blocks 310, 318, and 313 respectively. The experimental scheme was shown in the table below, the experimental steps were the same as those in Comparative Examples 1 and 2, and the quantitative determination method for Palbociclib was the same as before.

TABLE 18
Experimental scheme for Example 28

| | Target | Mass percentage | | Mass percentage of | | |
|---|---|---|---|---|---|---|
| Example | guest | of polymer (%) | | natural building unit (%) | | |
| No | molecule | 102 H | 104 | 310 | 318 | 313 |
| 28 | Palbociclib | 0.25 | — | 0.25 | — | — |
| | | 0.25 | — | — | 0.25 | — |
| | | 0.25 | — | — | — | 0.25 |
| | | 0.25 | 0.25 | 0.25 | — | — |
| | | 0.25 | 0.25 | — | 0.25 | — |
| | | 0.25 | 0.25 | — | — | 0.25 |
| | | — | — | 0.25 | — | — |
| | | — | — | — | 0.25 | — |
| | | — | — | — | — | 0.25 |

The experimental results are shown in FIG. 28. From Comparative Example 2, compared with initial medium, binary supramolecular self-assembly systems built by 0.5% 102H or 0.5% 104 and the guest molecule Palbociclib had strong synergistic regulation effects on guest molecule stacking, and the concentrations of the guest molecule in their solutions measured at 6 hours were 154.0 μg/mL and 144.8 μg/mL, which were 4.9 times and 4.6 times that of the initial medium, respectively. 0.25% 310, 318 and 313 and the guest molecule built binary supramolecular self-assembly systems, the concentrations of the guest molecule in their solutions measured at 6 hours were 279.8 μg/mL, 60.5 μg/mL and 70.0 μg/mL, respectively, and all the systems had certain synergistic regulation effects on guest molecule stacking, where ligand 310 showed the most significant effect. Among ternary supramolecular self-assembly systems built by 0.25% 102H with the guest molecule and 0.25% 310, 318 and 313 respectively, the ternary supramolecular self-assembly system built by 310, 102H and the guest molecule did not exhibit any synergistic regulation advantage, and the concentration of the guest molecule in its solution measured at 6 hours was 44.8 μg/mL, which was significantly lower than that of the binary supramolecular self-assembly system built by only 0.25% 310; the ternary supramolecular self-assembly systems built by 0.25% 102H with 318 or 313 and the guest exhibited significant synergistic regulation effects, the concentrations of the guest molecule in their solutions measured at 6 hours were 181.8 μg/mL and 361.6 μg/mL, and the ternary systems remained stable within 6 hours. Quaternary supramolecular self-assembly systems built by 0.25% composite polymer 205 with 310, 318 or 313 and the guest molecule all exhibited significant synergistic regulation effects. The concentrations of the guest molecule in the solutions of the systems built by 0.25% 318 or 313 and 0.25% composite polymer 205, measured at 6 hours, were 257.8 μg/mL and 250.7 μg/mL, which were 8.2 times and 7.9 times that of the initial medium, respectively.

### Example 29

Enzalutamide as a target molecule, composite polymer 202 (102H+107), and natural building blocks 312, 318, 313 or 321 built supramolecular self-assembly systems respectively. The experimental scheme was shown in the table below, the experimental steps were the same as those in Comparative Examples 1 and 2, and the quantitative determination method for Enzalutamide was the same as before.

TABLE 20
Experimental scheme for Example 29

| | | Mass percentage of each building | | | | | |
|---|---|---|---|---|---|---|---|
| Example | Target guest | unit added (%) | | | | | |
| No | molecule | 102H | 107 | 312 | 318 | 313 | 321 |
| 29 | Enzalutamide | 0.25 | 0.25 | 0.25 | — | — | — |
| | | 0.25 | 0.25 | — | 0.25 | — | — |
| | | 0.25 | 0.25 | — | — | 0.25 | — |
| | | 0.25 | 0.25 | — | — | — | 0.25 |
| | | — | — | 0.25 | — | — | — |
| | | — | — | — | 0.25 | — | — |
| | | — | — | — | — | 0.25 | — |
| | | — | — | — | — | — | 0.25 |

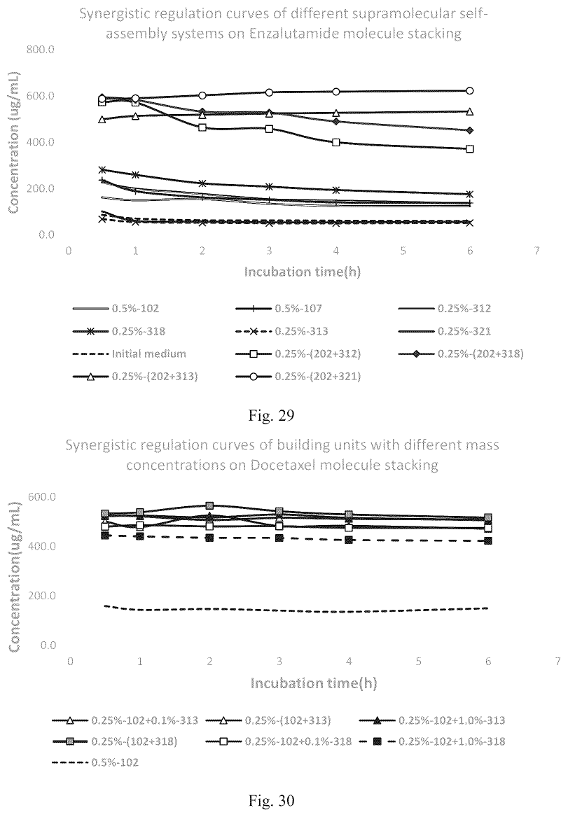

The experimental results are shown in FIG. 29:

Compared with initial medium, in binary supramolecular self-assembly systems built by 0.5% 102H and 107 with the guest molecule, the polymers had certain synergistic regulation ability on Enzalutamide guest molecule stacking, the guest molecule slowly packed over time, and the concentrations of the guest molecule in their solutions measured at 6 hours were 1.7 times and 1.9 times that of the initial medium, respectively. Binary supramolecular self-assembly systems built by 0.25% 320 and 321 with the guest molecule respectively did not show any synergistic regulation effect on guest molecule stacking. In binary supramolecular self-assembly systems built by 0.25% 312 and 318 with the guest molecule respectively, the concentrations of the guest molecule in their solutions measured at 6 hours were 135 μg/mL and 175 μg/mL respectively, and their synergistic regulation abilities were equivalent to those of the binary systems built by 0.5% 102H and 107 respectively. When the guest molecule, 0.25% composite polymer 202, and natural building blocks 312, 318, 313, or 321 built quaternary supramolecular self-assembly systems respectively, the quaternary supramolecular self-assembly systems built by 0.25% 313 and 321 with the composite polymer 202 respectively exhibited significant synergistic regulation effects, and the concentrations of the guest molecule in their solutions measured at 6 hours were 533 μg/mL and 623 μg/mL, which were 9 to 10 times that of the initial medium, respectively; the concentrations of the guest molecule in the solutions of the quaternary supramolecular self-assembly systems built by 0.25% 312 and 318 with 0.25% composite polymer 202, measured at 6 hours, were 371 μg/mL and 451 μg/mL, and their synergistic regulation effects were significantly better than those using a single polymer as the building unit.

### Examples 30-31

Docetaxel or Paclitaxel was used as a target molecule. Experiments were employed according to the following experimental scheme, the experimental steps were the same as those in Comparative Examples 1 and 2, and the quantitative determination method for Docetaxel and Paclitaxel was the same as before.

TABLE 21
Experimental design scheme for Examples 30-31

| | Target | Mass concentration of each building | | | | | |
|---|---|---|---|---|---|---|---|
| Example | guest | unit added (%) | | | | | |
| No | molecule | 102-H | 102-M | 313 | 313 | 318 | 318 |
| 30 | Docetaxel | 0.25 | — | 0.1 | — | — | — |
| | | 0.25 | — | — | 1.0 | | — |
| | | 0.25 | — | — | — | 0.1 | — |
| | | 0.25 | — | — | — | — | 1.0 |
| 31 | Paclitaxel | 0.25 | — | 0.1 | — | — | — |
| | | 0.25 | — | — | 1.0 | | — |
| | | 0.25 | — | 0.25 | | | |
| | | 0.25 | — | | | 0.25 | |
| | | 0.25 | — | — | — | 0.1 | |
| | | 0.25 | — | — | — | — | 1.0 |

The experimental results of Example 30 are shown in FIG. 30.

According to Comparative Example 2, in a binary supramolecular self-assembly system built by 0.5% 102H as a polymer, building unit and a guest molecule, the concentration of the guest molecule measured at 6 hours was 149.6 μg/mL; ternary supramolecular self-assembly systems built by the guest molecule, 0.25% 102H, and 0.1% and 1.0% 313 or 318 as building blocks respectively, where the concentrations of the guest molecule of the ternary systems at 6 hours, were 471.3 μg/mL and 474.1 μg/mL respectively; and the concentrations of the guest molecule in the ternary supramolecular self-assembly systems built by 1.0% 313 or 318 as building blocks and 0.25% 102H respectively, measured at 6 hours, were 505.4 μg/mL and 422.3 μg/mL respectively. According to the results of Example 20, the 6-hour concentrations of the guest molecule in the ternary supramolecular self-assembly systems built by 0.25% 313 or 318 combined with 0.25% 102H respectively, were 508.8 μg/mL and 516.3 μg/mL, which were higher than those of the ternary supramolecular self-assembly systems built by 0.1% and 1.0% 313 or 318 with 0.25% 102H respectively, and were significantly better than that of the binary supramolecular self-assembly system built by 0.5% 102H and the guest molecule.

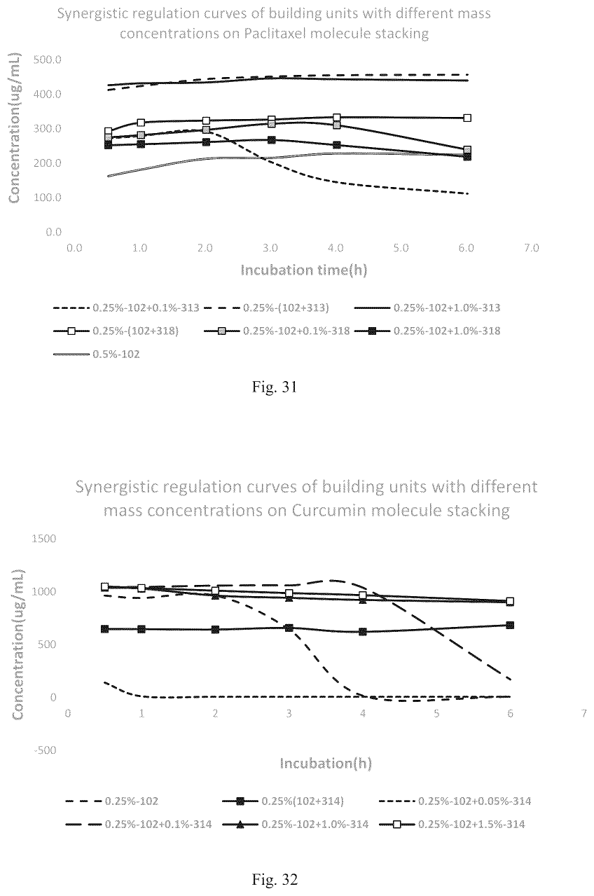

The experimental results of Example 31 are shown in FIG. 31.

Compared with Docetaxel (Log P: 2.92), Paclitaxel (Log P: 3.54) had stronger hydrophobicity. 0.25% 102H, the guest molecule, and 0.1%, 0.25%, and 1.0% 313 or 318 as building blocks to built ternary supramolecular self-assembly systems respectively, where the ternary supramolecular self-assembly systems containing 0.25% 313 or 318 had the strongest synergistic regulation effect on paclitaxel molecule stacking, the 6-h concentrations of the guest molecule were 456.8 μg/mL and 331.2 μg/mL, and their regulation abilities were gradually enhanced over time. The 6-h concentrations of the guest molecule in the solutions of the ternary supramolecular self-assembly systems built by 0.1% 313 or 318 and 0.25% 102H were 111.7 μg/mL and 239.2 μg/mL respectively; and the 6-h concentrations of the guest molecule in the the ternary supramolecular self-assembly systems built by 1.0% 313 or 318 and 0.25% 102H were 440.0 μg/mL and 218.6 μg/mL respectively. According to the above results, the effect of the natural building unit 313 or 318 did not depend on the mass concentration of 313 or 318 in the system.

### Example 32

Curcumin as a target guest, 0.25% 102 M type polymer, building block 314 at different mass concentrations built ternary supramolecular self-assembly systems. The initial theoretical concentration of Curcumin in the systems was 1200 μg/mL. The experimental steps were the same as those in Comparative Examples 1 and 2, and the experimental scheme was designed as follows:

TABLE 22
Experimental design scheme for Example 32

| | Target | Mass percentage of each building | | | | | | |
|---|---|---|---|---|---|---|---|---|
| Example | guest | unit added (%) | | | | | | |
| No | molecule | 102-M | 102-H | 314 | 314 | 314 | 314 | 314 |
| 33 | Curcumin | 0.25 | — | 0.05 | — | — | — | — |
| | | 0.25 | — | — | 0.1 | — | — | — |
| | | 0.25 | — | — | — | 0.25 | — | — |
| | | 0.25 | — | — | — | — | 1.0 | — |
| | | 0.25 | — | — | — | — | — | 1.5 |
| | | 0.25 | — | — | — | — | — | — |

The experimental results are shown in FIG. 32.

In a binary supramolecular self-assembly system composed of 0.25% 102M and the guest molecule Curcumin, the guest molecule stacked little within the first 2 hours, the concentration of Curcumin measured at this time was 962.6 μg/mL, but the molecule stacked very severely afterwards, and the concentration of the guest molecule measured at 6 hours was 6.4 μg/mL. In a ternary supramolecular self-assembly system built by 0.05% 314 as building block, polymer 102M and the guest molecule, a plurality of hydrogen donors and acceptors provided by 314 molecules competed with the guest molecule for interactions with the polymer building block and water, resulting in fewer available free water or effective functional groups on the polymer building block for the guest molecule, which accelerated rapid stacking of the guest molecule, where the concentrations of the guest molecule in the solution measured at 0.5 hours and 6 hours were 140.0 μg/mL and 5.2 μg/mL. When the mass concentration of 314 increased to 0.1%, the synergistic regulation ability was significantly enhanced, and the concentration of the guest molecule in the solution measured at 4 hours was 1036.7 μg/mL, which was significantly higher than that of a binary system built by the guest molecule and polymer 102M. When the mass concentration of 314 increased to 0.25%, the concentrations of the guest molecule in the solution measured at 0.5 hour to 6 hours were 646.3 to 683 μg/mL, and showed a slow increasing trend. When the concentration of 314 further increased to 1.0% or 1.5%, the concentration of the guest molecule in the 0.5 h-4.0 h solution can substantially be maintained at 1000 μg/mL, then the concentration slightly decreased from 4 to 6 hours, and the concentration of the guest molecule in the solution measured at 6 hours was still maintained at 900 μg/mL, showing a significantly different regulation mechanism from other supramolecular self-assembly systems.

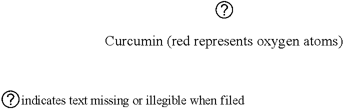

Intermolecular hydrogen bonds of the Curcumin molecules are shown above, with an oil-water partition coefficient Log P of 4.12, no dissociable groups, and a symmetrical molecular structure. As a natural triterpenoid glycoside, 314 had a hydrophobic end of triterpenoid and a hydrophilic disaccharide structure and was also a food additive (sweetener) approved by the European Union. The characteristics of amphiphilicity and multiple hydrogen donors, hydrogen acceptors, and chiral centers made 314 exhibit different behaviors in supramolecular self-assembly systems. Through hydrophobic interactions between molecules, it was easy to form a hydrophobic cavity with the hydrophilic end exposed, and the Curcumin molecule can easily enter the hydrophobic cavity to block the formation of intermolecular hydrogen bonds. When pH 6.8 phosphate buffer as initial medium, the carboxylic acid group on the succinyl group of the polymer building unit 102M was in a dissociated state to provide more hydrogen donors and hydrogen acceptors, and its acetyl group also provided a certain number of hydrogen acceptors, so that hydrogen donors and acceptors on saccharides similar to carbon nanotubes formed by multiple molecules of 314 were bonded to hydrogen donors or acceptors on 102M to form a strong synergistic regulation effect.

### Example 33

Nintedanib was a guest molecule, and a polymer was 102H. Experiments were conducted according to the following experimental scheme, the experimental steps were the same as those in Comparative Examples 1 and 2, and the quantitative determination method for Nintedanib was the same as before.

TABLE 23
Experimental design scheme for Example 33

| | Target | Mass concentration of each building | | | | | |
|---|---|---|---|---|---|---|---|
| Example | guest | unit added (%) | | | | | |
| No | molecule | 102-H | 102-M | 314 | 314 | 319-1 | 319-1 |
| 33 | Nintedanib | 0.25 | — | 0.1 | — | — | — |
| | | 0.25 | — | — | 1.0 | — | — |
| | | 0.25 | — | — | — | 0.1 | — |
| | | 0.25 | — | — | — | — | 1.0 |

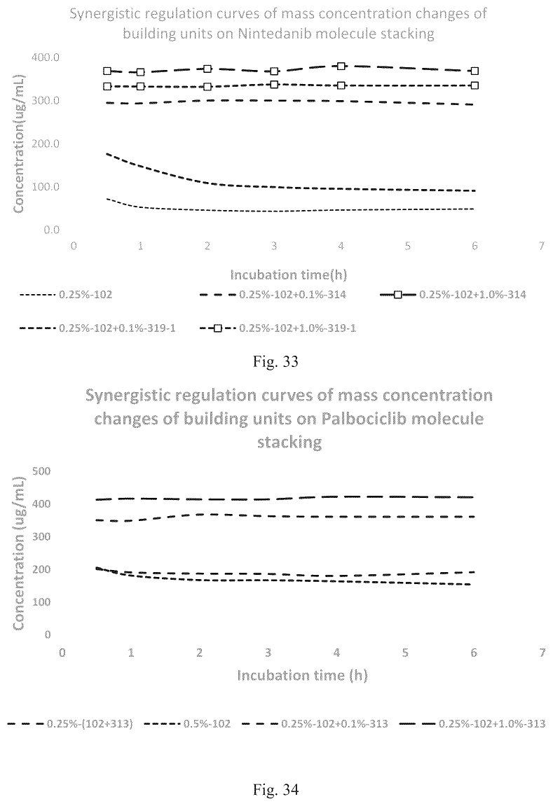

The results of Example 33 are shown in FIG. 33.

The Nintedanib molecule has very strong hydrophobicity and also exhibits intermolecular hydrogen bonding interactions. When Nintedanib and 0.25% 102H built a binary supramolecular self-assembly system, the concentration of the guest molecule at 6 hours was about 48.7 μg/mL, which was about 3 times that of initial medium; when 0.1% 314 or 319-1 was added to the system, the concentrations of the guest molecule in their 6-hour solutions were 291.0 μg/mL and 90.9 μg/mL respectively, and their synergistic regulation abilities were stronger than that of a system containing only polymer 102H; when the mass concentration of 314 or 319-1 in the ternary system was further increased to 1.0%, the concentrations of the guest molecule in the solutions measured at 6 hours were 368.8 μg/mL and 335.1 μg/mL respectively, with significant synergistic regulation effects; as the mass concentrations of 314 and 319-1 in the ternary systems changed, the ternary systems exhibited significant different synergistic regulation abilities on guest molecule stacking, where compared with 319-1, the natural building block 314 had a strong synergistic regulation effect even at a mass concentration of 0.1%. Nintedanib was a weakly alkaline drug with multiple dissociable groups in its molecular structure, so it was inferred that the ion interaction of 314 provided stronger non-covalent interaction.

### Examples 34-35

Palbociclib and Felodipine were used as target guest molecules respectively. The experimental scheme was shown in the table below, the experimental steps were the same as those in Comparative Examples 1 and 2, and the quantitative determination method for Palbociclib and Felodipine was the same as before.

TABLE 24
Experimental design scheme for Examples 34-35

| | | Mass concentration of different building | | | | | | |
|---|---|---|---|---|---|---|---|---|
| Example | Target | blocks added (%) | | | | | | |
| No | molecule | 102-H | 102-M | 313 | 313 | 313 | 319-1 | 319-1 |
| 34 | Palbociclib | 0.25 | — | 0.1 | — | — | — | — |
| | | 0.25 | — | — | 0.25 | — | — | — |
| | | 0.25 | — | — | — | 1.0 | — | — |
| 35 | Felodipine | 0.25 | — | — | — | — | 0.1 | — |
| | | 0.25 | — | — | — | — | — | 1.0 |
| | | 0.25 | — | 0.1 | — | — | — | — |
| | | 0.25 | — | — | — | 1.0 | — | — |
| | | 0.25 | — | — | — | — | — | — |

The experimental results of Example 34 are shown in FIG. 34.

Compared with initial medium, the concentrations of the guest molecule in ternary supramolecular self-assembly systems built by Palbociclib with 0.1%, 0.25% and 1.0% natural building block 313 and 0.25% 102H, measured at 6 hours, were 191.5 μg/mL, 361.6 μg/mL and 420.8 μg/mL, which were 6.1 times, 11.4 times and 13.3 times that of the initial medium, respectively. When the mass concentration of polymer 102H in the ternary supramolecular self-assembly systems was fixed to 0.25%, and the mass concentration of the natural building block 313 increased from 0.1% to 0.25%, namely, increased by 2.5 times, the concentrations of the guest molecule in the solutions increased by about twice. But when the mass concentration of the natural building block increased to 1.0%, compared to the 0.25% mass concentration system, the concentration of the guest molecule increased only by 16.4%, indicating that the mass concentration of the added natural building block had an optimal value when the mass concentration of polymer 102H in the system was fixed.

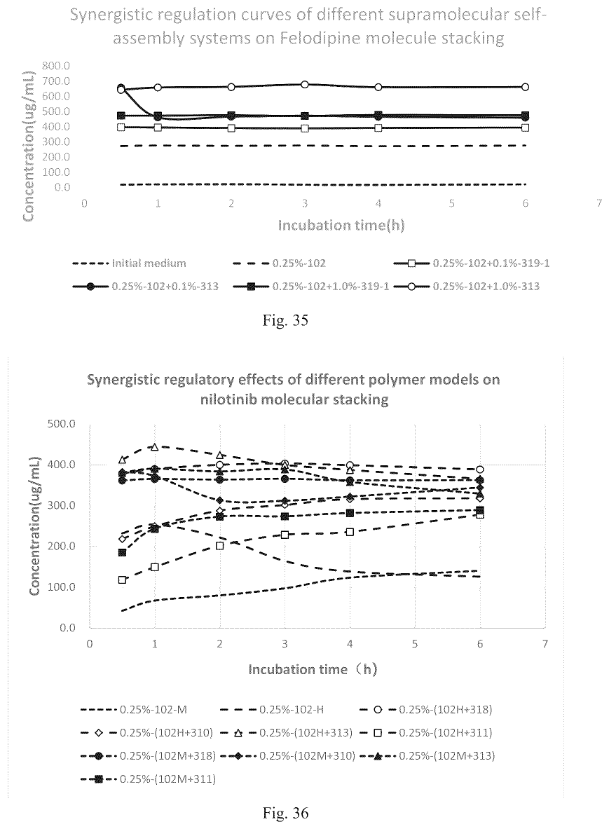

The experimental results of Example 35 are shown in FIG. 35.

Compared with initial medium, a binary supramolecular system built by the guest Felodipine and 0.25% 102H can significantly regulate Felodipine molecule stacking and rapidly become stable, and the concentration of the guest molecule in the system measured at 6 hours was 276 μg/mL, which was about 13.5 times that of the initial medium. Felodipine, 0.25% 102H, and 0.1% or 1.0% 313 or 319-1 built ternary supramolecular self-assembly systems. Due to different structures of 313 and 319, different synergistic regulation groups provided, and different molecular recognition, the regulation effects of the ternary supramolecular self-assembly system built by them on the guest molecule were also different. Under the same mass concentration, the ternary supramolecular self-assembly system built by 0.1% 313 had a higher encapsulation rate of the guest molecule. When the mass concentration of 313 in the ternary supramolecular self-assembly system increased from 0.1% to 1.0%, the encapsulation rate of the guest molecule in the built ternary supramolecular self-assembly system was significantly improved, and the concentration of Felodipine in the system measured at 6 hours was 662 μg/mL, which was about 32.5 times that of the initial medium. The encapsulation rate in the ternary supramolecular self-assembly system built by 319-1 did not further increase with the increase of the mass concentration of 319-1, so it did not have mass dependence.

### Example 36

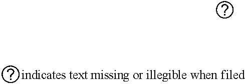

### Nilotinib Molecule (Red Represents Oxygen Atoms; Blue Represents Nitrogen Atoms; Green Represents Fluorine Atoms)

According to the molecular structure of Nilotinib, Nilotinib can form both intramolecular hydrogen bonds and intermolecular hydrogen bonds in an aqueous solution. The interaction of intermolecular hydrogen bonds enables rapid stacking of the Nilotinib molecule in the aqueous solution to form a self-assembled solid and precipitate.

Nilotinib was used as a target guest molecule. The experimental scheme was shown in the table below, the experimental steps were the same as those in Comparative Examples 1 and 2, and the quantitative determination method for Nilotinib was the same as before.

TABLE 25
Experimental design scheme for Example 36

| | | Mass concentration of different building | | | | | |
|---|---|---|---|---|---|---|---|
| Example | Target | blocks added (%) | | | | | |
| No | molecule | 102-M | 102-H | 310 | 318 | 313 | 311 |
| 36 | Nilotinib | — | 0.25 | 0.25 | — | — | — |
| | | — | 0.25 | — | 0.25 | — | — |
| | | — | 0.25 | — | — | 0.25 | — |
| | | — | 0.25 | — | — | — | 0.25 |
| | | 0.25 | — | 0.25 | — | — | — |
| | | 0.25 | — | — | 0.25 | — | — |
| | | 0.25 | — | — | — | 0.25 | — |
| | | 0.25 | — | — | — | — | 0.25 |

The results of Example 36 are shown in FIG. 36.

Nilotinib had a Log D6.8 of 5.27 and was very hydrophobic under pH 6.8 conditions. However, Nilotinib was a basic drug with a dissociation constant of 5.92. In addition to providing some hydrophobic and hydrophilic groups, the 102 molecule can also form ionic interactions with the basic drug. Compared with 102H type and M type, the hydrophobic interaction between H type and Nilotinib molecule was stronger, and the M type provided more dissociable groups than the H type. Ternary supramolecular self-assembly systems built by the guest molecule and 102 M or H with 310, 311, 313, and 318 respectively had significant synergistic regulation effects on guest molecule stacking compared with binary systems built by 0.25% 102M or 102H. Because the ratio of hydrophobic and hydrophilic groups on the 102H or 102M polymer molecule decreased from 1.7 to 0.8, there were also some subtle differences in the synergistic regulation effect with similar natural building blocks. For example, among the ternary supramolecular self-assembly systems built by 102H or 102M and 310, the concentration of the guest molecule in the system built by 102H showed a slow increasing trend within the incubation time of 0.5 hour to 6 hours and the 6-hour solution concentration was 317.6 μg/mL, while the concentration of the guest molecule in the system built by 102M showed a trend of high on two sides and low in the middle during investigation, but the 6-hour solution concentration still remained at 344.0 μg/mL, which was slightly higher than that of the 102H type. Among ternary supramolecular self-assembly systems built by 102H or 102M polymer molecule and 313 respectively showed a slow decrease in the concentration of the guest molecule during investigation, the solution concentrations of the guest molecule measured at 6 hours were 365.2 μg/mL and 328.9 μg/mL respectively, with H type slightly higher than M type. This was mainly due to the fact that 313 provided more hydrophilic groups compared to 310, while the greater hydrophobicity provided by 120H type compensated for some hydrophobicity provided by 313 compared to M type.

### Example 37

Apixaban was used as a target guest molecule. The experimental scheme was shown in the table below, the experimental steps were the same as those in Comparative Examples 1 and 2, and the quantitative determination method for Apixaban was the same as before.

TABLE 26
Experimental design scheme for Example 37

| | | Mass concentration of each building | | | | | | |
|---|---|---|---|---|---|---|---|---|
| Example | | unit added (%) | | | | | | |
| No | Target | 102H | 103E5 | 104 | 310 | 311 | 312 | 318 |
| 37 | Apixaban | 0.25 | — | 0.25 | 0.1 | — | — | — |
| | | 0.25 | — | 0.25 | — | 0.1 | — | — |
| | | 0.25 | — | 0.25 | — | — | 0.1 | — |
| | | 0.25 | — | 0.25 | — | — | — | 0.1 |
| | | 0.25 | 0.25 | — | 0.1 | — | — | — |
| | | 0.25 | 0.25 | — | — | 0.1 | — | — |
| | | 0.25 | 0.25 | — | — | — | 0.1 | — |
| | | 0.25 | 0.25 | — | — | — | — | 0.1 |
| | | — | 0.25 | — | — | — | — | — |
| | | — | — | 0.25 | — | — | — | — |

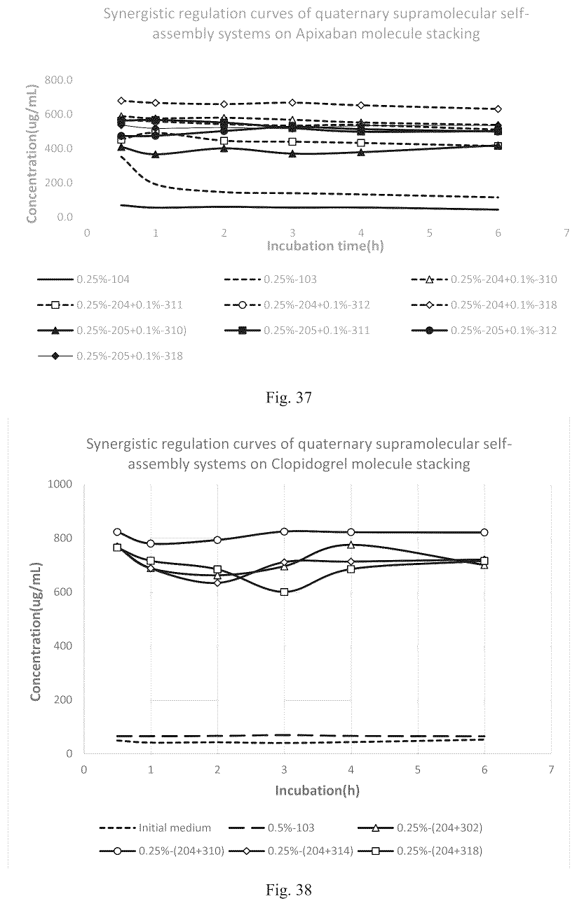

The experimental results of Example 37 are shown in FIG. 37.

0.25% 103E5 and 0.25% 104 built binary supramolecular self-assembly systems with the guest molecule respectively, where the binary supramolecular self-assembly system built by 0.25% 103E5 had certain synergistic regulation effect on guest molecule stacking, and the concentration of the guest molecule in the system measured at 6 hours was about twice that of initial medium. The concentration of the guest molecule in the solution of the binary system built by 0.25% 104, measured at 6 hours, was lower than that of the initial medium. Quaternary supramolecular self-assembly systems built by the guest molecule and 0.25% composite polymer 204 (102H+103E5) or 0.25% 205 (102H+104) with 0.1% 310, 311, 312, or 318 respectively had significant synergistic regulation effects compared with the binary systems and can become stable, and the concentrations of the guest molecule in the system solutions measured at 6 hours were 414 to 618 μg/mL, which were 8.6 to 12.9 times that of the initial medium.

### Example 38

Clopidogrel bisulfate was used as a target guest molecule. The experimental scheme was shown in the table below, the experimental steps were the same as those in Comparative Examples 1 and 2, and the quantitative determination method for Clopidogrel bisulfate was the same as before.

TABLE 27
Experimental design scheme for Example 38

| | | Mass concentration of different building | | | | | | |
|---|---|---|---|---|---|---|---|---|
| Example | | blocks added (%) | | | | | | |
| No | Target | 102M | 103E3 | 104 | 310 | 302 | 314 | 318 |
| 38 | Clopidogrel | 0.25 | 0.25 | — | 0.25 | — | — | — |
| | bisulfate | 0.25 | 0.25 | — | — | 0.25 | — | — |
| | | 0.25 | 0.25 | — | — | — | 0.25 | — |
| | | 0.25 | 0.25 | — | — | — | — | 0.25 |
| | | — | 0.5 | — | — | — | — | — |

The experimental results of Example 38 are shown in FIG. 38:

Compared with initial medium, a binary supramolecular self-assembly system composed of 0.5% 103E3 and Clopidogrel bisulfate had a relatively weak synergistic regulation effect on guest molecule stacking, and the concentration of the guest molecule in the solution measured at 6 hours was 64.37 μg/mL, which was slightly higher than that of the initial medium (51.97 μg/mL). Compared with the binary supramolecular self-assembly system, quaternary supramolecular self-assembly systems built by 0.25% composite polymer 204 (102M+103E3), Clopidogrel bisulfate, and 0.25% 302, 310, 314, or 318 had molecular recognition and significant synergistic regulation effects on guest molecule stacking and can be stable, and the concentrations of the guest molecule in the systems measured at 6 hours were 700 to 821 μg/mL, which were 13-17 times that of the initial medium.

### Examples 39-40

Naringenin and Posaconazole were used as target guest molecules respectively. The experimental scheme was shown in the table below, the experimental steps were the same as those in Comparative Examples 1 and 2, and the quantitative determination method for Naringenin and Posaconazole was the same as before.

TABLE 28
Scheme design for Examples 39-40

| | | Mass concentration of different building | | | | | | | |
|---|---|---|---|---|---|---|---|---|---|
| Example | | blocks added (%) | | | | | | | |
| No | Targets | 101 | 102H | 103E5 | 315 | 315-1 | 314 | 319-1 | 319 |
| 39 | Naringenin | — | 0.25 | 0.25 | 0.1 | — | — | — | |
| | | — | 0.25 | 0.25 | — | 0.5 | — | — | |
| | | — | 0.25 | 0.25 | — | — | 0.1 | — | |
| | | — | 0.25 | 0.25 | | | | | 0.1 |
| | | — | 0.25 | 0.25 | — | — | — | 0.5 | — |
| 40 | Posaconazole | 0.25 | 0.25 | — | 0.1 | — | — | — | — |
| | | 0.25 | 0.25 | — | — | 0.25 | — | — | — |
| | | 0.25 | 0.25 | — | — | — | 0.1 | — | — |
| | | 0.25 | 0.25 | — | — | — | — | | 0.1 |

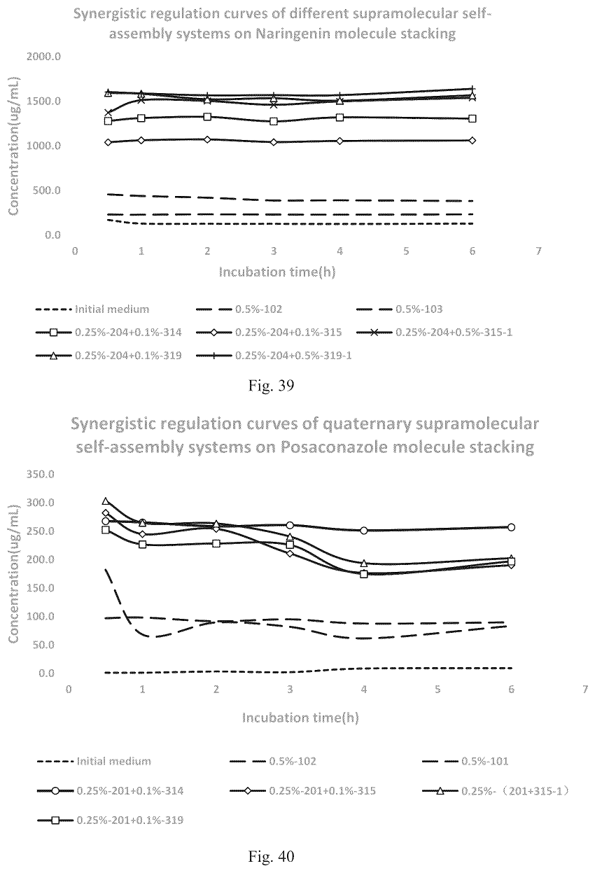

The experimental results of Example 39 are shown in FIG. 39:

The concentrations of the guest molecule in binary supramolecular self-assembly systems built by the guest molecule with 0.5% 102H or 0.5% 103E5, measured after incubation for 6 hours, were 383.3 μg/mL and 233.5 μg/mL, which were 3.0 times and 1.8 times that of initial medium, respectively. Quaternary supramolecular self-assembly systems can be built by the guest with 0.25% 204 (102H+103E5) and 0.1% 314 or 0.1% 315 or 0.5% 315-1 or 0.1% 319 or 0.5% 319-1 respectively quaternary supramolecular self-assembly system can be built through molecular recognition and synergistic regulation within 0.5 hours, the concentrations of the guest molecule in the systems measured at 6 hours were 1060.9 to 1640.5 μg/mL, which were about 8.2 to 12.7 times that of the initial medium and significantly better than the effect achieved by the aforementioned binary supramolecular self-assembly systems.

The results of Example 40 are shown in FIG. 40:

Compared with initial medium, binary supramolecular self-assembly systems built by the guest molecule and 0.5% 101 or 0.5% 102H had significant synergistic regulation effects on the molecular stacking of the guest molecule Posaconazole, and the concentrations of the guest molecule in the systems measured at 6 hours were 83.4 μg/mL and 89.6 μg/mL, which were 9.3 times and 10 times that of the initial medium, respectively. Quaternary supramolecular self-assembly systems built by composite polymer 0.25% 201 (102H+101), the guest molecule, and 0.1% 314 or 0.1% 315 or 0.1% 319 or 0.25% 315-1 respectively had significant synergistic regulation effects compared to the initial medium and the binary supramolecular self-assembly systems, where except for 0.1% 314, the other three quaternary supramolecular self-assembly systems formed stable supramolecular self-assembly systems after 4 hours, while the quaternary supramolecular self-assembly system built by 0.1% 314 can quickly form a stable supramolecular self-assembly system within 0.5 hour and had a significantly higher encapsulation rate of the guest molecule than the other three groups, and the concentration of the guest molecule in the system measured at 6 hours was 257.1 μg/mL, which was about 28.6 times that of the initial medium.

### Example 41

Warfarin as a target guest molecule, 0.25% composite polymer building unit 207 (101+103E5), and natural building blocks selected from 0.1% 313, 314, 315 and 320 built quaternary supramolecular self-assembly systems, where the theoretical concentration of the guest molecule Warfarin in the systems was 1600 μg/mL. The experimental steps were the same as those in Comparative Examples 1 and 2, and the quantitative determination method for Warfarin was the same as before.

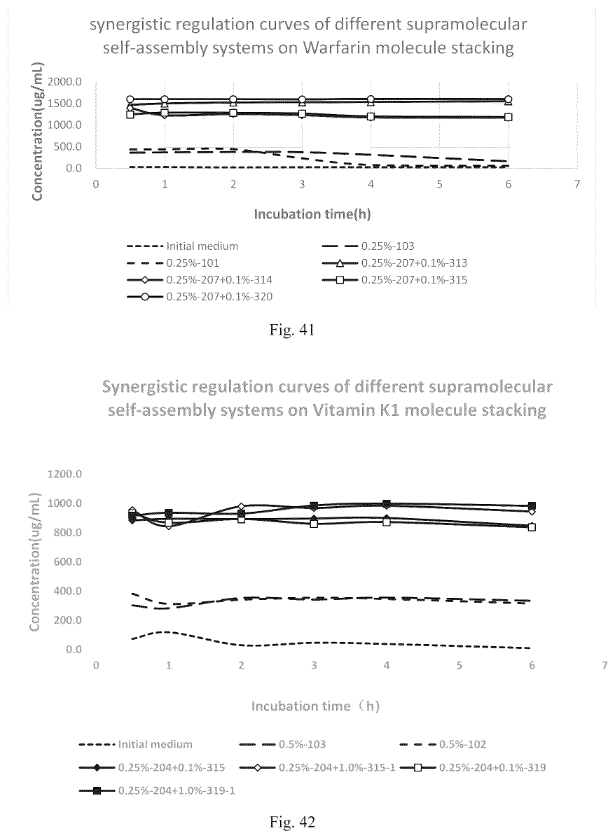

The experimental results are shown in FIG. 41.

Compared with the initial medium, binary supramolecular self-assembly systems built by 0.25% 103E5 or 0.25% 101 with the guest molecule had certain synergistic regulation effects on Warfarin molecule stacking, but neither can form stable self-assembly systems, and the guest molecule slowly packed over incubation time. Compared with the binary supramolecular self-assembly systems, quaternary supramolecular self-assembly systems built by the guest, 0.25% composite polymer building unit 207 (101+103E5), and 0.1% natural building unit 313 or 314 or 315 or 320 respectively can quickly become stable. The quaternary supramolecular self-assembly system built by the guest, 0.25% 207, and 0.1% 320 had a maximum encapsulation rate of Warfarin, which was close to 100%; the quaternary supramolecular self-assembly system built by the guest, 0.25% 207, and 0.1% 313 had a slightly low encapsulation rate; but all the quaternary supramolecular self-assembly systems had very significant synergistic regulation effects compared with the binary supramolecular self-assembly systems.

### Example 42

Vitamin K1 as a target molecule, a polymer building block selected from 0.25% 204 (102M+103E3), a natural building unit selected from 1.0% 319-1 or 0.1% 319 or 0.1% 315 or 1.0% 315-1, and a guest molecule built a quaternary supramolecular self-assembly system. The experimental steps were the same as those in Comparative Examples 1 and 2, and the quantitative determination method for Vitamin K1 was the same as before.

The experimental results are shown in FIG. 42.

Compared with initial medium, binary supramolecular self-assembly systems built by 0.5% 103 and 0.5% 102 with the guest molecule respectively had significant synergistic regulation effects on guest molecule stacking and can quickly form stable supramolecular self-assembly systems, and the concentrations of the guest molecule in the systems measured at 6 hours were 313 to 333 μg/mL, which were 39 to 42 times that of the initial medium. The quaternary supramolecular self-assembly systems built by 0.25% composite polymer 204, 0.1% 315 or 1.0% 315-1 or 0.1% 319 or 1.0% 319-1, and the guest had significant synergistic regulation effects on guest molecule stacking and can quickly form stable supramolecular self-assembly systems. Compared with the initial medium and the binary supramolecular self-assembly systems, the encapsulation rates of the quaternary supramolecular self-assembly systems built by 319-1 and 315-1 were higher than those of the 319 and 315 systems. The concentrations of the guest molecule in the quaternary supramolecular self-assembly systems measured at 6 hours were 836 to 983 μg/mL, which were 104.5 to 122.9 times that of the initial medium.

### Example 43

Eltrombopag was used as a target molecule, polymers were selected from 0.25% 203 [102M+106 K30 type] and 0.25% 202 [102M+107], and natural building blocks were selected from 0.1% 310, 312, 317, and 318, to investigate the regulation effects of built quaternary supramolecular self-assembly systems on guest molecule stacking. Operation followed the experimental steps in Comparative Examples 1 and 2, and the quantitative analysis method for Eltrombopag was the same as before.

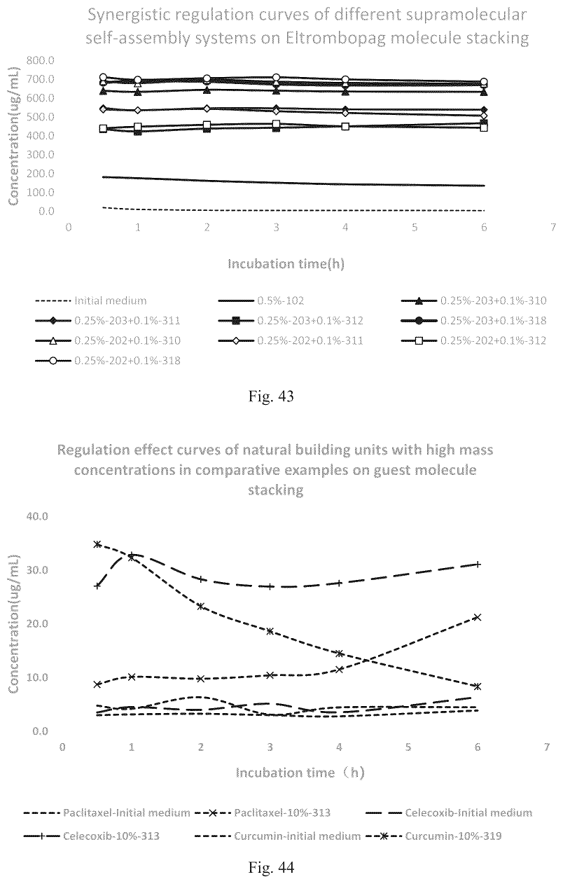

The experimental results are shown in FIG. 43.

Compared with initial medium, a binary supramolecular self-assembly system built by 0.5% 102M and the guest molecule had a significant synergistic regulation effect on guest molecule stacking, and the concentration of the guest molecule in its solution measured at 6 hours was 135.9 g/mL, which was about 30.2 times that of the initial medium. Quaternary supramolecular self-assembly systems built by the guest, 0.25% 203 or 0.25% 202, and 0.1% 310 or 311 or 312 or 318 respectively had significant molecular recognition and synergistic regulation effects compared to the binary system, the quaternary supramolecular self-assembly systems can form stable self-assembly systems, and their synergistic regulation effects on the molecular stacking of the guest Eltrombopag were significantly better than that achieved by the binary system built by 0.5% 102M. Among the quaternary supramolecular self-assembly systems built by 0.25% 202 and 203, the synergistic regulation effects were in an order of 318>310>311>312 from strong to weak, the concentration of Eltrombopag in each system measured at 6 hours was 444 to 690 μg/mL, and compared with the initial medium, the concentration of the guest molecule in each quaternary supramolecular self-assembly system was 99 to 153 times that of the initial medium.

### Comparative Example 3

According to US2012/0121696A1, measured in a system using 10% (W/V) Rubusoside A as a building block, the concentration of Paclitaxel in the solution was 26 μg/mL, and the concentration of Celecoxib in the solution was 109 μg/mL; and when 10% (W/V) Mogroside V was selected as building block, the concentration of Curcumin in the solution was only 44 μg/mL.

An initial medium of each above target molecule was used to prepare a solution containing 10% Rubusoside A (313) or 10% (W/V) Mogroside V (319), the solution was sonicated at 37° C. to prepare a solution containing about 100 mg/mL building block 313 or 319 per 1 mL of the initial medium, the clear medium containing the natural building blocks was incubated in a 37° C. constant temperature air bath shaker for 1 hour, and subsequent operations and sample detection followed the steps of Comparative Examples 1 and 2. The results are shown in FIG. 44. Considering that each natural building block had different UV absorption and was added at a high concentration, the more specific HPLC method was still used for quantitative determination on the solution concentration of each guest molecule, although UV spectrophotometry was employed in this patent.

According to the above results, compared with the detected concentration of each target guest in the initial medium, 10% 313 and 10% 319 as building blocks had certain regulation effect on the stacking of each target molecules, but it was very weak. And the mass concentration and achieved effect of the natural building block used in the system did not show a mass dependent relationship, namely, the higher the mass concentration of the natural building block added, the stronger the synergistic regulation effect, and its dosage was far more than the daily acceptable dosage of 0-5 mg/kg body weight approved for use as sweeteners 313 and 319 (calculated as 60 kg for adult body weight, no more than 300 mg daily). If human intestinal fluid was calculated as 100 mL, the natural building unit to be added to a unit preparation was 100 mg/mL*100 mL=10 g, which was very limited in practical applications.

### Example 44

Curcumin, Paclitaxel, and Celecoxib were selected as target. Ternary supramolecular self-assembly systems were built with 0.25% 102, 0.1% 313 or 319, or 0.5% 313 or 319, respectively. The operation steps followed Comparative Examples 1, 2 and 3, and the initial medium were listed under every target items and kept consistent with Comparative Example 3.

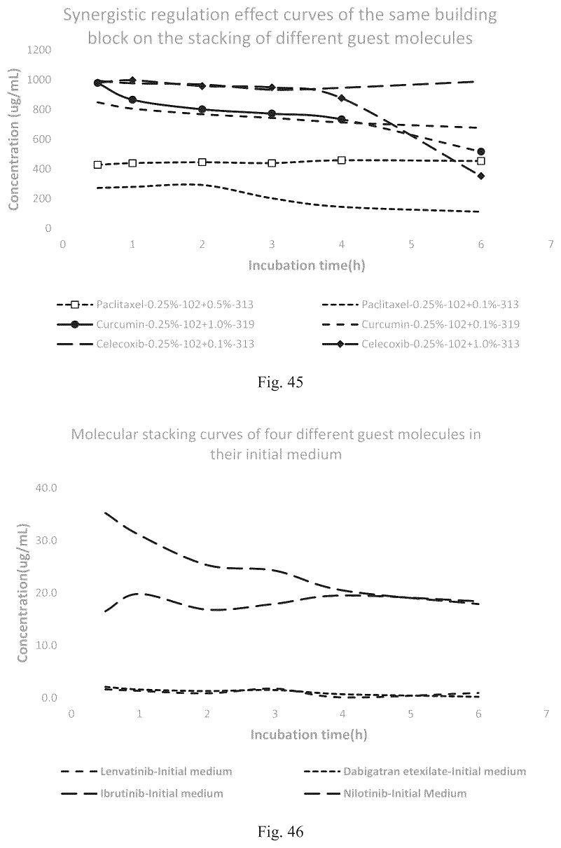

The experimental results are shown in FIG. 45.

According to the above results, in the ternary supramolecular self-assembly systems built by 0.25% 102 and 313 or 319 respectively. When the concentration of 313 or 319 in the ternary supramolecular self-assembly systems was changed, the synergistic regulation effects on the molecular stacking of different guest molecules were significantly different. In the ternary supramolecular self-assembly system built by 0.25% 102H and 0.1% 313, the concentration of Paclitaxel in its solution measured at 6 hours was 111.7 μg/mL, which was 5 times that in Comparative Example 3. In the ternary supramolecular self-assembly system built by 0.25% 102H and 0.5% 313, the concentration of Paclitaxel in its solution measured at 6 hours was 453.2 μg/mL, which was 21.6 times that in Comparative Example 3, but the mass concentrations of 313 were only 1% and 5% of the concentration used in Comparative Example 3. In the ternary supramolecular self-assembly systems of 0.25% 102H and 0.1% 319 and 0.25% 102H and 1.0% 319, the concentrations of Curcumin in their solutions measured at 6 hours were 676.6 μg/mL and 516.3 μg/mL, which were 81.5 times and 62.2 times those in Comparative Example 3 (6 h: 8.3 μg/mL), while the mass concentrations of 319 were only 1% and 10% of that in Comparative Example 3. In the 0.25% 102L and 0.1% 313 and 0.25% 102L and 1.0% 313 systems, the concentrations of Celecoxib in their solutions measured at 6 hours were 988.1 μg/mL and 352.3 μg/mL, which were 31.9 times and 11.4 times those in Comparative Example 3 (6 h: 31.3 μg/mL). When the mass concentration of 313 increased from 0.1% to 1.0%, the synergistic regulation ability of 313 with low mass concentration instead made the built supramolecular self-assembly system more stable over time. When 1.0% 313 was contained, the guest molecule stacked severely after 4 hours, but the concentration of the guest molecule in the solution was still significantly higher than that achieved in Comparative Example 3. Similar results can also be observed in the Curcumin system, but different hydrophobicity of different targets, intermolecular interaction of the guest, or synergistic regulatory ability of the guest to form hydrogen bonds with polymer building blocks and natural building blocks in the system determined the stability of the formed ternary supramolecular self-assembly system, which directly affected the severity of guest molecule stacking. In the system built by the combination of 0.25% 102 and 0.1% 313, the guest molecule stacked slowly in the later stage. When the mass concentration of 313 in the system was further increased, the guest molecule did not further stack throughout the entire investigation.

### Example 45

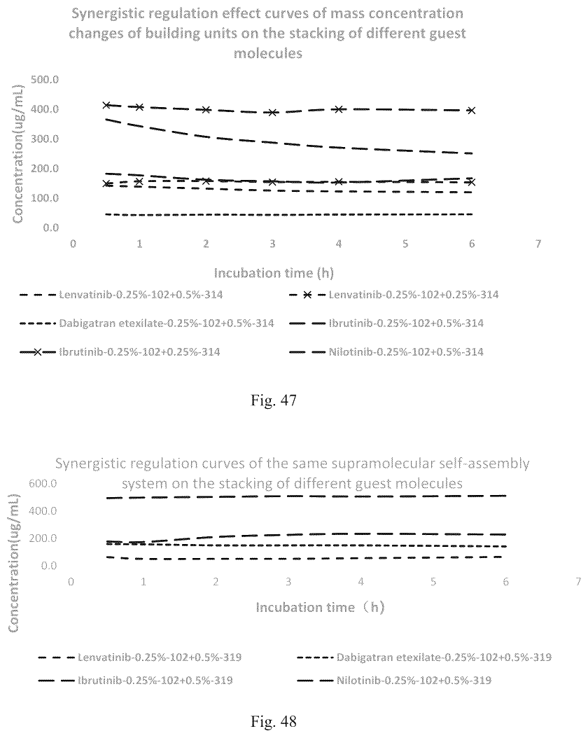

Lenvatinib, Nilotinib, Dabigatran Etexilate, and Ibrutinib were selected as target guest molecules, supramolecular self-assembly systems were built with 0.25% 102H and 314 or 319 at different concentrations respectively, to investigate the synergistic regulation ability of the same building block on the stacking of different guest molecules. The experimental steps followed Comparative Examples 1 and 2, and the detection method was the same as before. The experimental results are shown in FIGS. 46-48.

According to FIG. 48, the initial concentrations of the guests Lenvatinib, Dabigatran Etexilate, Ibrutinib, and Nilotinib added to their initial media were 150 μg/mL, 500 μg/mL, 500 μg/mL, and 450 μg/mL, respectively. After incubated 0.5 hours, all the guest molecules rapidly stacked and precipitated from the media. Compared with the initial media, ternary supramolecular self-assembly systems built by 0.25% 102H and 0.5% 319, significant synergistic regulation effects were noticed on the stacking of each guest molecule when different mass concentration of building block was employed. Among of them, the regulation on Lenvatinib guest molecule stacking was the weakest, followed by Dabigatran Etexilate and Nilotinib; the synergistic regulation on Ibrutinib guest molecule stacking was the strongest, and a stable supramolecular self-assembly system can be formed, with an encapsulation rate close to 100%; but the synergistic regulation on the Nilotinib molecule was slowly enhanced over time until a stable supramolecular self-assembly system was formed. Compared with the initial media, ternary supramolecular self-assembly systems built by 0.25% 102H and 0.5% 314, each target guest had significant synergistic regulation effects on the stacking of each guest molecule, but when the mass concentration of 314 in the ternary supramolecular self-assembly systems was increased from 0.25% to 0.5%, the guest molecules of Lenvatinib and Ibrutinib gradually stacked over time, while 0.25% 314 and guest Lenvatinib or Ibrutinib can form stable ternary supramolecular self-assembly systems. According to the above results, different supramolecular self-assembly systems achieved the most stable state through molecular recognition and synergistic regulation, and the same supramolecular self-assembly system had significantly different synergistic regulation effects on different guests.

Comparative Example 4: Macitentan was selected as a guest molecule, the supramolecular self-assembly systems were built by 0.25%-201 combination polymer, and 0.25%-305 or 0.25%-309 respectively, and the others were the same as Example 26. The experimental scheme was shown in the table below, the experimental steps were the same as those in Comparative Examples 1 and 2, and the quantitative determination method for Macitentan was the same as before.

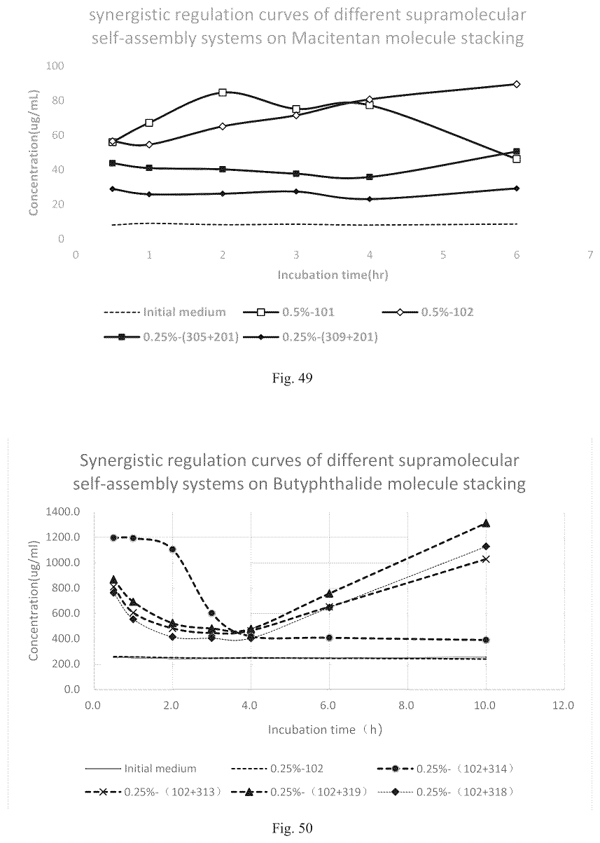

The experimental results are shown in FIG. 49:

According to the experimental results, the concentrations of the Macitentan molecule in quaternary supramolecular self-assembly systems built by 0.25%-305 or 0.25%-309 and 0.25%-201, the concentration measured at 6 hours, were 59.5 μg/mL and 29.2 μg/mL, which were lower than that of system built by 0.5%-101 or 0.5%-102H, where 309 or 305 and the combination polymer did not show any synergistic regulation effect on Macitentan molecule stacking.

### Example 46

Butylphthalide was selected as a target guest, a polymer 102M, and natural building blocks 314, 313, 319, or 318 were used to build ternary supramolecular self-assembly systems. The operation procedures followed the experimental steps in Comparative Examples 1 and 2, and the quantitative analysis method for Butylphthalide was the same as before.

The experimental results are shown in FIG. 50:

Compared with initial medium, a binary supramolecular self-assembly system built by 0.25% polymer 102M and the guest showed that 0.25% 102M had no regulation effect on Butylphthalide molecule stacking, and the concentration of Butylphthalide in the solution measured at each incubation time point was equivalent to that of the initial medium. When 0.25% 102M, natural building blocks 314, 313, 319 or 318 and the guest built ternary supramolecular self-assembly systems respectively, the 2-hour concentration of the system built by 0.25% 314 was 1103.4 μg/mL, but the concentration of Butylphthalide in the system solution was 406.8 μg/mL as the incubation time was further extended to 6 hours, and continued to decrease slowly over time. The solution concentrations of the ternary supramolecular self-assembly systems built by 0.25% 102M and 0.25% 313, 318, or 319 were 480.3 μg/mL, 521.5 μg/mL, and 413.5 μg/mL measured at 2 hours, but slowly increased at 6 hours to 650.6 μg/mL, 754.1 μg/mL, and 643.7 μg/mL, which were significantly better than those achieved after the same incubation time as 314; when the incubation time was further extended to 10 hours, the concentrations of the guest molecule in the above solutions were 1026.3 μg/mL, 1310.2 μg/mL, and 1125.6 μg/mL, showing an advantage of synergistic regulation.

### Example 47

Coenzyme Q10 as a target guest, polymer 102H or composite polymer 201 (102H+101), and natural building blocks 314 or 313 or 319-1 or 318 or 303 were used to build ternary or quaternary supramolecular self-assembly systems. The operation procedure followed the experimental steps in Comparative Examples 1 and 2, and the quantitative analysis method for Coenzyme Q10 was the same as before.

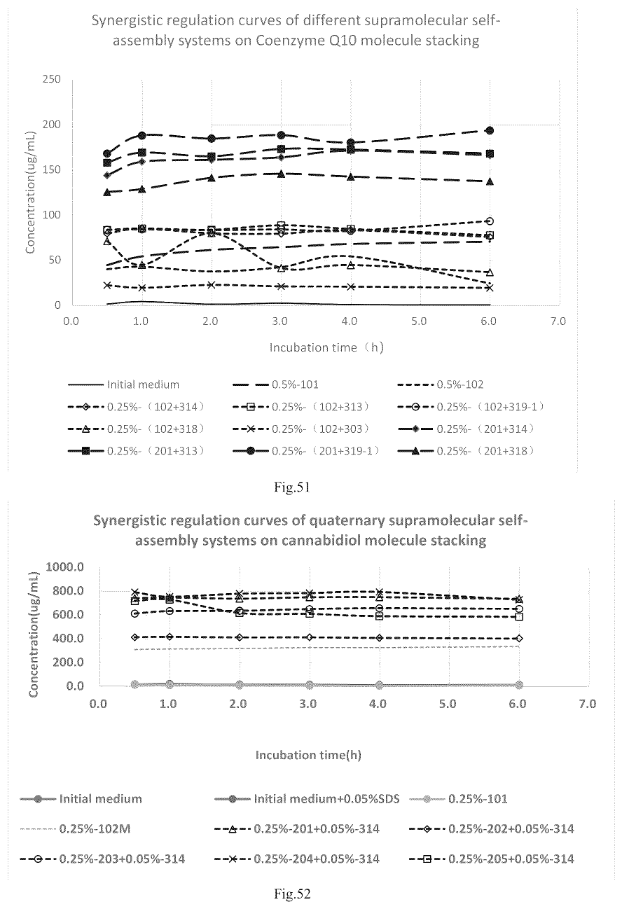

The experimental results are shown in FIG. 51.

Compared with initial medium, the ternary supramolecular self-assembly systems built by 0.25% polymer 102H, 0.25% 314 or 313 or 319-1 or 318 or 303, and the guest molecule respectively had significant synergistic regulation effects on guest molecule stacking, and the concentrations of Coenzyme Q10 in the solutions of the systems measured at 6 hours were 75.6 μg/mL, 77.6 μg/mL, 93.1 μg/mL, 36.7 μg/mL, and 19.3 μg/mL, which were 189 times, 194 times, 233 times, 92 times, and 48 times that of the initial medium at 6 hours, respectively, showing an advantage of synergistic regulation. The quaternary supramolecular self-assembly systems, which built by 0.25% composite polymer 201 (102H and 101), 0.25% 314 or 313 or 319-1 or 318, and the guest molecule respectively, had significant synergistic regulation effects on guest molecule stacking compared to the ternary supramolecular self-assembly systems, and the concentrations of Coenzyme Q10 in the solutions of the systems measured at 6 hours were 166.0 μg/mL, 168.0 μg/mL, 195.5 μg/mL, and 139.1 μg/mL, which were 415 times, 420 times, 489 times, and 348 times that of the initial medium at 6 hours, respectively. Due to the extremely strong hydrophobicity of Coenzyme Q10 (Log P of about 17), the addition of polymer 101 compensated for the limited hydrophobicity of 102H to improve the stability of the quaternary supramolecular self-assembly systems.

### Examples 48-50

Cannabidiol, a representative of cannabidiol derivatives, was used as a target molecule. The experimental scheme was shown in the table below, the experimental steps were the same as those in Comparative Examples 1 and 2, and the quantitative determination method for Cannabidiol was the same as before.

TABLE 29
Scheme design for Examples 48-50

| | Mass concentration of different building | | | | | | | | | | | |
|---|---|---|---|---|---|---|---|---|---|---|---|---|
| Example | blocks added (%) | | | | | | | | | | | |
| No | 101 | 102M | 103E3 | 104 | 106-K30 | 107 | 314 | 318 | 310 | 312 | 313 | 319 |
| 48 | — | 0.25 | 0.25 | — | — | — | 0.05 | — | — | — | — | — |
| | — | 0.25 | | 0.25 | — | — | 0.05 | — | — | — | — | — |
| | — | 0.25 | — | — | 0.25 | — | 0.05 | — | | — | — | — |
| | — | 0.25 | — | — | — | 0.25 | 0.05 | — | — | | — | — |
| | 0.25 | 0.25 | — | — | — | — | 0.05 | — | — | — | — | — |
| 49 | — | 0.25 | — | — | — | — | 0.1 | — | — | — | — | — |
| | — | 0.25 | — | — | — | — | 0.25 | — | — | — | — | — |
| | — | 0.25 | — | — | — | — | — | 0.1 | — | — | — | — |
| | — | 0.25 | — | — | — | — | — | 0.25 | — | — | — | — |
| | — | 0.25 | — | — | — | — | — | — | 0.25 | — | — | — |
| | — | 0.25 | — | — | — | — | — | — | 0.1 | — | — | — |
| | — | 0.25 | — | — | — | — | — | — | — | 0.25 | — | — |
| | — | 0.25 | — | — | — | — | — | — | — | 0.1 | — | — |
| | — | 0.25 | — | — | — | — | — | — | — | — | 0.25 | — |
| | — | 0.25 | — | — | — | — | — | — | — | — | — | 0.1 |
| | — | 0.25 | — | — | — | — | — | — | — | — | — | 0.25 |
| | Mass concentration of different building blocks added (%) | | | | | | | | | | | |
| | 101 | 102M | 103E3 | 104 | 106-K30 | 107 | 314 | 318 | 310 | 312 | 301 | 302 |
| 50 | — | — | 0.25 | — | — | — | — | 0.25 | — | — | — | — |
| | — | — | 0.25 | — | — | — | — | — | 0.25 | — | — | — |
| | — | — | 0.25 | — | — | — | — | — | — | 0.25 | — | — |
| | — | — | 0.25 | — | — | — | — | — | — | — | 0.25 | — |
| | — | — | 0.25 | — | — | — | — | — | — | — | — | 0.25 |

The experimental results of Example 48 are shown in FIG. 52.

According to the results, the initial medium, 0.05% SDS in pH6.8 phosphate buffer showed no significant difference in the effect on Cannabidiol molecule stacking compared to pH6.8 phosphate buffer as initial medium.

Compared with the initial medium, quaternary supramolecular self-assembly systems built by 0.05% carrier 314, 0.25% polymers, and the target guest respectively, had significantly better synergistic regulation ability on Cannabidiol molecule stacking than binary supramolecular self-assembly systems built by 0.25% 102M or 0.25% 101 and the target guest, respectively. In order of combination of polymers 201, 202, 203, 204, and 205, the concentrations of Cannabidiol in the solutions of the quaternary supramolecular self-assembly systems incubated for 6 hours were 732.2 μg/mL, 399.4 μg/mL, 648.3 μg/mL, 725.0 μg/mL, and 581.7 μg/mL, which were 86 times, 47 times, 76 times, 85 times, and 68 times that of the initial medium at 6 hours, respectively.

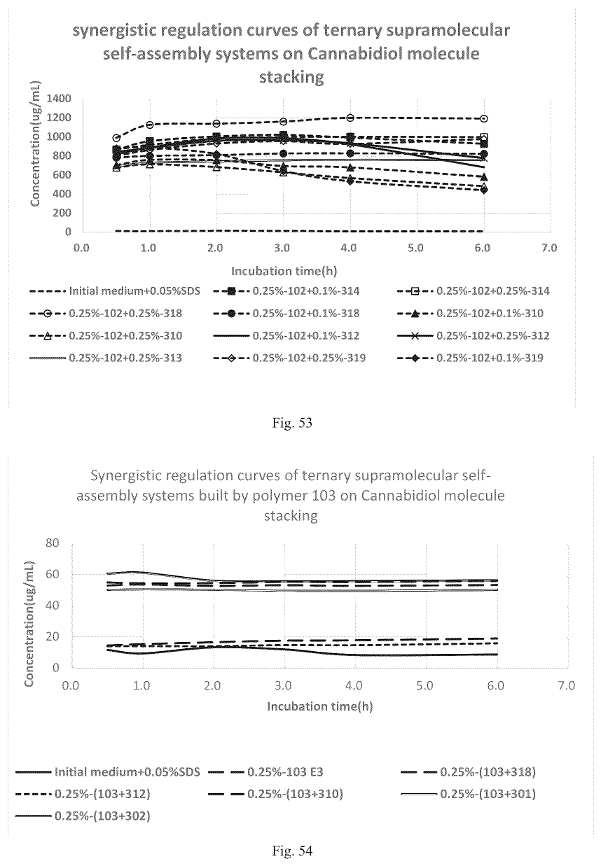

According to Example 49 and FIG. 53, 102M as a polymer and building blocks 314, 318, 312, 310, 313, and 319 at different concentrations were used to build ternary supramolecular self-assembly systems respectively, which had significant synergistic regulation ability compared to a binary supramolecular self-assembly system built by 0.25% 102M and the target guest, where the ternary supramolecular self-assembly system built by building block 310 had slightly weak synergistic regulation ability for Cannabidiol, and the concentration of Cannabidiol measured in the solution showed a slow decreasing trend over incubation time; but the concentrations of Cannabidiol in the ternary systems containing 0.1% and 0.25% 310, measured at 6 hours, were 580.1 μg/mL and 481.2 μg/mL, which were significantly higher than the best result 150 μg/mL reported by the literature [International Journal of Pharmaceutics 589 (2020) 119812]. The concentrations of Cannabidiol in the ternary supramolecular self-assembly systems built by 314, 318, 312, 313, 319, and 102M measured at 6 hours were from 680.9 μg/mL to 1190.2 μg/mL, and the systems remained stable after being incubated for 6 hours, without molecular stacking.

According to Example 50 and FIG. 54, 103E3 as a polymer and building blocks 318, 312, 310, 301, and 302 at different concentrations were selected to build ternary supramolecular self-assembly systems, which did not show any synergistic regulation ability on guest molecule stacking compared to a binary supramolecular self-assembly system built by 0.25% 103E3 and the target guest. The concentration of Cannabidiol in each system measured at 6 hours was 15.7 to 56.2 μg/mL, while the concentration of Cannabidiol in the binary supramolecular self-assembly system built by 103E3 and the target guest measured at 6 hours was 55.7 μg/mL.

The chemical structural formulas of Cannabidiol derivatives are as follows:

TABLE 30
Chemical structures of Cannabidiol derivatives

| | | Log P or H- | English chemical | Molecular | Molecular |
|---|---|---|---|---|---|
| Number | Structures | D,H-R | name | formula | weight |
| 1 | 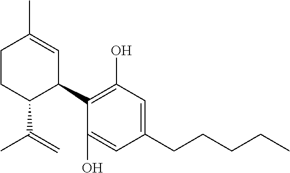 [SMILES: C=C(C)[C@@H]1CCC(C)=C[C@H]1c1c(O)cc(CCCCC)cc1O] | Log P = 6.32, H-D:2,H-R = 2 | ((1′R,2′R)-5′- methyl-4-pentyl-2′- (prop-1-en-2-yl)- 1′,2′,3′,4′-tetrahydro- [1,1′-biphenyl]-2,6- diol) | C₂₁H₃₀ O₂ | 314.46 |
| 2 | 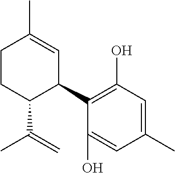 [SMILES: C=C(C)[C@@H]1CCC(C)=C[C@H]1c1c(O)cc(C)cc1O] | Log P = 4.55, H-D:2,H-R = 2 | ((1′R,2′R)-4,5′- dimethyl-2′-(prop-1- en-2-yl)-1′,2′,3′,4′- tetrahydro-[1,1′- biphenyl]-2,6-diol) | C₁₇H₂₂ O₂ | 258.36 |
| 3 | 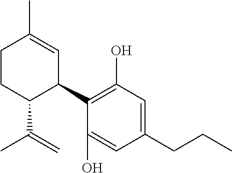 [SMILES: C=C(C)[C@@H]1CCC(C)=C[C@H]1c1c(O)cc(CCC)cc1O] | Log P = 5.44 H-D:2,H-R = 2 | ((1′R,2′R)-5′- methyl-2′-(prop-1- en-2-yl)-4-propyl- 1′,2′,3′,4′-tetrahydro- [1,1′-biphenyl]-2,6- diol) | C₁₉H₂₆ O₂ | 286.41 |
| 4 | 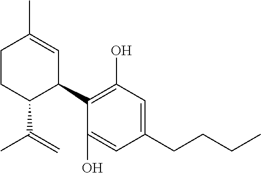 [SMILES: C=C(C)[C@@H]1CCC(C)=C[C@H]1c1c(O)cc(CCCC)cc1O] | Log P = 5.88 H-D:2,H-R = 2 | ((1′R,2′R)-4-butyl- 5′-methyl-2′-(prop- 1-en-2-yl)-1′,2′,3′,4′- tetrahydro-[1,1′- biphenyl]-2,6-diol) | C₂₀H₂₈ O₂ | 300.44 |
| 5 | 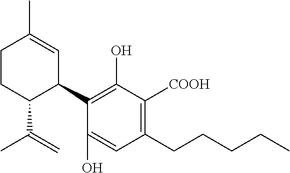 [SMILES: C=C(C)[C@@H]1CCC(C)=C[C@H]1c1c(O)cc(CCCCC)c(C(=O)O)c1O] | Log P = 6.63 D:3,H-R = 4 Log D7.4 = 3.15 | ((1′R,2′R)-2,6- dihydroxy-5′- methyl-4-pentyl-2′- (prop-1-en-2-yl)- 1′,2′,3′,4′-tetrahydro- [1,1′-biphenyl]-3- carboxylic acid) | C₂₂H₃₀ O₄ | 358.47 |
| 6 | 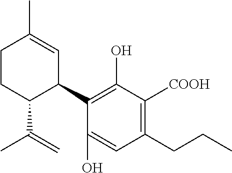 [SMILES: C=C(C)[C@@H]1CCC(C)=C[C@H]1c1c(O)cc(CCC)c(C(=O)O)c1O] | Log P = 5.74 H-D:3,H-R = 4 Log D7.4 = 2.26 | ((1′R,2′R)-2,6- dihydroxy-5′- methyl-2′-(prop-1- en-2-yl)-4-propyl- 1′,2′,3′,4′-tetrahydro- [1,1′-biphenyl]-3- carboxylic acid) | C₂₀H₂₆ O₄ | 330.42 |
| 7 | 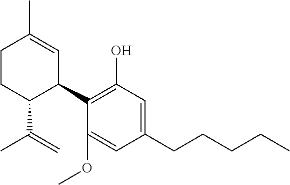 [SMILES: C=C(C)[C@@H]1CCC(C)=C[C@H]1c1c(O)cc(CCCCC)cc1OC] | Log P = 6.47 H-D:1,H-R = 2 | ((1′R,2′R)-6- methoxy-5′-methyl- 4-pentyl-2′-(prop-1- en-2-yl)-1′,2′,3′,4′- tetrahydro-[1,1′- biphenyl]-2-ol) | C₂₂H₃₂ O₂ | 328.49 |
| 8 | 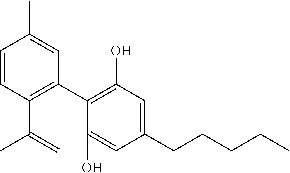 [SMILES: C=C(C)c1ccc(C)cc1-c1c(O)cc(CCCCC)cc1O] | Log P = 6.85 H-D:2,H-R = 2 | (5′-methyl-4-pentyl- 2′-(prop-1-en-2-yl)- [1,1′-biphenyl]-2,6- diol) | C₂₁H₂₆ O₂ | 310.43 |
| 9 | 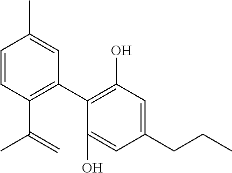 [SMILES: C=C(C)c1ccc(C)cc1-c1c(O)cc(CCC)cc1O] | Log P = 5.97 H-D:2,H-R = 2 | (5′-methyl-2′-(prop- 1-en-2-yl)-4-propyl- [1,1′-biphenyl]-2,6- diol) | C₁₉H₂₂ O₂ | 282.38 |
| 10 | 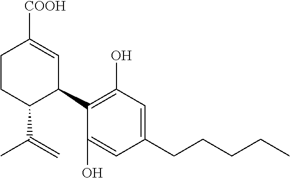 [SMILES: C=C(C)[C@@H]1CCC(C(=O)O)=C[C@H]1c1c(O)cc(CCCCC)cc1O] | Log P = 5.52 H-D:3,H-R = 4 Log D7.4 = 2.40 | ((1R,6R)-2′,6′- dihydroxy-4′-pentyl- 6-(prop-1-en-2-yl)- 1,4,5,6-tetrahydro- [1,1′-biphenyl]-3- carboxylic acid) | C₂₁H₂₈ O₄ | 344.44 |
| 11 | 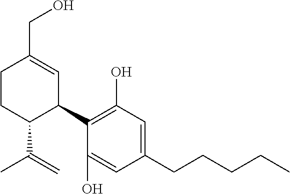 [SMILES: C=C(C)[C@@H]1CCC(CO)=C[C@H]1c1c(O)cc(CCCCC)cc1O] | Log P = 5.04 H-D:3,H-R = 3 | ((1′R,2′R)-5′- (hydroxymethyl)-4- pentyl-2′-(prop-1- en-2-yl)-1′,2′,3′,4′- tetrahydro-[1,1′- biphenyl]-2,6-diol) | C₂₁H₃₀ O₃ | 330.46 |
| 12 | 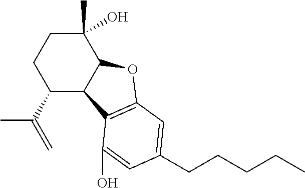 | Log P = 5.01 H-D:2,H-R = 3 | ((5aR,6S,9R,9aR)- 6-methyl-3-pentyl- 9-(prop-1-en-2-yl)- 5a,6,7,8,9,9a- hexahydrodibenzo [b,d]furan-1,6-diol) | C₂₁H₃₀ O₃ | 330.46 |
| 13 | 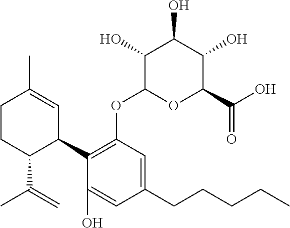 [SMILES: C=C(C)[C@@H]1CCC(C)=C[C@H]1c1c(O)cc(CCCCC)cc1OC1O[C@H](C(=O)O)[C@@H](O)[C@H](O)[C@H]1O] | Log P = 4.38 H-D:5,H-R = 8 | ((2S,3S,4S,5R)- 3,4,5-trihydroxy-6- ((1′R,2′R)-6- hydroxy-5′-methyl- 4-pentyl-2′-(prop-1- en-2-yl)-1′,2′,3′,4′- tetrahydro-[1,1′- biphenyl]-2- yl)oxy)tetrahydro- 2H-pyran-2- carboxylic acid) | C₂₇H₃₈ O₈ | 490.59 |
| 14 | 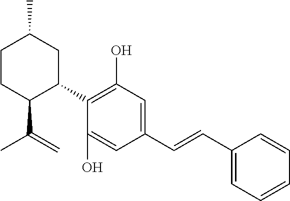 [SMILES: C=C(C)[C@H]1CC[C@H](C)C[C@@H]1c1c(O)cc(/C=C/c2ccccc2)cc1O] | Log P = 6.78 H-D:2,H-R = 2 | (2-((1S,2S,5S)-5- methyl-2-(prop-1- en-2-yl)cyclohexyl)- 5-((E)- styryl)phenyl-1,3- diol) | C₂₄H₂₈ O₂ | 348.48 |
| 15 | 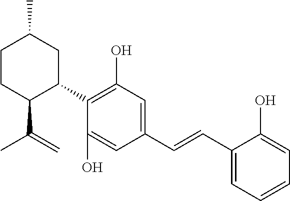 [SMILES: C=C(C)[C@H]1CC[C@H](C)C[C@@H]1c1c(O)cc(/C=C/c2ccccc2O)cc1O] | Log P = 6.48 H-D:3,H-R = 3 | (5-((E)-2- hydroxystyryl-2- (1S,2S,5S)-5- methyl-2-(prop-1- en-2- yl)cyclohexyl) phenyl-1,3-diol) | C₂₄H₂₈ O₃ | 364.48 |
| 16 | 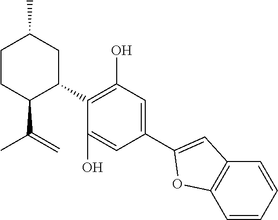 [SMILES: C=C(C)[C@H]1CC[C@H](C)C[C@@H]1c1c(O)cc(-c2cc3ccccc3o2)cc1O] | Log P = 6.17 H-D:2,H-R = 2 | (5-(benzofuran-2- yl)-2-(1S,2S,5S)-5- methyl-2-(prop-1- en-2- yl)cyclohexyl) phenyl-1,3-diol) | C₂₄H₂₆ O₃ | 362.46 |
| 17 | 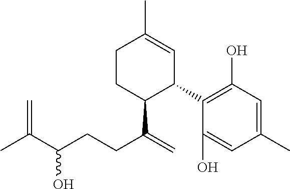 [SMILES: C=C(C)C(O)CCC(=C)[C@H]1CCC(C)=C[C@@H]1c1c(O)cc(C)cc1O] | Log P = 5.03 H-D:3,H-R = 3 | ((1′S,2′S)-2′-(5- hydroxy-6- methylhept-1,6- dien-2-yl)-4,5′- dimethyl-1′,2′,3′,4′- tetrahydro-[1,1′- biphenyl]-2,6-diol) | C₂₂H₃₀ O₃ | 342.47 |
| 18 | 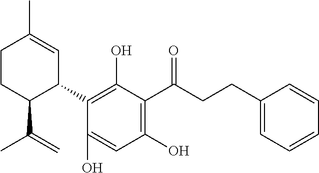 [SMILES: C=C(C)[C@H]1CCC(C)=C[C@@H]1c1c(O)cc(O)c(C(=O)CCc2ccccc2)c1O] | Log P = 6.87 H-D:3,H-R = 4 | (3-phenyl-1- ((1′S,2′S)-2,4,6- trihydroxy-5′- methyl-2′-(prop-1- en-2-yl)-1′,2′,3′,4′- tetrahydro-[1,1′- biphenyl]-3-yl)prop- 1-one) | C₂₅H₂₈ O₄ | 392.49 |
| 19 | 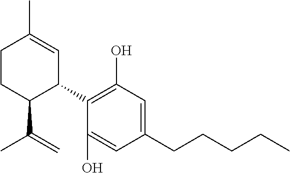 [SMILES: C=C(C)[C@H]1CCC(C)=C[C@@H]1c1c(O)cc(CCCCC)cc1O] | Log P = 6.33 H-D:2,H-R = 2 | ((1′S,2′S)-5′-methyl- 4-pentyl-2′- (propanediol-1-en- 2-yl)-1′,2′,3′,4′- tetrahydro-[1,1′- biphenyl]-2,6-diol) | C₂₁H₃₀ O₂ | 314.46 |
| 20 | 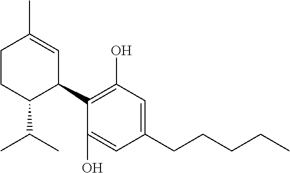 [SMILES: CCCCCc1cc(O)c([C@@H]2C=C(C)CC[C@H]2C(C)C)c(O)c1] | Log P = 6.67 H-D:2,H-R = 2 | ((1′S,2′S)-2′- isopropyl-5′-methyl- 4-pentyl-1′,2′,3′,4′- tetrahydro-[1,1′- biphenyl]-2,6-diol) | C₂₁H₃₂ O₂ | 316.48 |
| 21 | 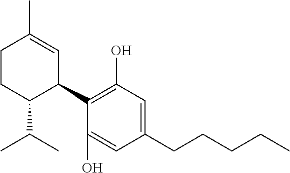 [SMILES: CCCCCc1cc(O)c([C@@H]2C=C(C)CC[C@H]2C(C)C)c(O)c1] | Log P = 7.08 H-D:2,H-R = 2 | (2-((1R,2S)-2- isopropyl-5- methylcyclohexyl)- 5-pentylbenzene- 1,3-diol) | C₂₁H₃₄ O₂ | 318.49 |
| 22 | 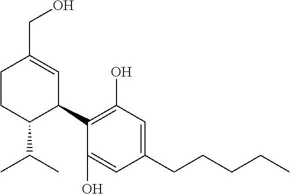 [SMILES: CCCCCc1cc(O)c([C@@H]2C=C(CO)CC[C@H]2C(C)C)c(O)c1] | Log P = 5.39 H-D:3,H-R = 3 | ((1′S,2′S)-5′- (hydroxymethyl)-2′- isopropyl-4-pentyl- 1′,2′,3′,4′-tetrahydro- [1,1′-biphenyl]-2,6- diol) | C₂₁H₃₂ O₃ | 332.48 |
| 23 | 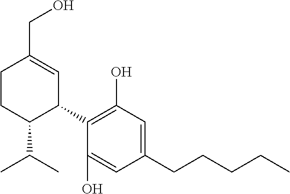 [SMILES: CCCCCc1cc(O)c([C@H]2C=C(CO)CC[C@H]2C(C)C)c(O)c1] | Log P = 5.39 H-D:3,H-R = 3 | ((1′R,2′S)-5′- (hydroxymethyl)-2′- isopropyl-4-pentyl- 1′,2′,3′,4′-tetrahydro- [1,1′-biphenyl]-2,6- diol) | C₂₁H₃₂ O₃ | 332.48 |
| 24 | 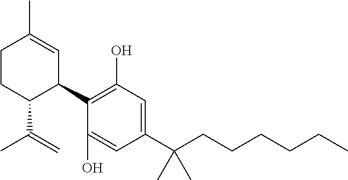 [SMILES: C=C(C)[C@@H]1CCC(C)=C[C@H]1c1c(O)cc(C(C)(C)CCCCCC)cc1O] | Log P = 7.38 H-D:2,H-R = 2 | ((1′R,2′R)-5′- methyl-4-(2- methyloctan-2-yl)- 2′-(prop-1-en-2-yl)- 1′,2′,3′,4′-tetrahydro- [1,1′-biphenyl]-2,6- diol) | C₂₅H₃₈ O₂ | 370.57 |
| 25 | 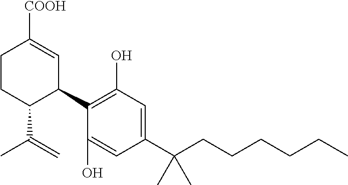 [SMILES: C=C(C)[C@@H]1CCC(C(=O)O)=C[C@H]1c1c(O)cc(C(C)(C)CCCCCC)cc1O] | Log P = 6.99 H-D:3,H-R = 4 | ((1R,6R)-2′,6′- dihydroxy-4′-(2- methyloctan-2-yl)- 6-(prop-1-en-2-yl)- 1,4,5,6-tetrahydro- [1,1′-biphenyl]-3- carboxylic acid) | C₂₅H₃₆ O₄ | 400.55 |
| 26 | 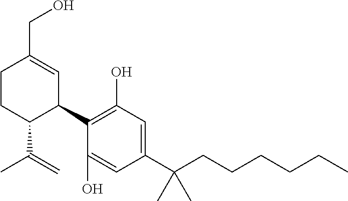 [SMILES: C=C(C)[C@@H]1CCC(CO)=C[C@H]1c1c(O)cc(C(C)(C)CCCCCC)cc1O] | Log P = 6.52 H-D:3,H-R = 3 | ((1′R,2′R)-5′- (hydroxymethyl)-4- (2-methyloctan-2- yl)-2′-(prop-1-en-2- yl)-1′,2′,3′,4′- tetrahydro-[1,1′- biphenyl]-2,6-diol) | C₂₅H₃₈ O₃ | 386.57 |
| 27 | 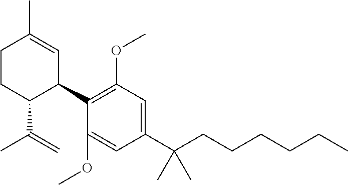 [SMILES: C=C(C)[C@@H]1CCC(C)=C[C@H]1c1c(OC)cc(C(C)(C)CCCCCC)cc1OC] | Log P = 8.09 H-D:0,H-R = 2 | ((1R,2R)-2′,6′- dimethoxy-5- methyl-4′-(2- methyloctan-2-yl)- 2-(prop-1-en-2-yl)- 1,2,3,4-tetrahydro- 1,1′-biphenyl) | C₂₇H₄₂ O₂ | 398.62 |
| 28 | 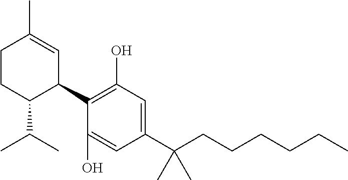 [SMILES: CCCCCCC(C)(C)c1cc(O)c([C@@H]2C=C(C)CC[C@H]2C(C)C)c(O)c1] | Log P = 8.15 H-D:2,H-R = 2 | isopropyl-5′-methyl- 4-(2-methyloctan-2- yl)-1′,2′,3′,4′- tetrahydro-[1,1′- biphenyl]-2,6-diol) | C₂₅H₄₀ O₂ | 372.58 |
| 29 | 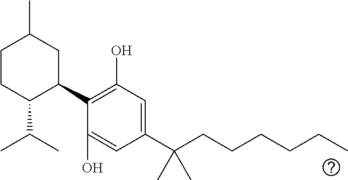 [SMILES: CCCCCCC(C)(C)c1cc(O)c([C@@H]2CC(C)CC[C@H]2C(C)C)c(O)c1] | Log P = 8.55 H-D:2,H-R = 2 | (2-((1R,2S)-2- isopropyl-5- methylcyclohexyl)- 5-(2-methyloctan-2- yl)benzene-1,3-diol) | C₂₅H₄₂ O₂ | 374.60 |
| 30 | 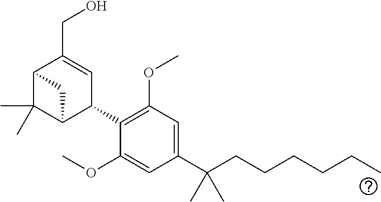 [SMILES: CCCCCCC(C)(C)c1cc(OC)c([C@H]2C=C(CO)[C@H]3C[C@@H]2C3(C)C)c(OC)c1] | Log P = 6.39 H-D:1,H-R = 3 | ((1S,4S,5S)-4-(2,6- dimethoxy-4-(2- methyloctan-2- yl)phenyl)-6,6- dimethylbicyclo [3.1.1]hept-2-en-2- yl)methanol) | C₂₇H₄₂ O₃ | 414.62 |
| 31 | 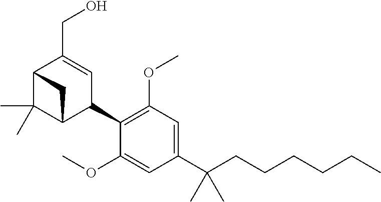 | Log P = 6.39 H-D:1,H-R = 3 | ((1S,4S,5S)-4-(2,6- dimethoxy-4-(2- methyloctan-2- yl)phenyl)-6,6- dimethylbicyclo [3.1.1]hept-2-en-2- yl)methanol) | C₂₇H₄₂ O₃ | 414.62 |
| 32 | 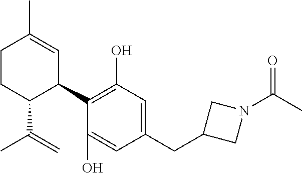 [SMILES: C=C(C)[C@@H]1CCC(C)=C[C@H]1c1c(O)cc(CC2CN(C(C)=O)C2)cc1O] | Log P = 3.42 H-D:2,H-R = 3 | (1-(3-((1′R,2′R)-2,6- dihydroxy-5′- methyl-2′-(prop-1- en-2-yl)-1′,2′,3′,4′- tetrahydro-[1,1′- biphenyl]-4- yl)methyl)azetidin- 1-yl)ethanone) | C₂₂H₂₉ NO₃ | 355.47 |
| 33 | 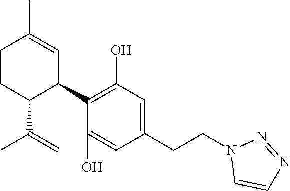 [SMILES: C=C(C)[C@@H]1CCC(C)=C[C@H]1c1c(O)cc(CCn2ccnn2)cc1O] | Log P = 4.09 H-D:2,H-R = 4 | ((1′R,2′R)-4-(2-(1H- 1,2,3-triazol-1- yl)ethyl)-5′-methyl- 2′-(prop-1-en-2-yl)- 1′,2′,3′,4′-tetrahydro- [1,1′-biphenyl]-2,6- diol) | C₂₀H₂₅ N₃O₂ | 339.43 |
| 34 | 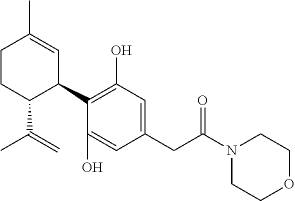 [SMILES: C=C(C)[C@@H]1CCC(C)=C[C@H]1c1c(O)cc(CC(=O)N2CCOCC2)cc1O] | Log P = 3.09 D:2,H-R = 4 Intrinsic solubility = 0.12 mg/mL | (2-((1′R,2′R)-2,6- dihydroxy-5′- methyl-2′-(prop-1- en-2-yl)-1′,2′,3′,4′- tetrahydro-[1,1′- biphenyl]-4-yl)-1- morpholinoethanone) | C₂₂H₂₉ NO₄ | 371.47 |
| 35 | 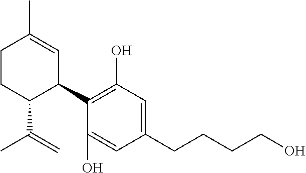 [SMILES: C=C(C)[C@@H]1CCC(C)=C[C@H]1c1c(O)cc(CCCCO)cc1O] | Log P = 4.44 H-D:3,H-R = 3 | ((1′R,2′R)-4-(4- hydroxybutyl)-5′- methyl-2′-(prop-1- en-2-yl)-1′,2′,3′,4′- tetrahydro-[1,1′- biphenyl]-2,6-diol) | C₂₀H₂₈ O₃ | 316.43 |
| 36 | 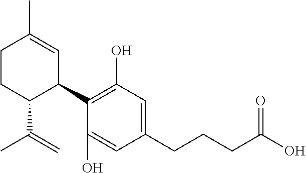 [SMILES: C=C(C)[C@@H]1CCC(C)=C[C@H]1c1c(O)cc(CCCC(=O)O)cc1O] | Log P = 4.59 H-D:3,H-R = 4 | (4-((1′R,2′R)-2,6- dihydroxy-5′- methyl-2′-(prop-1- en-2-yl)-1′,2′,3′,4′- tetrahydro-[1,1′- biphenyl]-4- yl)butyric acid) | C₂₀H₂₆ O₄ | 330.42 |
| 37 | 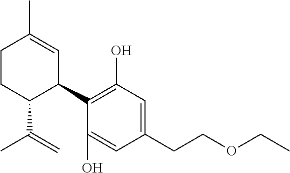 [SMILES: C=C(C)[C@@H]1CCC(C)=C[C@H]1c1c(O)cc(CCOCC)cc1O] | Log P = 4.55 H-D:2,H-R = 3 | ((1′R,2′R)-4-(2- ethoxyethyl)-5′- methyl-2′-(prop-1- en-2-yl)-1′,2′,3′,4′- tetrahydro-[1,1′- biphenyl]-2,6-diol) | C₂₀H₂₈ O₃ | 316.43 |
| 38 | 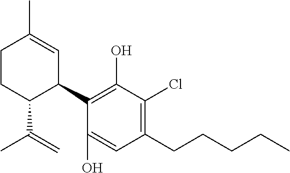 [SMILES: C=C(C)[C@@H]1CCC(C)=C[C@H]1c1c(O)cc(CCCCC)c(Cl)c1O] | Log P = 6.93 H-D:2,H-R = 2 | ((1′R,2′R)-3-chloro- 5′-methyl-4-pentyl- 2′-(prop-1-en-2-yl)- 1′,2′,3′,4′-tetrahydro- [1,1′-biphenyl]-2,6- diol) | C₂₁H₂₉ ClO₂ | 348.91 |
| 39 | 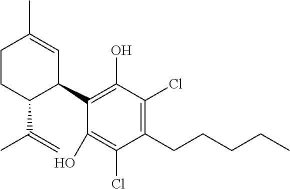 [SMILES: C=C(C)[C@@H]1CCC(C)=C[C@H]1c1c(O)c(Cl)c(CCCCC)c(Cl)c1O] | Log P = 7.53 H-D:2,H-R = 2 | ((1′R,2′R)-3,5- dichloro-5′-methyl- 4-pentyl-2′-(prop-1- en-2-yl)-1′,2′,3′,4′- tetrahydro-[1,1′- biphenyl]-2,6-diol) | C₂₁H₂₈ Cl₂O₂ | 383.35 |
| 40 | 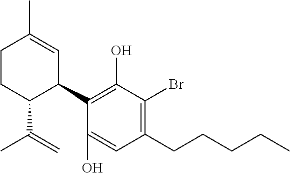 [SMILES: C=C(C)[C@@H]1CCC(C)=C[C@H]1c1c(O)cc(CCCCC)c(Br)c1O] | Log P = 7.09 H-D:2,H-R = 2 | ((1′R,2′R)-3-bromo- 5′-methyl-4-pentyl- 2′-(prop-1-en-2-yl)- 1′,2′,3′,4′-tetrahydro- [1,1′-biphenyl]-2,6- diol) | C₂₁H₂₉ BrO₂ | 393.36 |
| 41 | 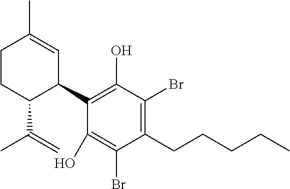 [SMILES: C=C(C)[C@@H]1CCC(C)=C[C@H]1c1c(O)c(Br)c(CCCCC)c(Br)c1O] | Log P = 7.86 H-D:2,H-R = 2 | ((1′R,2′R)-3,5- dibromo-5′-methyl- 4-pentyl-2′-(prop-1- en-2-yl)-1′,2′,3′,4′- tetrahydro-[1,1′- biphenyl]-2,6-diol) | C₂₁H₂₈ Br₂O₂ | 472.25 |
| 42 | 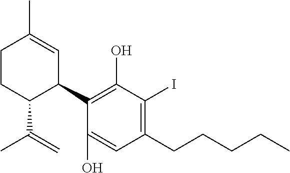 [SMILES: C=C(C)[C@@H]1CCC(C)=C[C@H]1c1c(O)cc(CCCCC)c(I)c1O] | Log P = 7.25 H-D:2,H-R = 2 | ((1′R,2′R)-3-iodo-5′- methyl-4-pentyl-2′- (prop-1-en-2-yl)- 1′,2′,3′,4′-tetrahydro- [1,1′-biphenyl]-2,6- diol) | C₂₁H₂₉I O₂ | 440.36 |
| 43 | 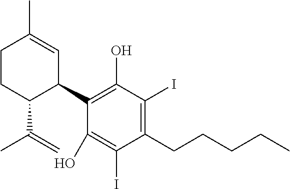 [SMILES: C=C(C)[C@@H]1CCC(C)=C[C@H]1c1c(O)c(I)c(CCCCC)c(I)c1O] | Log P = 8.81 H-D:2,H-R = 2 | ((1′R,2′R)-3,5- diiodo-5′-methyl-4- pentyl-2′-(prop-1- en-2-yl)-1′,2′,3′,4′- tetrahydro-[1,1′- biphenyl]-2,6-diol) | C₂₁H₂₈ I₂O₂ | 566.25 |
| 44 | 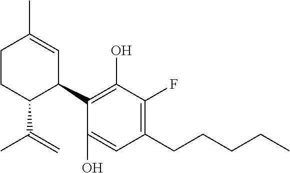 [SMILES: C=C(C)[C@@H]1CCC(C)=C[C@H]1c1c(O)cc(CCCCC)c(F)c1O] | Log P = 6.47 H-D:2,H-R = 2 | ((1′R,2′R)-3-fluoro- 5′-methyl-4-pentyl- 2′-(prop-1-en-2-yl)- 1′,2′,3′,4′-tetrahydro- [1,1′-biphenyl]-2,6- diol) | C₂₁H₂₉F O₂ | 332.45 |
| 45 | 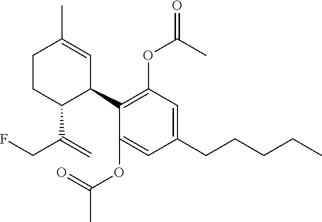 [SMILES: C=C(CF)[C@@H]1CCC(C)=C[C@H]1c1c(OC(C)=O)cc(CCCCC)cc1OC(C)=O] | Log P = 5.76 H-D:0,H-R = 2 | (3-(acetoxy)-2- [(1R,6R)-6-(3- fluoroprop-1-en-2- yl)-3- methylcyclohex-2- en-1-yl]-5- pentylphenyl acetate) | C₂₅H₃₃F O₄ | 416.53 |
| 46 | 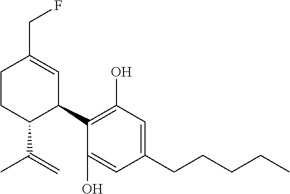 [SMILES: C=C(C)[C@@H]1CCC(CF)=C[C@H]1c1c(O)cc(CCCCC)cc1O] | Log P = 5.94 H-D:2,H-R = 2 | ((1′R,2′R)-5′- (fluoromethyl)-4- pentyl-2′-(prop-1- en-2-yl)-1′,2′,3′,4′- tetrahydro-[1,1′- biphenyl]-2,6-diol) | C₂₁H₂₉F O₂ | 332.45 |
| 47 | 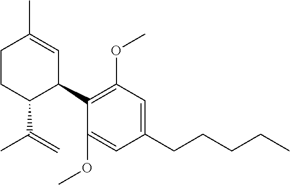 [SMILES: C=C(C)[C@@H]1CCC(C)=C[C@H]1c1c(OC)cc(CCCCC)cc1OC] | Log P = 6.62 H-D:0,H-R = 2 | (1,3-dimethoxy-2- [(1R,6R)-3-methyl- 6-prop-1-en-2- ylcyclohex-2-en-1- yl]-5- pentylbenzene) | C₂₃H₃₄ O₂ | 342.51 |
| 48 | 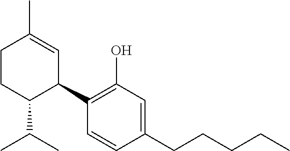 [SMILES: CCCCCc1ccc([C@@H]2C=C(C)CC[C@H]2C(C)C)c(O)c1] | Log P = 6.63 H-D:1, H-R = 1 | ((1′R,2′R)-5′- methyl-4-pentyl-2′- (prop-1-en-2-yl)- 1′,2′,3′,4′-tetrahydro- [1,1′-biphenyl]-2-ol) | C₂₁H₃₀ O | 298.46 |
| 49 | 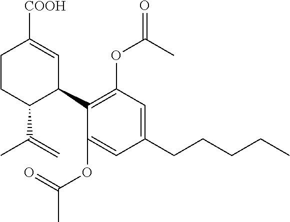 [SMILES: C=C(C)[C@@H]1CCC(C(=O)O)=C[C@H]1c1c(OC(C)=O)cc(CCCCC)cc1OC(C)=O] | Log P = 5.34 H-D:1,H-R = 4 | ((1R,6R)-2′,6′- diacetoxy-4′-pentyl- 6-(prop-1-en-2-yl)- 1,4,5,6-tetrahydro- [1,1′-biphenyl]-3- carboxylic acid) | C₂₅H₃₂ O₆ | 428.52 |
| 50 | 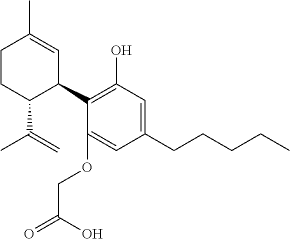 [SMILES: C=C(C)[C@@H]1CCC(C)=C[C@H]1c1c(O)cc(CCCCC)cc1OCC(=O)O] | Log P = 5.95 H-D:2,H-R = 4 | (2-((1′R,2′R)-6- hydroxy-5′-methyl- 4-pentyl-2′-(prop-1- en-2-yl)-1′,2′,3′,4′- tetrahydro-[1,1′- biphenyl]-2- yl)oxy)acetic acid) | C₂₃H₃₂ O₄ | 372.50 |
| 51 | 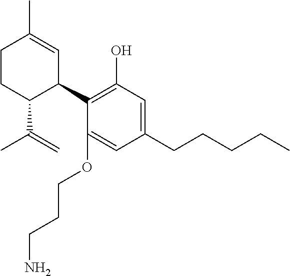 [SMILES: C=C(C)[C@@H]1CCC(C)=C[C@H]1c1c(O)cc(CCCCC)cc1OCCCN] | Log P = 5.73 H-D:2,H-R = 3 | ((1′R,2′R)-6-(3- aminopropoxy)-5′- methyl-4-pentyl-2′- (prop-1-en-2-yl)- 1′,2′,3′,4′-tetrahydro- [1,1′-biphenyl]-2-ol) | C₂₄H₃₇ NO₂ | 371.56 |
| 52 | 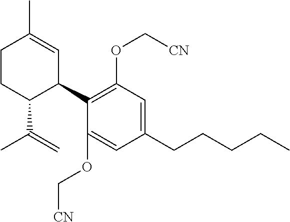 [SMILES: C=C(C)[C@@H]1CCC(C)=C[C@H]1c1c(OCC#N)cc(CCCCC)cc1OCC#N] | Log P = 5.69 H-D:0,H-R = 4 | (2-[3- (cyanomethoxy)-2- [(1R,6R)-3-methyl- 6-(prop-1-en-2- yl)cyclohex-2-en-1- yl]-5- pentylphenoxy] acetonitrile) | C₂₅H₃₂ N₂O₂ | 392.53 |
| 53 | 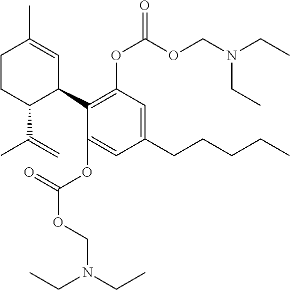 [SMILES: C=C(C)[C@@H]1CCC(C)=C[C@H]1c1c(OC(=O)OCN(CC)CC)cc(CCCCC)cc1OC(=O)OCN(CC)CC] | Log P = 9.07 H-D:0,H-R = 6 | (3- ({[(diethylamino) methoxy] carbonyl}oxy)- 2-[(1R,6R)-3- methyl-6-(prop-1- en-2-yl)cyclohex-2- en-1-yl]-5- pentylphenyl (diethylamino) methyl carbonate) | C₃₃H₅₂ N₂O₆ | 572.78 |
| 54 | 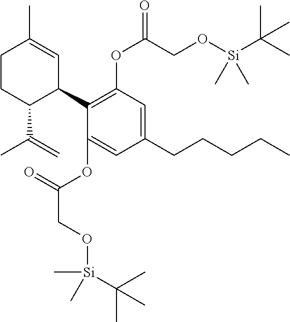 [SMILES: C=C(C)[C@@H]1CCC(C)=C[C@H]1c1c(OC(=O)CO[Si](C)(C)C(C)(C)C)cc(CCCCC)cc1OC(=O)CO[Si](C)(C)C(C)(C)C] | Log P = 9.55 H-D:0,H-R = 4 | (3-({2-[(tert- butyldimethylsilyl) oxy]acetoxy)-2- [(1R,6R)-3-methyl- 6-(prop-1-en-2- yl)cyclohex-2-en-1- yl]-5-pentylphenyl 2-[(tert- butyldimethylsilyl) oxy]acetate) | C₃₇H₆₂ O₆Si₂ | 659.06 |
| 55 | 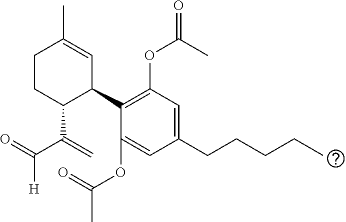 [SMILES: C=C(C=O)[C@@H]1CCC(C)=C[C@H]1c1c(OC(C)=O)cc(CCCCC)cc1OC(C)=O] | Log P = 5.18 H-D:0,H-R = 3 | (3-(acetoxy)-2- [(1R,6R)-3-methyl- 6-(3-oxoprop-1-en- 2-yl)cyclohex-2-en- 1-yl]-5- pentylphenyl acetate) | C₂₅H₃₂ O₅ | 412.52 |
| 56 | 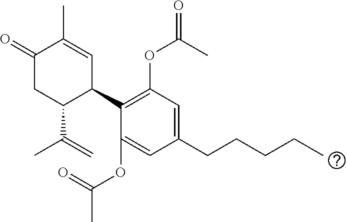 [SMILES: C=C(C)[C@@H]1CC(=O)C(C)=C[C@H]1c1c(OC(C)=O)cc(CCCCC)cc1OC(C)=O] | Log P = 5.48 H-D:0,H-R = 3 | (3-(acetoxy)-2- [(1R,6R)-3-methyl- 4-oxo-6-(prop-1-en- 2-yl)cyclohex-2-en- 1-yl]-5- pentylphenyl acetate) | C₂₅H₃₂ O₅ | 412.52 |
| 57 | 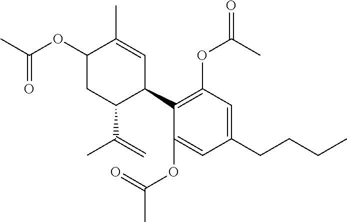 [SMILES: C=C(C)[C@@H]1CC(OC(C)=O)C(C)=C[C@H]1c1c(OC(C)=O)cc(CCCC)cc1OC(C)=O] | Log P = 5.36 H-D:0,H-R = 3 | (3-(acetyloxy)-2- [(1R,6R)-4- (acetyloxy)-3- methyl-6-(prop-1- en-2-yl)cyclohex-2- en-1-yl]-5- pentylphenyl acetate) | C₂₇H₃₆ O₆ | 456.57 |
| 58 | 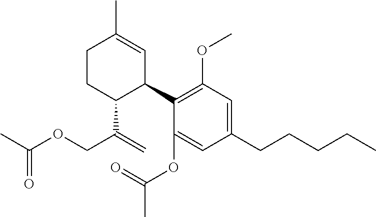 [SMILES: C=C(COC(C)=O)[C@@H]1CCC(C)=C[C@H]1c1c(OC)cc(CCCCC)cc1OC(C)=O] | Log P = 5.31 H-D:0,H-R = 3 | (2-[(1R,2R)-2-[2,6- di(acetoxy)-4- pentenyl]-4- methylcyclohex-3- en-1-yl]prop-2-en- 1-yl acetate) | C₂₇H₃₆ O₆ | 456.57 |
| 59 | 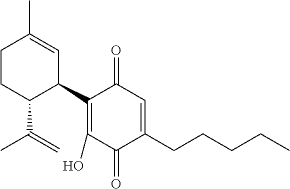 [SMILES: C=C(C)[C@@H]1CCC(C)=C[C@H]1C1=C(O)C(=O)C(CCCCC)=CC1=O] | Log P = 5.23 H-D:1,H-R = 3 | (3-hydroxy-2- [(1R,6R)-3-methyl- 6-prop-1-en-2- ylcyclohex-2-en-1- yl]-5- pentylcyclohex-2,5- diene-1,4-dione) | C₂₁H₂₈ O₃ | 328.45 |
| 60 | 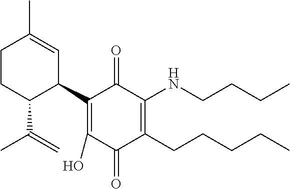 [SMILES: C=C(C)[C@@H]1CCC(C)=C[C@H]1C1=C(O)C(=O)C(CCCCC)=C(NCCCC)C1=O] | Log P = 5.83 H-D:2,H-R = 4 | (2,5- cyclohexadiene-1,4- dione, 2-hydroxy-3- ((1R,6R)-3-methyl- 6-(1-methylvinyl)- 2-cyclohexen-1-yl)- 6-pentyl-5- (butamino)) | C₂₅H₃₇ NO₃ | 399.57 |
| 61 | 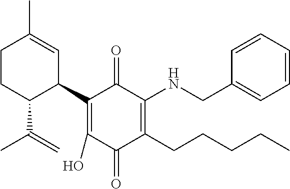 [SMILES: C=C(C)[C@@H]1CCC(C)=C[C@H]1C1=C(O)C(=O)C(CCCCC)=C(NCc2ccccc2)C1=O] | Log P = 6.23 H-D:2,H-R = 4 | (2,5- cyclohexadiene-1,4- dione, 2-hydroxy-3- ((1R,6R)-3-methyl- 6-(1-methylvinyl)- 2-cyclohexen-1-yl)- 6-pentyl-5- ((benzyl)amino)) | C₂₈H₃₅ NO₃ | 433.55 |
| 62 | 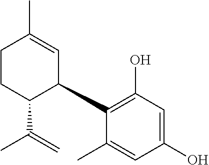 [SMILES: C=C(C)[C@@H]1CCC(C)=C[C@H]1c1c(C)cc(O)cc1O] | Log P = 4.55 H-D:2,H-R = 2 | (5-methyl-4- [(1R,6R)-3-methyl- 6-prop-1-en-2- ylcyclohex-2-en-1- yl]benzene-1,3-diol) | C₁₇H₂₂ O₂ | 258.36 |
| 63 | 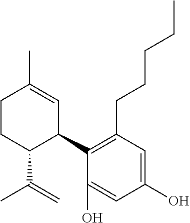 [SMILES: C=C(C)[C@@H]1CCC(C)=C[C@H]1c1c(O)cc(O)cc1CCCCC] | Log P = 6.33 H-D:2,H-R = 2 | (4-[(1R,6R)-3- methyl-6-prop-1-en- 2-ylcyclohex-2-en- 1-yl]-5- pentylbenzene-1,3- diol) | C₂₁H₃₀ O₂ | 314.46 |
| 64 | 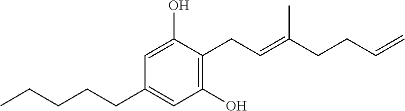 [SMILES: C=CCC/C(C)=C/Cc1c(O)cc(CCCCC)cc1O] | Log P = 7.05 H-D:2,H-R = 2 | (2-[(2E)-3,7- dimethylocta-2,6- dienyl]-5- pentylbenzene-1,3- diol) | C₂₁H₃₂ O₂ | 316.48 |
| 65 | 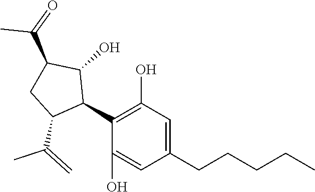 [SMILES: C=C(C)[C@@H]1C[C@@H](C(C)=O)[C@H](O)[C@H]1c1c(O)cc(CCCCC)cc1O] | Log P = 4.40 H-D:3,H-R = 4 | (1-[(1R,2R,3R,4R)- 3-(2,6-dihydroxy-4- pentylphenyl)-2- hydroxy-4-prop-1- en-2- ylcyclopentyl] ethanone) | C₂₁H₃₀ O₄ | 346.46 |
| 66 | 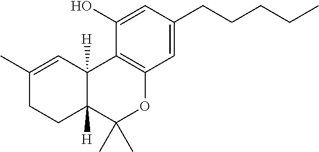 [SMILES: CCCCCc1cc(O)c2c(c1)OC(C)(C)[C@@H]1CCC(C)=C[C@@H]21] | Log P = 5.73 H-D:1,H-R = 2 | Tetrahydrocannabinol | C₂₁H₃₀ O₂ | 314.46 |

From the derivative structures in the table above, most of the structures of the derivatives started from the basic structure of Cannabidiol. In order to improve its solubility in water or pharmacological and pharmacokinetic behaviors, hydrophilic groups were introduced or the alkyl chain of Cannabidiol was shortened or dissociable groups were added. No matter how modified, the Log P of the compounds was still between 3 and 10, showing very lipophilic. Although the molecular weights of the derivatives were controlled below 500 daltons, due to the fixed structure of the skeleton Cannabidiol, except for compound 13, the number of hydrogen acceptors or hydrogen donors in each structure was not more than 4, making it difficult for these molecules to form hydrogen bonds with water. In order to improve the solubility of these substances, Cannabidiol was made into self-microemulsion, but it was difficult to solve the chemical stability during storage. International Journal of Pharmaceuticals 589 (2020) 119812 employed cyclodextrin derivatives such as methyl-beta-cyclodextrin, hydroxypropyl-beta-cyclodextrin, and hydroxypropyl-gama-cyclodextrin inclusion complexes, as well as copovidone VA64, PVP 12PF or Soluplus to prepare solid dispersions of Cannabidiol, in order to reduce the molecular stacking of Cannabidiol in aqueous media. Although cyclodextrins had a significant inhibitory effect on the molecular stacking of Cannabidiol, provided a hydrophobic cavity through their special structure, and formed complexes with Cannabidiol, their effect was significantly dependent on the concentration of cyclodextrin. For example, when the concentration of methyl-beta-cyclodextrin reached 200 mM, the concentration of Cannabidiol in the system can reach 25.5 mg/mL, which was 406699 times the intrinsic solubility of Cannabidiol. At this moment, the mass concentration of cyclodextrin reached 26%, which was obviously far beyond the ADI of cyclodextrins. From the curve, it can be seen that when the concentration of methyl-beta-cyclodextrin was about 5-10 mM, namely, when the mass concentration of methyl-beta-cyclodextrin was 6.5% to 13%, the concentration of Cannabidiol was much lower than 1 mg/mL. The dosage 6.5% of methyl-beta-cyclodextrin was also far beyond the ADI of cyclodextrins, and the daily intake of Cannabidiol reached 10 mg to 20 mg per kg body weight, namely, 500 mg to 1000 mg for adults weighing 50 kg to 60 kg. This was obviously unrealistic.

The Log P of Cannabidiol was 6.32. The main structures of the above derivatives 2, 3, 4, 8, 9, 19, 20, 21, 45, 46, and 63 were the same as Cannabidiol, except that the alkyl chain was shortened and the lipophilicity or hydrophobicity was reduced on the basis of Cannabidiol, so self-assembly systems applicable to Cannabidiol had no lower synergistic regulation ability on the molecular stacking of the above substances than Cannabidiol. Derivatives 5, 6, 10, 11, 12, 13, 16, 17, 22, 23, 32, 33, 34, 35, 36, 37, 49, 50, 51, 52, 55, 59, 60, 61, 62, 65, and 66 were added with dissociable or hydrophilic groups on the basis of Cannabidiol, which increased the opportunity for molecules to form ionic or non-covalent bonds with polymers, carriers, or water, making it easier to form stable supramolecular self-assembly systems than Cannabidiol, so the above supramolecular self-assembly systems applicable to Cannabidiol were also applicable to the target guests. Derivatives 14, 15, 18, 24, 25, 26, 27, 28, 29, 30, 31, 38, 39, 40, 41, 42, 43, 44, 47, 48, 53, 54, 61, and 64 enhanced the hydrophobicity on their side chains on the basis of Cannabidiol, or were added with halogen elements to their main structure to improve the chemical stability of molecules, but the hydrophobicity of the molecules was stronger, so the relatively hydrophilic 102M was replaced with 102H in the above supramolecular self-assembly systems for Cannabidiol, to improve the hydrophobicity of the systems and increase the synergistic regulation of polymers and carriers on the stacking of such molecules.

### Example 51

Cannabidiol, Nintedanib, and Lurasidone hydrochloride as target guest molecules, polymers selected from 101, 103, 104, 106, 107, or 111, and carrier 314 built binary or ternary self-assembly systems respectively. Experiments were conducted according to the following table, the experimental steps were the same as those in Comparative Examples 1 and 2, and the quantitative determination methods for Cannabidiol, Nintedanib, and Lurasidone hydrochloride were the same as before.

TABLE 30
Scheme design for Examples 51-54

| Example | | Mass concentration of different building blocks added (%) | | | | | | | |
|---|---|---|---|---|---|---|---|---|---|
| No | Target guest | 101 | 102 | 103 | 104 | 106 | 107 | 111 | 314 |
| 51 | Cannabidiol | 0.25 | — | — | — | — | — | — | 0.25 |
| | | — | — | — | — | 0.25 | — | — | 0.25 |
| | | — | — | — | — | — | 0.25 | — | 0.25 |
| | | — | — | — | — | — | — | 0.25 | 0.25 |
| 52 | Nintedanib | 101 | 102 | 103 | 104 | 106 | 107 | 115 | 314 |
| | | 0.25 | — | — | — | — | — | — | 0.25 |
| | | — | — | 0.25 | — | — | — | — | 0.25 |
| | | — | — | — | — | 0.25 | — | — | 0.25 |
| | | — | — | — | — | — | — | 0.02 | 0.25 |
| | | — | — | — | — | — | 0.25 | — | 0.25 |
| 53 | Lurasidone | 101 | 102 | 103 | 104 | 106 | 107 | 314 | 319 |
| | hydrochloride | 0.25 | — | — | — | — | — | 0.25 | — |
| | | 0.25 | — | — | — | — | — | 0.5 | — |
| | | — | — | — | — | — | — | 0.25 | — |
| | | — | — | — | — | — | — | 0.5 | — |
| | | — | — | — | — | — | — | — | 0.25 |
| | | 0.25 | — | — | — | — | — | — | 0.25 |
| 54 | Posaconazole | 102 H | 301 | 302 | 310 | 312 | 314 | 313 | 319-1 |
| | | 0.25 | 0.25 | — | — | — | — | — | — |
| | | 0.25 | — | 0.25 | — | — | — | — | — |
| | | 0.25 | — | — | 0.25 | — | — | — | — |
| | | 0.25 | — | — | — | 0.25 | — | — | — |
| | | 0.25 | — | — | — | — | 0.25 | — | — |
| | | 0.25 | — | — | — | — | — | 0.25 | — |
| | | 0.25 | — | — | — | — | — | — | 0.25 |

From Example 51 and FIG. 54, among binary supramolecular self-assembly systems built by Cannabidiol, 0.25% polymer 101 or 106 (K30) or 107 or 111, compared with initial medium, except that polymer 111 had no synergistic regulation effect on Cannabidiol molecule stacking, others had varying degrees of synergistic regulation, where the binary self-assembly systems built by 0.25% polymer 101 and 106 had relatively strong synergistic regulation ability and can maintain stability during 6 hours, and the concentrations of Cannabidiol in the systems measured at 6 hours were 342.5 μg/mL and 155.7 μg/mL, which were 40 times and 18 times that of the initial medium, respectively. Ternary supramolecular self-assembly systems built by said polymers with 0.25% carrier 314 and Cannabidiol did not have further synergistic regulation effects on the basis of the binary systems.

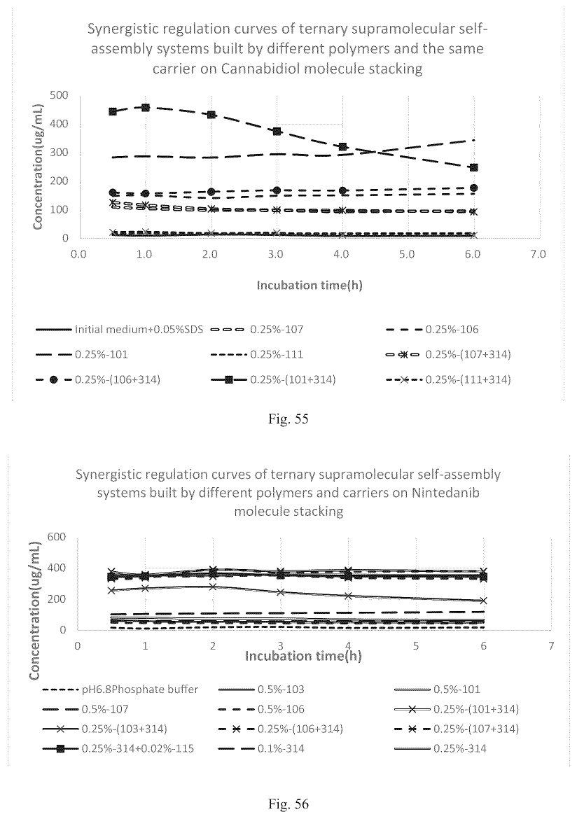

From Example 52 and FIG. 55, binary supramolecular self-assembly systems built by 0.1% and 0.25% carrier 314 and Nintedanib respectively had significant synergistic regulation effects on Nintedanib molecule stacking, and the concentrations of Nintedanib in the systems measured at 6 hours were 116.5 μg/mL and 354.7 μg/mL, which were 7.1 times and 21.5 times that of the initial medium, respectively. The system containing 0.1% building block 314 showed a slow increasing trend within the incubation time, indicating that the system had not reached equilibrium. The system containing 0.25% carrier 314 remained stable from 0.5 hour to 6 hours, indicating that its synergistic regulation ability had reached the strongest. Among ternary supramolecular self-assembly systems built by 0.25% carrier 314 and 0.25% polymer 101, 103, 106, or 107, or 0.02% 115 respectively, except that the synergistic regulation ability of the ternary supramolecular self-assembly system built by 0.25% polymer 103 was slightly lower than that of the binary system built by 0.25% 314, the ternary systems built by the other polymers and 0.25% 314 maintained the level of the binary system built by 0.25% 314.

From Example 53 and FIG. 56, the concentrations of Lurasidone hydrochloride in binary supramolecular self-assembly systems built by 0.25% and 0.5% carrier 314 and Lurasidone hydrochloride, measured at 6 hours, were 56.6 μg/mL and 198.8 μg/mL, which were 81 times and 284 times that of the initial medium, respectively; when 0.25% polymer 101 was added to the binary systems with the same carrier concentration, the concentrations of Lurasidone hydrochloride in the ternary systems measured at 6 hours did not further increase. 0.5% carrier 319 and Lurasidone hydrochloride, or 0.25% carrier 319, 0.25% polymer 101 and Lurasidone hydrochloride built a binary or ternary supramolecular self-assembly system respectively, and the concentrations of Lurasidone hydrochloride in the systems measured at 6 hours were 2.3 μg/mL and 11.2 μg/mL, showing certain synergistic regulation effect compared with the initial medium, but the effect was not significant.

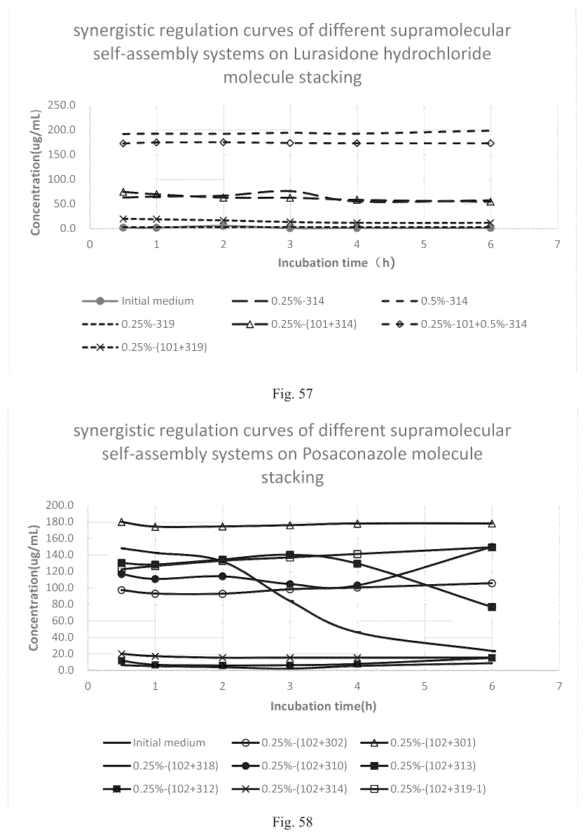

From Example 54 and FIG. 57, Posaconazole and 0.25% polymer 102H with 0.25% carrier 301, 302, 310, 312, 313, 314, or 319-1 built ternary supramolecular self-assembly systems respectively, where the ternary supramolecular self-assembly systems built by 0.25% carrier 312 or 314 had relatively weak synergistic regulation effects on Posaconazole molecule stacking; the concentrations of Posaconazole in the ternary supramolecular self-assembly systems built by 0.25% carriers 313 and 318, measured at 4 hours, were 129.2 μg/mL and 45.9 μg/mL, which were 25 times and 8.8 times that of the initial medium measured at the same incubation time, but their concentrations decreased slightly at 6 hours; and the concentrations of Posaconazole in the ternary supramolecular self-assembly systems built by 0.25% carriers 301, 302, 310, and 319-1 respectively, measured at 6 hours, were 105.5 μg/mL to 178.0 g/mL, which were approximately 12 times and 21 times that of the initial medium.

### Example 55

A target guest molecule selected from Tafluprost in prost derivatives, a polymer selected from 102H, and building blocks selected from 310, 312, 314, 318, 320, and 321 to build supramolecular self-assembly systems respectively. The experimental steps were the same as those in Comparative Examples 1 and 2, and the detection method was the same as before.

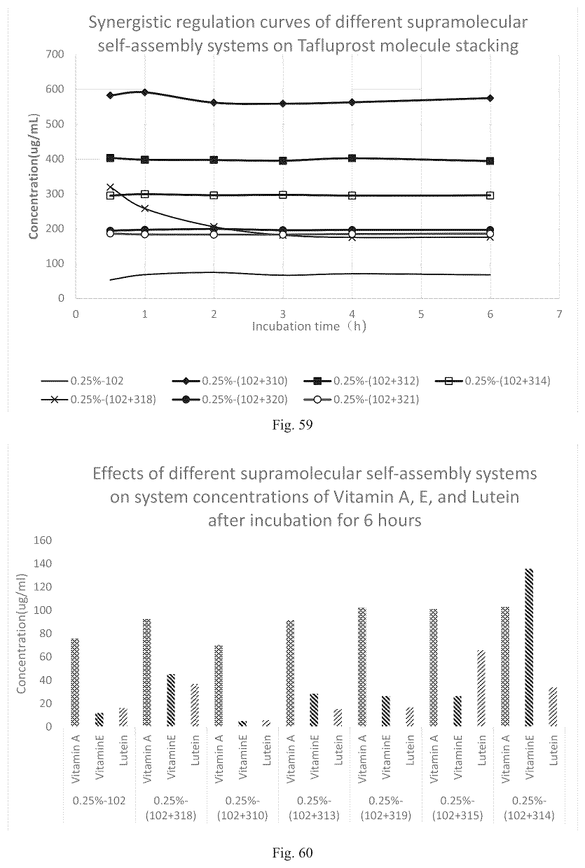

The experimental results are shown in FIG. 59.

Relevant information of the prost derivatives was shown in the table below.

TABLE 31
Relevant information of prost derivatives

| | | English | | | |
|---|---|---|---|---|---|
| General | | chemical | Log P/D- | Molecular | Molecular |
| name | Structures | name | H, R-H | formula | weight |
| Alprostadil | 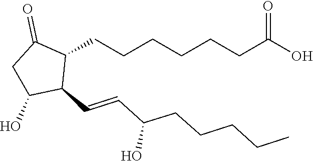 [SMILES: CCCCC[C@H](O)/C=C/[C@H]1[C@H](O)CC(=O)[C@@H]1CCCCCCC(=O)O] | (7- [(1R,2R,3R)- 3-hydroxy-2- [(E,3S)-3- hydroxy-1- enyl]-5- oxocyclopentyl] heptanoic acid) | Log P 3.58 H,3 R-H 5 | C₂₀H₃₄ O₅ | 354.48 |
| Dinoprost | 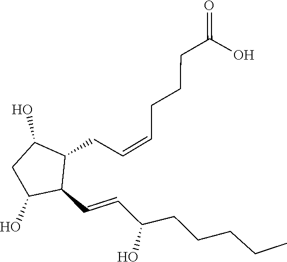 [SMILES: CCCCC[C@H](O)/C=C/[C@@H]1[C@@H](C/C=C\CCCC(=O)O)[C@@H](O)C[C@H]1O] | ((Z)-7- [(1R,2R,3R,5S)- 3,5- dihydroxy-2- [(E,3S)-3- hydroxy-1- enyl] cyclopentyl] hept-5- enoic acid) | Log P 2.61 D-H,4 R-H 5 | C₂₀H₃₄ O₅ | 354.48 |
| Travoprost | 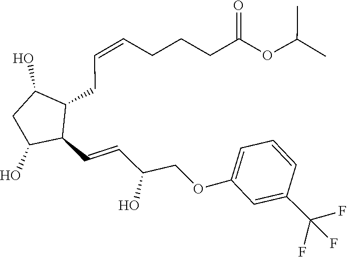 [SMILES: CC(C)OC(=O)CCC/C=C\C[C@@H]1[C@@H](/C=C/[C@@H](O)COc2cccc(C(F)(F)F)c2)[C@H](O)C[C@@H]1O] | (Propan-2- yl(Z)-7- [(1R,2R,3R,5S)- 3,5- dihydroxy-2- [(E,3R)-3- hydroxy-4-[3- (trifluoromethyl) phenoxy] tan-1- enyl] cyclopentyl] hept-5- enoate) | Log P:3.84 D-H: 3 R-H: 5 | C₂₆H₃₅ F₃O₆ | 500.55 |
| Travoprost acid | 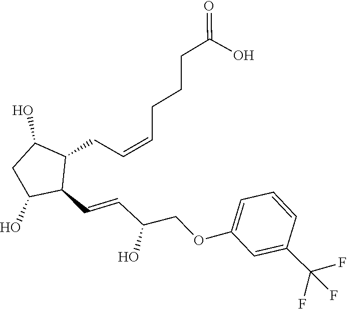 [SMILES: O=C(O)CCC/C=C\C[C@@H]1[C@@H](/C=C/[C@@H](O)COc2cccc(C(F)(F)F)c2)[C@H](O)C[C@@H]1O] | ((Z)-7- [(1R,2R,3R,5S)- 3,5- dihydroxy-2- [(E,3R)-3- hydroxy-4-[3- (trifluoromethyl) phenoxy] tan-1- enyl] cyclopentyl] hept-5- enoic acid) | Log P:2.92 D-H: 4 R-H: 6 | C₂₃H₂₉ F₃O₆ | 458.47 |
| Latanoprost | 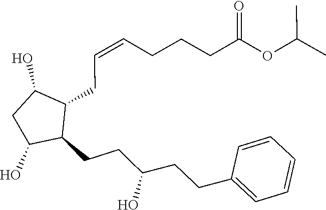 [SMILES: CC(C)OC(=O)CCC/C=C\C[C@@H]1[C@@H](CC[C@@H](O)CCc2ccccc2)[C@H](O)C[C@@H]1O] | (Prop-2-yl(Z)- 7- [(1R,2R,3R,5S)- 3,5- dihydroxy-2- [(3R)-3- hydroxy-5- phenylpentyl] cyclopentyl]hept- 5-enoate) | Log P:3.98 D-H: 3 R-H: 4 Intrinsic solubility: 0.03 mg/ml (37° C.) | C₂₆H₄₀ O₅ | 432.59 |
| Latanoprost lactone diol | 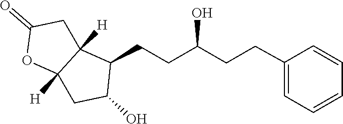 [SMILES: O=C1C[C@@H]2[C@@H](CC[C@@H](O)CCc3ccccc3)[C@H](O)C[C@@H]2O1] | ((3aR,4R,5R, 6aS)-5- hydroxy-4- [(3R)-3- hydroxy-5- phenylpentyl]- 3,3a,4,5,6,6a- hexahydro- cyclo- pentadiene[b] furan-2-one) | Log P: 1.87 D-H: 2 R-H: 3 | C₁₈H₂₄ O₄ | 304.38 |
| Carboprost | 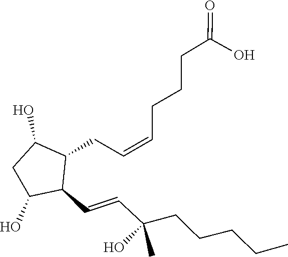 [SMILES: CCCCC[C@](C)(O)/C=C/[C@@H]1[C@@H](C/C=C\CCCC(=O)O)[C@@H](O)C[C@H]1O] | ((Z)-7- [(1R,2R,3R,5S)- 3,5- dihydroxy-2- [(E,3S)-3- hydroxy-3- methyl-1- enyl]cyclopentyl] hept-5- enoic acid) | Log P:2.89 D-H: 4 R-H: 5 | C₂₁H₃₆ O₅ | 368.51 |
| Bimatoprost | 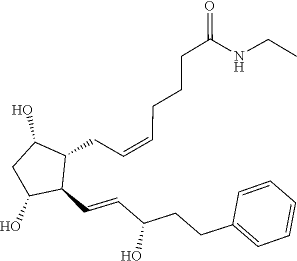 [SMILES: CCNC(=O)CCC/C=C\C[C@@H]1[C@@H](/C=C/[C@@H](O)CCc2ccccc2)[C@H](O)C[C@@H]1O] | ((Z)-7- [(1R,2R,3R,5S)- 3,5- dihydroxy-2- [(E,3S)-3- hydroxy-5- phenylpent-1- enyl] cyclopentyl]- N- ethylhept-5- enamide) | Log P:2.65 D-H: 4 R-H: 4 Intrinsic solubility: 0.05 mg/ml (37° C.) | C₂₅H₃₇ NO₄ | 415.57 |
| Gemeprost | 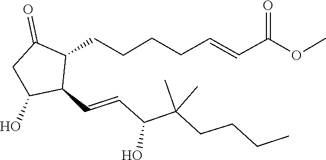 [SMILES: CCCCC(C)(C)[C@H](O)/C=C/[C@H]1[C@H](O)CC(=O)[C@@H]1CCCC/C=C/C(=O)OC] | (Methyl(E)-7- [(1R,2R,3R)- 3-hydroxy-2- [(E,3R)-3- hydroxy-4,4- dimethyl-1- enyl]-5- oxocyclopentyl] hept-2- enoate) | Log P:4.71 D-H: 2 R-H:4 | C₂₃H₃₈ O₅ | 394.54 |
| Tafluprost | 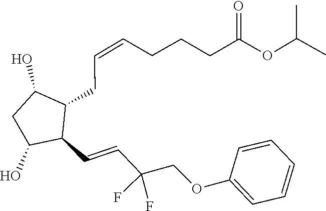 [SMILES: CC(C)OC(=O)CCC/C=C\C[C@@H]1[C@@H](/C=C/C(F)(F)COc2ccccc2)[C@H](O)C[C@@H]1O] | (Propan-2- yl(Z)-7- [(1R,2R,3R,5S)- 2-[(E)-3,3- difluoro-4- phenoxy-1- enyl]-3,5- dihydroxy- cyclopentyl] hept- 5-enoate) | Log P:4.29 D-H: 2 R-H:4 | C₂₅H₃₄ F₂O₅ | 452.53 |
| Misoprostol | 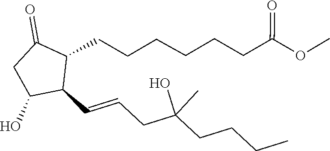 [SMILES: CCCCC(C)(O)C/C=C/[C@H]1[C@H](O)CC(=O)[C@@H]1CCCCCCC(=O)OC] | (7- [(1R,2R,3R)- 3-hydroxy-2- [(E)-4- hydroxy-4- methyl-1- enyl]-5- oxocyclopenty 1]methyl heptanoate) | Log P:3.86 D-H: 2 R-H:4 | C₂₂H₃₈ O₅ | 382.53 |

Among them, the clinical dose of Tafluprost was high, its molecule was highly hydrophobic, Log P4.29, and there were no dissociable groups in its molecular structure.

The concentration of Tafluprost in the binary supramolecular self-assembly system built by Tafluprost and 0.25% 102H, measured after incubation for 6 hours, was 67.3 μg/mL, which was much lower than that of clinical eye drops. The ternary supramolecular self-assembly systems built by Tafluprost, 0.25% polymer 102H, and 0.25% carrier 310 or 312 or 314 or 318 or 320 or 321 respectively showed significant synergistic regulation effects compared to the binary systems, and the concentration of Tafluprost in each ternary supramolecular self-assembly system measured at 6 hours was 175.4 to 574.3 μg/mL, where the supramolecular self-assembly system built by carrier 310 was the most stable and had the strongest synergistic regulation ability. When other prosts are applied in practice, the type of the polymer in the system, such as 102M or 102L, can be adjusted according to clinical dose, chemical structure, Log P, number of hydrogen donors or acceptors, presence of dissociable groups, etc., and different carriers can be selected, to achieve the strongest synergistic control ability.

### Example 56

Target guests selected from Lutein, Vitamin A and Vitamin E, a polymer 0.25% 102H type, and carriers selected from 0.25% 318, 310, 313, 319, 314, or 315 built ternary supramolecular self-assembly systems respectively, the concentrations of the target guests in the systems after incubation for 6 hours were tested, and the experimental steps followed Comparative Examples 1 and 2. The test methods for Lutein, Vitamin A, and Vitamin E are shown in Table 8.

The experimental results are shown in FIG. 60.

According to the experimental results, binary supramolecular self-assembly systems built by each target guest and 0.25% polymer 102H had certain synergistic regulation effects on the stacking of target guest molecules, but none of them reached the dose required for clinical practice of each target guest. The ternary supramolecular self-assembly systems built by each target guest, 0.25% polymer 102H, and 0.25% carrier 318, 310, 313, 319, 314, or 315 respectively had different molecular recognition for each target guest. According to the results of measurement after incubation for 6 hours, the ternary self-assembly systems built by carriers 313, 315, 319, and 314 respectively had the strongest synergistic regulation effect on Vitamin A molecule; the ternary self-assembly systems built by carriers 318, 313, 319, and 314 respectively had the strongest synergistic regulation effect on Vitamin E molecule; and the ternary self-assembly systems built by 318, 315, and 314 respectively had the strongest synergistic regulation effect on Lutein molecule.

It should be noted that the protection scope of the meanings or significances of the numerical values or numerical endpoints involved in the technical solution of the present invention is not limited to the numbers. Those skilled in the art can understand that the numerical values or numerical endpoints include acceptable error ranges widely accepted in the art, such as experimental errors, measurement errors, statistical errors, and random errors, and these error ranges are all included within the scope of the present invention.

The above examples and descriptions are for the convenience of enabling other technical personnel in the technical field to understand and use the present invention, and technical personnel and researchers who are familiar with the art to make modifications to these implementation examples based on their understanding, so as to improve efficiency and reduce costs. Therefore, the present invention includes but is not limited to the above implementation examples, and any modifications and improvements made by other skilled persons in the art based on the provided content of the present invention without departing from the scope of the present invention fall within the protection scope of the present invention.

1. A supramolecular self-assembly system, characterized by comprising the following ingredients:
(1) one or more carriers (or building blocks), which are water-soluble or at least soluble under pH≤8 conditions, wherein at least one carrier is amphiphilic with a hydrophobic group and a hydrophilic group; and
(2) one or more targets, preferably the targets are active ingredients such as drugs, diagnostic agents, biomarkers, vaccines, nutrients, or cosmetic active ingredients, and preferably in a free, salt, hydrate, or solvate form, wherein preferably, the carrier is a compound with a flavonoid or terpenoid structure (preferably from natural sources).

2. The supramolecular self-assembly system according to claim 1, characterized in that the supramolecular self-assembly system further comprises hydroxypropyl methyl cellulose derivatives, preferably hydroxypropyl methyl cellulose acetate succinate (HPMCAS) or hydroxypropyl methyl cellulose (HPMC), and preferably, the supramolecular self-assembly system further comprises one or more additional polymers A, which provide various non-covalent bond interactions for the targets, the carriers, and/or the hydroxypropyl methyl cellulose derivatives (such as HPMCAS), comprising but not limited to ion interaction, hydrogen bonding, hydrophobic interaction, dipole interaction, x-x stacking, Van der Waals force, and are dissoluble within a range of 1.0≤pH≤8.0.

3. The supramolecular self-assembly system according to claim 2, characterized in that the carrier with the flavonoid or terpenoid structure from natural sources has at least 4, preferably at least 6 rotatable chemical bonds, at least 7 or more hydrogen donors, and at least 8 or more hydrogen acceptors, and more preferably, the carrier has at least 1 saccharide structure, such as monosaccharide, disaccharide, trisaccharide, tetrasaccharide, pentasaccharide, hexasaccharide, or a combination thereof.

4. The supramolecular self-assembly system according to claim 2, characterized in that the carrier with the flavonoid structure is selected from the group consisting of flavonoids, flavonols, flavanones (also known as dihydroflavones), flavanonols, isoflavones, anthocyanins, isoflavanones, chalcones, dihydrochalcones, aurones, flavans, and flavanols; the compound with the terpenoid structure refers to a compound derived from mevalonic acid and having a molecular skeleton based on an isoprene unit, such as a monoterpene, sesquiterpene, diterpene, triterpene, or tetraterpene compound.

5. The supramolecular self-assembly system according to claim 2, characterized in that the polymer is selected from natural high molecular polymers and modified materials thereof, or artificially synthesized or semi-synthetic high molecular polymers, comprising but not limited to celluloses, homopolymers or copolymers, surfactants or emulsifiers.

6. The supramolecular self-assembly system according to claim 2, characterized in that the target is selected from one or more of peptide drugs (such as cyclosporine, vitamin B12, voclosporin, 6-[(2S,3R,4R)-10-(acetylamino)-3-hydroxy-4-methyl-2-(methylamino) decanoic acid]-8-(N-methyl-D-alanine)cyclosporin A, reltecimod, balixafortide, relamorelin, 4F-benzoyl-TN14003, motixafortide, cyclo(L-arginyl-L-glutamyl-L-glutamylamido-L-serinyl-L-prolyl-L-α-glutamyl-L-histidine-L-glutamine), (5S,8S,10aR)-N-benzoyl-5-[(2S)-2-(methylamino) propionyl]amino) 3-(3-methylbutyryl)-6-oxo-1,2,4,5,8,9,10,10a-octahydropyrrole[1,2-a][1,5]diazocin-8-carboxamide, L-arginyl-L-isoleucine-L-histidine-L-methyl-L-alanyl-L-tyrosine-L-serine-L-lysyl-L-arginyl-O-phosphono-L-serine glycine-L-lysyl-L-prolyl-L-arginyl glycine-L-tyrosine-L-alanyl-L-phenylalanine-L-isoleucine-L-α-glutamyl-L-tyrosine (Forigerimod), leuprorelin, batifiban, L-threonine-L-α-aspartic acid-L-leucine-L-glutamylamido-L-α-glutamyl-L-arginylglycine-L-α-aspartyl-L-asparaginyl-L-α-aspartyl-L-isoleucine-L-serinyl-L-prolyl-L-phenylalaninyl-L-serinylglycinyl-L-aspartyl-L-glutamylamido-L-prolyl-L-phenylalaninyl-L-lysyl-L-aspartic acid (Dentonin), (2S,5S,8S,11R,14S,20R)-N—((S)-1-amino-6-isopropylamino)-1-oxohexan-2-yl)-2-benzyl-11-(3-guanidinopropyl)-5-(4-hydroxybenzyl)-8-(4-(isopropylamino)butyl)-14-(naphth-2-ylmethyl)-3,6,9,12,15,18,23-heptyloxy-1,4,7,10,16,19-heptaazacyclotrichlorosilane-20-formamide (LY-2510924), disitertide, (3S)-4-[[((2S)-5-amino-1-[[(2S,3R)-1-[[(2R)-1-[[(2R)-1-amino-1-oxoprop-2-yl]amino]-1-oxoprop-2-yl]amino]-3-hydroxy-1-oxobut-2-yl]amino]-1,5-dioxopent-2-yl]amino]-3-[(2S)-2-[(2S)-1-[(2S,3S)-2-[(2S)-1-[(2S)-2-[(2R)-2-[[(2R)-2-aminopropionyl]amino]propionyl]amino]-4-methylpentanoyl]pyrrolidine-2-carbonyl]amino]-3-methylvaleryl]pyrrolidine-2-carbonyl]amino]-4-methylvaleryl]amino]-4-oxobutyric acid (SPX-101), disitertide, birinapant, glycyl-L-arginylglycyl-3-sulfo-L-alanyl-L-threonine-L-proline, cibinetide, veldoreotide, ozarelix, edratide, (2S)-2-[[[(2S)-4-carboxy-2-[[(2R)-2-[2-[[(2S)-3-carboxy-2-[[(2S)-2-formamido-4-methylthioalkylbutyryl]amino]propionyl]amino]acetyl]amino]-3-thioalkylpropionyl]amino]butyryl]amino]-4-methylvaleric acid, (2S)-2-[[((2S)-2-[(2S)-2-[[(2S,3R)-2-[[(2S)-2-[[(2S)-2-[[(2S)-2-[(2S)-2-[[(2S)-2-[(2-acetamidoacetyl)amino]propionyl]amino]-5-amino-5-oxopentanoyl]amino]-3-phenylpropionyl]amino]-3-hydroxypropionyl]amino]-6-aminohexanoyl]amino]-3-hydroxybutyryl]amino]propionyl]amino]propionyl]amino]-6-aminohexanoic acid, (3S,6S,9S,12R,15S,18S,21S,24S,27R,30S,33S)-27-{[2-(dimethylamino)ethyl]thioalkyl}-30-ethyl-33-[(1R,2R,4E)-1-hydroxy-2-methylhexyl-4-alken-1-yl]-24-(2-hydroxy-2-methylpropyl)-1,4,7,10,12,15,19,25,28-nonylmethyl-6,9,18-tri(2-methylpropyl)-3,21-bis(prop-2-yl)-1,4,7,10,13,16,19,22,25,28,31-undecanoazatricyclododecane-2,5,8,11,14,17,20,23,26,29,32-undecene, (S)-1-((2S,5S,5S,8S,11S,14S)-18-amido-11-ethylpyrrolidine-2-carbonyl) pyrrolidine-2-carbonyl)-N-((2S,5S,5S,8S,11S,11S,14S)-18-amino-11-11-(S-sec-butyl)-14-carbamoyl-14-carbamoyl-8-8-(3-nitro-guanidyl)-1-(1-(1H-imidazol-5-yl-yl)-5-methyl-3-3,6,6,12-12-tetraoxoxy-4,4,7,7,10,13-tetraoctanooctadecane-13-octadecanooctan-2-2-yl-2-yl)amidomethyl-2-methyl-2-alk-alk-2-alk-yl)-3-(1H-imidazol-5-yl)-1-oxoprop-2-yl) pyrrolidine-2-carboxamide, cyclo[L-alanyl-L-serinyl-L-isoleucyl-L-prolyl-L-glutamylamido-L-lysyl-L-tyrosinyl-D-prolyl-L-prolyl-(2S)-2-aminodecanoyl-L-α-glutamyl-L-threonine], (4S)-4-{[((1S)-1-{[(1S)-1-{[(2S)-1-[(2S)-2-{[(1S)-1-{[(1S)-5-amino-1-{[((1S)-1-{[(1S)-1-{[(2S)-1-[(2S)-2-{[(1S)-4-carbamate-1-carboxybutyl]carbamoyl}pyrrolidin-1-yl]-4-methyl-1-oxopent-2-yl]carbamoyl}-2-carboxyethyl]carbamoyl}-2-methylpropyl]carbamoyl}pentyl]carbamoyl}-2-hydroxyethyl]carbamoyl}pyrrolidin-1-yl]-3-(1H-imidazol-5-yl)-1-oxoprop-2-yl]carbamoyl}-3-methylbutyl]carbamoyl}-2-phenylethyl]carbamoyl}-4-[(2S)-2,6-diaminohexamido]butyric acid, (2S)-1-[[(2S)-2-cyclohexyl-2-[((2S)-2-(methylamino) propionyl]amino]acetyl]-N-[2-(1,3-oxazol-2-yl)-4-phenyl-1,3-thiazol-5-yl]pyrrolidine-2-carboxamide, bortezomib, cyclo[L-alanyl-L-cysteinyl-L-serinyl-L-alanyl-D-prolyl-(2S)-2,4-diaminobutyryl-L-arginyl-L-tyrosinyl-L-cysteinyl-L-tyrosinyl-L-glutamylamido-L-lysinyl-D-prolinyl-L-prolinyl-L-tyrosinyl-L-histidine], (2→9)-disulfides, anidulafungin, atosiban, capreomycin, carbetocin, caspofungin, actinomycin, dalbavancin, romidepsin, octreotide, semaglutide, liraglutide, glucagon-like peptide 1, insulin calcitonin, central nervous system peptides, and protein drugs), BCS II class (low soluble and high osmotic) and BCS IV class (low soluble and low osmotic) drugs in biopharmaceutical classification systems (comprising but not limited to: aripiprazole, emtricitabine, bictegravir, lenalidomide, brexpiprazole, clotrimazole, clopidogrel, duloxetine, dapoxetine, dicyclomine, flecainide, indinavir, lamotrigine, lansoprazole, meclizine, nelfinavir, nevirapine, pioglitazone, chlorpromazine, quetiapine, raloxifen, rifabutin, ziprasidone, risperidone, rifampicin, selpercatinib, pemigatinib, ozanimod, osilodrostat, dasatinib, ruxolitinib, acalabrutinib, cediranib, dovitinib, sotorasib, adagrasib, motesanib, pazotinib, vardenafil, loperamide, lurasidone, alectinib, nintedanib, N-((7R,8R)-8-((2S,5S,8R,11S,14S,17S,20S,23R,26S,29S,32S)-5-ethyl-11,17,26,29-tetraisobutyl-14,32-diisopropyl-1,7,8,10,16,20,23,25,28,31-dodemethyl-3,6,9,12,15,18,21,24,27,30,33-undecyloxy-1,4,7,10,13,16,19,22,25,28,31-undecylazacyclotriazapolyglycos-2-yl)-8-hydroxy-7-methyloctyl) acetamide, ketoconazole, bosutinib, nilotinib, dabigatran etexilate, palbociclib, fingolimode, vincristine, vincamine, vinpocetine, edoxaban, pralsetinib, berotralstat, tirbanibulin, relugolix, pexidartinib, entrectinib, vandetanib, trilaciclib, tivozanib, rucaparib, ribociclib, tofacitinib, infigratinib, lorlatinib, niratinib, tepotinib, glasdegib, dacomitinib, enasidenib, cobimetinib, brigatinib, fedratinib, rimegepant, rosuvastatin, ethyl (3S)-8-{2-amino-6-[(1R)-1-(5-chloro[1,1′-biphenyl]-2-yl)-2,2,2-trifluoroethoxy]pyrimidin-4-yl}-2,8-diazaspiro[4.5]decane-3-carboxylate, tazemetostat, afatinib, tucatinib, abemaciclib, carvedilol, nebivolol, irbesartan, telmisartan, losartan, olanzapine, rupatadine, desloratadine, ritonavir, and verapamil; ripretinib, opicapone, vismodegib, vemurafenib, loratadine, riociguat, zanubrutinib, axitinib, orelabrutinib, mebendazole, norelgestromin, venetoclax, ticagrelor, ibrutinib, posaconazole, itraconazole, lenvatinib, macitentan, eltrombopag, donafenib, regorafenib, sorafenib, carfilzomib, rilpivirine, camptothecin, hydroxycamptothecin, methoxycamptothecin, nitrocamptothecin, aprepitant, selinexor, upadacitinib, umbralisib, sonidegib, sotorasib, talazoparib, lonafarnib, icotinib, dabrafenib, duvelisib, carfilzomib, capmatinib, bortezomib, binimetinib, avatrombopag, selumetinib, amprenavir, dexamethasone, methylprednisolone, prednisolone, cortisone, hydrocortisone, betamethasone, ivacaftor, teriflunomide, icaritin, olaparib, tolvaptan, pomalidomide, voriconazole, fluconazole, apixaban, vitamin K1, vitamin A, vitamin E, enzalutamide, chlorthalidone, etoposide, dutasteride, isradipine, butyphthalide, progesterone, rivaroxaban, tipranavir, spironolactone, warfarin, medroxyprogesterone, latanoprost, travoprost, bimatoprost, tafluprost, misoprostol, gemeprost, carboprost, latanoprost lactone diol, travoprost acid, travoprost, dinoprost, alprostadil, ezetimibe, felodipine, nifedipine, fenofibrate, celecoxib, tacrolimus, everolimus, rapamycin, carisoprodol, carbamazepine, paricalcitol, eldecalcitol, tacalcitol, doxercalciferol, calcipotriol, budesonide, vitamin D2, calcifediol, calciferol, calcitriol, alfacalcidol, seocalcitol, inecalcitol, falecalcitriol, maxacalcitol, griseofulvin, lopinavir, nabumetone, erdafitinib, allopregnenolone, afamelanotide, solriamfetol, pretomanid, oliceridine, foseltamivir, lurbinectedin, triheptanoin, tocotrienol, 4-[(1E,3S)-3-vinyl-3,7-dimethyl-1,6-octadien-1-yl]phenol, 7-hydroxy-3-[4-hydroxy-3-(3-methyl-2-buten-1-yl)phenyl]-4H-1-benzopyran-4-one, 3-[3-[(2E)-3,7-dimethyl-2,6-octadien-1-yl]-4-hydroxyphenyl]-7-hydroxy-4H-1-benzopyran-4-one, (2E)-1-[2,4-dihydroxy-3-(3-methyl-2-butenyl)phenyl]-3-(4-hydroxyphenyl)-2-propen-1-one, (6E,8E,10E,12E,14E,16E,18E,20E,22E,24E,26E)-2,6,10,14,19,23,27,31-octamethyl-2,6,8,10,12,14,16,18,20,22,24,26,30-diisoamyltriene, 2-[6-(2,4-dihydroxybenzoyl)-5-(2,4-dihydroxyphenyl)-3-methyl-2-cyclohexen-1-yl]-5a,10a-dihydro-1,3,5a,8-tetrahydroxy-10a-(3-methyl-2-buten-1-yl)-11H-benzofuran[3,2-b][1]benzopyran-11-one, (5aR,10aS)-2-[(1S,5S,6R)-6-(2,4-dihydroxybenzoyl)-5-(2,4-dihydroxyphenyl)-3-methyl-2-cyclohexen-1-yl]-5a,10a-dihydro-1,3,8,10a-tetrahydroxy-5a-(3-methyl-2-buten-1-yl)-11H-benzofuran[3,2-b][1]benzopyran-11-one, (2E)-3-(4-hydroxy-2-methoxyphenyl)-1-(4-methoxyphenyl)-2-propen-1-one, 2′,4,4′-trihydroxychalcone 4-(β-D-glucopyranoside), (E)-1-(2,4-dihydroxyphenyl)-3-(4-hydroxyphenyl) propyl-2-ene-1-one, (2E)-3-[5-(1,1-dimethyl-2-propen-1-yl)-4-hydroxy-2-methoxyphenyl]-1-(4-hydroxyphenyl)-2-propen-1-one, (2E)-3-[5-[(1S)-1,2-dimethyl-2-propen-1-yl]-4-hydroxy-2-methoxyphenyl]-1-(4-hydroxyphenyl)-2-propen-1-one, (2E)-3-(3,4-dihydroxy-2-methoxyphenyl)-1-[4-hydroxy-3-(3-methyl-2-buten-1-yl)phenyl]-2-propen-1-one, (2S)-2,3-dihydro-7-hydroxy-2-(4-hydroxyphenyl)-4H-1-benzopyran-4-one, 4′,7-dihydroxyflavanone 4′-β-D-glucopyranoside, 4-[5,7-dimethoxy-6-(3-methyl-2-buten-1-yl)-2H-1-benzopyran-3-yl]-1,3-benzenediyl, 4-[5,7-dimethoxy-6-(3-methyl-2-buten-1-yl)-2H-1-benzopyran-3-yl]-1,3-benzenediol, (2S)-2-[4-(β-D-glucopyranosyl)phenyl]-2,3-dihydro-7-hydroxy-4H-1-benzopyran-4-one, brassinin, carbamoylthioacid (1H-indol-3-ylmethyl)-methyl ester, 2-[3,4-dihydroxy-2,5-di(3-methyl-2-buten-1-yl)phenyl]-2,3-dihydro-5,7-dihydroxy-4H-1-benzopyran-4-one [UNK] (2R,3R)-2-(3,4-dihydroxyphenyl)-2,3-dihydro-3,5,7-trihydroxy-4H-1-benzopyran-4-one, (2R,3R)-2-(3,4-dihydroxyphenyl)-2,3-dihydro-3,5,7-trihydroxy-4H-1-benzopyran-4-one, (3S)-3-[2,4-dihydroxy-3-(3-methyl-2-buten-1-yl)phenyl]-2,3-dihydro-5,7-dihydroxy-4H-1-benzopyran-4-one, 4-[(3R)-3,4-dihydro-7-hydroxy-5-methoxy-6-(3-methyl-2-buten-1-yl)-2H-1-benzopyran-3-yl]-2-(3-methyl-2-buten-1-yl)-1,3-phenyldiol, 4-[(3R)-3,4-dihydro-8,8-dimethyl-2H,8H-benzo[1,2-b: 3,4-b′]-bipyran-3-yl]-1,3-benzenediol, 4-[(3R)-3,4-dihydro-5,7-dimethoxy-6-(3-methyl-2-buten-1-yl)-2H-1-benzopyran-3-yl]-2-(3-methyl-2-buten-1-yl)-1,3-benzenediol, and 5,7-dihydroxy-3-(5-hydroxy-2,2-dimethyl-2H-1-benzopyran-6-yl)-4H-1-benzopyran-4-one; atorvastatin, simvastatin, lovastatin, pravastatin, fluvastatin, rosuvastatin, fosamprenavir, atovaquone, valsartan, candesartan cilexetil, fimasartan, eprosartan, olmesartan, diclofenac sodium, etodolac, furosemide, gemfibrozil, glimepiride, glipizide, glibenclamide, ibuprofen, indomethacin, meloxicam, naproxen, oxaprozin, doxorubicin, tafamidis, and eltrombopag), terpene lactones in natural products (such as artemisinin, parthenolide, thapsigargin, macrocarpal lactones A, B, C, D, and K, andrographolide, neoandrographolide, ginkgolides A, B, C, J, and K, bilobalide, jolkinolide B, nagilactone E, bruceantin, dichapetalin, limonin, triptolide, tripdiolide, celastrol, and celastrol), 7-ethyl-10-hydroxycamptothecin, irinotecan, paclitaxel, docetaxel, tanshinones (such as tanshinone IIA, dihydrotanshinone, cryptotanshinone, miltirone, and tanshinone I), curcumin, demethoxycurcumin, bis(demethoxycurcumin), flavonoids and biflavones (such as wogonin, baicalein, ginkgotin, ginkgetin, isoginkgetin, hinokiflavone, amentoflavone, xanthohumol, isoxanthohumol, demethylxanthohumol, naringenin, 8-isopentenyl naringenin, forskolin, 6-prenyl naringenin, 6,8-diprenyl naringenin, 6-geranyl naringenin, kurarinone, isokurarinone, and kurarinol), eurycomanone, 3,9-ethanol-1H,3H,7H-furan[3′,4′:2,3] cyclopentane[1,2-b]pyran-7-one, 4-(2,5-dihydro-3-methyl-5-oxo-2-furyl) hexahydro-3,8,9,11-tetrahydroxyl-4-methyl-10-methylene-, [3R-[3α,3αβ,4β(S*),5aα,8a,9a,9aR*,11R*]-, isobutyrylshikonin, acetylshikonin, deoxyshikonin, hesperidin, nobiletin, bavachinin, anwuligan, indirubin, psoralen, isopsoralen, psoralen dihydroflavone, psoralen isoflavone, vitamin A2, tretinoin, retinol derivatives, ponicidin, oridonin, scutellarin, tocopherol, artemisinin, gambogic acid, germacrone, curcumenone, curzerenone, neogambogic acid, isogambogic acid, betulinic acid, oleanolic acid, glycyrrhetinic acid, gymnemic acid IV, arjunolic acid, corosolic acid, ursolic acid, asiatic acid, 3-epicorosolic acid, pomolic acid, euscaphic acid, maslinic acid, ganoderic acid, tormentic acid, coenzyme Q10, cryptoxanthin, vitamin E, vitamin D, fullerene, icariin, icariin I, icariin II, icariin C, icariin B, and icariin A; cannabinols (such as cannabidiol, tetrahydrocannabinol, cannabinol, cannabichromene, (1′R,2′R)-4,5′-dimethyl-2′-(prop-1-en-2-yl)-1′,2′,3′,4′-tetrahydro-[1,1′-biphenyl]-2,6-diol, (1′R,2′R)-5′-methyl-2′-prop-1-en-2-yl)-4-propyl-1′,2′,3′,4′-tetrahydro-[1,1′-biphenyl]-2,6-diol, (1′R,2′R)-4-butyl-5′-methyl-2′-(prop-1-en-2-yl)-1′,2′,3′,4′-tetrahydro-[1,1′-biphenyl]-2,6-diol, (1′R,2′R)-2,6-dihydroxy-5′-methyl-4-pentyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-2′-(prop-1-en-2-yl)-4-propyl-1′,2′,3′,4′-tetrahydro-[1,1′-biphenyl]-3-carboxylic acid, (1′R,2′R)-6-methoxy-5′-methyl-4-pentyl-2′-(prop-1-en-2-yl)-1′,2′,3′,4′-tetrahydro-[1,1′-biphenyl]-2-ol, 5′-methyl-4-pentyl-2′-(prop-1-en-2-yl)-[1,1′-biphenyl]-2,6-diol, 5′-methyl-2′-(prop-1-en-2-yl)-4-propyl-[1,1′-biphenyl]-2,6-diol, (1R,6R)-2′,6′-dihydroxy-4′-pentyl-6-(prop-1-en-2-yl)-1,4,5,6-tetrahydro-[1,1′-biphenyl]-3-carboxylic acid, (1′R,2′R)-5′-(hydroxymethyl)-4-pentyl-2′-(prop-1-en-2-yl)-1′,2′,3′,4′-tetrahydro-[1,1′-biphenyl]-2,6-diol, (5aR,6S,9R,9aR)-6-methyl-3-pentyl-9-(prop-1-en-2-yl)-5a,6,7,8,9,9a-hexahydrodibenzo[b,d]furan-1,6-diol, (2S,3S,4S,5R)-3,4,5-trihydroxy-6-((1′R,2′R)-6-hydroxy-5′-methyl-4-pentyl-2′-(prop-1-en-2-yl)-1′,2′,3′,4′-tetrahydro-[1,1′-biphenyl]-2-yl)oxy)tetrahydro-2H-pyran-2-carboxylic acid, 2-((1S,2S,5S)-5-methyl-2-(prop-1-en-2-yl)cyclohexyl)-5-((E)-styryl)phenyl-1,3-diol, 5-((E)-2-hydroxystyryl)-2-((1S,2S,5S)-5-methyl-2-(prop-1-en-2-yl)cyclohexyl)phenyl-1,3-diol, 5-(benzofuran-2-yl)-2-(1S,2S,5S)-5-methyl-2-(prop-1-en-2-yl)cyclohexyl)phenyl-1,3-diol, (1'S,2'S)-2′-(5-hydroxy-6-methylheptyl-1,6-dien-2-yl)-4,5′-dimethyl-1′,2′,3′,4′-tetrahydro-[1,1′-biphenyl]-2,6-diol, 3-phenyl-1-((1'S,2'S)-2,4,6-trihydroxy-5′-methyl-2′-(prop-1-en-2-yl)-1′,2′,3′,4′-tetrahydro-[1,1′-biphenyl]-3-yl) propan-1-one, (1'S,2'S)-5′-methyl-4-pentyl-2′-(propanediol-1-en-2-yl)-1′,2′,3′,4′-tetrahydro-[1,1′-biphenyl]-2,6-diol, (1'S,2'S)-2′-isopropyl-5′-methyl-4-pentyl-1′,2′,3′,4′-tetrahydro-[1,1′-biphenyl]-2,6-diol, 2-((1R,2S)-2-isopropyl-5-methylcyclohexyl)-5-pentylphenyl-1,3-diol, (1'S,2'S)-5′-(hydroxymethyl)-2′-isopropyl-4-pentyl-1′,2′,3′,4′-tetrahydro-[1,1′-biphenyl]-2,6-diol, (1′R,2'S)-5′-(hydroxymethyl)-2′-isopropyl-4-pentyl-1′,2′,3′,4′-tetrahydro-[1,1′-biphenyl]-2,6-diol, (1′R,2′R)-5′-methyl-4-(2-methyloctan-2-yl)-2′-(prop-1-en-2-yl)-1′,2′,3′,4′-tetrahydro-[1,1′-biphenyl]-2,6-diol, (1R,6R)-2′,6′-dihydroxy-4′-(2-methyloctan-2-yl)-6-(prop-1-en-2-yl)-1,4,5,6-tetrahydro-[1,1′-biphenyl]-3-carboxylic acid, (1′R,2′R)-5′-(hydroxymethyl)-4-(2-methyloctan-2-yl)-2′-(prop-1-en-2-yl)-1′,2′,3′,4′-tetrahydro-[1,1′-biphenyl]-2,6-diol, (1R,2R)-2′,6′-dimethoxy-5-methyl-4′-(2-methyloctan-2-yl)-2-(prop-1-en-2-yl)-1,2,3,4-tetrahydro-1,1′-biphenyl, (1'S,2'S)-2′-isopropyl-5′-methyl-4-(2-methyloctan-2-yl)-1′,2′,3′,4′-tetrahydro-[1,1′-biphenyl]-2,6-diol, 2-((1R,2S)-2-isopropyl-5-methylcyclohexyl)-5-(2-methyloctan-2-yl)phenyl-1,3-diol, ((1S,4S,5S)-4-(2,6-dimethoxy-4-(2-methyloctan-2-yl)phenyl)-6,6-dimethylbicyclo[3.1.1]hept-2-en-2-yl) methanol, ((1R,4R,5R)-4-(2,6-dimethoxy-4-(2-methyloctan-2-yl)phenyl)-6,6-dimethylbicyclo[3.1.1]hept-2-en-2-yl) methanol, 1-(3-((1′R,2′R)-2,6-dihydroxy-5′-methyl-2′-(prop-1-en-2-yl)-1′,2′,3′,4′-tetrahydro-[1,1′-biphenyl]-4-yl)methyl) azetidin-1-yl) ethanone, (1′R,2′R)-4-(2-(1H-1,2,3-triazol-1-yl)ethyl)-5′-methyl-2′-(prop-1-en-2-yl)-1′,2′,3′,4′-tetrahydro-[1,1′-biphenyl]-2,6-diol, 2-((1′R,2′R)-2,6-dihydroxy-5′-methyl-2′-(prop-1-en-2-yl)-1′,2′,3′,4′-tetrahydro-[1,1′-biphenyl]-4-yl)-1-morpholinoethanone, (1′R,2′R)-4-(4-hydroxybutyl)-5′-methyl-2′-(prop-1-en-2-yl)-1′,2′,3′,4′-tetrahydro-[1,1′-biphenyl]-2,6-diol, 4-((1′R,2′R)-2,6-dihydroxy-5′-methyl-2′-(prop-1-en-2-yl)-1′,2′,3′,4′-tetrahydro-[1,1′-biphenyl]-4-yl) butyric acid, (1′R,2′R)-4-(2-ethoxyethyl)-5′-methyl-2′-(prop-1-en-2-yl)-1′,2′,3′,4′-tetrahydro-[1,1′-biphenyl]-2,6-diol, (1′R,2′R)-3-chloro-5′-methyl-4-pentyl-2′-(prop-1-en-2-yl)-1′,2′,3′,4′-tetrahydro-[1,1′-biphenyl]-2,6-diol, (1′R,2′R)-3,5-dichloro-5′-methyl-4-pentyl-2′-(prop-1-en-2-yl)-1′,2′,3′,4′-tetrahydro-[1,1′-biphenyl]-2,6-diol, (1′R,2′R)-3-bromo-5′-methyl-4-pentyl-2′-(prop-1-en-2-yl)-1′,2′,3′,4′-tetrahydro-[1,1′-biphenyl]-2,6-diol, (1′R,2′R)-3,5-dibromo-5′-methyl-4-pentyl-2′-(prop-1-en-2-yl)-1′,2′,3′,4′-tetrahydro-[1,1′-biphenyl]-2,6-diol, (1′R,2′R)-3-iodo-5′-methyl-4-pentyl-2′-(prop-1-en-2-yl)-1′,2′,3′,4′-tetrahydro-[1,1′-biphenyl]-2,6-diol, (1′R,2′R)-3,5-diiodo-5′-methyl-4-pentyl-2′-(prop-1-en-2-yl)-1′,2′,3′,4′-tetrahydro-[1,1′-biphenyl]-2,6-diol, (1′R,2′R)-3-fluoro-5′-methyl-4-pentyl-2′-(prop-1-en-2-yl)-1′,2′,3′,4′-tetrahydro-[1,1′-biphenyl]-2,6-diol, 3-(acetoxy)-2-[(1R,6R)-6-(3-fluoroprop-1-en-2-yl)-3-methylcyclohex-2-en-1-yl]-5-pentylphenyl acetate, (1′R,2′R)-5′-(fluoromethyl)-4-pentyl-2′-(prop-1-en-2-yl)-1′,2′,3′,4′-tetrahydro-[1,1′-biphenyl]-2,6-diol, 1,3-dimethoxy-2-[(1R,6R)-3-methyl-6-prop-1-en-2-ylcyclohex-2-en-1-yl]-5-pentylbenzene, (1′R,2′R)-5′-methyl-4-pentyl-2′-(prop-1-en-2-yl)-1′,2′,3′,4′-tetrahydro-[1,1′-biphenyl]-2-ol, (1R,6R)-2′,6′-diacetoxy-4′-pentyl-6-(prop-1-en-2-yl)-1,4,5,6-tetrahydro-[1, l′-biphenyl]-3-carboxylic acid, 2-((1′R,2′R)-6-hydroxy-5′-methyl-4-pentyl-2′-(prop-1-en-2-yl)-1′,2′,3′,4′-tetrahydro-[1,1′-biphenyl]-2-yl)oxy) acetic acid, (1′R,2′R)-6-(3-aminopropoxy)-5′-methyl-4-pentyl-2′-(prop-1-en-2-yl)-1′,2′,3′,4′-tetrahydro-[1,1′-biphenyl]-2-ol, 2-[3-(cyanomethoxy)-2-[(1R,6R)-3-methyl-6-(prop-1-en-2-yl)cyclohex-2-en-1-yl]-5-pentylphenoxy]acetonitrile, 3-({[(diethylamino)methoxy]carbonyl}oxy)-2-[(1R,6R)-3-methyl-6-(prop-1-en-2-yl)cyclohex-2-en-1-yl]-5-pentylphenyl(diethylamino)methyl carbonate, 3-({2-[(tert-butyldimethylsilyl)oxy]acetoxy)-2-[(1R,6R)-3-methyl-6-(prop-1-en-2-yl)cyclohex-2-en-1-yl]-5-pentylphenyl 2-[(tert-butyldimethylsilyl)oxy]acetate, 3-(acetoxy)-2-[(1R,6R)-3-methyl-6-(3-oxoprop-1-en-2-yl)cyclohex-2-en-1-yl]-5-pentylphenyl acetate, 3-(acetoxy)-2-[(1R,6R)-3-methyl-4-oxo-6-(prop-1-en-2-yl)cyclohex-2-en-1-yl]-5-pentylphenyl acetate, 3-(acetoxy)-2-[(1R,6R)-4-(acetoxy)-3-methyl-6-(prop-1-en-2-yl)cyclohex-2-en-1-yl]-5-pentylphenyl acetate, 2-[(1R,2R)-2-[2,6-di(acetoxy)-4-pentenyl]-4-methylcyclohex-3-en-1-yl]prop-2-en-1-yl acetate, 3-hydroxy-2-[(1R,6R)-3-methyl-6-prop-1-en-2-ylcyclohex-2-en-1-yl]-5-pentylcyclohex-2,5-dien-1,4-dione, 2,5-cyclohexadien-1,4-dione, 2-hydroxy-3-((1R,6R)-3-methyl-6-(1-methylvinyl)-2-cyclohexen-1-yl)-6-pentyl-5-(butamino), 2,5-cyclohexadien-1,4-dione, 2-hydroxy-3-((1R,6R)-3-methyl-6-(1-methylvinyl)-2-cyclohexen-1-yl)-6-pentyl-5-((benzyl)amino), 5-methyl-4-[(1R,6R)-3-methyl-6-prop-1-en-2-ylcyclohex-2-en-1-yl]phenyl-1,3-diol, 4-[(1R,6R)-3-methyl-6-prop-1-en-2-ylcyclohex-2-en-1-yl]-5-pentylphenyl-1,3-diol, 2-[(2E)-3,7-dimethylocta-2,6-dienyl]-5-pentylphenyl-1,3-diol, 1-[(1R,2R,3R,4R)-3-(2,6-dihydroxy-4-pentylphenyl)-2-hydroxy-4-prop-1-en-2-ylcyclopentyl]ethanone.

7. The supramolecular self-assembly system according to claim 2, characterized in that a mass ratio of the carrier (preferably the carriers with the flavonoid or terpenoid structure) to the target is 0.003:1 to 250:1, preferably 0.01:1 to 200:1, and more preferably 0.015:1 to 150:1.

8. The supramolecular self-assembly system according to claim 2, characterized in that a mass ratio of the carrier (preferably the carriers with the flavonoid or terpenoid structure) to the polymer is 1:0 to 1:100, preferably 1:0 to 1:75, and more preferably 1:0 to 1:50.

9. The supramolecular self-assembly system according to claim 2, characterized in that the carrier with the flavonoid structure is selected from hesperetin, naringenin, quercetin, kaempferol, isorhamnetin, myricetin, apigenin, luteolin, eriodictyol, diosmetin, genistein, baicalein, catechin, epicatechin, puerarin, isoprimin, tannic acid, chrysin, pelargonidin, cyanidin, delphinidin, peonidin, petunidin, malvidin, and saccharide derivatives thereof, such as flavonoid glycosides formed by connection with monosaccharides, disaccharides, trisaccharides, acylated saccharides, or tetrasaccharides, chalcones, dihydrochalcones, flavonols, isoprene compounds, and derivatives with saccharides.

10. The supramolecular self-assembly system according to claim 2, characterized in that the carrier with the terpenoid structure is selected from compounds containing isoprene or isopentane, comprising but not limited to monoterpenes, cycloalkene ether terpenes, sesquiterpenes, diterpenes, triterpenes, and tetraterpenes.

11. The supramolecular self-assembly system according to claim 2, characterized in that the polymer is selected from one or more of cellulose, starch, soluble starch, wheat starch, potato starch, cassava starch, gellan gum, maltodextrin, hyaluronic acid, zein, corn starch, tragacanth gum, arabic gum, alginic acid, sodium alginate, pectin, chitosan, arabinogalactan, polysaccharide or polysaccharide extract, xanthan gum, cyclodextrin, and derivatives thereof; the artificially synthesized or semi-synthesized polymer is selected from one or more of hydroxypropyl methyl cellulose, methyl cellulose, cellulose acetate, ethyl cellulose, hydroxypropyl cellulose, low-substituted hydroxypropyl cellulose, microcrystalline cellulose, carboxymethyl cellulose, carboxymethyl starch sodium, hydroxypropyl methyl cellulose acetate succinate, hydroxypropyl methyl cellulose phthalate, cross-linked carboxymethyl cellulose sodium or calcium, and silicified microcrystalline cellulose; and the polymer A is selected from one or more of polyethylene caprolactam-polyvinyl acetate-polyethylene glycol graft copolymer, copovidone, polyvinylpyrrolidone series, polyethylene glycol series, ethyl acrylate-methyl methacrylate-trimethylamine ethyl methacrylate chloride (1:2:0.2) copolymer, ethyl acrylate-methyl methacrylate-trimethylamine ethyl methacrylate chloride (1:2:0.1) copolymer, methacrylic acid-ethyl acrylate (1:1) copolymer, methacrylic acid-methyl methacrylate (1:1) copolymer, methacrylic acid-methyl methacrylate (1:2) copolymer, butyl methacrylate-dimethylaminoethyl methacrylate-methyl methacrylate (1:2:1) copolymer, ethyl acrylate-methyl methacrylate (2:1) copolymer, glycolide lactide copolymer series, carbomer, carbomer copolymer, polylactic acid-hydroxyglycolic acid copolymer, polylactic acid-glycollic acid copolymer, sorbitan trioleate, lauroyl polyoxyethylene glyceride, oleoyl polyoxyethylene glyceride, oleic acid polyoxyethylene ester, polysorbates (Tween20 and 80), poloxamer, vitamin E succinate polyethylene glycol ester (TPGS), stearic acid polyoxometalate, polyvinyl alcohol, polyammonium methacrylate, polyoxyethylene, polyoxyethylene castor oil, and polyoxyethylene hydrogenated castor oil.

12. The supramolecular self-assembly system according to claim 2, characterized in that the target has a Log P or Log D7.4 of 0.8-17, 0-7 hydrogen donors, and 1-12 hydrogen acceptors, and is dissociated or non-dissociated; in the presence of a plurality of targets, there is an intermolecular interaction and/or an intramolecular interaction or no such interactions between the targets; preferably, the target is selected from the group consisting of nilotinib, nintedanib, lenvatinib, sorafenib, ticagrelor, apixaban, rivaroxaban, warfarin, lurasidone, curcumin, vitamin K1, macitentan, tacrolimus, cyclosporine, paclitaxel, docetaxel, ibrutinib, clopidogrel, fingolimode, enzalutamide, posaconazole, dabigatran etexilate, venetoclax, alectinib, palbociclib, naringenin, celecoxib, itraconazole, eltrombopag, griseofulvin, acalabrutinib, ezetimibe, felodipine, scutellarin, candesartan cilexetil, regorafenib, butyphthalide, coenzyme Q10, cannabidiol, tafluprost, lutein, vitamin E, vitamin A, and salts, hydrates, solvates, or eutectics thereof.

13. The supramolecular self-assembly system according to claim 2, characterized in that the carrier with the flavonoid structure is selected from naringenin, hesperetin, catechin, epicatechin, quercetin, isoquercitrin, myricetin, eriodictin, and/or flavonoid glycosides, flavonol glycosides, and flavanols formed by connecting them to saccharides with a number of N (wherein N is greater than or equal to 1) and acylated saccharides, and/or chalcones (such as dihydrochalcones) and saccharide derivatives of chalcones (such as dihydrochalcones), such as derivatives formed by connecting them to saccharides with a number of N (wherein Nis greater than or equal to 1).

14. The supramolecular self-assembly system according to claim 2, characterized in that the carrier with the flavonoid structure is selected from naringin, hesperidin, epicatechin gallate, isoquercitrin, quercetin, myricetrin, epigallocatechin, tannic acid, neohesperidin dihydrochalcone, trilobatin, naringin dihydrochalcone, quercetin 3-rutinoside, and neohesperidin.

15. The supramolecular self-assembly system according to claim 2, characterized in that the carrier with the terpenoid structure is selected from sweet tea, rubusoside, rebaudioside A, rebaudioside B, rebaudioside C, rebaudioside D, rebaudioside M, stevia, soyasaponin A1, soyasaponin Ba, soyasaponin I, soyasaponin II, soyasaponin III, glycyrrhizic acid and salts thereof, glycyrrhetinic acid, stevioside, stevioside ingredient extract (stevioside content ≥95%, wherein rebaudioside A ≥25), mogroside V, mogroside ingredient extract (containing mogroside V≥30%, HPLC), asiaticoside, asiaticoside A, asiaticoside B, asiaticoside E, asiaticoside F, ginsenoside Rg1, ginsenoside Rb1, dioscin, mogroside IV, mogroside V, oat saponin A, oat saponin B, platycodin A, platycodin B, platycodin D, platycodin D2, platycodin D3, tenuigenin A, tenuigenin D, and tenuigenin D2.

16. The supramolecular self-assembly system according to claim 2, characterized in that the polymer A is selected from one or more of polyethylene caprolactam-polyvinyl acetate-polyethylene glycol graft copolymer, hydroxypropyl methyl cellulose acetate succinate and polyethylene caprolactam-polyvinyl acetate-polyethylene glycol graft copolymer, hydroxypropyl methyl cellulose, sodium carboxymethyl cellulose, calcium carboxymethyl cellulose, polyvinyl pyrrolidone, copovidone, polyethylene glycol, cellulose acetate, hyaluronic acid, xanthan gum, methacrylic acid-methyl methacrylate copolymer (1:1), methacrylic acid-ethyl methacrylate copolymer (1:1), hydroxypropyl cellulose, polyoxyethylene-polyoxypropylene block copolymer, sodium dodecyl sulfate, TPGS, and polyacrylic acid.

17. A composition, comprising the supramolecular self-assembly system according to claim 1, and preferably further comprising one or more of fillers, disintegrants, adhesives, lubricants, flow aids, emulsifiers, flavor enhancers or masking agents, surfactants, co-surfactants, and preservatives.

18. The composition according to claim 17, being tablets, capsules, suspension, patch, cream, gel, emulsion, eye drops, injection, oral capsules, suppository, implants, powder; or being contained in parenteral nutrition liquid, enteral nutrition liquid, health products, functional beverages, and preservative and fresh-keeping products in the food and beverage industry; or being contained in perfume, gel, cream, emulsion, masks, and lipsticks in the cosmetics industry; or being contained in toothpaste, shampoo, conditioners, and hair cream in the field of fine chemicals; or being contained in diagnostic products, implant materials, and biosensors in the field of biomedicine.

19. (canceled)
